(19) United States
(12) Patent Application Publication
(10) Pub. No.: US 2025/0109171 A1
(43) Pub. Date: Apr. 3, 2025

(54) COMPOUNDS AND COMPOSITIONS FOR TARGETED THERAPY OF RENAL DISEASES

(71) Applicant: SHANGHAI MICURX PHARMACEUTICALS CO., LTD., New District, Shanghai (CN)

(72) Inventors: Mikhail Fedorovich Gordeev, Foster City, CA (US); Jinqian Liu, Foster City, CA (US); Xinghai Wang, Shanghai (CN)

(21) Appl. No.: 18/580,926

(22) PCT Filed: Jul. 21, 2022
(86) PCT No.: PCT/CN2022/106975

§ 371 Date: Jan. 19, 2024

Related U.S. Application Data
(60) Provisional application No. 63/224,402, filed on Jul. 21, 2021.

(51) Int. Cl. C07K 7/62; A61K 47/64; A61P 13/12

(52) U.S. Cl. CPC C07K 7/62; A61K 47/64; A61P 13/12

(57) ABSTRACT

Provided herein are therapeutic compounds, for example compounds of Formula I, or pharmaceutically acceptable salts, hydrates, or solvates thereof that are therapeutic or anticancer agents, pharmaceutical compositions thereof, methods for their use, and methods for preparing these compounds.

### CROSS-REFERENCE TO RELATED APPLICATIONS

This application claims the benefit of U.S. Provisional Application No. 63/224,402, filed Jul. 21, 2021, the entirety of which is hereby incorporated by reference for all purposes.

### FIELD

Provided herein are novel bioactive compounds, pharmaceutical compositions thereof, methods for their use, and methods for preparing of the same. These novel agents and compositions thereof possess therapeutic activities useful in the therapy of inflammation-associated renal disorders, in particular, the chronic kidney disease (CKD), systemic lupus erythematosus (SLE), diabetic nephropathy, chronic glomerulonephritis, and acute kidney injury (AKI).

### BACKGROUND

Kidney diseases comprise a diverse class of serious renal disorders with a profound impact on human lives worldwide. Renal inflammation may result due to a variety of exogenous and endogenous factors, including exposure to nephrotoxic substances, surgical procedures (such as kidney transplantation), chemotherapy (such as in a treatment of cancer, diabetes, or infection), infections (such as urinary tract infections and pyelonephritis), and certain chronic conditions. Among these, acute and chronic kidney diseases such as CKD are reported as reaching epidemic proportions globally (as reviewed, for example, by Levey et al. in Kidney Int., 2005, pp. 2089-2100; and by Stenvinkel in J. Intern. Med. 2010, 268, pp. 456-467). Unfortunately, a long-term therapy of CKD required for management of this medical condition is typically accompanied by serious adverse effects commonly resulting in unsatisfactory outcomes and inadequate patient compliance.

Systemic lupus erythematosus (SLE) is another common chronic inflammatory disease that often affects the kidney (as reviewed, for example, by Almaani et al. in Clin. J. Am. Soc. Nephrol. 2017, 12, pp. 825-835). SLE can lead to the debilitating disorder lupus nephritis (LN), which must be treated with high-dose corticosteroids, toxic cyclophosphamide, or strong immunosuppressants, such as cyclosporin. Such chemotherapy is commonly limited by series adverse effects.

Furthermore, the development of novel improved therapies targeting such kidney diseases is difficult. For example, several drug candidates have failed in clinic trials due to safety issues and/or poor efficacy, including agents intended for therapy of CKD, autosomal dominant polycystic kidney disease (ADPKD), and diabetic nephropathy, as reviewed by Wischnjow et al. in Bioconjugate Chemistry, 2016, pp. 1050-1057.

Importantly, many adverse effects limiting the use of current drugs and the development of novel agents are associated with unduly broad systemic exposure to such bioactive molecules. In other words, a bioactive agent targeting a kidney disease administered as an oral or intravenous drug is necessarily circulated in a blood stream of a mammal or a patient in need of such therapy before it could reach the kidneys. Such undesired, but unavoidable distribution of a bioactive molecule throughout various bodily compartments and into organs other than kidneys results in an off-target effect, typically manifested as an adverse event.

Therefore, a targeted renal drug delivery comprising a selective transport of a bioactive molecule into the kidneys is preferred to conventional therapies that entail an exposure of other organs to a drug that may induce an adverse effect in non-kidney compartments. This would allow for the application of the bioactive effect of the agent directly at the site of a kidney disease, thus broadening the therapeutic window of drugs and reducing systemic adverse effects, which in turn improve the efficiency of such therapies.

Several renal drug delivery systems directing a structure into kidney tissues (also referred to as renal vectors or molecular guiding systems) have been described. For example, peptide carrier structures were described in publications Molecular Therapy, 2007, pp. 1647-1654, Bioconjugate Chem. 2012, pp. 1200-1210, Bioconjugate Chemistry, 2016, pp. 1050-1057, and in the U.S. Pat. No. 10,413,614. Other molecular guiding systems acting as renal vectors include macromolecules such as polyvinylpyrrolidone (Nat. Biotechnol. 2003, pp. 399-404), chitosans (J. Drug Target, 2007, pp. 269-278), glucosamines (J. Controlled Release, 2013, pp. 148-156), and proteins (as described, for example, J. Med. Chem., 1992, pp. 1246-1259; and Int. J. Nanomedicine, 2017, pp. 5673-5686).

Separate category of agents for targeted therapy of kidney disorders comprises prodrugs that selectively release a bioactive drug structure by kidney-associated enzymes (see, for example, J. Controlled Release, 2013, pp. 148-156; and RSC Adv. 4, 2014, pp. 50828-50831).

Among serious kidney disorders, CKD is recognized as a global public health issue. Over time, this chronic condition can progress towards the debilitating end-stage renal disease. Eventually, life-preserving options would be limited to time-consuming and costly renal dialysis and/or kidney transplantation. The dire situation is exacerbated by the absence or scarcity of the dialysis stations in many world regions. Therefore, there is an unmet medical need for CKD treatment capable of preventing the end-stage renal disease through improved therapy and disease management (as reviewed, for example, by Perico et al. in Kidney Int. 2005, 68, Suppl. 98, pp. S21-S24).

CKD is a complex disease associated with multiple contributing parameters. Among the chief factors is inflammation, often resulting from an overexpression of pro-inflammation mediators such as endothelin-1, monocyte chemoattractant protein-1, normal T cell and osteopontin, for example as described in Perico et al. in Kidney Int. 2005, 68, Suppl. 98, pp. S21-S24. Acute kidney injury (AC) is another risk factor associated with CKD (as described, for example, by Campbell et al. in J. Clin. Hypertension, 2015, 17, pp. 514-527). Furthermore, AKI may also manifest separately from CKD, resulting, for example, from an exposure to renal toxins such as a nephrotoxic pro-inflammatory substance. In kidney transplantation, renal ischemia-reperfusion injury is a major aetiology factor in AKI. Analogously to aetiology for CKD, renal inflammation plays a major role in the pathophysiology of ischemic AKI (as described, for example, by Bonventre and Zuk in Kidney Int. 2004, 66, pp. 480-485).

Subsequently, certain anti-inflammatory agents are used in the therapy of CKD and AKI. For example, anti-inflammatory therapy with corticosteroid dexamethasone was described by Moonen et al. in BMC Nephrology. 2018, 19:343.

Separately, a supporting anti-inflammatory therapy is often used during treatment of cancers, for example, to attenuate toxic effects of anticancer drugs (as described, for example, by Shih et al. in J. Pain Palliative Core Pharmacother. 2007, 21 pp. 69-76; and by Vogelzang et al. in J. Clin. Oncol. 2003, 21, pp. 2636-2644), or to induce a beneficial immunomodulatory effect (see, for example, Cook et al. in Oncoimmunology, 2016, 5, e1066062).

However, a long-term exposure to most anti-inflammatory agents such as steroids and NSAIDs (non-steroidal anti-inflammatory agents) is associated with serious adverse effects, including bone loss, cataracts, or muscle weakness. Therefore, safer and more effective agents for therapy of CKD, SLE, LN, AKI, and various other inflammation-associated renal diseases or renal injuries are needed. Such therapies must possess an improved selectivity, wherein bioactive effects of a drug are directed towards the site of a disease, thus minimizing or eliminating adverse effects from an off-target toxicity resulting from an exposure of healthy tissues and organs.

One recent approach to achieve improved selectivity of a drug is a targeted delivery of highly active agents directly to kidneys (as reviewed, for example, by Wischnjow et al. in Bioconjugate Chemistry, 2016, pp. 1050-1057). Therein, an active agent (such as antibacterial drug) is connected to (or conjugated with) an-organ targeting structure with high affinity towards kidney cells. As a result, the active agent or a drug is selectively delivered to the organ in need of a therapy.

Unique conjugates of certain anti-inflammatory agents with acyclic and cyclic peptides (cyclopeptides) suitable for a targeted therapy of various kidney diseases are described herein. The conjugation (a covalent bonding) of bioactive agents with the peptides is achieved through deliberate design of appropriate linking groups (linkers) and additional structural elements (such as connectivity-altering spacers) serving to maximize the targeted delivery and therapeutic bioactivity. Agents provided herein are particularly suitable for therapy of CKD, AKI, and other non-cancer renal disorders.

Various cyclopeptides have been described, for example, in publications WO 2021/150792, WO 2019136298, WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687. Certain conjugates of acyclic peptide structures for renal-targeted delivery of protective agents against nephrotoxic substances have been described, for example, in U.S. Pat. No. 10,413,614. None of these references specifically describe or generally contemplate the composition provided herein.

### SUMMARY

Provided herein are novel compounds and compositions useful for targeted therapy of inflammation-associated renal disorders.

These novel compounds are surprisingly able to target kidney tissues and, in particular, kidney cells affected by inflammation, including an inflammation resulting from overexpression of innate pro-inflammation mediators, or an inflammation induced by a foreign nephrotoxic substance, or an inflammation caused by other therapeutic agents (such as a cytotoxic anticancer drug, or a nephrotoxic antibacterial agent). This unique affinity of the compositions described herein for kidney tissues affected by renal disorders achieves a selective delivery to and accumulation of such molecules at the site of the disease, with minimal or no accumulation in other tissues unaffected by such medical condition.

As a result, a selective and generally safer renal therapy is achieved, with significantly minimized adverse effect(s) on other normal organs of a mammal under therapy, as compared to current standard-of-care drugs used to treat inflammation-associated kidney diseases, such as steroids and NSAID agents.

In one aspect, a therapeutic action of the compounds described herein is achieved by release of one or more of an anti-inflammatory element(s) (bioactive payloads and/or drugs) incorporated into such designer molecules. The active payload (drug) may comprise a steroid structure, NSAID structure, or an immunomodulating structure. These are selected, for example, from bioactive structures with the ability to inhibit or counter an inflammation (such as inflammation due to AKI or in a transplantation surgery, or that induced by cytokines), or with the alike ability to activate immunomodulation of a therapeutic anti-inflammatory response.

Generally, the compounds provided herein are comprised of a peptide, cyclopeptide, or another “target seeker” (ligand) structure with a high affinity (ability to bind) towards kidney cells, along with the active drug(s) substructure, within a single conjugate molecule. The active drug(s) (payloads) is(are) connected to a kidney-affinity structure via a framework of uniquely designed linker(s) and spacer(s). This unique design allows for an efficient release of an active drug (payload) directly into kidney tissues, resulting in a therapeutic anti-inflammatory effect.

In another aspect, said composition possesses anti-inflammatory property(ies) in a molecular form of an intact conjugate, without a release of an active drug payload (comprised within the structure) at the site of a kidney inflammation. Upon accumulation at the site of a kidney inflammation, such compound(s) exert an anti-inflammatory action directly. In a related aspect, the conjugate breaks down into generally non-toxic metabolites after exerting a desired bioactive effect.

In yet another aspect, the anti-inflammatory effect is achieved (upon accumulation at the cancer site) through a combined effect of (i) direct anti-inflammatory effect of said compound(s) and (ii) a release of an active payload drug comprised within the structure.

In an additional aspect, either an intact conjugate and/or the agent released from said conjugate in kidneys exhibits a protective effect against nephrotoxic substances, such as cytotoxic anticancer drugs.

Surprisingly, certain compounds and compositions provided herein are devoid of significant antibiotic and/or other biological activity, and only exert the desired anti-inflammatory effect on kidneys affected by a renal disorder.

Furthermore, while certain compositions provided herein incorporate cyclopeptide moieties (structures) of chemical classes generally known to cause renal toxicity (such as polymyxins), the therapeutic compounds described herein exhibit little or no renal toxicity at the therapeutic dosing levels required from treatment of inflammation-associated kidney disorders.

One skilled in art would readily appreciate that not every molecular construct incorporating anti-inflammatory element(s) (payload) with “heat-seeker” affinity structure (ligand targeting kidney cells) with appropriate linkers and deliberately positioned spacers (strategically placed between a ligand and the bioactive payload) is suitable for use as a therapeutic agent. Surprisingly, compounds and composition provided herein possess a good pharmacological profile, with appropriate stability in blood plasma that precludes premature bioactive action, coupled with preferential accumulation thereof in renal cells and/or in kidneys affected by inflammation-associated kidney disorder(s).

Even more surprisingly, certain compounds provided herein exert their bioactive effect by self-targeted delivery thereof either directly into kidney cells affected by inflammation, or only in close proximity of inflammation-affected tissues. In part, this composition comprises a class of molecules capable of specifically releasing anti-inflammatory payloads (incorporated within their structures) as a result of metabolic cleavage by classes of enzymes either specific to or overexpressed (enriched) within the renal cells affected by an inflammation (such as cathepsin, glutaminase, glutathione transferase, and peptide deformylase or PDF, peptidases, reductases, and alike known enzymes).

In addition to metabolic degradation by classes of enzymes overexpressed in renal cells affected by an inflammation (such as cathepsin, glutaminases, PDF, or alike), certain compounds provided herein are degraded in vivo through a chemical cleavage, such as pH-dependent self-cleavage known for molecules bearing both a cleavable group (such as an ester, an amide, or a carbamate group) and a free nucleophilic group (such as amine, alcohol, or thiol group). When these two types of cleavable and nucleophilic groups are in certain spacial proximity to each other, and the nucleophile group is essentially free (for example, amine group under neutral, basic, or physiological pH conditions), the nucleophilic group may be acylated by the ester group, resulting in the acyl group transfer onto the nucleophilic atom (such as nitrogen atom in amine group). Alternative or concomitant to above process(es), the free amine may activate an adjacent to the carbamate group amide functionality, inducing the carbamate reaction with the latter, to result in a conversion of the native amide into a bis-acylated imide group. In some compositions described herein, the cleavage of a chemical designer linker takes place after initial enzymatic metabolism of an auxiliary enzyme-cleavable linker (e.g., peptide substructure, or alike), to release the anti-inflammatory payload in kidney tissues.

In one aspect, provided herein is a compound of Formula I-P-1:

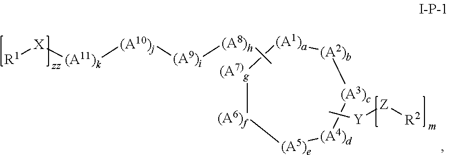

or a pharmaceutically acceptable salt, solvate, or hydrate thereof wherein:
R¹ and R² are optional groups, with at least one of the groups R¹ and R² being present in the Formula I-P-1; and
R¹ and R² are independently selected from alkyl, aryl, biaryl, heteroaryl, heteroarylaryl, and arylheteroaryl; or
R¹ and R² are residues independently attached to X and Z, respectively, by subtracting a single or multiple H atom(s) from respective parent (precursor) structure(s) (H)ₙR¹ and (H)ₒR² at any one of the following optional H-containing group(s) independently selected from NH, OH, SH, C(═O)OH, CONH, SO₂NH, and S(═O)NH when present in (H)ₙR¹ and (H)ₒR²; and wherein
a) (H)ₙR¹ and (H)ₒR² are independently an anti-inflammatory, an immunomodulating, a nephro-protective, or a compound(s) possessing an activity or capable of inducing an activity that modulates gluococorticoid receptor (GR) activity, or modulates a cytokine release, or otherwise exhibits a biological activity against inflammation-associated kidney diseases, or a kidney injury; or
b) (H)ₙR¹ and (H)ₒR² are independently selected from aldosterone, AN3485 (6-(4-(aminomethyl)-2-chlorophenoxyl)benzo[c][1,2]oxaborol-1(3H)-ol), beclomethasone, beclomethasone dipropionate, betamethasone, budesonide, a budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, valdecoxib; a heteroaromatic vanin inhibitor compound (selected from compounds described in PCT WO 2020/114943 and US2020/0354338); a heterocyclic TYK2 inhibitor compound (selected from compounds described in PCT WO2020/86616 and US2020/0354338); and INF-alpha inhibitor compound (selected from compounds described in PCT WO 2020/159904 and EP3917912); or a variant thereof;
c) (H)ₙR¹ and (H)ₒR² are independently compound(s) active against an inflammation-associated kidney disease; or
d) (H)ₙR¹ is a glucocorticoid structure connected to X at one of the optional O or N atom(s), with at least one of these atoms present within the structure (H)ₙR¹; and
when an optional group R¹ is absent, then the fragment R¹X is replaced with R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is replaced with R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂ heteroarylC(═O), heteroarylOC(═O), heteroarylNHC(═O), heteroaryl N(C₁₋₁₂alkyl)C(═O), heteroarylSO₂, and heteroarylNHSO₂; or wherein
integers n and o are independently selected from 0, 1, 2, 3, 4, 5, 6, and 7, such that [n+o]≥1; and
A¹ through A¹¹ are optional amino acid residues unsubstituted or substituted at any N atom and independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers a through k, m, and zz are independently selected from 0, 1, and 2, and wherein
[m+zz]≥1; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, or heterocyclyl; or the group A⁸ is directly connected to the group Y; and
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O)N(R⁵)(C₁₋₁₂alkylene),
C(═O)(CR⁵R⁶)ₚ(CR⁷R⁸)ᵣ(CR⁹R¹⁰)ₛC(═O),
C(═O)(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A′)]CH₂CH₂COOH}CH₂CH₂C(═O), and
C(═O)N[CH₂CH₂NHC(═O)CH(NH₂)CH₂CH₂COOC₆alkyl]CH₂CH₂C(═O);
or any variant of the above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein
R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁶ and R⁷, and ii) R⁹ and R¹⁰, together with the atom to which they are attached form a saturated or unsaturated C₃₋₆ cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³, with the following structures:
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O)N(R⁵)(C₁₋₁₂alkylene),
C(═O)(CR⁵R⁶)ₚ(CR⁷R⁸)ᵣ(CR⁹R¹⁰)ᵣC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ᵣC(═O), C(═O)N(R⁵)(CR⁵R⁶)ₚ(CR⁷R⁸)(CR⁹R¹⁰)ᵣC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O), C(═O)N(R⁵)(CR⁵R⁶)ₚ(CR⁷R⁸)(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)(CR⁹R¹⁰)ᵣC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A′)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O), or
C(═O)N[CH₂CH₂NHC(═O)CH(NH₂)CH₂CH₂COOC₁₋₆alkyl]CH₂CH₂C(═O);
or any variant of the above X groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates additional divalent groups selected from C₁₋₁₂alkylene, C₂₋₂alkenylene, C₂₋₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups;
optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O)N(R⁵)(C₁₋₁₂alkylene),
C(═O)(CR⁵R⁶)ₚ(CR⁷R⁸)ᵣ(CR⁹R¹⁰)ₛC(═O),
C(═O)(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ₛP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁵R⁶)(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ᵣC(═O), C(═O)N(R⁵)(CR⁵R⁶)ₚ(CR⁸)ᵣ(CR⁹R¹⁰)ₛC(═O),
C(═O)(CR⁵R⁶)ᵣ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁷R⁸)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ᵣC(═O),
C(═O)(CR⁵R⁶)ₚS—S(CR⁷R⁸)(CR⁹R¹⁰)ₛOC(═O), C(═O)(CR⁵R⁶)ₚ(CR⁷R⁸)ᵣ(CR⁹R¹⁰)N(R⁵)ₛC(═O),
C(═O)(CR⁵R⁶)ᵣ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O), and
C(═O)N[CH₂CH₂NHC(═O)CH(NH₂)CH₂CH₂COOC₁₋₆alkyl]CH₂CH₂C(═O);
or any variant of above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein R⁵-R¹⁰ are as defined above; or
alternatively, the optional group Z is comprised of the following structures, optionally connected to one to two amino acid residue(s) A¹² or A¹³, with the following structures:
(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ᵣ(CR⁹R¹⁰))ₛP(═O)(OCR⁵R⁶)ₘ,
P(═O)(NHCR⁵R⁶)ₚ(CR⁷R⁸)ᵣ(CR⁹R¹⁰)ᵣC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A)]CH₂CH₂COOH}CH₂CH₂C(═O), or
C(═O)N[CH₂CH₂NHC(═O)CH(NH₂)CH₂CH₂COOC₁₋₆alkyl]CH₂CH₂C(═O);
or any variant of the above Z groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the left side of above groups to comprise the group Z, then residues A¹² or A¹³ are interconnected with a peptide bond A¹²-A¹³.

In an alternative embodiment of Formula I-P-1, R⁵ and R⁶ together with the atom to which they are attached form a saturated or unsaturated C₃₋₆cycloalkylene.

In an alternative embodiment of Formula I-P-1, fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present and [p+r+s]≥1.

In an alternative embodiment of Formula I-P-1, fragments (CR⁵R⁶)ₚ(CR⁷R⁸)ᵣ or (OCR⁵R⁶)ₚ(CR⁷R⁸)ᵣ are present and [p+r]≥1.

In an alternative embodiment of Formula I-P-1, each optional divalent group X is selected from N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O),
C(═O)(CR⁵R⁶)ᵣ(CR⁷R⁸)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ᵣC(═O), C(═O)CR⁵═CR⁷—(CR⁹R¹⁰)SOC(═O), C(═O)CR⁵═CR⁷—(CR⁹R¹⁰)ₛN(R⁴)C(═O), and any variant of the above X groups formed by the repositioning, addition, or deletion of the fragment N(R⁴)C(═O) or C(═O)N(R⁴)SO₂; or
X is independently selected from C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁷R⁸)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ₛP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R⁸)ₛP(═O)(NHCR⁵R⁶),
C(═O)(CR⁵R⁶)ₚ(CR⁷R⁸)ᵣ(CR⁹R¹⁰)ₛOC(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ᵣC(═O),
C(═O)(CR⁵R⁶)ᵣ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ᵣC(═O),
C(═O)(CR⁵R⁶)ᵣS—S(CR⁷R⁸)ᵣS—S(CR⁹R¹⁰)ₛOC(═O), and
C(═O)(CR⁵R⁶)ₚ(CR⁷R⁸)ᵣ(CR⁹R¹⁰)ₛN(R⁵)C(═O);
wherein R⁸ is independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl; or
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle.

In an alternative embodiment of Formula I-P-1, each optional divalent group X is selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR²—(CR⁹R¹⁰)ₛN(R⁴)C(═O), and any variant of the above X groups formed by the repositioning, addition, or deletion of the fragment N(R⁴)C(═O) or C(═O)N(R⁴)SO₂;
wherein the group X is optionally connected to one to two amino acid residue(s) A¹² or A¹³; or
X is independently selected from C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)SP(═O)(NHCR⁵R⁶),
C(═O)O(CR⁵R⁶)ₚ(CR⁷R⁸)ᵣ(CR⁹R¹⁰)ᵣC(═O),
C(═O)(CR⁵R⁶)ₚ(CR⁷R⁸)ᵣ(CR⁹R¹⁰)ₛOC(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)(CR⁵R⁶)ₚS—S(CR⁷R⁸)ᵣS—S(CR⁹R¹⁰)SOC(═O),
wherein the group X is optionally connected to one to two amino acid residue(s) A¹² or A¹³.

In an alternative embodiment of Formula I-P-1, each optional divalent group X is S(CR⁵R⁶)ₚ(CR⁷R⁸)ᵣ(CR⁹R¹⁰)ₛOC(═O) or a group selected from

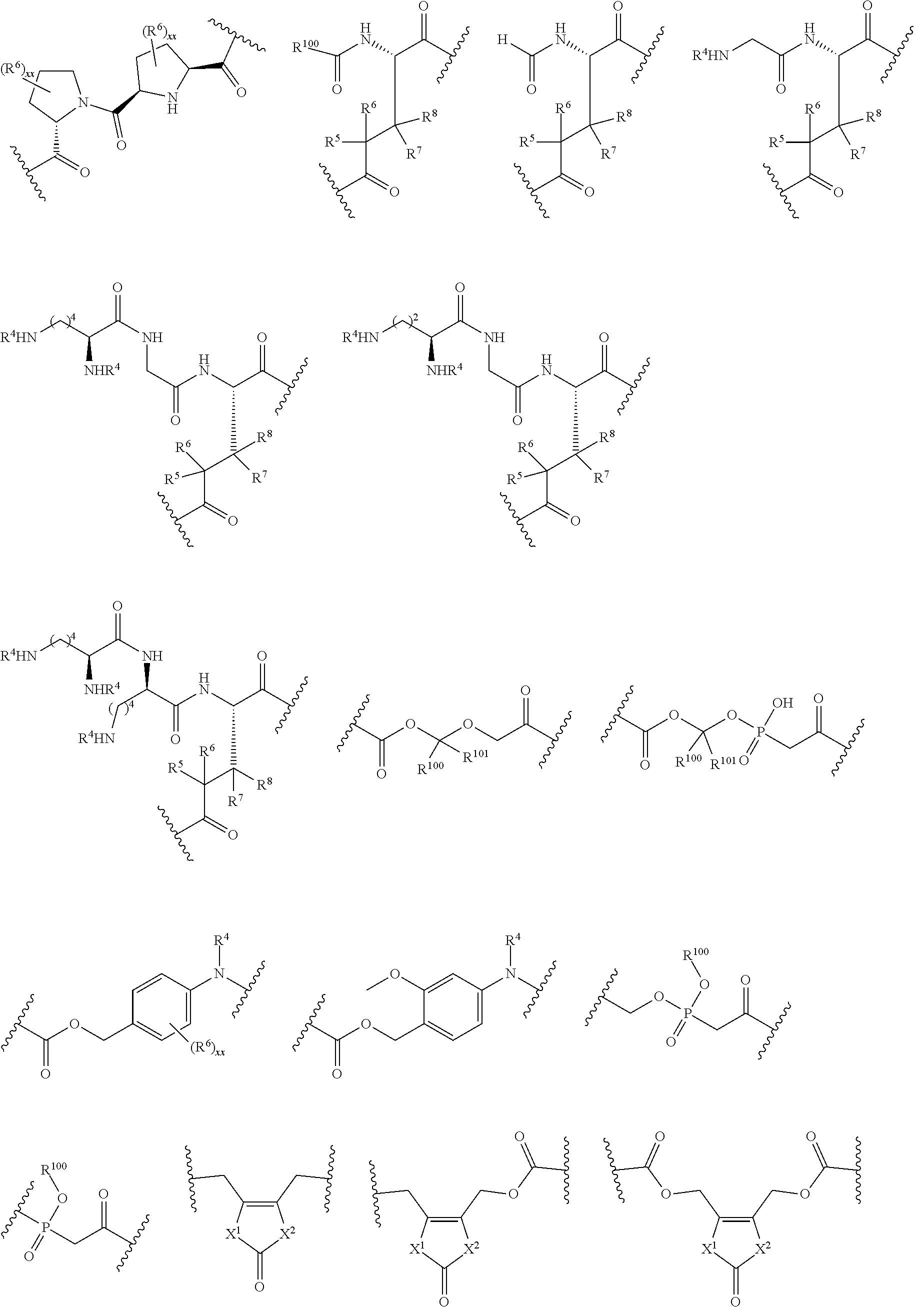

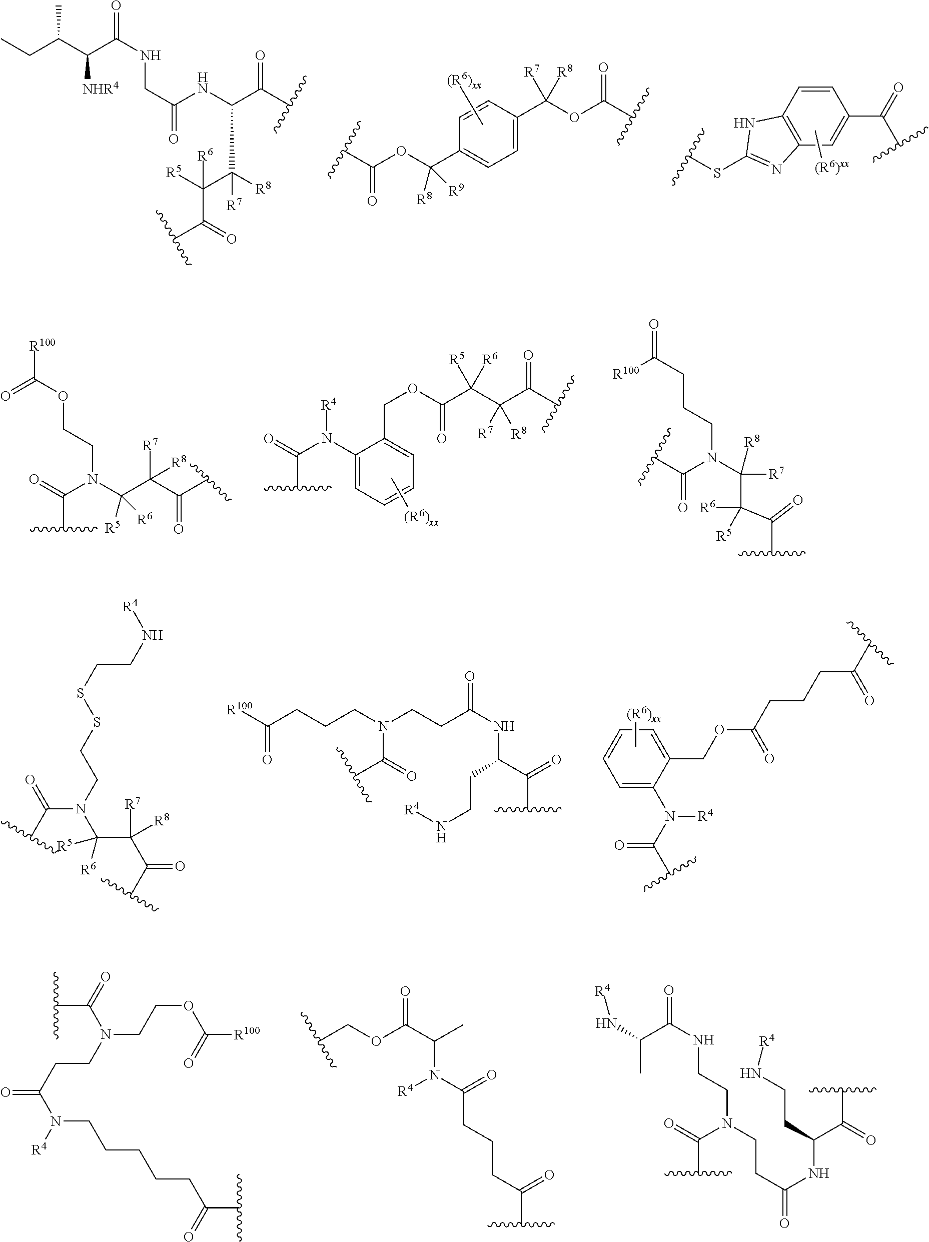

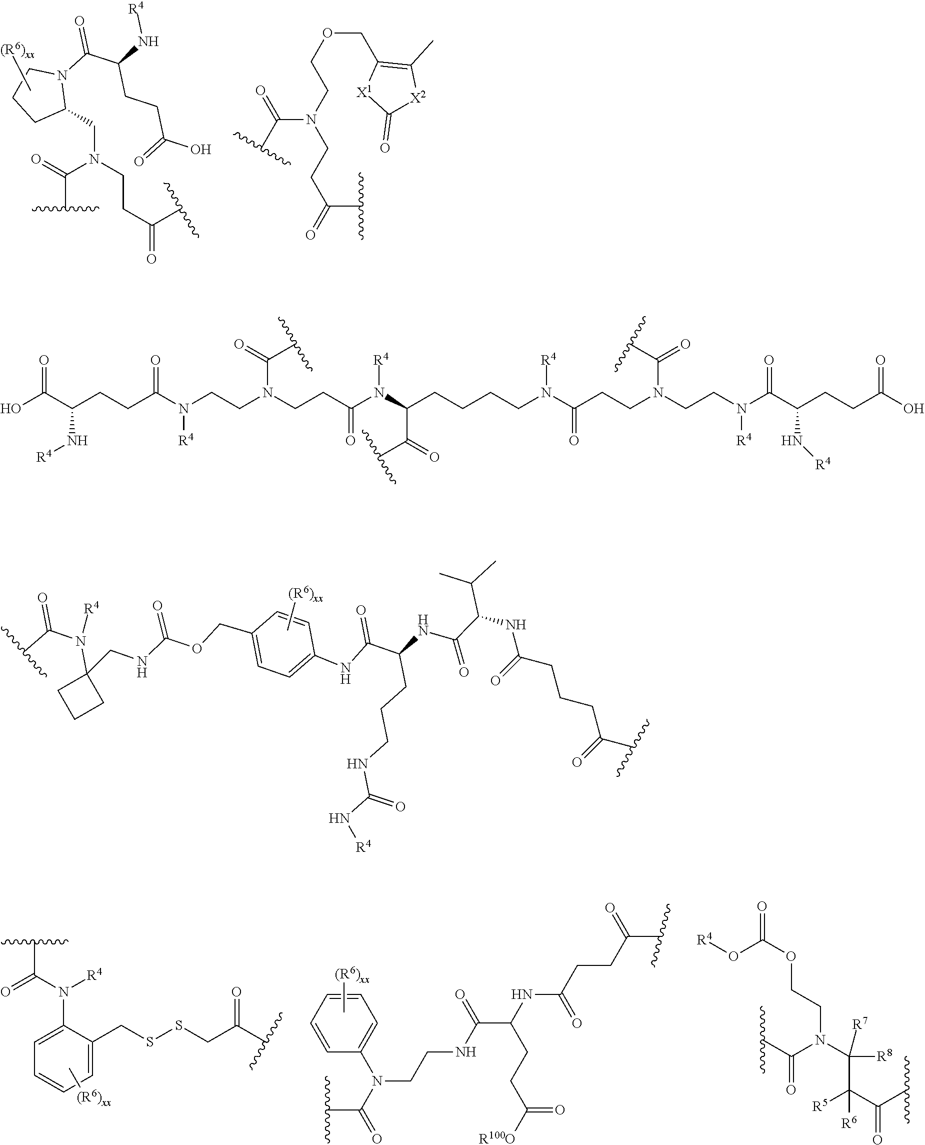

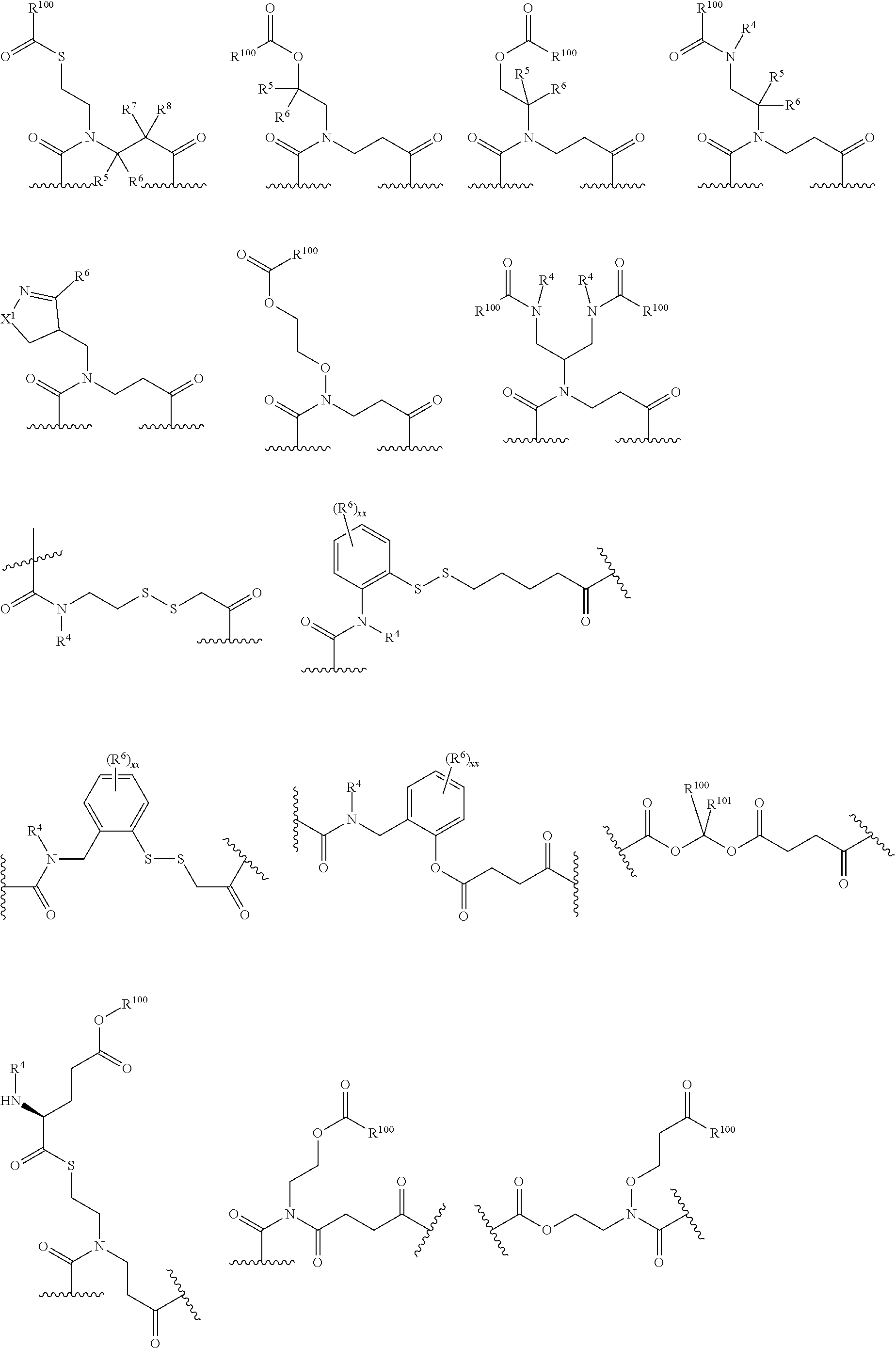

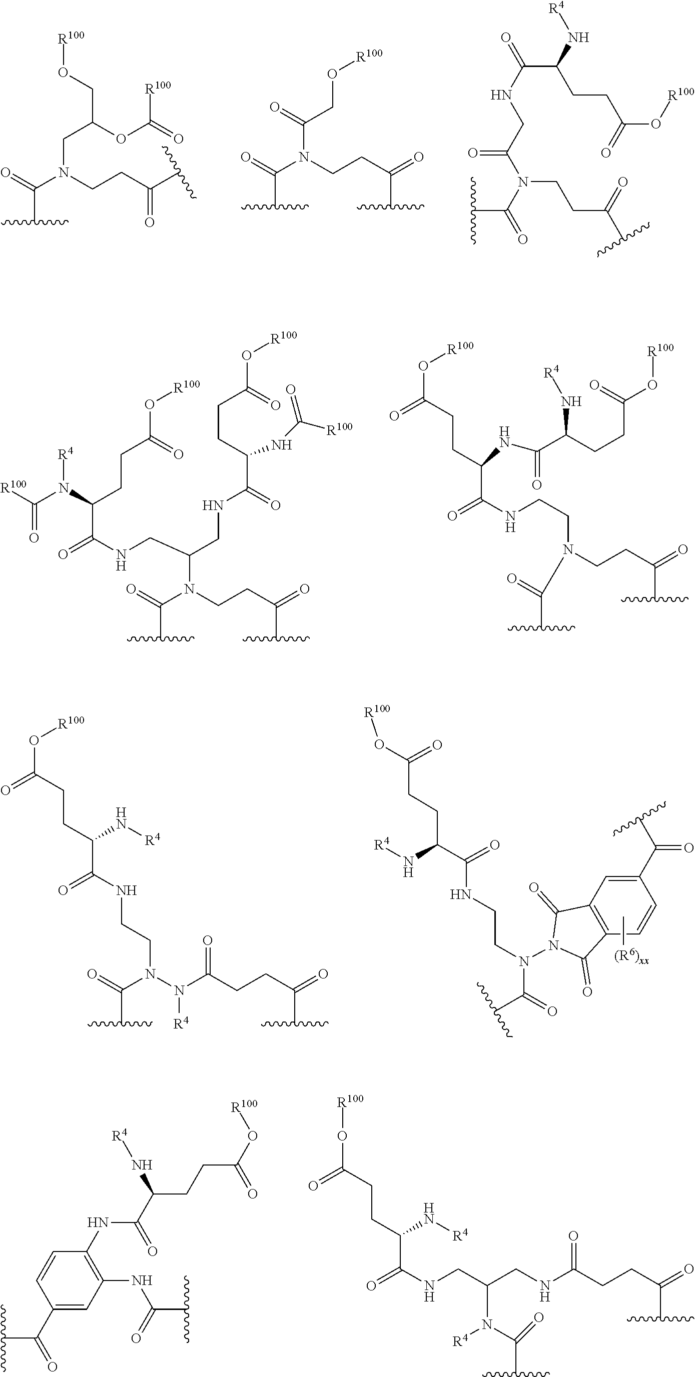

and any variant of the above X groups formed by the repositioning, addition, or deletion of the fragment N(R⁴)C(═O); or
wherein the group X is optionally connected to one to two amino acid residue(s) A¹² or A¹³;
wherein xx is 1, 2, or 3;
R¹⁰⁰ and R¹⁰¹ are independently selected from hydrogen and alkyl;
X¹ and X² are independently selected from O, NH, and CR⁵R⁶; and
all other variables are as defined herein in Formula I-P-1.

In an alternative embodiment of Formula I-P-1, each optional divalent group Y and Z are independently selected from N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O), C(═O)(CR⁵R⁶)ₚ(CR²R′)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), and any variant of the above groups formed by the repositioning, addition, or deletion of the fragment N(R⁴)C(═O) or C(═O)N(R⁴)SO₂; or
each optional divalent group Y and Z are independently selected from
C(═O)(CR⁵R⁶)ₚ(CR⁷R⁸)ᵣ(CR⁹R¹⁰)ᵣC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶),
C(═O)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O); and
C(═O)(CR⁵R⁶)ₚS—S(CR⁷R⁸)ᵣS—S(CR⁹R¹⁰)ₛOC(═O).

In an alternative embodiment of Formula I-P-1, each optional divalent group Z is independently selected from N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), and any variant of the above groups formed by the repositioning, addition, or deletion of the fragment N(R⁴)C(═O) or C(═O)N(R⁴)SO₂;
wherein the group Z is optionally connected to one to two amino acid residue(s) A¹² or A¹³; or
each optional divalent group Z is independently selected from
C(═O)(CR⁵R⁶)ₚ(CR⁷R⁸)ᵣ(CR⁹R¹⁰)ᵣC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ₛC(═O),
C(═O)(CR⁷R⁸)ᵣ(CR⁹R¹⁰)ₛP(═O)(NHCR⁵R⁶),
C(═O)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)(CR⁵R⁶)ₚS—S(CR⁷R⁸)ᵣS—S(CR⁹R¹⁰)ₛOC(═O), and
C(═O)(CR⁵R⁶)ₚ(CR⁷R⁸)ᵣ(CR⁹R¹⁰)ₛN(R⁵)C(═O);
wherein the group Z is optionally connected to one to two amino acid residue(s) A¹² or A¹³.

In another aspect, provided is a compound of the Formula I-P-2

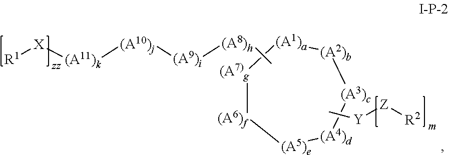

or a pharmaceutically acceptable salt, solvate, or hydrate thereof wherein:
R¹ and R² are optional groups, with at least one of the groups R¹ and R² being present in the Formula I-P-2; and
R¹ and R² are independently selected from alkyl, aryl, biaryl, heteroaryl, heteroarylaryl, steroidal, and arylheteroaryl; or
R¹ and R² are residues independently attached to X and Z, respectively, by subtracting a single or multiple H atom(s) from respective parent (precursor) structure(s) (H)ₙR¹ and (H)ₒR² at any one of the following optional H-containing group(s) independently selected from NH, OH, SH, C(═O)OH, CONH, SO₂NH, and S(═O)NH present in (H)ₙR¹ and (H)ₒR²; and wherein
a) (H)ₙR¹ and (H)ₒR² are independently an anti-inflammatory, an immunomodulating, or a nephro-protective, or a compound(s) possessing an activity or capable of inducing an activity that modulates gluococorticoid receptor (GR) activity, or modulates a cytokine release, or otherwise exhibits a biological activity against inflammation-associated kidney diseases, or a kidney injury; or
b) (H)ₙR¹ and (H)ₒR² are independently selected from aldosterone, AN3485, beclomethasone, beclomethasone dipropionate, betamethasone, budesonide, a budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, valdecoxib; a heteroaromatic vanin inhibitor compound (selected from compounds described in PCT WO 2020/114943 and US2020/0354338); a heterocyclic TYK2 inhibitor compound (selected from compounds described in PCT WO2020/86616 and US2020/0354338); and INF-alpha inhibitor compound (selected from compounds described in PCT WO 2020/159904 and EP3917912); or a variant thereof;
c) (H)ₙR¹ and (H)ₒR² are independently compound(s) active against an inflammation-associated kidney disease; or
d) (H)ₙR¹ is a glucocorticoid structure connected to X at one of the optional O or N atom(s), with at least one of these atoms present within the structure (H)ₙR¹; or a variant thereof; and
when an optional group R¹ is absent, then the fragment R¹X is replaced with R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂ heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is replaced with R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂ alkyl)C(═O), arylC(═O), arylOC(═O), arylNHC(═O), aryl N(C₁₋₁₂alkyl)C(═O), arylSO₂, arylNHSO₂ heteroarylC(═O), heteroarylOC(═O), heteroarylNHC(═O), heteroaryl N(C₁₋₁₂alkyl)C(═O), heteroarylSO₂, and heteroarylNHSO₂; or wherein
integers n and o are independently selected from 0, 1, 2, 3, 4, 5, 6, and 7, such that [n+o]≥1; and
A¹ through A¹¹ are optional amino acid residues independently selected from unsubstituted or substituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers a through k, m, and zz are independently selected from 0, 1, and 2, and wherein [m+zz]≥1; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴ wherein R³ and R are independently selected from H, alkyl, aryl, heteroaryl, or heterocyclyl; or the group A⁸ is directly connected to the group Y; and
optional divalent groups X, Y, and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O)N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ᵣ(CR⁹R¹⁰))ᵣP(═O)(OCR⁵R⁶)ₘ,
P(═O)(NHCR⁵R⁶)ₚ(CR⁷R⁸)ᵣ(CR⁹R′)ᵣC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O), or
C(═O)N[CH₂CH₂NHC(═O)CH(NH₂)CH₂CH₂COOC₁₋₆alkyl]CH₂CH₂C(═O);
or any variant of above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein
R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁶ and R⁷, and ii) R⁹ and R¹⁰, together with the atom to which they are attached form a C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein
when fragments (CR⁷R⁸)ᵣ(CR⁹R¹⁰), or (OCR⁷R⁸)ᵣ(CR⁹R¹⁰)ₛ are present, then [r+s]≥1; or
X, Y, and Z are independently selected from one to four amino acid residue(s) A¹², A¹³, A¹⁴, and A¹⁵ connected to each other with peptide bonds; wherein
A¹², A¹³, A¹⁴, or A¹⁵ are independently unsubstituted or substituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid, or 5-amino-4-arylpentanoic acid, or similar natural or unnatural amino acid residues; or
X is a group comprised of the following structures, additionally connected to one to two amino acid residue(s) A¹² or A¹³, at the right side of the following structures:
(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ᵣC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ᵣC(═O),
C(═O)O(CR⁵R⁶)ₚ(CR⁷R⁸)ᵣS—S(CR⁹R¹⁰)ᵣC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ᵣC(═O),
C(═O)OCR⁵═CR⁷—(CR⁹R¹⁰)ᵣC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O), or
C(═O)N[CH₂CH₂NHC(═O)CH(NH₂)CH₂CH₂COOC₁₋₆alkyl]CH₂CH₂C(═O);
or any variant of these groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
Z is a group comprised of the following structures, additionally connected to one to two amino acid residue(s) A¹² or A¹³, at the left side of the following structures:
(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ₛP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ᵣC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ᵣC(═O),
C(═O)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)(CR⁹R¹⁰)ₛC(═O),
C(═O)(CR⁵R⁶)ₚS—S(CR²R⁸)S(CR⁹R¹⁰)ₛOC(═O), C(═O)(CR⁵R⁶)ₚ(CR⁷R⁸)ᵣ(CR⁹R¹⁰)N(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁷R⁸)ₚ(CR²R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A′)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O), or
C(═O)N[CH₂CH₂NHC(═O)CH(NH₂)CH₂CH₂COOC₁₋₆alkyl]CH₂CH₂C(═O);
or any variant of these groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸; or wherein
an optional group X, either at its left or right side therein, incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ᵣNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), or similar linear groups.

In an alternative embodiment of Formula I-P-2, R⁵ and R⁶ together with the atom to which they are attached form a C₃₋₆cycloalkylene.

In an alternative embodiment of Formula I-P-2, fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present and [p+r+s]≥1.

In an alternative embodiment of Formula I-P-2, fragments (CR⁵R⁶)ₚ(CR¹R⁸)ᵣ or (OCR⁵R⁶)ₚ(CR⁷R⁸)ᵣ are present and [p+r]≥1.

In an alternative embodiment of Formula I-P-2, optional divalent groups X, Y, and Z are independently selected from
(C₁₋₁₂alkylene)C(═O)N(R⁴), N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛN(R⁴)C(═O),
C(═O)N(R⁴)SO₂(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O), and
C(═O)CR⁵═CR⁷—(CR⁹R¹⁰)ₛN(R⁴)C(═O),
and any variant of the above groups formed by straightforward repositioning, addition, or deletion of the fragment N(R⁴)C(═O) or C(═O)N(R⁴)SO₂; or
optional divalent groups X, Y, and Z are independently selected from
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ᵣC(═O),
C(═O)(CR⁷R⁸)ᵣ(CR⁹R¹⁰))ᵣP(═O)(OCR⁵R⁶)ₘ,
P(═O)(NHCR⁵R⁶)ₚ(CR⁷R⁸)ᵣ(CR⁹R¹⁰)ᵣC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)N[(CR⁵R⁶)ₚ(CR⁷R⁸)ᵣNHC(═O)A¹⁴](CR⁹R¹⁰)ₛ)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁵)ᵣN(C₁₋₆alkyl)C(═O)A¹⁴](CR⁹R¹⁰)NHC(═O),
C(═O)N[(CR⁵R⁶)(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R¹)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)C(═O),
C(═O)N[(CR⁵R⁶)ₚ(CR⁷R¹)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ᵣC(═O),
C(═O)N[CH₂CH₂NHC(═O)CH₂CH₂CH(NH₂)COOH](CR⁵R⁶)ₚ(CR⁷R⁸)ᵣO(C═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), and
C(═O)N[CH₂CH₂NHC(═O)CH₂CH₂CH(NH₂)COOH](CR⁵R⁶)ₚ OC(═O)(CR⁷R⁸)ᵣ(CR⁹R¹⁰)ₛC(═O);
wherein R⁸ is independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl; or
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle.

In an alternative embodiment of Formula I-P-2, optional divalent group X is independently selected from
(C₁₋₁₂alkylene)C(═O)N(R⁴), N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O),
and any variant of the above groups formed by straightforward repositioning, addition, or deletion of the fragment N(R⁴)C(═O) or C(═O)N(R⁴)SO₂;
wherein X is additionally connected to one to two amino acid residue(s) A¹² or A¹³; or each optional divalent group X is independently selected from
C(═O)(CR⁵R⁶)ₚ(CR⁷R⁸)ᵣ(CR⁹R¹⁰)ᵣC(═O),
C(═O)(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ₛC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ᵣC(═O), C(═O)N(R⁵)(CR⁵R⁶)ₚ(CR⁷R⁸)ᵣ(CR⁹R¹⁰)ᵣC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚS—S(CR⁷R⁸)ᵣ(CR⁹R¹⁰)ᵣC(═O),
C(═O)O(CR⁵R⁶)ₚS—S(CR⁷R⁸)ᵣ(CR⁹R¹⁰)ᵣC(═O),
C(═O)(CR⁵R⁶)ᵣS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ᵣNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ᵣNHC(═O),
C(═O)N[(CR⁵R⁶)ₚ(CR⁷R⁸)ᵣN(C₁₋₆alkyl)C(═O)A¹⁴](CR⁹R¹⁰)ᵣC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)C(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)(CR⁷R⁸)ₛOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ₚC(═O)O(CR⁹R¹⁰)ᵣC(═O),
C(═O)N[CH₂CH₂NHC(═O)CH₂CH₂CH(NH₂)COOH](CR⁵R⁶)ₚ(CR⁷R⁸)ₛOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚ OC(═O)(CR⁷R⁸)ᵣ
(CR⁹R¹⁰)ₛC(═O); wherein X is additionally connected to one to two amino acid residue(s) A¹² or A¹³.

In an alternative embodiment of Formula I-P-2, each optional divalent group X is S(CR⁵R⁶)ₚ(CR⁹R⁵)ᵣ(CR⁹R¹⁰)ₛOC(═O) or a group selected from

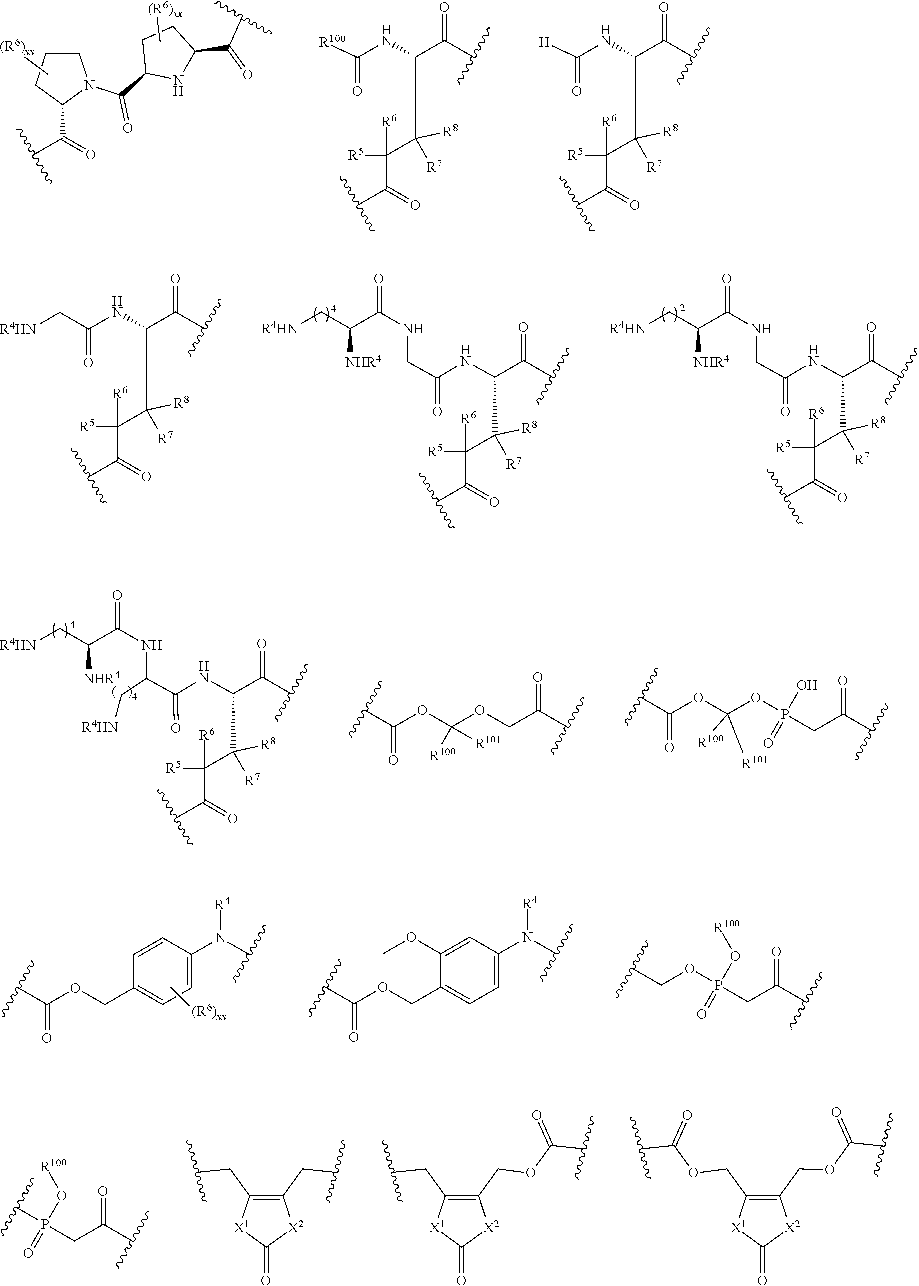

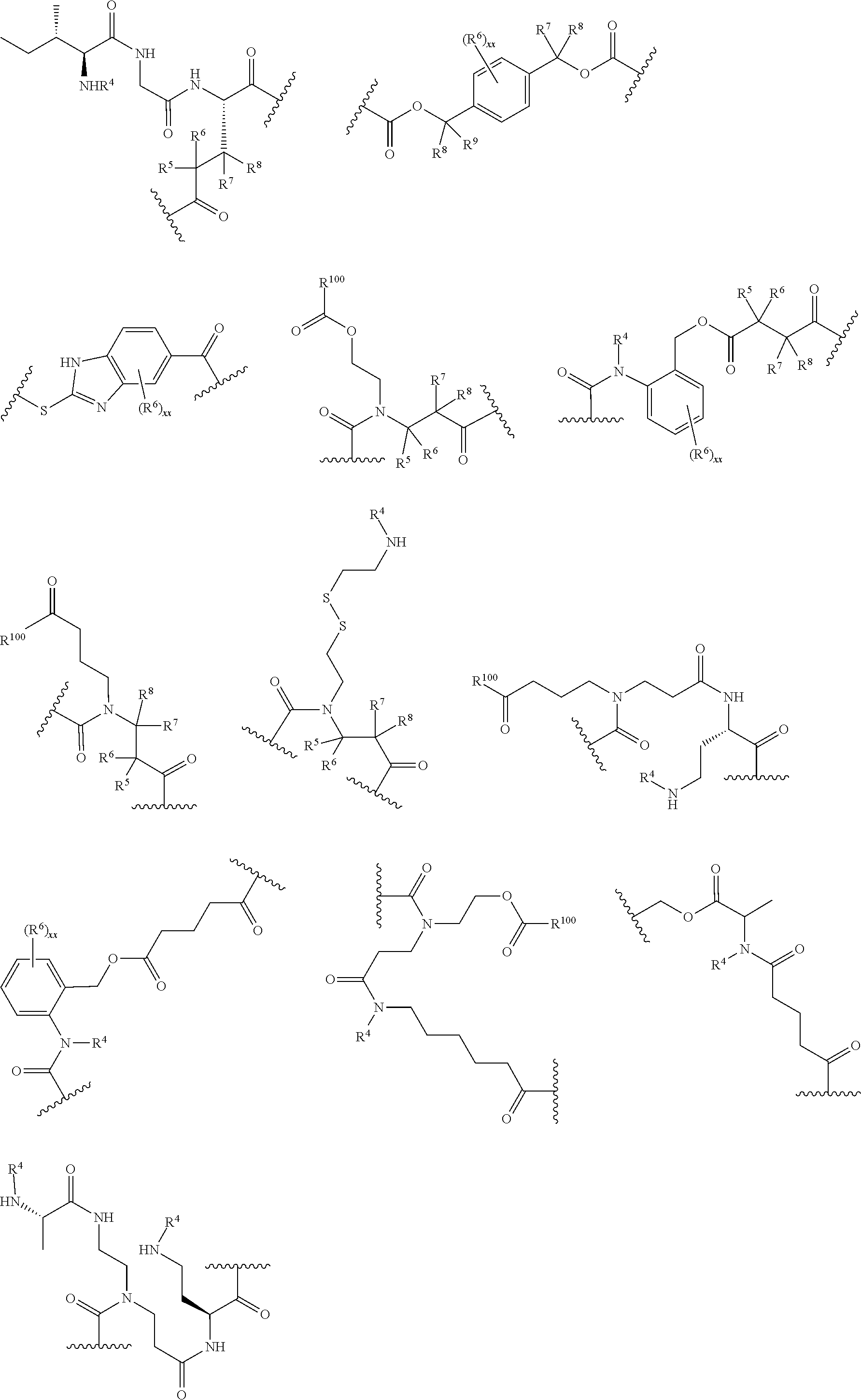

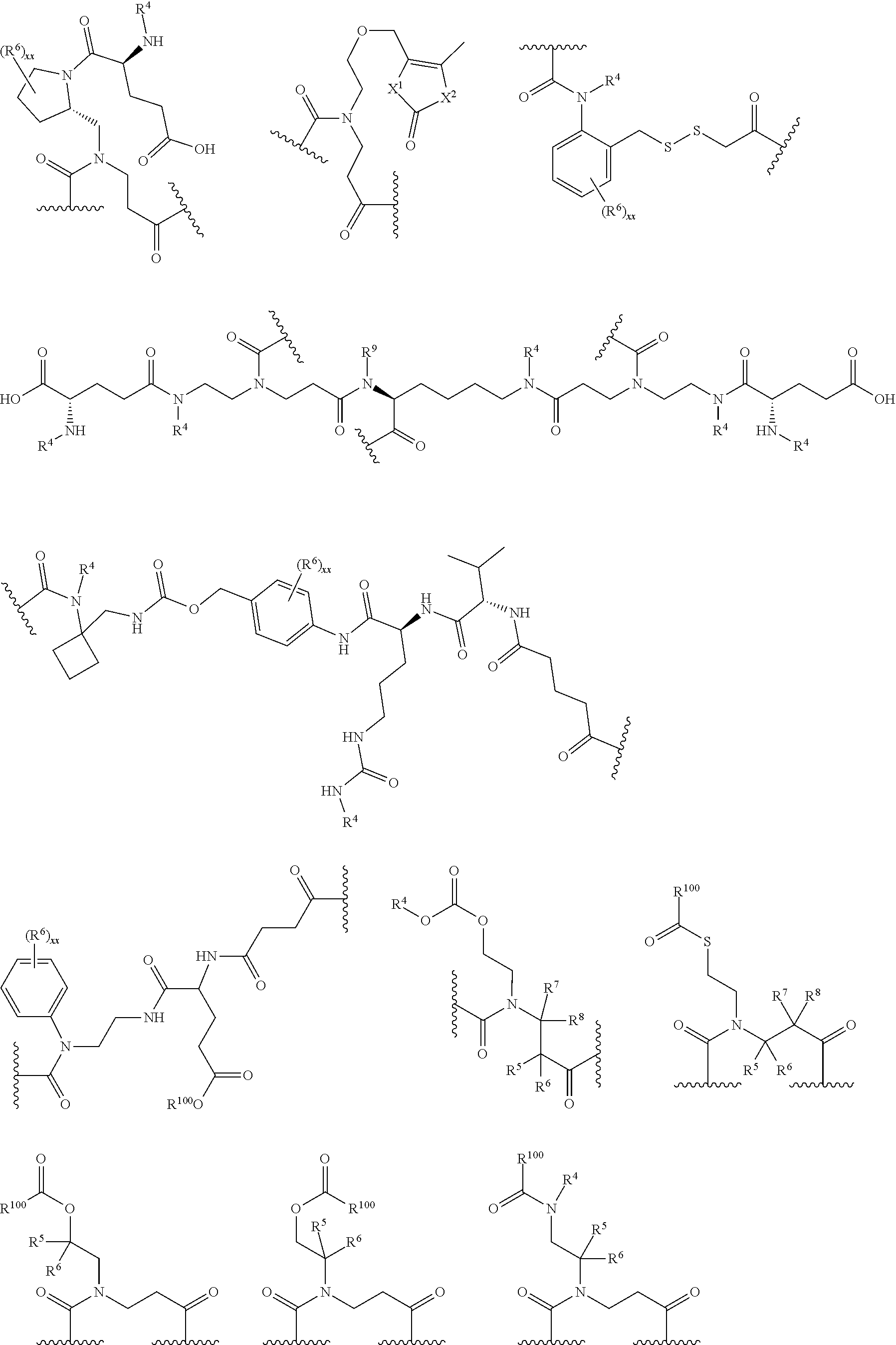

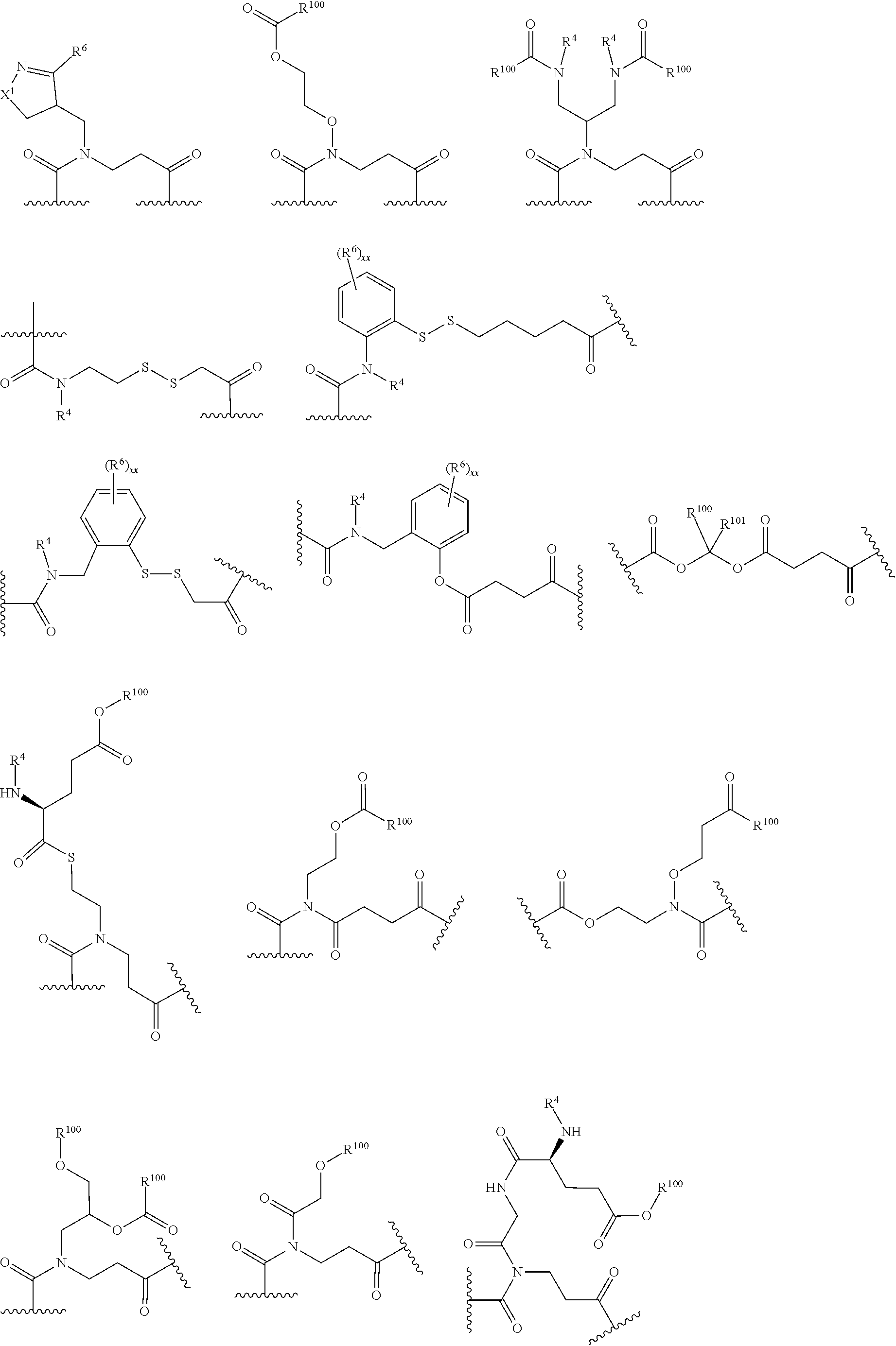

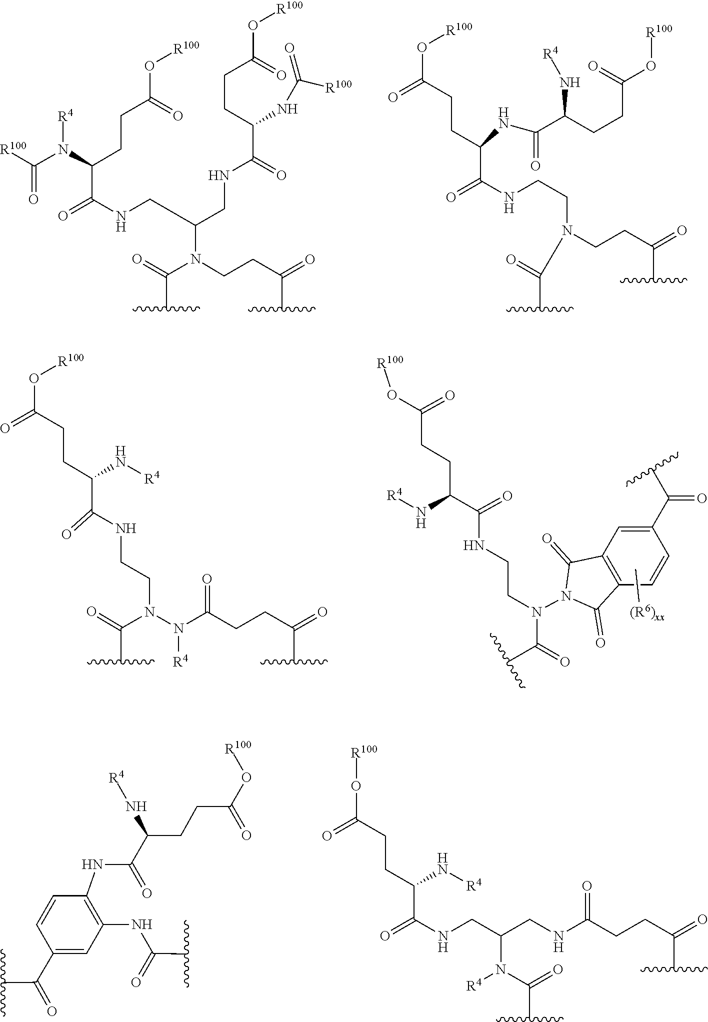

and any variant of the above X groups formed by the repositioning, addition, or deletion of the fragment N(R⁴)C(═O); or
wherein the group X is optionally connected to one to two amino acid residue(s) A¹² or A¹³;
wherein xx is 1, 2, or 3;
R¹⁰⁰ and R¹⁰¹ are independently selected from hydrogen and alkyl;
X¹ and X² are independently selected from O, NH, and CR⁵R⁶; and
all other variables are as defined herein in Formula I-P-2.

In an alternative embodiment of Formula I-P-2, optional divalent group Z is independently selected from
(C₁₋₁₂alkylene)C(═O)N(R⁴), N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O), C(═O)(CR⁵R⁶)ₚ(CR²R′)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O),
C(═O)N(R⁴)SO₂(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ᵣC(═O),
C(═O)CR⁵═CR⁷—(CR⁹R¹⁰)ₛN(R⁴)C(═O),
and any variant of the above groups formed by straightforward repositioning, addition, or
deletion of the fragment N(R⁴)C(═O) or C(═O)N(R⁴)SO₂.
wherein Z is additionally connected to one to two amino acid residue(s) A¹² or A¹³; or
each optional divalent group Z is independently selected from
C(═O)(CR⁵R⁶)ₚ(CR⁷R⁸)ᵣ(CR⁹R¹⁰)ᵣC(═O),
C(═O)O(CR⁵R⁶)O(CR⁷R⁸)(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁷R⁸)ᵣP(═O)(OCR⁵R⁶),
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)N(R⁵)SO₂(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ᵣC(═O),
C(═O)O(CR⁵R⁶)ₚS—S(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ₛOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)C(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R²)ₛC(═O),
C(═O)N[CH₂CH₂NHC(═O)CH₂CH₂CH(NH₂)COOH](CR⁵R⁶)ₚ(CR⁷R⁸)ᵣOC(═O)(CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸),
(CR⁹R¹⁰)ᵣC(═O), and
C(═O)N[CH₂CH₂NHC(═O)CH₂CH₂CH(NH₂)COOH](CR⁵R⁶)ₚ OC(═O)(CR⁷R⁸)ᵣ
(CR⁹R¹⁰)ₛC(═O); wherein Z is additionally connected to one to two amino acid residue(s) A¹² or A¹³.

In an alternative embodiment of Formula I-P-2, R⁵ and R⁶ together with the atom to which they are attached form a saturated or unsaturated C₃₋₆cycloalkylene

In one aspect, the compound of Formula I-P-1 or Formula I-P-2 is a compound of Formula I-1:

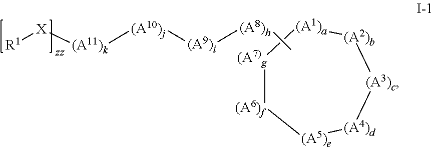

or a pharmaceutically acceptable salt, solvate, or hydrate thereof;
wherein the integer zz is 1 or 2.

In one aspect, the compound of Formula I-1 is a compound of Formula I-2:

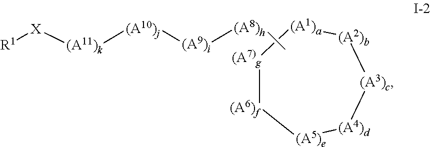

or a pharmaceutically acceptable salt, solvate, or hydrate thereof wherein:
R¹ is a residue formed by subtracting a H atom from a respective parent (precursor) structure HR¹; and wherein HR¹ is selected from:
a) an anti-inflammatory compound, an immunomodulating compound, a nephro-protective compound, and a compound possessing an activity or capable of inducing an activity that modulates gluococorticoid receptor (GR) activity, or reduces the levels of proteinuria/creatinine, or modulates a cytokine release, or otherwise exhibits a biological activity against inflammation-associated kidney diseases, or a kidney injury; and
b) a structure selected from aldosterone, AN3485, beclomethasone, beclomethasone dipropionate, betamethasone, budesonide, a budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, and valdecoxib; and a heteroaromatic vanin inhibitor compound (selected from compounds described in PCT WO 2020/114943 and US2020/0354338); or a heterocyclic TYK2 inhibitor compound (selected from compounds described in PCT WO2020/86616 and US2020/0354338); or INF-alpha inhibitor compound (selected from compounds described in PCT WO 2020/159904 and EP3917912); or a variant thereof; and
c) a glucocorticoid structure connected to X through a residue of an OH group present within the structure HR¹;
integers a through k are independently selected from 0, 1, and 2;
A¹ through A¹¹ are optional amino acid residues unsubstituted or substituted at any N atom and independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid;
wherein when any of integers a through k is 0, then any of the two groups adjacent to a respective absent group (according to the integer 0 at said absent group) are connected to each other directly; and wherein
a) optional divalent group X is independently selected from
—C(═O)(CR²R³)ₚ(CR⁴R⁵)ᵣ(CR⁶R⁷)ₛC(═O)—,
—C(═O)(Aryl)ₚ(CR⁴R⁵)ᵣ(CR⁶R⁷)ₛC(═O)—,
—C(═O)(CR²R³)ₚ(Aryl)ᵣ(CR⁶R⁷)ₛC(═O)—,
—C(═O)O(CR²R³)O(CR⁴R⁵)ᵣ(CR⁶R⁷)ₛC(═O)—,
—C(═O)O(CR²R³)ₚ(CR⁴R⁵)ᵣ(CR⁶R⁷)ₛC(═O)—, —C(═O)N(R⁵)(CR²R³)ₚ(CR⁴R⁵) (CR⁶R⁷)ₛC(═O)—,
—C(═O)(CR²R³)ₚ(CR⁴R⁵)ᵣ(CR⁶R⁷)ₛOC(═O)—, —C(═O)(CR²R³)ₚ(CR⁴R⁵)ᵣ(CR⁶R⁷)ₛN(R⁴)C(═O)—,
—C(═O)CR²═CR⁴—(CR⁶R⁷)ᵣC(═O)—,
—C(═O)OCR²═CR⁴—(CR⁶R⁷)ₛC(═O)—, —C(═O)N(R⁵)CR²═CR⁴—(CR⁶R⁷))C(═O)—,
—C(═O)CR²═CR⁴—(CR⁶R⁷)ₛOC(═O)—, —C(═O)CR²═CR⁴—(CR⁶R⁷)ₛN(R⁴)C(═O)—,
—C(═O)(CR²R³)ₚS—S(CR⁴R⁵)ᵣ(CR⁶R⁷)ₛC(═O)—,
—C(═O)O(CR²R³)ₚS—S(CR⁴R⁵)ᵣ(CR⁶R⁷)ₛC(═O)—,
—C(═O)(CR²R³)ₚS—S(CR⁴R⁵)(CR⁶R⁷)ₛOC(═O)—,
—C(═O)(CR²R³)ₚ(CR⁴R⁵)ᵣ(CR⁶R⁷)ₛN(R⁵)C(═O)—,
—C(═O)(CR²R³)ₚ(CR⁴R⁵)ᵣS—S(CR⁶R⁷)ₛC(═O)—,
—C(═O)(CR²R³)ₚ(CR⁴R⁵)ᵣS—S(CR⁶R⁷)ₛC(═O)—,
—C(═O)(CR²R³)ₚS—S(CR⁴R⁵)ᵣS—S(CR⁶R⁷)ₛOC(═O)—,
—C(═O)N[(CR²R³)ₚ(CR⁴R⁵)ᵣNHC(═O)(CR⁶R⁷)ₛNCH(NH₂)COOH]CH₂CH₂C(═O)—,
—C(═O)N[(CR²R³)ₚ(CR⁴R⁵)ᵣN(C₁₋₆alkyl)C(═O)—(CR⁶R⁷)NCH(NH₂)COOH]CH₂CH₂C(═O)—,
—C(═O)N[CH₂CH₂NHC(═O)CH(NH₂)CH₂CH₂COOH]CH₂CH₂C(═O)—,
—C(═O)N{CH₂CH₂NHC(═O)—[(S)—CH(NH₂)]CH₂CH₂COOH}CH₂CH₂C(═O)—,
—C(═O)N{CH₂CH₂NHC(═O)—[(R)—CH(NH₂)]CH₂CH₂COOH}CH₂CH₂C(═O)—,
—C(═O)N[CH₂CH₂OC(═O)CH(NH₂)CH₂CH₂COOH]CH₂CH₂C(═O)—,
—C(═O)N[CH₂CH₂NHC(═O)CH(OH)CH₂CH₂COOH]CH₂CH₂C(═O)—,
—C(═O)N[CH₂CH₂NHC(═O)CH(NH₂)CH₂COOH]CH₂CH₂C(═O)—,
—C(═O)N{CH₂CH₂NHC(═O)—[(S)—CH(NH₂)]CH₂COOH}CH₂CH₂C(═O)—,
—C(═O)N{CH₂CH₂NHC(═O)—[(R)—CH(NH₂)]CH₂COOH}CH₂CH₂C(═O)—,
—C(═O)N{CH₂CH₂NHC(═O)CH[NHC(═O)H]CH₂CH₂COOH}CH₂CH₂C(═O)—,
—C(═O)N{CH₂CH₂NHC(═O)CH[NHC(═O)Me]CH₂CH₂COOH}CH₂CH₂C(═O)—,
—C(═O)N[CH₂CH₂NHC(═O)—{(S)—CH[NHC(═O)H]}CH₂CH₂COOH]CH₂CH₂C(═O)—,
—C(═O)N{CH₂CH₂NHC(═O)CH[NHC(═O)C₁₋₆alkyl]CH₂CH₂COOH}CH₂CH₂C(═O)—,
—C(═O)N[CH₂CH₂NHC(═O)CH(NH₂)CH₂CH₂COOC₁₋₆alkyl]CH₂CH₂C(═O)—,
—C(═O)N[CH₂CH₂NHC(═O)CH(NH₂)CH₂CH₂COOH]CH₂CH₂C(═O)OCH₂CH₂C(═O)—,
—C(═O)N[CH₂CH₂NHC(═O)CH(NH₂)CH₂CH₂COOH]CH₂CH₂C(═O)OCH(Me)CH(Me)C(═O)—,
—C(═O)N[CH₂CH₂NHC(═O)CH(NH₂)CH₂CH₂COOH]CH₂CH₂C(═O)OC(Me)₂C(Me)₂C(═O)—,
—C(═O)N[CH₂CH₂NHC(═O)CH(NH₂)CH₂CH₂COOH]CH₂CH₂C(═O)O—C₃₋₆cycloalkylene-C(═O)—,
—C(═O)N[CH₂CH₂NHC(═O)CH(NH₂)CH₂CH₂COOH]CH₂CH₂OC(═O)CH₂CH₂C(═O)—,
—C(═O)N[CH₂CH₂NHC(═O)CH(NH)CH₂CH₂COOH]CH₂CH(Me)OC(═O)CH₂CH₂C(═O)—,
—C(═O)N[CH₂CH₂NHC(═O)CH(NH₂)CH₂CH₂COOH]CH₂C(Me)₂OC(═O)CH₂CH₂C(═O)—,
—C(═O)N[CH₂CH₂NHC(═O)CH(NH₂)CH₂CH₂COOH]CH₂CH₂OC(═O)—C₃₋₆cycloalkylene-C(═O)—,
—C(═O)N[CH₂CH₂NHC(═O)CH(NH₂)CH₂CH₂COOH]CH₂CH₂OC(═O)C(Me)CH(Me)C(═O)—,
—C(═O)N[CH₂CH₂NHC(═O)CH(NH₂)CH₂CH₂COOH]CH₂CH₂OC(═O)C(Me)₂C(Me)₂C(═O)—,
—C(═O)N[CH₂CH₂NHC(═O)CH₂CH₂CH(NH₂)COOH]CH₂CH₂C(═O)—,
—C(═O)N[CH₂CH₂NHC(═O)CH₂CH₂CH(NH₂)COOC₁₋₆alkyl]CH₂CH₂C(═O)—,
—C(═O)N[CH₂CH₂OC(═O)CH₂CH₂CH(NH₂)COOH]CH₂CH₂C(═O)—,
—C(═O)N[CH₂CH₂NHC(═O)CH₂CH₂CH(NH₂)COOH](CR²R³)ₚ(CR⁴R⁵)ₛC(═O)O(CR⁶R⁷)ₛ
C(═O)—,
—C(═O)N[CH₂CH₂NHC(═O)CH₂CH₂CH(NH₂)COOH](CR²R³)ₚ(CR⁴R⁵)ᵣ OC(═O) (CR⁶R⁷)ₛC(═O)—,
—C(═O)N[CH₂CH₂NHC(═O)CH₂CH₂CH(NH₂)COOH](CR²R³)ₚC(═O)O(CR⁴R⁵), (CR⁶R⁷)ₛC(═O)—,
—C(═O)N[CH₂CH₂NHC(═O)CH₂CH₂CH(NH₂)COOH](CR²R³)ₚ OC(═O)(CR⁴R⁵)ᵣ(CR⁶R⁷)ₛC(═O)—,
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁴)COOH]CH₂CH₂C(═O),
(R)—C(═O)N[CH₂CH₂NHC(═O)CH₂CH₂CH(NH(C═O)R⁴)COOH]CH₂CH₂C(═O),
—C(═O)CH₂CH₂C(═O)—, and
—C(═O)CHMeCHMeC(═O)—; or
b) optional divalent group X is S(CR²R³)ₚ(CR⁴R⁵)ᵣ(CR⁶R⁷)ₛOC(═O) or a group selected from

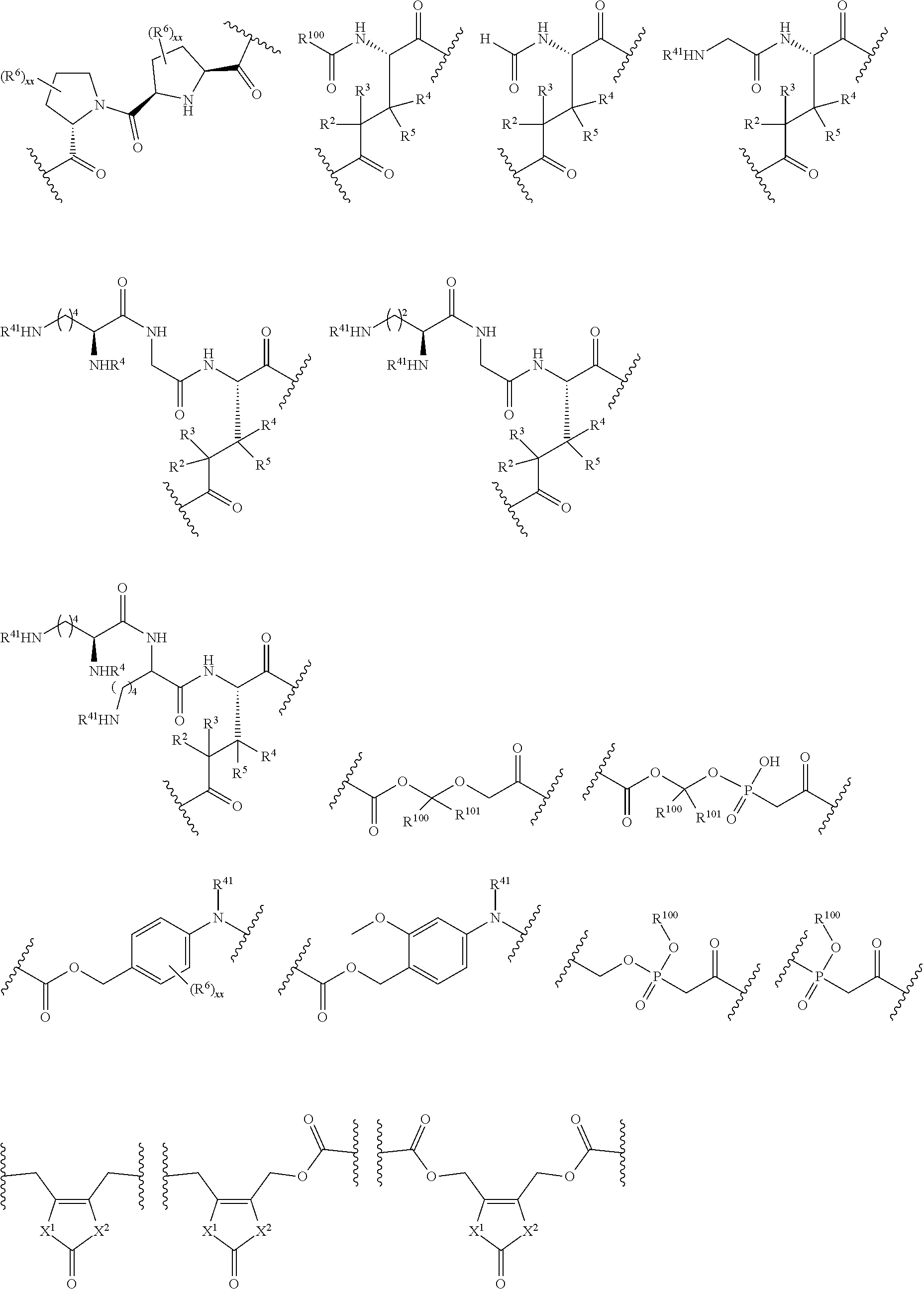

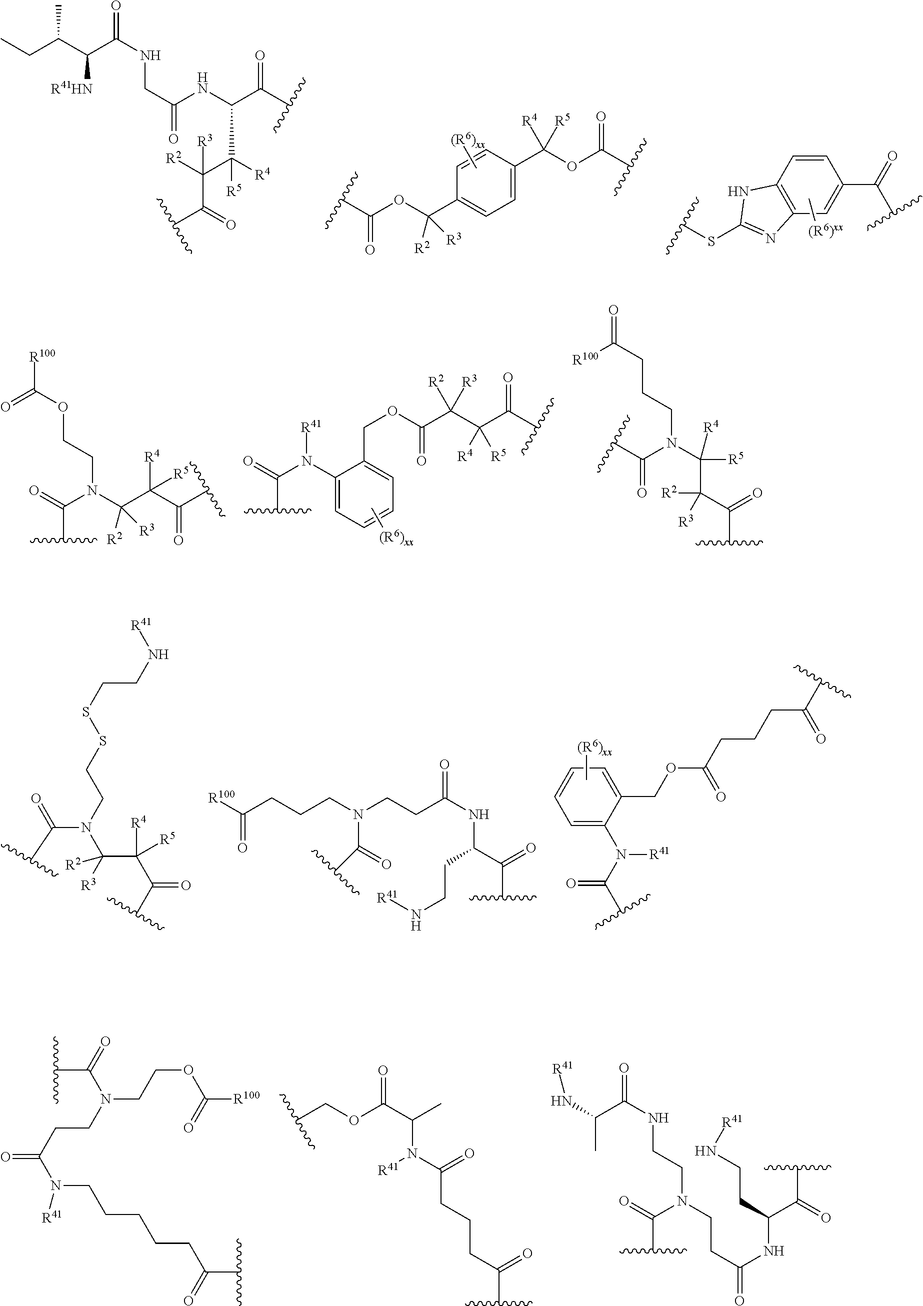

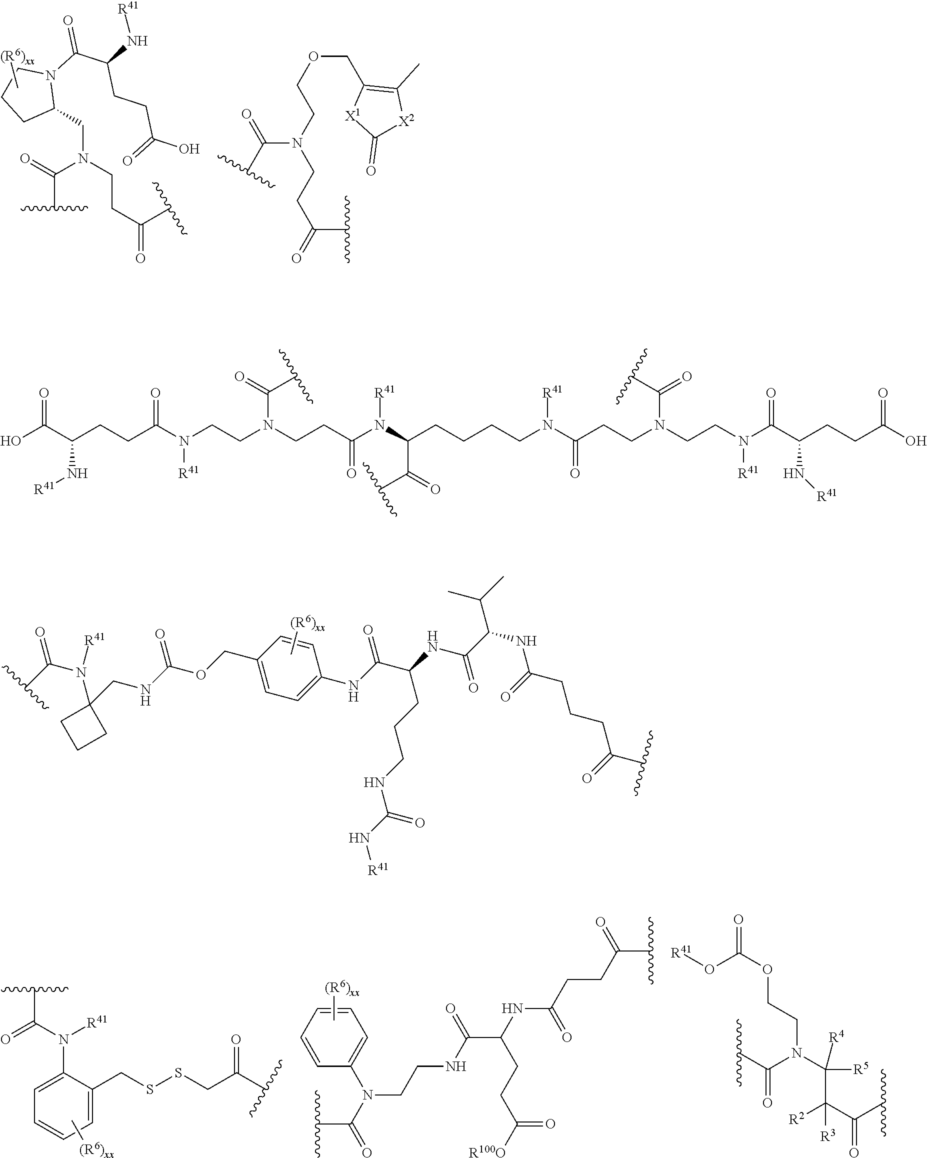

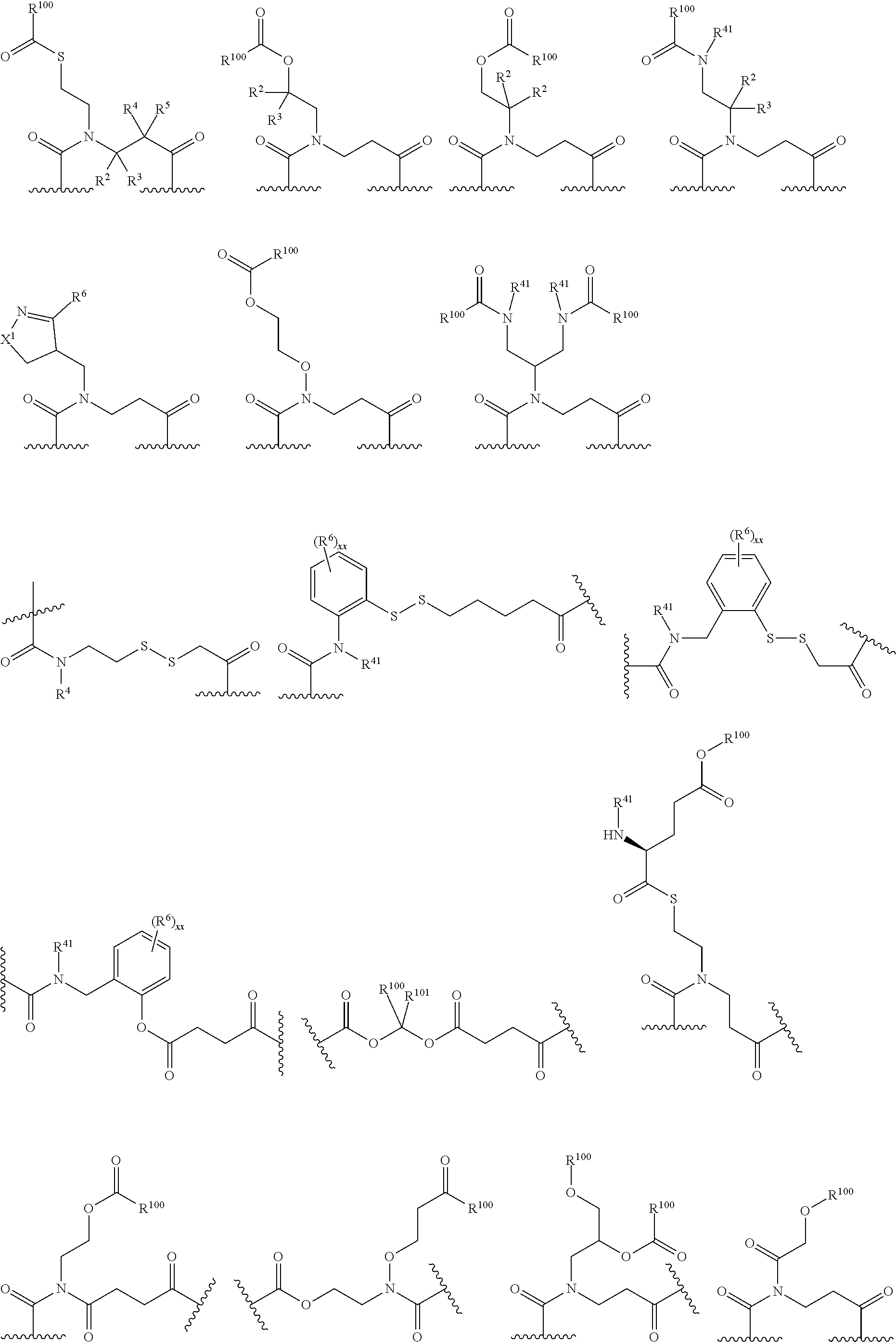

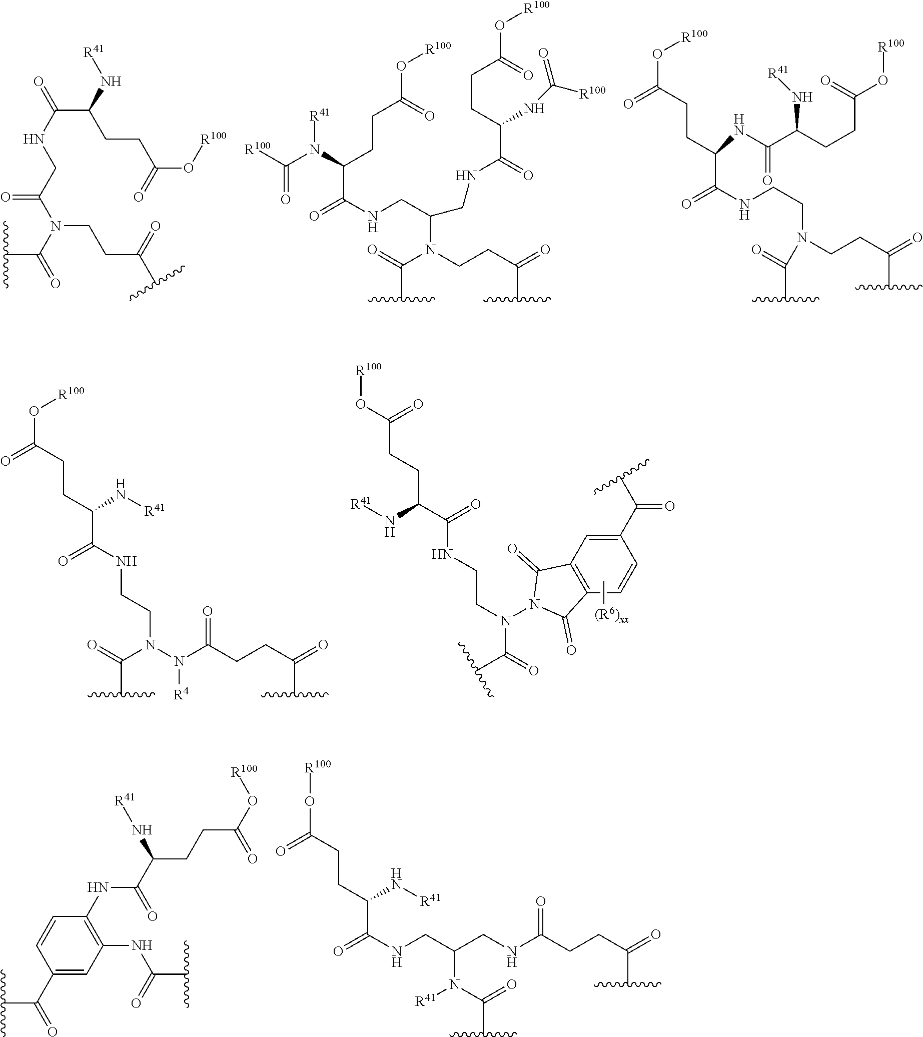

xx is 1, 2, or 3;
R¹⁰⁰ and R¹⁰¹ are independently selected from hydrogen and alkyl;
X¹ and X² are independently selected from O, NH, and CR⁵R⁶;
R⁴¹ is independently selected from H, alkyl, aryl, heteroaryl, and heterocyclyl;
R², R⁴, R⁵, R⁶, and Rare independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl;
R³ is selected from H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl; or
any two of R² through R⁷, together with the atom(s) to which they are attached form a 4 to 7-member saturated or unsaturated heterocycle containing at least one O atom, or containing one O atom and an additional heteroatom independently selected from N and S and wherein remaining atoms are carbon atoms; or
any two of R² through R¹, together with the carbon atom(s) to which they are attached form a 4 to 7-member saturated or unsaturated C₃₋₆cycloalkylene; or any of i) R² and R³, ii) R⁴ and R⁵, iii) R⁶ and R⁷, together with the atom to which they are attached form a saturated or unsaturated C₃₋₆cycloalkylene; or
any two of R² through R¹ together with the atom(s) to which they are attached form a 5 to 7-member saturated or unsaturated heterocycle wherein the ring optionally comprises an additional heteroatom selected from N, O, and S, and wherein the remaining atoms are carbon; or
R³ and R⁵ together with the atom to which they are attached form a 4 to 6-member saturated heterocycle containing at least one O atom and optionally comprising an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein
when fragments (CR²R³)ₚ(CR⁴R⁵)ᵣ(CR⁶R⁷)ₛ or (OCR²R³)ₚ(CR⁴R⁵)(CR⁶R⁷)S are present, then [p+r+s]≥1; and wherein
when fragments (CR²R³)ₚ(CR⁴R⁵)ᵣ or (OCR²R³)ₚ(CR⁴R⁵)ᵣ are present, then [p+r]≥1; and wherein
when fragments (CR⁴R⁵)ᵣ(CR⁶R⁷)ₛ or (OCR⁴R⁵)ᵣ(CR⁶R⁷)ₛ are present, then [r+s]≥1; and
A¹ through A¹¹ are optional amino acid residues, unsubstituted or substituted at any N atom, and when present are independently selected from alpha-, beta-, and gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid;
integers a through k are independently selected from 0, 1, and 2, and wherein when any of integers a through k is 0, then any of the two groups adjacent to a respective absent group (according to the integer 0 at said absent group) are connected to each other directly; and
when an optional group X is absent, then group R¹ is directly connected to one of groups A⁸, A⁹, A¹⁰, or A¹¹.

In one embodiment, provided is a compound of Formula I-P-1, Formula I-P-2, Formula I, Formula I-1, Formula I-2, or Formula II-P-1, wherein the integers a through g are each 1; and wherein A¹ is Thr or Ser; A², A³ A⁶, and A⁷ are independently selected from Dab, Dap, Ser, or Thr; A⁴ is Leu or Ile; and A⁵ is Phe, D-Phe, Bip, D-Bip, Val, and D-Val.

In another embodiment, the cyclic peptide structure in a compound of Formula I-P1, Formula I-P2, Formula I, Formula I-1, Formula 1-2, or Formula II-P-1, comprised of optional amino acid residues A¹ through A⁷, is a cyclic peptide structure identical to that present in polymyxin A, polymyxin B, polymyxin B nonapeptide (H-Thr-Dab-cyclo[Dab-Dab-D-Phe-Leu-Dab-Dab-Thr]), polymyxin B heptapeptide (H-cyclo[Dab-Dab-D-Phe-Leu-Dab-Dab-Thr]), polymyxin E, or octapeptin, or a similar structure.

In an additional embodiment, the integers a through g in a compound of Formula I-P1, Formula I-P2, Formula I, Formula I-1, Formula 1-2, or Formula II-P-1 are each 1.

In another embodiment, A¹ is Thr or Ser; A², A³ A⁶, and A⁷ are independently selected from Dab, Dap, Ser, and Thr; A¹⁴ is Leu or Ile; and A⁵ is Phe, D-Phe, Bip, D-Bip, Val, or D-Val.

In another aspect, provided is a compound of any of the Formulas I-P-1, I-P-2, Ia, Ib, Ic, I-1, I-2, II-P-1, II-P-2, III-P, III, or IV-P that exerts a therapeutic effect after administration into a mammal by releasing a bioactive agent HR¹.

In another aspect, provided is a compound of any of the Formulas I-P-1, I-P-2, Ia, Ib, Ic, I-1, I-2, II-P-1, II-P-2, II-P, III, or IV-P possessing anti-inflammatory activity, as determined by alleviating or slowing of kidney cytokine(s) release such as TNF-α, IL-6, IL-12, or one or more biomarkers such as blood urea nitrogen, and serum creatinine, or by testing of said compounds in animal models.

In another aspect, provided is a compound of any of the Formulas I-P-1, I-P-2, Ia, Ib, Ic, I-1, 1-2, II-P-1, II-P-2, III-P, III, or IV-P possessing anti-inflammatory activity, and wherein the inflammation is a renal inflammation, or an inflammation in a kidney injury, or an inflammation induced by a nephrotoxic substance, including a pharmaceutical nephrotoxic substance, such as anticancer, anti-infective, or another chemotherapeutic substance.

In another aspect, provided is a compound of any of the Formulas Ia, Ib, Ic, I-1, I-2, II-P-2, III-P, III, or IV-P possessing an enhanced anti-inflammatory, immunomodulating, or nephro-protective effect when compared to a similar dose of a free (unconjugated) agent or drug (H)ₙR¹ incorporated into said compound, as determined by in vitro or in vivo test(s) for anti-inflammatory, immunomodulating, or nephro-protective activity. In another aspect, provided is a compound of Formula I-P-1 I-P-2, or II-P-1, possessing an enhanced anti-inflammatory, immunomodulating, or nephro-protective effect when compared to a similar dose of a free (unconjugated) agent or drug (H)ₐR¹ and/or (H)ₒR² incorporated into said compound, as determined by in vitro or in vivo test(s) for anti-inflammatory, immunomodulating, or nephro-protective activity.

In yet another aspect, provided is a compound of any of the Formulas I-P-1, I-P-2, Ia, Ib, Ic, 1-1, 1-2, II-P-1, II-P-2, III-P, III, or IV-P, wherein when administered to a mammal, said compound exhibits preferential accumulation in kidneys, with a ratio for its molar concentration in kidneys compared to that in blood of between about 5 and 500.

In additional aspect, provided is a compound of any of the Formulas Ia, Ib, Ic, I-1, I-2, II-P-2, III-P, III, or IV-P, wherein when administered to a mammal at a dosing (expressed in molar amount) equal to a standard therapeutic dosing (in molar amount) of an agent HR¹, said compound exhibits about 1.5- to 15-fold higher loading (tissue concentration) and/or drug exposure (area-under-the-curve, AUC) of agent HR¹ in kidney, as compared to the standard therapeutic dosing of free (unconjugated) drug HR¹. In additional aspect, provided is a compound of Formulas I-P-1, I-P-2, or II-P-1, wherein when administered to a mammal at a dosing (expressed in molar amount) equal to a standard therapeutic dosing (in molar amount) of an agent HR¹ and/or (H)ₒR², said compound exhibits about 1.5- to 15-fold higher loading (tissue concentration) and/or drug exposure (area-under-the-curve, AUC) of agent HR¹ and/or (H)ₒR² in kidney, as compared to the standard therapeutic dosing of free (unconjugated) drug HR and/or (H)ₒR².

In additional aspect, provided is a compound of any of the Formulas I-1, I-2, Ia, Ib, Ic, II-P-2, III-P, III, or IV-P, wherein when administered to a mammal at a dosing (expressed in molar amount) equal to a standard therapeutic dosing (in molar amount) of agent HR¹, exhibits at least 2-fold higher efficacy, as compared to the standard therapeutic dosing of agent HR¹, with said therapeutic effect determined as a slowed, stopped, or reversed progression of inflammation or a kidney injury (as determined, for example, by levels of a cytokine release, and/or by using biochemical biomarkers for inflammation monitoring, or by radiography, magnetic resonance imaging, or alike). In additional aspect, provided is a compound of Formulas I-P-1, I-P-2, or II-P-1, wherein when administered to a mammal at a dosing (expressed in molar amount) equal to a standard therapeutic dosing (in molar amount) of agent HR¹ and/or (H)ₒR², exhibits at least 2-fold higher efficacy, as compared to the standard therapeutic dosing of agent HR¹ and/or (H)ₒR², with said therapeutic effect determined as a slowed, stopped, or reversed progression of inflammation or a kidney injury (as determined, for example, by levels of a cytokine release, and/or by using biochemical biomarkers for inflammation monitoring, or by radiography, magnetic resonance imaging, or alike).

In another aspect, provided is a compound of any of the Formulas Ia, Ib, Ic, I-1, I-2, II-P-2, III-P, II, or IV-P, wherein when administered to a mammal at a dosing (expressed in molar amount) equal to a standard therapeutic dosing (in molar amount) of an agent HR¹, exhibits at least 2-fold reduced rate of adverse effects and/or off-target toxicity manifestation, as compared to the standard therapeutic dosing of HR¹, as determined by medical observations of a mammal under therapy, a blood cells count, a tissue biopsy, and/or by analysis of biochemical biomarkers, or similar method. In another aspect, provided is a compound of any of the Formulas I-P-1, I-P-2, or 1-P-1, wherein when administered to a mammal at a dosing (expressed in molar amount) equal to a standard therapeutic dosing (in molar amount) of an agent HR¹ and/or (H)ₒR², exhibits at least 2-fold reduced rate of adverse effects and/or off-target toxicity manifestation, as compared to the standard therapeutic dosing of HR¹ and/or (H)ₒR², as determined by medical observations of a mammal under therapy, a blood cells count, a tissue biopsy, and/or by analysis of biochemical biomarkers, or similar method.

In one aspect, provided is a compound of any of the Formulas I-P-1, I-P-2, I, Ia, Ib, Ic, I-1, 1-2, II-P-1, II-P-2, III-P, III, or IV-P, for therapy of a renal inflammation, and wherein the inflammation is the chronic kidney disease (CKD), systemic lupus erythematosus (SLE), a nephritis, acute kidney injury (AKI), or an inflammation in a kidney transplant surgery.

In another aspect, provided is a method for the treatment of an inflammation-associated kidney disease in a mammal comprising administering to the mammal a therapeutically effective amount of a compound of any of the Formulas I-P-1, I-P-2, Ia, Ib, Ic, I-1, I-2, II-P-1, II-P-2, III-P, III, or IV-P.

In additional aspect, provided is a compound of any of the Formulas I-P-1, I-P-2, Ia, Ib, Ic, I-1, I-2, II-P-1, II-P-2, III-P, III, or IV-P, possessing an increased in vivo efficacy against an inflammation-associated kidney disease, when compared to a related (parent) anti-inflammatory, immunomodulating, or nephron-protective structure (compound) incorporated into said compound(s), as determined by in vivo test(s) in animal model(s) of such kidney disorders, wherein said compound and a related free drug (conjugated within a compound provided herein) are dosed at identical molar dose.

In additional aspect is provided a pharmaceutical composition comprising a compound of Formulas I-P-1, I-P-2, Ia, Ib, Ic, I-1, I-2, II-P-1, II-P-2, III-P, III, or IV-P, or a pharmaceutically acceptable salt, prodrug, solvate, or hydrate thereof, and a pharmaceutically acceptable carrier, excipient or diluent.

In an another aspect is provided a method for treating an inflammation-associated kidney disease in humans or other warm-blooded animals by administering to the subject in need a therapeutically effective amount of a compound of Formulas I-P-1, I-P-2, Ia, Ib, Ic, I-1, I-2, II-P-1, II-P-2, III-P, III, or IV-P or a pharmaceutically acceptable salt, prodrug, solvate, or hydrate thereof.

The compound of Formulas I-P-1, I-P-2, Ia, Ib, Ic, I-1, 1-2, II-P-1, II-P-2, III-P, III, or IV-P may be administered, for example, orally, parenterally, transdermally, topically, rectally, or intranasally, or via an intra-tumoral administration.

In yet another aspect is provided novel intermediates and processes for preparing compounds of Formulas I-P-1, I-P-2, Ia, Ib, Ic, I-1, I-2, II-P-1, II-P-2, III-P, III, or IV-P.

### BRIEF DESCRIPTION OF FIGURES

FIG. 1 shows the anti-inflammatory activity of the compounds of Examples 1 and 2 (per reduced CREA and BUN levels) as compared to dexamethasone comparator in a cisplatin-induced acute kidney injury mouse model. All exemplary compounds were dosed at 4 mg/kg and all doses were uncorrected for solvated water content. In FIG. 1, Cis is cisplatin; Dex is dexamethasone.

### DETAILED DESCRIPTION

Unless otherwise stated, the following terms used in the specification and Claims have the meanings given below.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₁₄ alkyl refers to alkyl of one to fourteen carbon atoms, inclusive.

The term alkyl refers to both straight and branched saturated hydrocarbon groups. Reference to an individual radical such as “propyl” embraces only the straight chain radical, and a branched chain isomer such as “isopropyl” being specifically referred to. Unless specified otherwise “alkyl” contains 1-12 carbon atoms. In addition to any group specifically recited in any of the embodiments or claims, the alkyl group is optionally substituted with one, two, three, or four substituents selected from the group consisting of halo, hydroxy, cyano, C₁₋₁₂ alkyl, C₃₋₇cycloalkyl, aryl, biaryl, heterocyclic, and heteroaryl (Het) group. In some embodiments, alkyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂-phenyl, biphenylmethyl, and the like. In some embodiments, alkyl is unsubstituted. Alkyl groups distinguished as “alkyl” and “alkyl” or “alkyl²” refer to independently selected alkyl groups that may be different from each other, or independently equal to each other. If the term “alkyl” is used more than once in the same group, then each “alkyl” is independent of another “alkyl,” at each appearance.

The term “Alk” refers to alkyl, as defined herein.

The term “alkylene” refers to a divalent alkyl group. Unless specified otherwise “alkylene” contains 1-12 carbon atoms. In some embodiments, “alkylene” is linear. The alkylene group is optionally substituted as described for alkyl. In some embodiments, alkylene is unsubstituted. Alkylene groups distinguished as “alkylene” and “alkylene¹” or “alkylene²” refer to independently selected alkylene groups that may be different from each other, or independently equal to each other.

The term alkenyl refers to both straight and branched hydrocarbon groups containing at least one double bond, and in some embodiments 1, 2, or 3 double bonds. Unless specified otherwise “alkenyl” contains 2-12 carbon atoms. In addition to any group specifically recited in any of the embodiments or claims, the alkenyl is optionally substituted with one, two, or three substituents selected from the group consisting of halo, C₁₋₁₂ alkyl, C₃₋₇cycloalkyl, aryl, biaryl, Het¹, and Het². In some embodiments, alkenyl is unsubstituted. In some embodiments, alkenyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂— phenyl, biphenylmethyl, and the like.

The term alkenylene refers to a divalent alkenyl group. Unless specified otherwise “alkenylene” contains 2-12 carbon atoms. The alkenylene group is optionally substituted as described for alkenyl. In some embodiments, the alkenylene group is unsubstituted.

The term “cycloalkyl” means a cyclic saturated, monovalent, monocyclic or bicyclic, saturated or unsaturated hydrocarbon group of three to 18 (in some embodiments, three to six) carbon atoms. In some embodiments, cycloalkyl includes but is not limited to cyclopropyl, cyclohexyl, cyclododecanoyl, and the like. In addition to any group specifically recited in any of the embodiments or claims, the cycloalkyl group is optionally substituted with one, two, or three substituents selected from the group consisting of halo, C₁₋₁₂ alkyl, C₃₋₇cycloalkyl, aryl, Het¹, Het², and heteroaryl. In some embodiments, cycloalkyl is unsubstituted.

The term “cycloalkylene” means a divalent cycloalkyl group. In addition to any group specifically recited in any of the embodiments or claims, the cycloalkylene group is optionally substituted as described for cycloalkyl. In some embodiments, the cycloalkylene is unsubstituted. In some or any embodiments, the C₃₋₆cycloalkylene group formed by any two of R⁵ through R¹⁰ is optionally substituted with one or two groups independently selected from C₁₋₆alkyl and aryl.

The term “heteroalkyl” means an alkyl or cycloalkyl group, as defined above, having a substituent containing a heteroatom selected from N, O, and S(O)ₙ, where n is an integer from 0 to 2, where in some embodiments the substituent includes, hydroxy (OH), C₁₋₁₄ alkoxy, amino, thio (—SH), and the like. Said heteroatom may be incorporated in any part of the heteroalkyl group [e.g., heteroalkyl can be C₁₋₄alkylC(═O)O C₃₋₆cycloalkylNH₂], or contain a heterocyclic substituent [e.g., heteroalkyl can be 2-(4-morpholino)ethyl]. In some embodiments, substituents include —NR^{a1}R^{b1}, —OR^{a1}, and —S(O)ₙR^{c1}, wherein each R^{a1} is independently hydrogen, C₁₋₄alkyl, C₃₋₆cycloalkyl, optionally substituted aryl, optionally substituted heterocyclic, or —C(O)R (where R is C₁₋₁₄alkyl); each R_{b1} is independently hydrogen, C₁₋₁₄ alkyl, —SO₂R (where R is C₁₋₄alkyl or C₁₋₄hydroxyalkyl), —SO₂NRR′ (where R and R′ are independently of each other hydrogen or C₁₋₄alkyl), or —CONR′R″ (where R′ and R″ are independently of each other hydrogen or C₁₋₄alkyl); n is an integer from 0 to 2; and each R^{c1} is independently hydrogen, C₁₋₄alkyl, C₃₋₆cycloalkyl, optionally substituted aryl, or NR^{a1}R^{b1} where R^{a1} and R^{b1} are as defined above. In some embodiments, heteroalkyl includes, but is not limited to 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to substituted or unsubstituted phenyl, biphenyl, triphenyl, or naphthyl. In addition to any group specifically recited in any of the embodiments or claims, the aryl is optionally substituted with 1 to 3 substituents independently selected from halo, —C₁₋₁₂alkyl (unsubstituted or substituted, in one embodiment with 1, 2, or 3 halo), aryl, —OH, —OC₁₋₁₂alkyl, —S(O)ₙC₁₋₄alkyl (wherein n is 0, 1, or 2), —C₁₋₄alkylNH₂, —NHC₁₋₄alkyl, —C(═O)H, C(═O)OR^{a1}, OC(═O)R^{a1}, OC(═O)NR^{a1}R^{c1}, OC(═O)heteroaryl, OC(═O)(heterocyclic ring) and —C═N—OR_{d} wherein R_{d} is hydrogen or —C₁₋₄alkyl. Two adjacent substituents in the aryl group may be connected to form a C₄₋₇cycloalkyl or 4- to 7-member heterocyclic group fused to said aryl group. Aryl groups distinguished as “aryl” and “aryl¹” or “aryl²” refer to independently selected aryl groups that may be different from each other, or independently equal to each other. If the term “aryl” is used more than once in the same group, then each “aryl” is independent of another “aryl,” at each appearance.

The term “arylalkyl” refers to an alkyl group substituted with an aryl group, each as defined herein, including where the aryl and alkyl are optionally substituted as described in their respective definitions.

The term “arylheteroaryl” refers to an aryl group substituted with a heteroaryl group, each as defined herein, including where the aryl and heteroaryl are optionally substituted as described in their respective definitions.

The term “heteroarylaryl” refers to a heteroaryl group substituted with an aryl group, each as defined herein, including where the aryl and heteroaryl are optionally substituted as described in their respective definitions.

The term “biaryl” refers to an aryl group as defined herein substituted with another aryl group as defined herein, including where the aryl groups are independently optionally substituted as described in the definition.

The term “biarylalkyl” refers to an alkyl group substituted with an aryl group which is substituted with another aryl group, each as defined herein, including where each aryl independently and alkyl are optionally substituted as described in their respective definitions

The terms “heterocyclic ring”, “heterocyclic ring” or “heterocycle” refer to a monocyclic or bicyclic aromatic ring or a saturated or unsaturated, monocyclic or bicyclic ring that is not aromatic comprising 3 to 12 carbon atoms and 1 to 4 heteroatoms independently selected from the group consisting of oxygen, nitrogen, P(═O), and S(O)ₘ within the ring, wherein m is an integer from 0 to 2. In addition to any group specifically recited in any of the embodiments or claims, the heterocyclic ring is optionally substituted with one, two, or three halo, C(═O)OR^{a1}, OC(═O)R^{a1}, OC(═O)NR^{a1}R^{b1}, —C₁₋₂₀alkyl, —OH, —NH₂, —OC₁₋₂₀alkyl, —S(O)ₘC₁₋₂₀alkyl (wherein m is 0, 1, or 2), —C₁₋₁₂alkyl-NH₂, —NHC₁₋₁₄alkyl, —C(═O)H, or —C═N—OR^{d1} wherein each R^{a1}, R^{b1} and R^{d1} is independently hydrogen or C₁₋₂₀alkyl. In some embodiments, the heterocyclic ring is unsubstituted. In some or any embodiments, the 4 to 7 or 5 to 7 membered ring formed by any two of R through R¹⁰ and/or formed by R⁶ and R⁸ is optionally substituted as described herein for heterocycle. In some or any embodiments, the 5 to 7 membered ring formed by R¹¹ and R¹² and/or formed by R⁴ and R¹¹ and/or formed by R⁶ and R¹² is optionally substituted with one or two groups independently selected from C₁₋₆alkyl and aryl.

In some embodiments, heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one, azabicyclo[3.1.0]hexane and the like, and N-oxides of said nitrogen heterocycles. In addition to any group specifically recited in any of the embodiments or claims, heterocyclic rings include substituted and unsubstituted rings, including those substituted with groups selected from C(═O)OR^{a1}, OC(═O)R^{a1}, OC(═O)NR^{a1}R^{b1} where each R^{a1} and R^{b1} are independently hydrogen or C₁₋₆alkyl.

The term “heteroaryl” refers to a five- (5) or six- (6) membered C- or N-linked heterocyclic ring, optionally fused to a benzene or to another heterocyclic ring (wherein at least one of the heterocyclic rings is aromatic). Heterocyclic ring fused to a benzene ring is also referred to as benzo-heterocyclic group. In some embodiments, heteroaryl includes, but is not limited to, pyridine, thiophene, furan, pyrazole, indole, benzimidazole, quinoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, and 1,2,4-dithiazolone. In addition to any group specifically recited in any of the embodiments or claims, heteroaryl groups include substituted and unsubstituted rings, including those substituted with groups selected from C(═O)OR^{a1}, OC(═O)R^{a1}, and OC(═O)NR^{a1}R^{b1} where each R^{a1} and R^{b1} are independently hydrogen or C₁₋₆alkyl. In some embodiments, heteroaryl is unsubstituted. Heteroaryl groups distinguished as ‘heteroaryl” and “heteroaryl¹” or “heteroaryl²” refer to independently selected heteroaryl groups that may be different from each other, or independently equal to each other. If the term “heteroaryl” is used more than once in the same group, then each “heteroaryl” is independent of another “heteroaryl,” at each appearance.

The term “heteroarylalkyl” refers to an alkyl group substituted with an heteroaryl group, each as defined herein.

Het¹ at each occurrence is independently a C-linked 5- or 6-membered heterocyclic ring having 1 to 4 heteroatoms selected from the group consisting of oxygen, nitrogen, and sulfur within the ring.

Het² at each occurrence is independently a N-linked 5 or 6 membered heterocyclic ring having 1 to 4 nitrogen and optionally having one oxygen or sulfur within the ring.

The term “mono-substituted” refers to a group having at least one substituent in said group, not counting the point of connection of this group to the main structure or general formula. The term “multiply-substituted” refers to a group having at least two substituents in said group, not counting the point of connection of this group to the main structure or general formula.

The term “unsaturated” in the context of the term cycloalkyl, cycloalkylene, and heterocycle refers to a partially unsaturated, but not aromatic ring.

Unless specified otherwise, “carbon atom” means the atom of element carbon optionally substituted with H, halo, NR^{a1}R^{b1}, C₁₋₁₂alkyl, C₃₋₇ cycloalkyl, aryl, heteroaryl, or with a heterocyclic ring. Carbon atom comprises atoms with sp3, sp2, and sp electronic hybridization.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds provided herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include all individual enantiomers and any mixtures, racemic, partially racemic, or otherwise, thereof. The methods for the determination of stereochemistry and the separation of stereoisomers are well-known in the art (see discussion in Chapter 4 of “Advanced Organic Chemistry,” 4th edition J. March, John Wiley and Sons, New York, 1992).

A hydrogen (H), carbon (C), or nitrogen (N) substitution for compounds of described herein include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application, or stability enhancement. Optionally, a compound described herein may incorporate a known in the art radioactive isotope or radioisotope, such as any number of ³H, ¹⁵O, ¹²C, or ¹³N isotopes, to afford a respective radiolabeled compound of those described herein.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating,” “treatment,” or “therapy” of a disease includes:
(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound that, when administered to a mammal for treating a disease, is sufficient to affect such treatment for the disease. The “therapeutically effective amount” will vary depending on the compound, the disease and its severity and the age, weight, etc., of the mammal to be treated.

“Leaving group” has the meaning conventionally associated with it in synthetic organic chemistry, i.e., an atom or group capable of being displaced by a nucleophile and includes halogen, C₁₋₄alkylsulfonyloxy, ester, or amino such as chloro, bromo, iodo, mesyloxy, tosyloxy, trifluorosulfonyloxy, methoxy, N,O-dimethylhydroxyl-amino, and the like.

“Prodrug” means any compound which releases an active parent drug according to a compound provided herein in vivo when such prodrug is administered to a mammalian subject. Prodrugs of a compound of provided herein are prepared by modifying functional groups present in a compound provided herein in such a way that the modifications may be cleaved in vivo to release the parent compound. Prodrugs include compounds provided herein wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl) of hydroxy functional groups in compounds provided herein, and the like. Prodrugs of compounds provided herein may be used for particular therapeutic application, such as for pulmonary delivery of an aerosol containing a prodrug of such a compound, or to improve tolerance to same agent. For example, a methanesulfonate prodrug form of polymyxin drug colistin (described, for example by Bergen et al. in Antimicrob. Agents Chemother. 2006, vol. 50, p. 1953) is used to reduce neurotoxic effects of colistin, and is used for aerosol administration of this drug. This and other known forms of prodrugs could be likewise used to further improve pharmaceutical properties of the compounds provided herein.

The term “mammal” refers to all mammals including humans, livestock, and companion animals.

The compounds described herein are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature).

### Illustrative Embodiments

Within the broadest definition, certain compounds of the compounds of Formulas I-P-1, I-P-2, Ia, Ib, Ic, I-1, I-2, II-P-1, II-P-2, III-P, III, or IV-P may be preferred. Specific and preferred values listed herein for radicals, substituents, and ranges, are for illustration only; they do not exclude other defined values or other values within defined ranges for the radicals and substituents.

In some preferred compounds described herein C₁₋₁₄alkyl can be methyl, ethyl, propyl, isopropyl, butyl, iso-butyl, sec-butyl, octyl, nonyl, decyl, and isomeric forms thereof.

In some preferred compounds described herein C₂₋₁₂alkenyl can be vinyl, propenyl, allyl, butenyl, and isomeric forms thereof (including cis and trans isomers).

In some preferred compounds described herein C₃₋₇cycloalkyl can be cyclopropyl, cyclobutyl, cyclopentyl, cyclohexyl, and isomeric forms thereof.

In some preferred compounds described herein C₁₋₁₄heteroalkyl can be hydroxymethyl, hydroxyethyl, 2-(N,N-dimethylamino)ethyl, 2-(4-morpholino)ethyl, and 2-methoxyethyl.

In some preferred compounds described herein halo can be fluoro (F) or chloro (Cl).

It will also be appreciated by those skilled in the art that compounds described herein may have additional chiral centers and be isolated in optically active and racemic forms. Included herein are any racemic, optically active, tautomeric, geometric, or stereoisomeric form, or mixture thereof, of a compound described herein.

Any embodiment described herein can be combined with any other embodiment described herein.

In one embodiment the compound of Formula I-P-1 is a compound of Formula II-P-1:

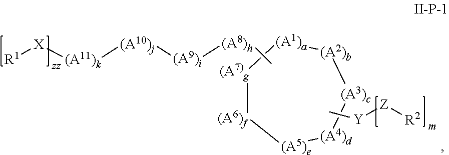

or a pharmaceutically acceptable salt, solvate, or hydrate thereof wherein:
R¹ and R² are optional groups, with at least one of the groups R¹ and R² being present in the Formula II-P-1; and
R¹ and R² are independently selected from alkyl, aryl, biaryl, heteroaryl, heteroarylaryl, and arylheteroaryl; or
R¹ and R² are residues independently attached to X and Z, respectively, by subtracting a single or multiple H atom(s) from the respective parent (precursor) structure(s) (H)ₙR¹ and (H)ₒR² at any one of the following optional H-containing group(s) independently selected from NH, OH, SH, C(═O)OH, CONH, SO₂NH, and S(═O)NH when present in (H)ₙR¹ and (H)ₒR²; and wherein
a) (H)ₙR¹ and (H)ₒR² are independently an anti-inflammatory, an immunomodulating, or a nephro-protective, or a compound possessing an activity or capable of inducing an activity that modulates gluococorticoid receptor (GR) activity, or reduces the levels of proteinuria/creatinine, or modulates a cytokine release, or otherwise exhibits a biological activity against inflammation-associated kidney diseases, or a kidney injury; and
b) (H)ₙR¹ and (H)ₒR² are selected from aldosterone, AN3485, beclomethasone, beclomethasone dipropionate, betamethasone, budesonide, budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, or valdecoxib; or a heteroaromatic vanin inhibitor (selected from compounds described in PCT WO 2020/114943 and US2020/0354338); or a heterocyclic TYK2 inhibitor (selected from compounds described in PCT WO2020/86616 and US2020/0354338); or INF-alpha inhibitor (selected from compounds described in PCT WO 2020/159904 and EP3917912); or a variant thereof; and
c) (H)ₙR¹ and (H)ₒR² are independently compound(s) effective for a kidney disease therapy; and
d) (H)ₙR¹ is a glucocorticoid structure connected to X at an O or N atom(s), with at least one of these atoms present within the structure (H)ₙR¹; and
when an optional group R¹ is absent, then the fragment R¹X is selected from R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is selected from R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂ heteroarylC(═O), heteroarylOC(═O), heteroarylNHC(═O), heteroaryl N(C₁₋₁₂alkyl)C(═O), heteroarylSO₂, and heteroarylNHSO₂, or wherein
integers n and o are independently selected from 0, 1, 2, 3, 4, 5, 6, and 7, such that [n+o]≥1; and
A¹ through A¹¹ are optional amino acid residues independently selected from unsubstituted or substituted at any N atom alpha-, beta-, or gamma-amino acids, Ala, Arg, Asn, Asp, Cys, Glu, Gin, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integer zz is 1 or 2;
integers a through k and m are independently selected from 0, 1, and 2; wherein
[m+zz]≥1; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, or heterocyclyl; or the group A⁸ is directly connected to the group Y; and
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O)N(R⁴)(C₁₋₁₂alkylene),
C(═O)(CR⁵R⁶)ₚ(CR⁷R⁸)ᵣ(CR⁹R¹⁰)ₛC(═O),
C(═O)(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ᵣ(CR⁹R¹⁰)ᵣP(═O)(OCR⁵R⁶)ₚ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)_{T}(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ᵣC(═O), C(═O)N(R⁴)(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ᵣC(═O),
C(═O)OCR⁵═CR⁷—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ᵣNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ᵣNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ₛOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₚC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ₛOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ₛOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)(CR⁷R⁸)ₛN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O);
or any variant of above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein
R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁴ and R⁵, ii) R⁶ and R⁷, iii) R⁴ and R⁶, and iv) R⁹ and R⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein
when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and wherein
when fragments (CR⁵R⁶)ₚ(CR⁷R⁸)ᵣ or (OCR⁵R⁶)ₚ(CR⁷R⁸)ᵣ are present, then [p+r]≥1; and wherein
when fragments (CR⁷R⁸)(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³, with the following structures:
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ᵣC(═O),
C(═O)O(CR⁵R⁶)O(CR⁷R⁸)(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷)ₛC(═O),
P(═O)(OH)CF₂, P(═O)(OH)CF₂(CR⁷R⁸)ᵣC(═O),
C(═O)(CR⁷R⁸)—(CR⁹R¹⁰)ₛP(═O)(NHCR⁵R⁶
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰))ₛN(R⁴)C(═O),
C(═O)CR⁵═CR⁷—(CR⁹R¹⁰)ₛC(═O),
C(═O)OCR⁵═CR⁷—(CR⁹R¹⁰)ₚC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)N(R⁴)SO₂C(═O),
C(═O)(CR⁵R⁶)ₚS—S(CR⁷R⁸)(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ᵣC(═O),
C(═O)(CR⁵R⁶)ₚS—S(CR⁷R⁸)(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ᵣC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ᵣC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)(CR⁹R¹⁰)ₛN(R⁴)C(═O)
C(═O)CR⁵═CR⁷—S—S—(CR⁹R¹⁰)ₚC(═O),
C(═O)OCR⁵═CR—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)N(R⁴)C(═O),
C(═O)N[(CR⁵R⁶)ₚ(CR⁷R⁸)ᵣNHC(═O)A¹⁴](CR⁹R¹⁰)ₚC(═O),
C(═O)N[(CR⁵R⁶)ₚ(CR⁷R⁸)ᵣNHC(═O)A¹⁴](CR⁹R¹⁰)ᵣNHC(═O),
C(═O)N[(CR⁵R⁶)ₚ(CR⁷R⁸)ᵣNHC(═O)A¹⁴](CR⁷R⁸)ₛOC(═O),
C(═O)N[(CR⁵R⁶)ₚ(CR⁷R⁸)ᵣN(C₁₋₆alkyl)C(═O)A¹⁴](CR⁹R¹⁰)ᵣNHC(═O),
C(═O)N[(CR⁵R⁶)(CR⁷R⁸)ₛN(C₁₋₆alkyl)C(═O)A¹⁴](CR⁹R¹⁰)ₛC(═O),
C(═O)N[(CR⁵R⁶)(CR⁷R⁸)ₛN(C₁₋₆alkyl)C(═O)A¹⁴](CR⁹R¹⁰)ᵣNHC(═O),
C(═O)N[(CR⁵R⁶)(CR⁷R⁸)ₛN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵C](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ₛOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂OOCH₂C(═O),
C(═O)N{CH₂CH₂NHC(═O)—[(S)—CH(NH₂)]CH₂CH}CHCH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}HCH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂OOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ᵣC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O);
or any variant of the above X groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N (C₁₋₁₄alkyl)(CH₂)ₚO(CH₂)ᵣO(CH₂)ₛN(C₁₋₁₄alkyl)C(═O), and similar linear groups;
optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆ alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)C(CR⁷R⁸)ᵣ(CR⁹R¹⁰)ₛP(═O) (NHCR⁵R⁶)ₚ,
C(═O)O(CR⁵R⁶)ₚ(CR⁷R⁸)ᵣ(CR⁹R¹⁰)ᵣC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁷R⁸))ₚC(═O),
C(═O)OCR⁵═CR⁷—(CR⁹R¹⁰)ᵣC(═O)ₛC═O)N(R⁴)CR⁵═CR⁷—(CR⁹R¹⁰)ᵣC(═O),
C(═O)CR⁵═CR⁷—(CR⁹R¹⁰)ₛOC(═O)OC(═O)CR⁵═CR⁷—(CR⁹R¹⁰)N(R⁴)C(═O),
C(═O)N(R⁴)SO₂(CR⁵R⁶)ₚ(CR⁷R⁸))(CR⁹R¹⁰)ᵣC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ᵣS—S(CR⁷R⁸)(CR⁹R¹⁰)ₛC(═O),
C(═O)O(CR⁵R⁶)ₚS—S(CR⁷R⁸)(CR)ᵣC(═O), C(═O)N(R⁴)(CR⁵R⁶)ₚ(CR⁹R¹⁰)ᵣ(CR⁹R¹⁰)ᵣC(═O),
C(═O)(CR⁵R⁶)ₚS—S(CR⁷R⁸)₄CR⁹R¹⁰)ₛOC(═O), C(═O)(CR⁵R⁶)ₚ(CR⁷R⁸)₄CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC)C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)(CR⁷R⁸)ᵣNHC(═O)A¹⁴](CR⁹R¹⁰)ₛOC(═O),
C(═O)N[(CR⁵R⁶)(CR⁷R⁸)ₛN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)(CR⁷R⁸)ₛN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ᵣC(═O),
C(═O)N[(CR⁵R⁶)ₚ(CR⁷R⁸)ᵣNHC(═O)A¹⁴A¹⁵](CR⁹R¹⁰)N(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)(CR⁷R⁸)ₛOC(═O)A¹⁴A¹⁵](CR⁹R¹⁰)ₛC(═O),
C(═O)N[(CR⁵R⁶)(CR⁷R⁸)ᵣOC(═O)A¹⁴A¹⁵](CR⁹R¹⁰)N(C═O),
C(═O)N[(CR⁵R⁶)ₚ(CR⁷R⁸))ₛOC(═O)A¹⁴A¹⁵](CR⁹R¹⁰)(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)(CR⁷R⁸)ₛN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N {CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A′)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A′)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ᵣC(═O),
C(═O)N[CH₂CH₂NHC(═O)CH₂CH₂CH(NH₂)COOH](CR⁵R⁶)ₚ OC(═O)(CR⁷R⁸)ᵣ (CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O);
or any variant of above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein R⁵-R¹⁰ are as defined above; or
alternatively, the optional group Z is comprised of the following structures, optionally connected to one to two amino acid residue(s) A¹² or A¹³, with the following structures:
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR²R′)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ᵣNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ᵣNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C=o),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ₛOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N {CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A′)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ₛC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣ OC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚ OC(═O)(CR⁷R⁸)ᵣ (CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O); or any variant of the above Z groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and
wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸.

In an alternative embodiment of Formula II-P-1, each optional divalent group X is selected from N(R⁴)C(═O)(C₁₋₁₂alkylene),
(C₁₋₁₂alkylene)N(R⁴)C(═O), C(═O)N(R⁴)(C₁₋₁₂alkylene),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)N(R⁴)CR⁵═CR⁷—(CR⁹R¹⁰)ₛC(═O),
C(═O)CR⁵═CR⁷—(CR⁹R¹⁰)ₛN(R⁴)C(═O),
C(═O)(CR⁵R⁶)ₚ(CR⁷R⁸)ᵣ(CR⁹R¹⁰)ₛN(R⁴)SO₂C(═O),
C(═O)N(R⁴)(CR⁵R⁶)ₚ(CR⁷R⁸)ᵣ(CR⁹R¹⁰)ᵣC(═O),
C(═O)(CR⁵R⁶)ₚ(CR⁷R⁸)ᵣ(CR⁹R¹⁰)ₛN(R⁴)C(═O),
C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)C(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O), and any variant of the above X groups formed by the repositioning, addition, or deletion of the fragment N(R⁴)C(═O) or C(═O)N(R⁴)SO₂.

In an alternative embodiment of Formula II-P-1, each optional divalent group X is selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR²R′)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)₅N(R⁴)C(═O), and any variant of the above X groups formed by the repositioning, addition, or deletion of the fragment N(R⁴)C(═O) or C(═O)N(R⁴)SO₂;
wherein the group X is optionally connected to one to two amino acid residue(s) A¹² or A¹³.

In an alternative embodiment of Formula II-P-1, each optional divalent group X is S(CR⁵R⁶)ₚ(CR⁷R⁸)ᵣ(CR⁹R¹⁰)ₛOC(═O) or a group selected from

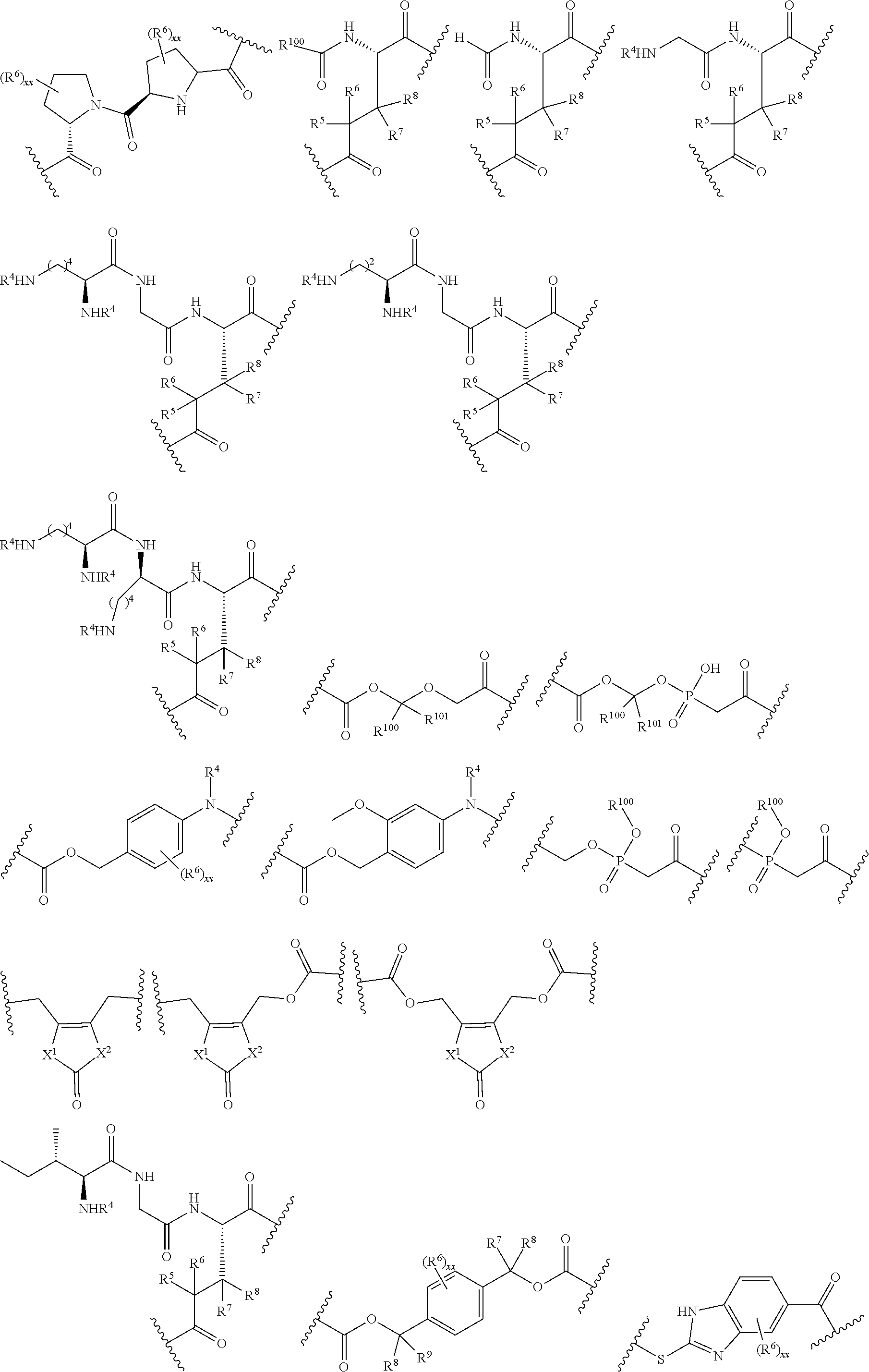

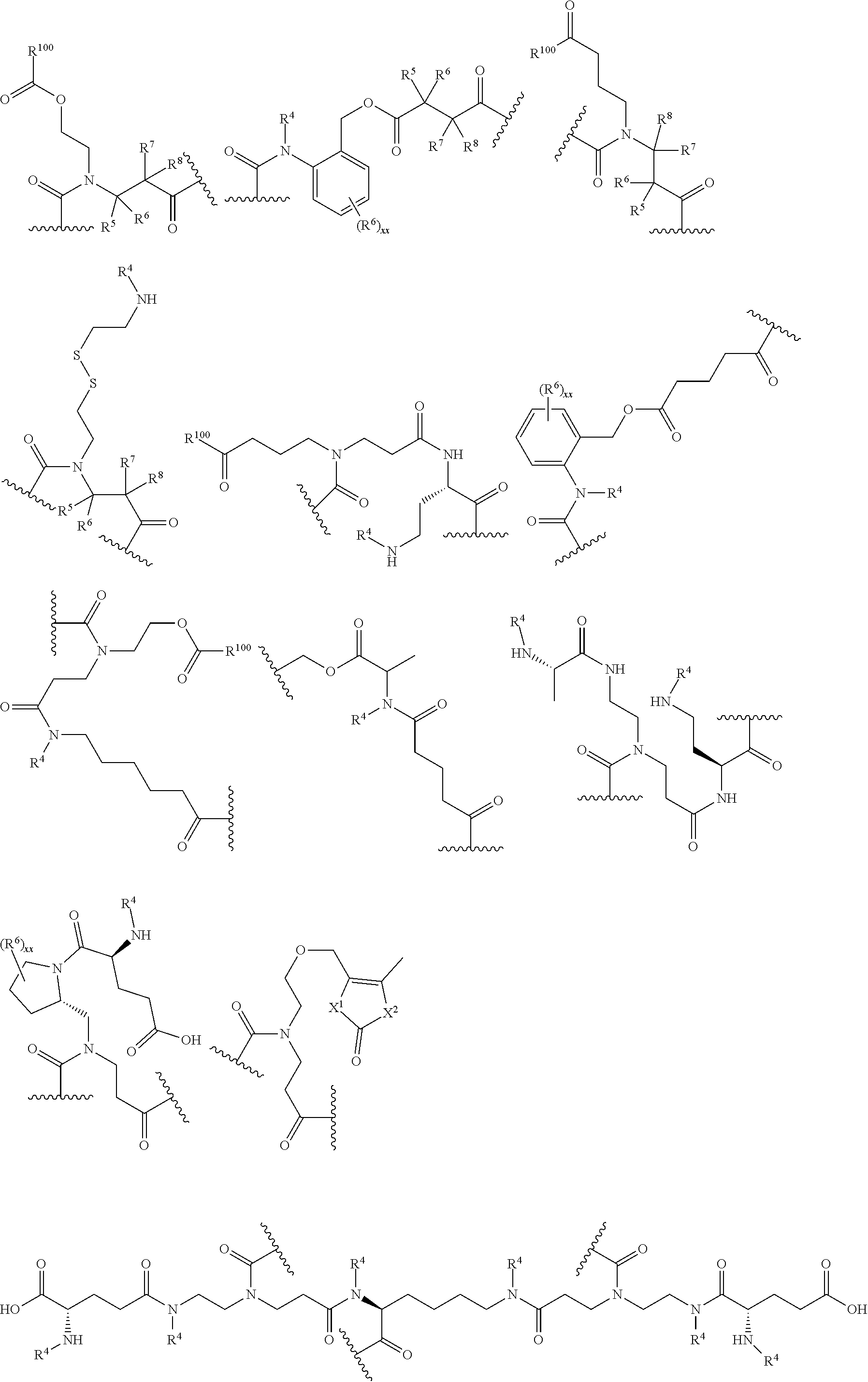

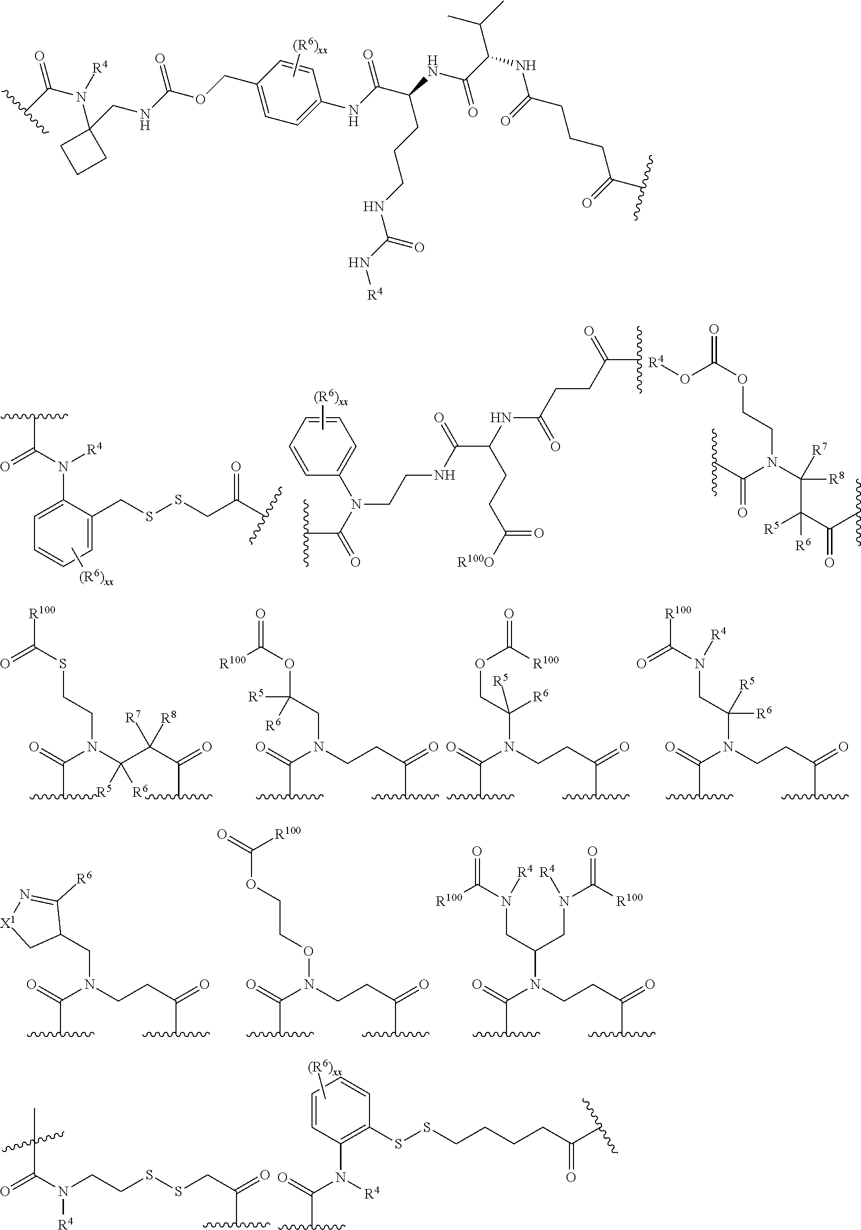

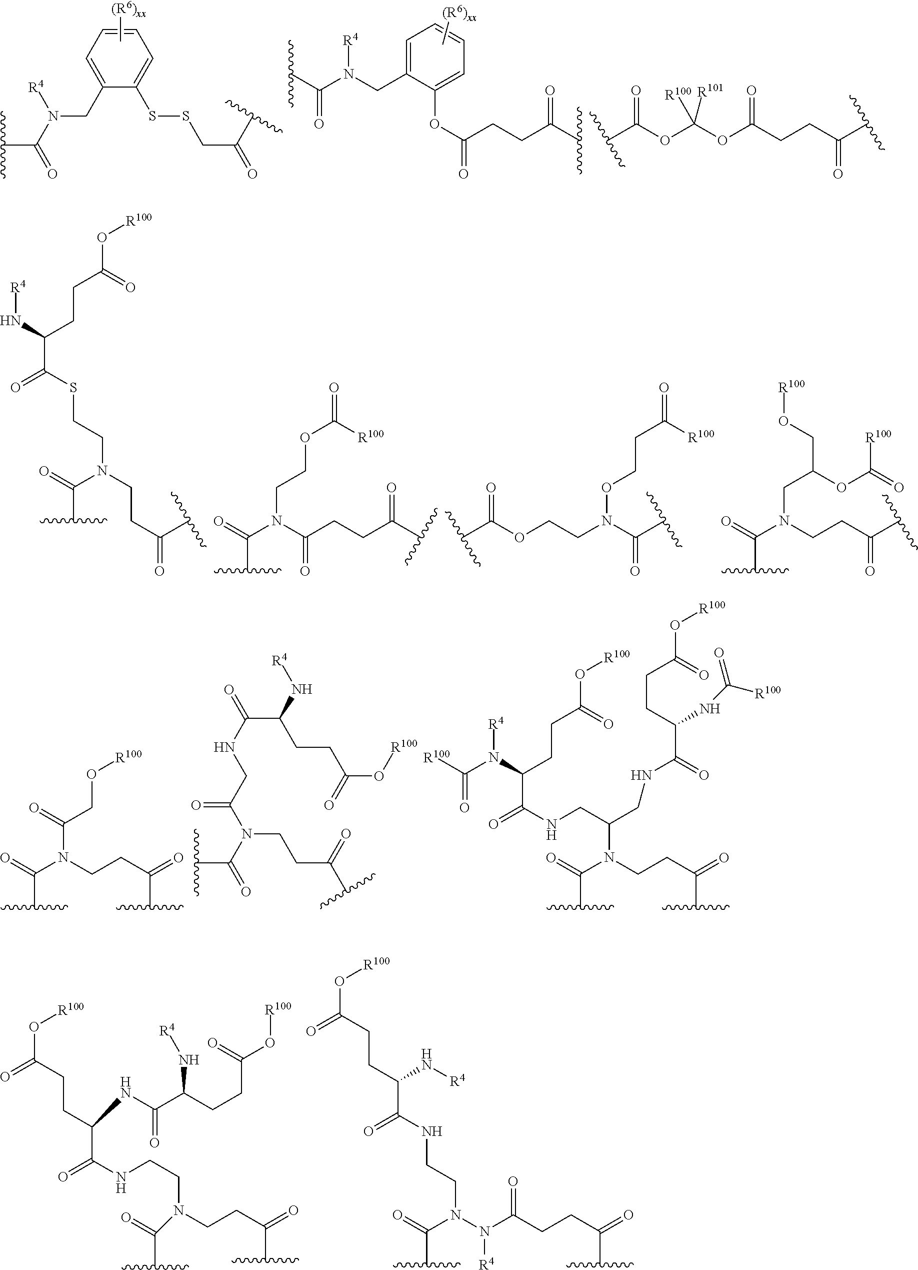

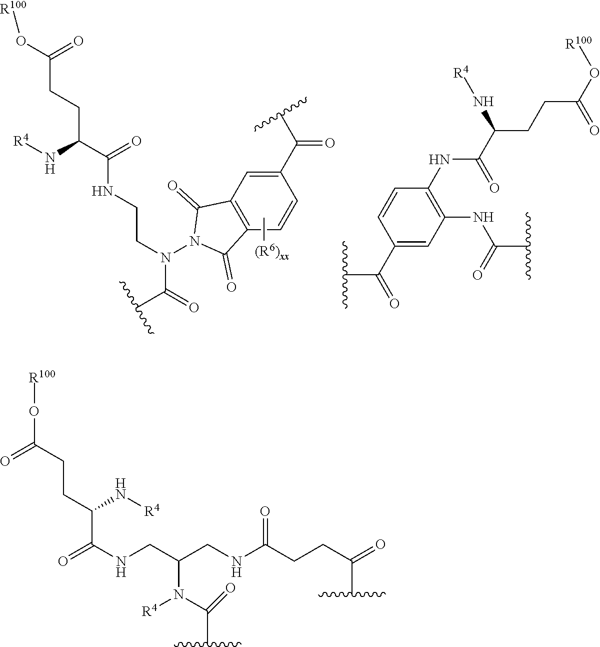

and any variant of the above X groups formed by the repositioning, addition, or deletion of the fragment N(R⁴)C(═O); or
wherein the group X is optionally connected to one to two amino acid residue(s) A¹² or A¹³;
wherein xx is 1, 2, or 3;
R¹⁰⁰ and R¹⁰¹ are independently selected from hydrogen and alkyl;
X¹ and X² are independently selected from 0, NH, and CR⁵R⁶; and
all other variables are as defined herein in Formula II-P-1.

In an alternative embodiment of Formula II-P-1, each optional divalent group Y and Z are independently selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR²R′)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), and any variant of the above groups formed by the repositioning, addition, or deletion of the fragment N(R⁴)C(═O) or C(═O)N(R⁴)SO₂.

In an alternative embodiment of Formula II-P-1, each optional divalent group Z is independently selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)₅C(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), and any variant of the above groups formed by the repositioning, addition, or deletion of the fragment N(R⁴)C(═O) or C(═O)N(R⁴)SO₂;
wherein the group Z is optionally connected to one to two amino acid residue(s) A¹² or A¹³.

In one embodiment is provided a compound of Formula I-P-1 of the following Formula II-P-2

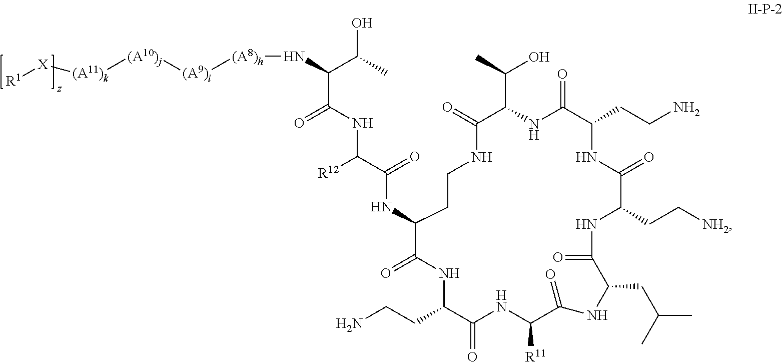

or a pharmaceutically acceptable salt, solvate, or hydrate thereof, wherein
R¹¹ is CH₂CH(CH₃)₂ or CH₂Ph; and R¹² is CH₂NH₂ or CH₂CH₂NH₂; and wherein other groups and integers in the compound of Formula II-P-2 are selected as defined above for a compound of Formula I-P-1.

In one embodiment of Formula II-P-2, R¹¹ is CH₂Ph; R¹² is CH₂NH₂ or CH₂CH₂NH₂; and, z is 1. In one embodiment, R¹² is hydrogen.

In one embodiment is provided a compound of Formula I-2 of the following Formula III:

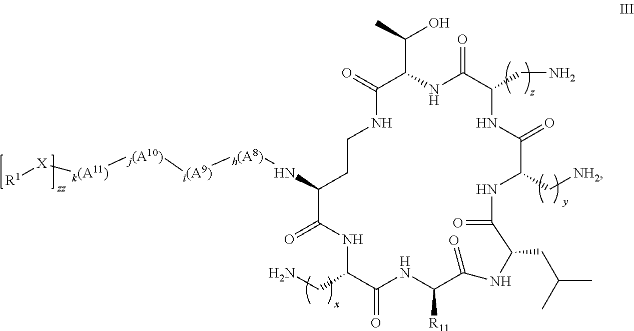

or a pharmaceutically acceptable salt, solvate, or hydrate thereof wherein:
integer x, y, z are independently selected from 1, 2 and 3;
integer zz is 1 or 2; and
R¹¹ is CH₂CH(CH₃)₂ or CH₂Ph.

In one embodiment, the compound of Formula 1-2 is a compound of Formula Ia:

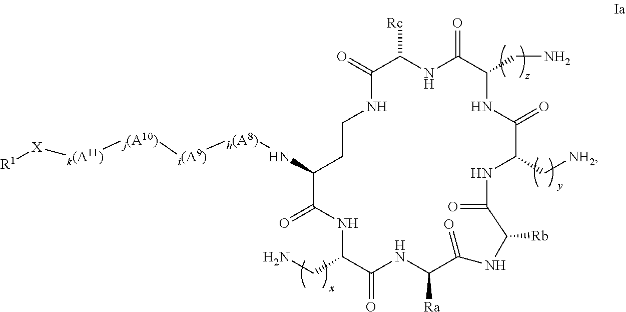

or a pharmaceutically acceptable salt, solvate, or hydrate thereof wherein
integers x, y, and z are independently selected from 1, 2 and 3;
Rₐ represents the side chain of an amino acid selected from D-leucine, D-isoleucine, D-phenylalanine, D-norleucine, D-valine, D-norvaline or D-t-butylglycine; and
R_{b} and R_{c} represent the side chain of an amino acid independently selected from serine, threonine, leucine, phenylalanine, norleucine, norvaline or t-butylglycine.

In another embodiment, the compound of Formula I-2 is a compound of Formula Ib:

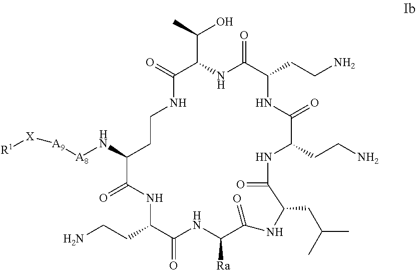

or a pharmaceutically acceptable salt, solvate, or hydrate thereof, wherein:
A⁸ is 2,3-diaminopropionic acid (Dap) or 2,4-diaminobutyric acid (Dab);
A⁹ is Thr, Glu, or Lys; and,
Rₐ is CH₂CH(CH₃)₂ or CH₂Ph.

In another embodiment, the compound of Formula I-2 is a compound of Formula Ic:

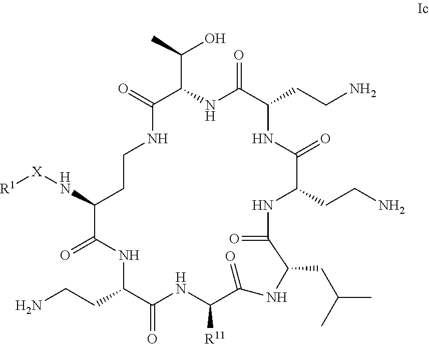

or a pharmaceutically acceptable salt, solvate, or hydrate thereof, wherein:
R¹¹ is CH₂CH(CH₃)₂ or CH₂Ph.

One preferred group of compounds of the Formulas I-P-1, I-P-2, I-1, I-2, Ia, Ib, Ic, II-P-1, II-P-2, III, or IV-P is illustrated below, wherein each X is independently selected from the structures below, wherein either the left side or the right side of X depicted below is connected to R¹.

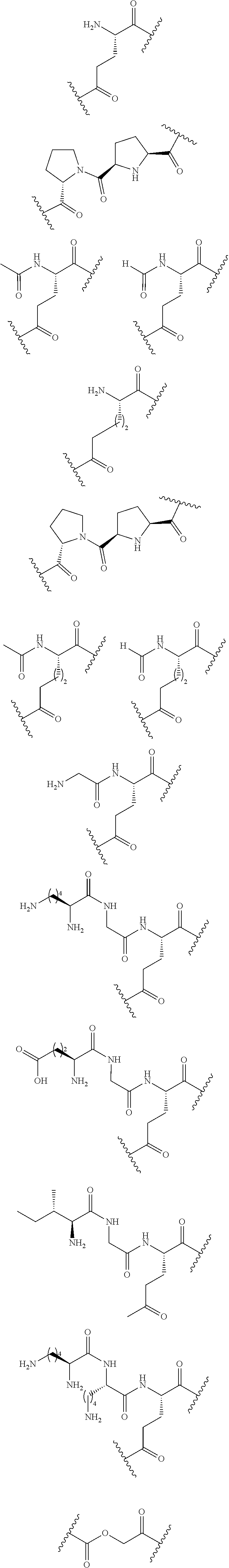

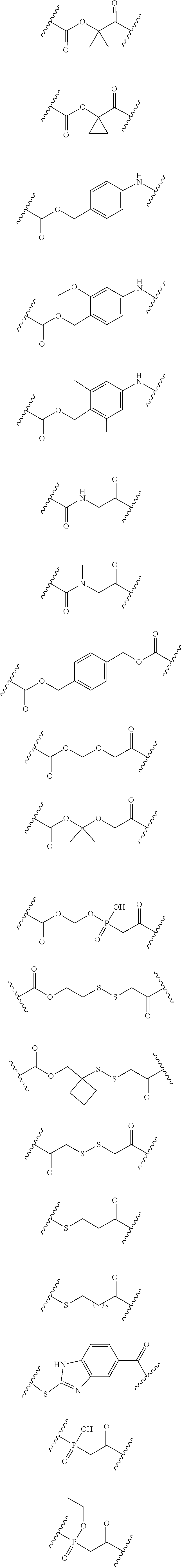

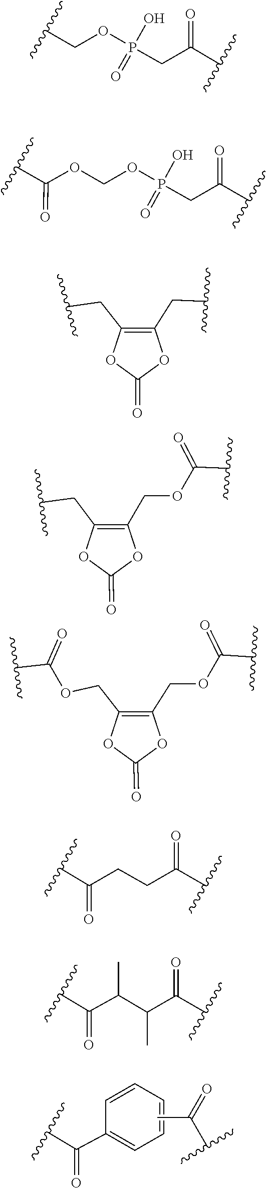

An alternative preferred group of compounds of the Formulas I-P-1, I-P-2, I-1, I-2, I, Ia, Ib, Ic, II-P-1, II-P-2, III, or IV-P is illustrated below, wherein each X is independently selected from the structures below, wherein either the left side or the right side of X depicted below is connected to R

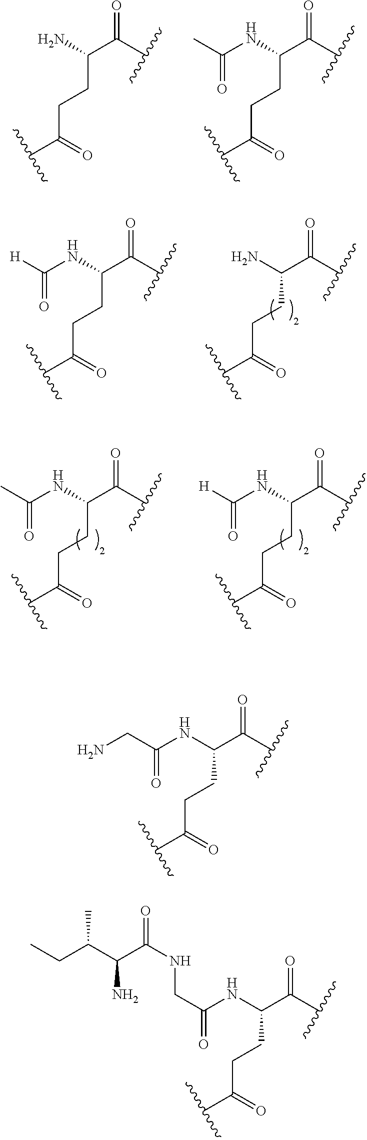

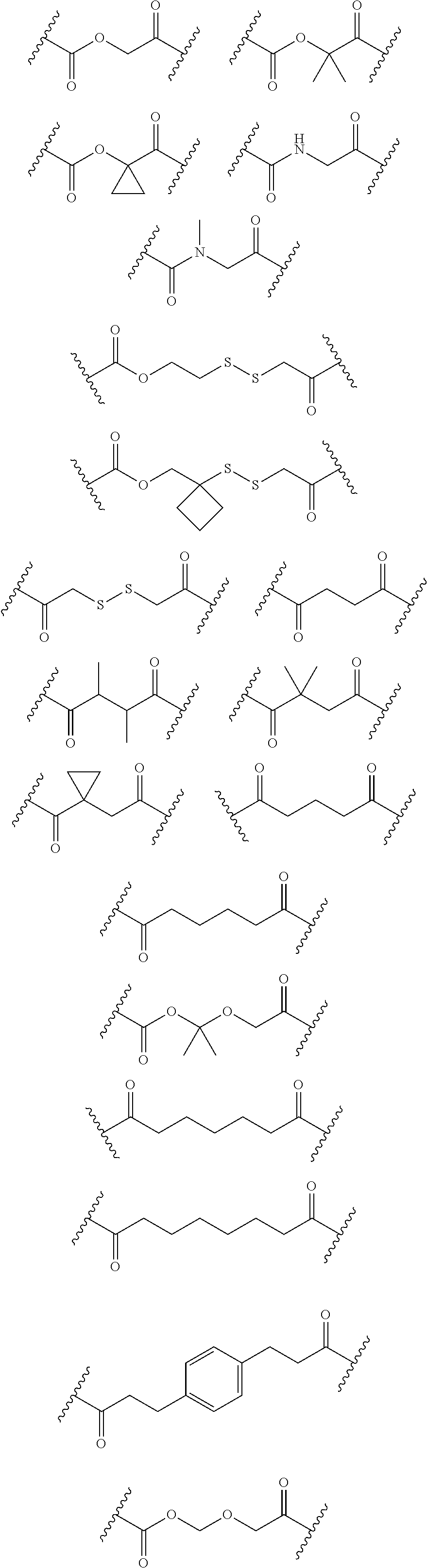

Another preferred embodiment of the Formulas I-P-1, I-P-2, I-1, I-2, Ia, Ib, Ic, II-P-1, II-P-2, III, or IV-P is illustrated below, wherein each X is independently selected from the following structures, connected to R¹ at the left side or the right side of group X therein,

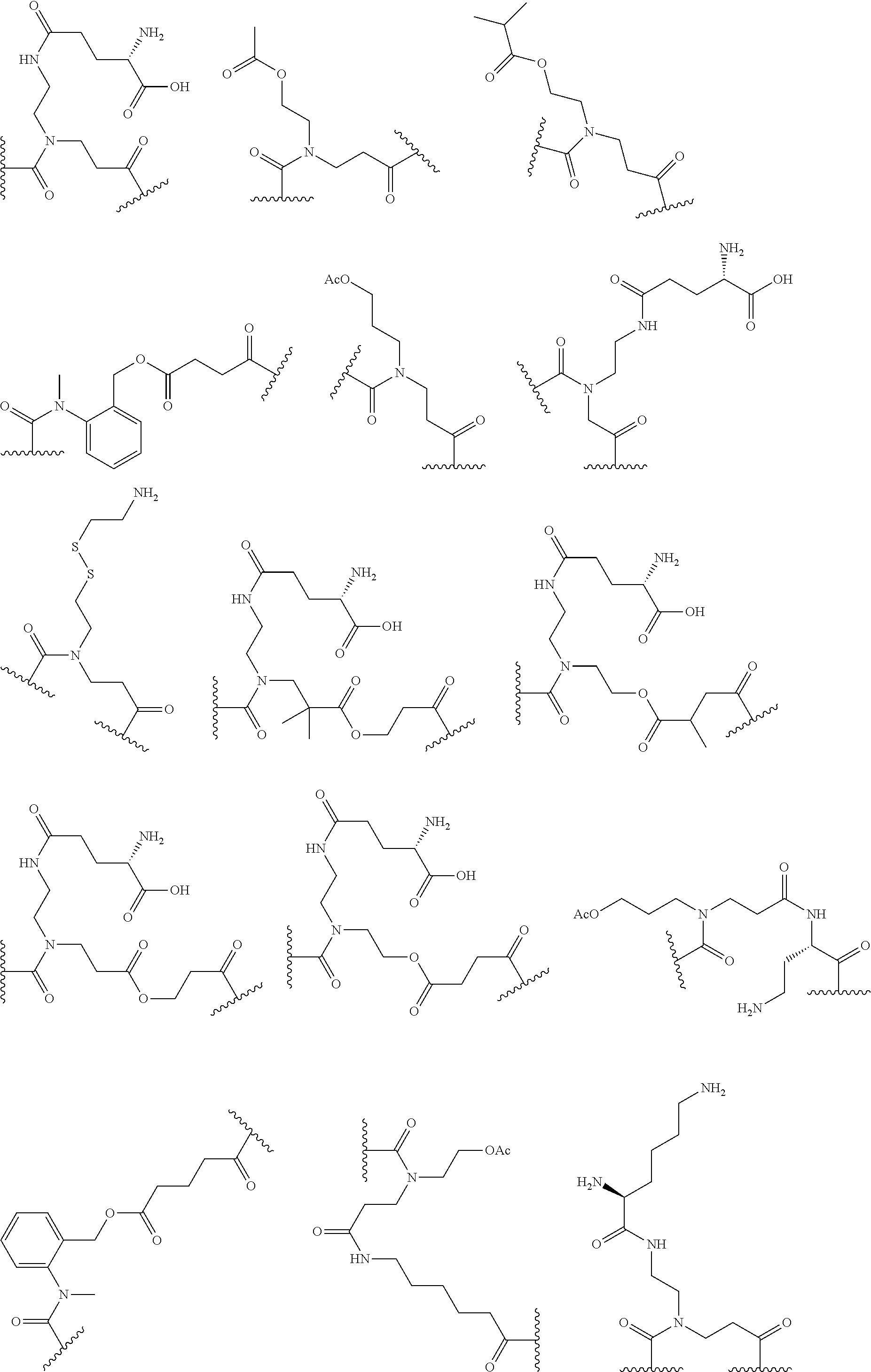

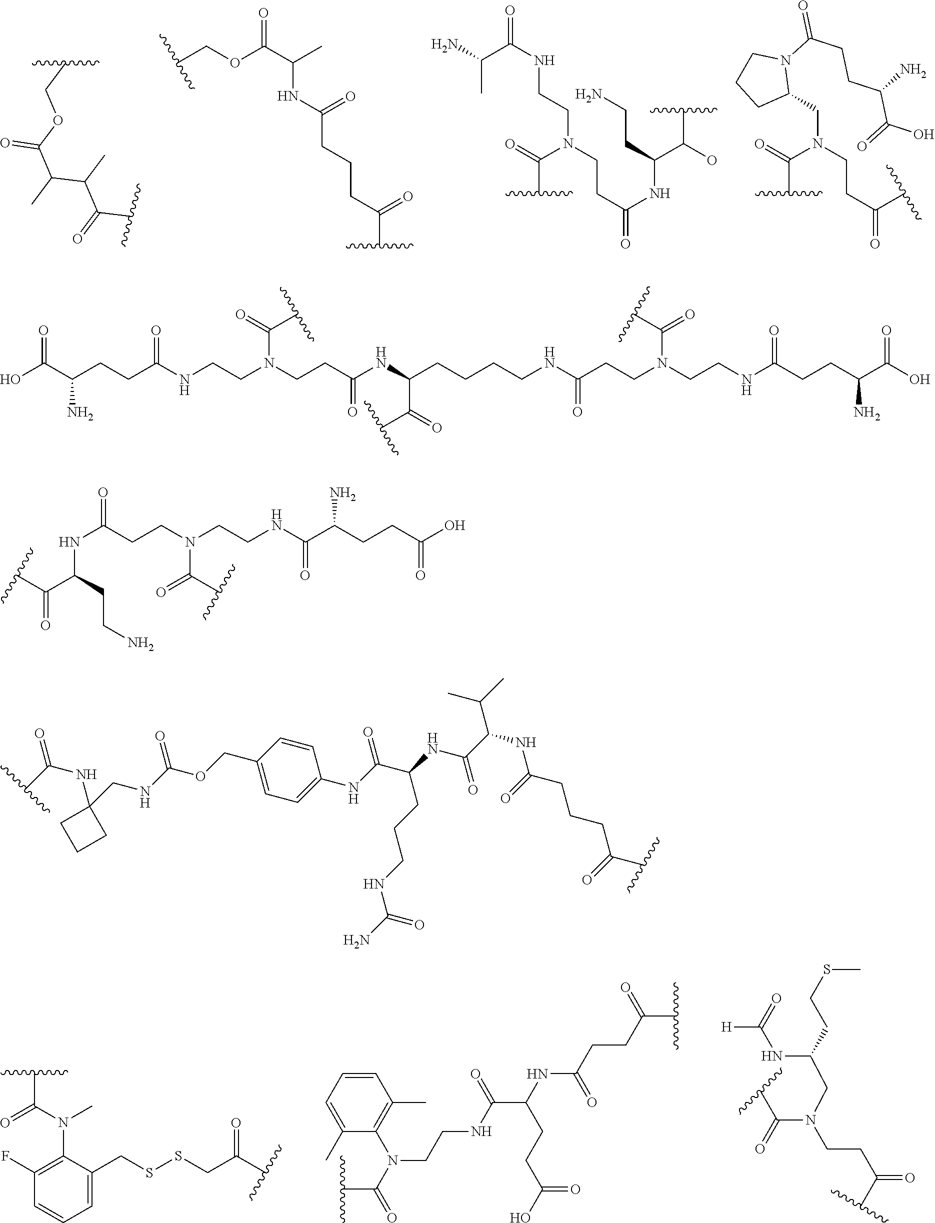

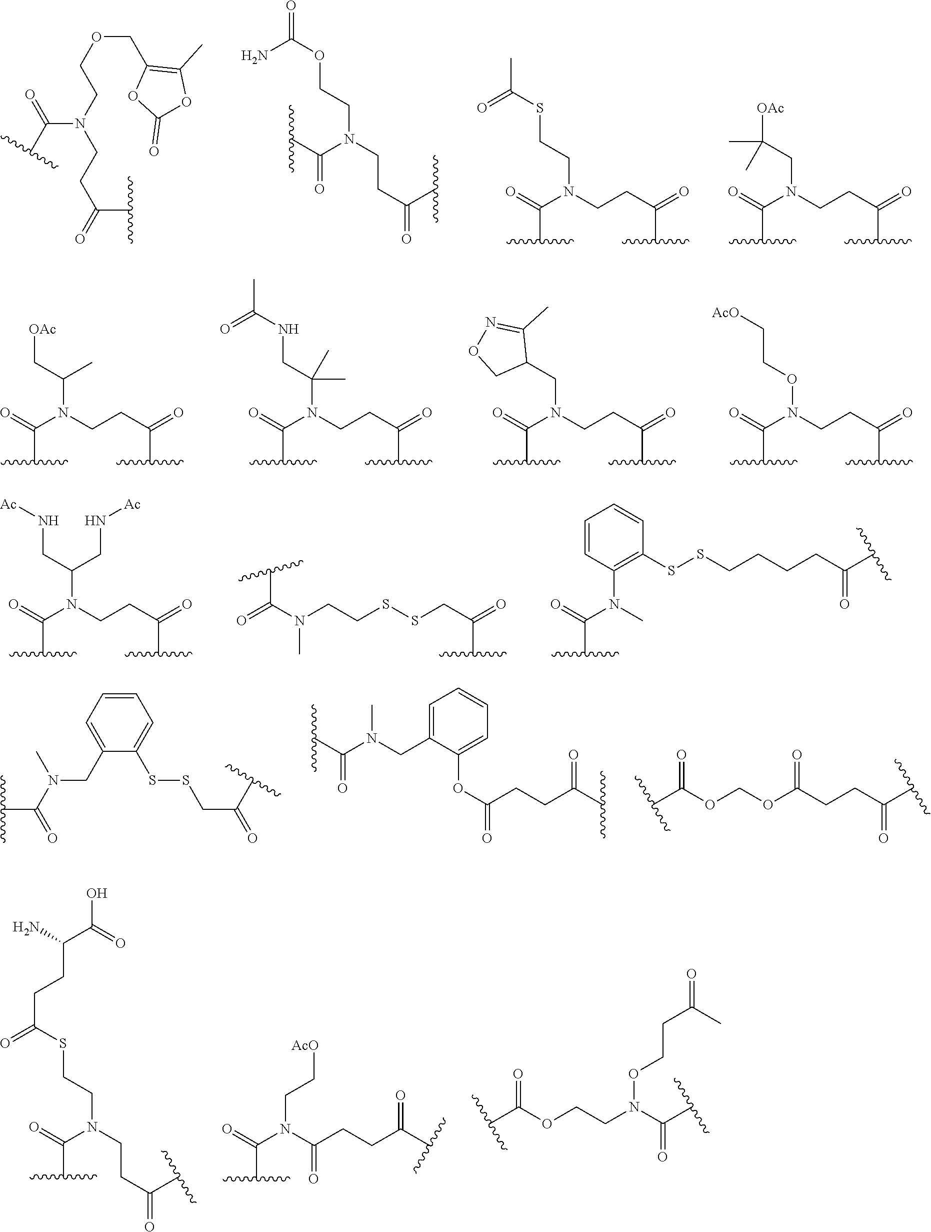

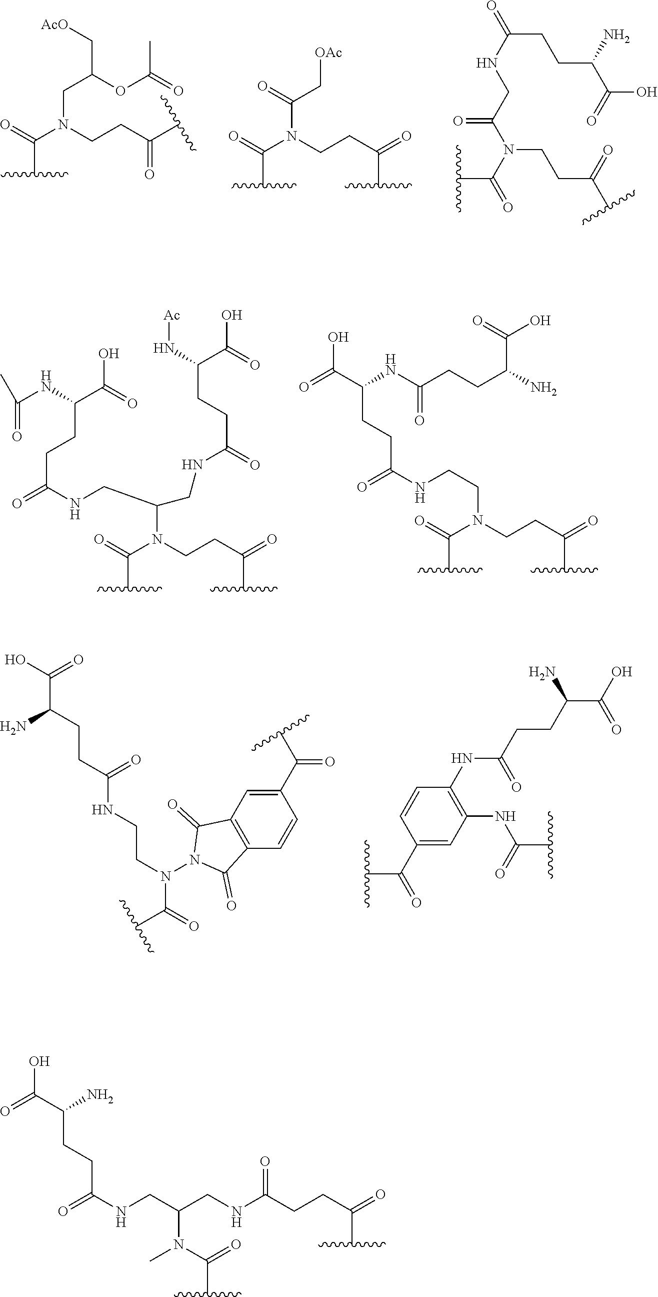

An alternative preferred embodiment of the Formulas I-P-1, I-P-2, I-1, I-2, Ia, Ib, Ic, II-P-1, II-P-2, III, or IV-P is illustrated below, wherein each X is independently selected from the following structures, connected to R at the left side or the right side of group X therein,

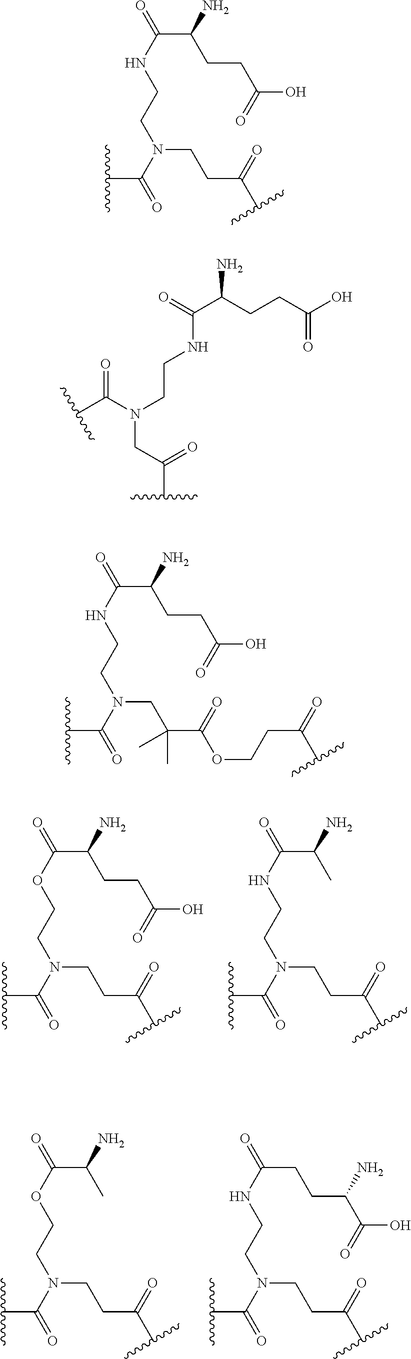

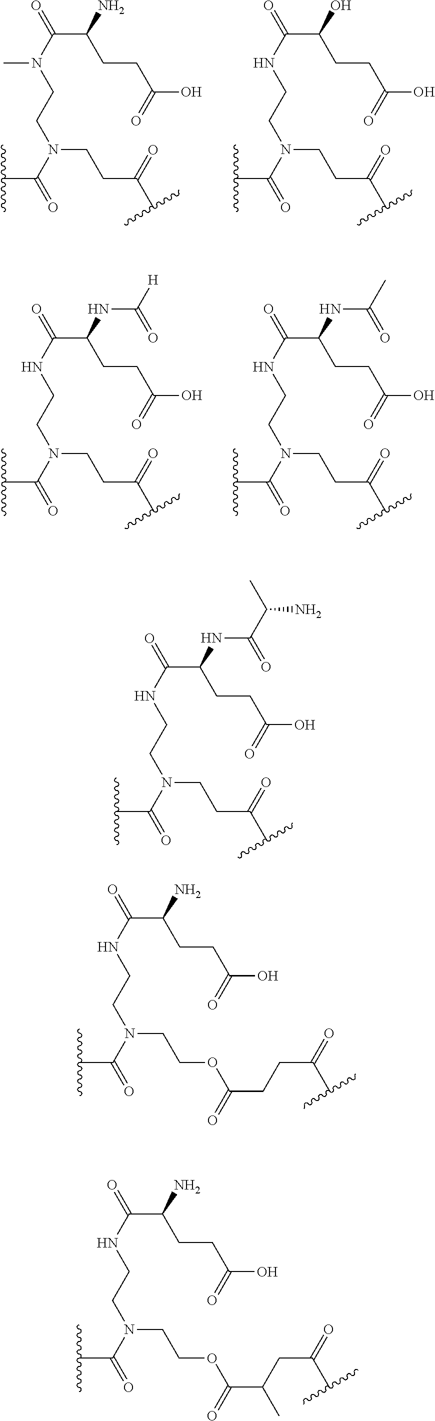

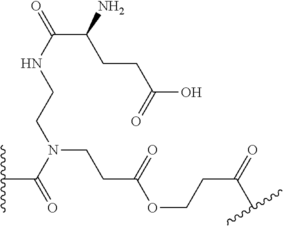 [SMILES: *C(=O)CCOC(=O)CCN(CCNC(=O)[C@@H](N)CCC(=O)O)C(*)=O]

In an alternative preferred embodiment, groups X illustrated above, either at its left or right side therein, incorporate additional divalent groups selected from C₁₋₁₂alkylene, C₂₋₁₂)alkenylene, C₂₋₁₂alkynylene, (CH₂)(CH₂)ᵣO(CH₂)ₛC(═O), (CH₂)ₚO(CH₂)ᵣO(CH₂)OC(O), (CH₂)ₚO(CH₂)ᵣ(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)C(═O), O(CH₂)ₚO(CH₂)ᵣO(CH₂)OC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), or alike linear groups.

In another embodiment, the compound of Formula I-P-1 is a compound of Formula III-P:

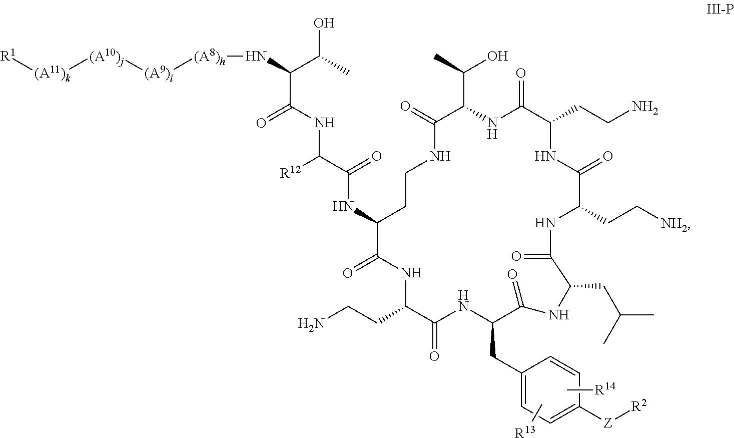

or a pharmaceutically acceptable salt, solvate, or hydrate thereof, wherein R¹³ and R¹⁴ are independently selected from H, halo, NH₂, CN, OH, OC₁₋₁₄alkyl, O-aryl, NH(C₁₋₆alkyl), NH(OC₁₋₆alkyl), C₁₋₁₂alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄alkyl, and wherein other groups and integers in the compound of Formula III-P are selected as defined above for the compound of Formula I-P-1.

An alternative preferred group of compounds of the Formulas I-P-1, I-P-2, I-1, I-2, Ia, Ib, Ic, II-P-1, II-P-2, or III-P is that wherein group Z is selected from the structures below, wherein the right side of group Z depicted below is connected to R².

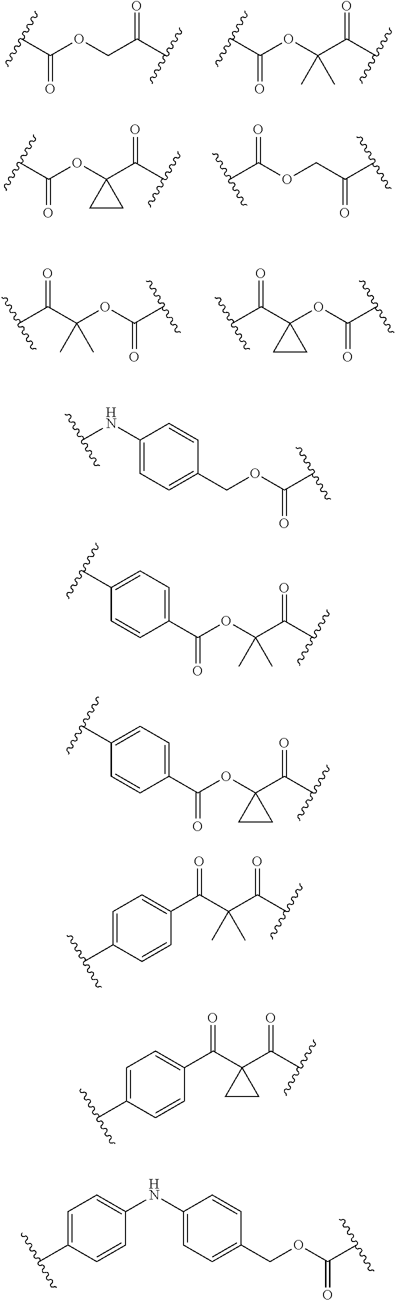

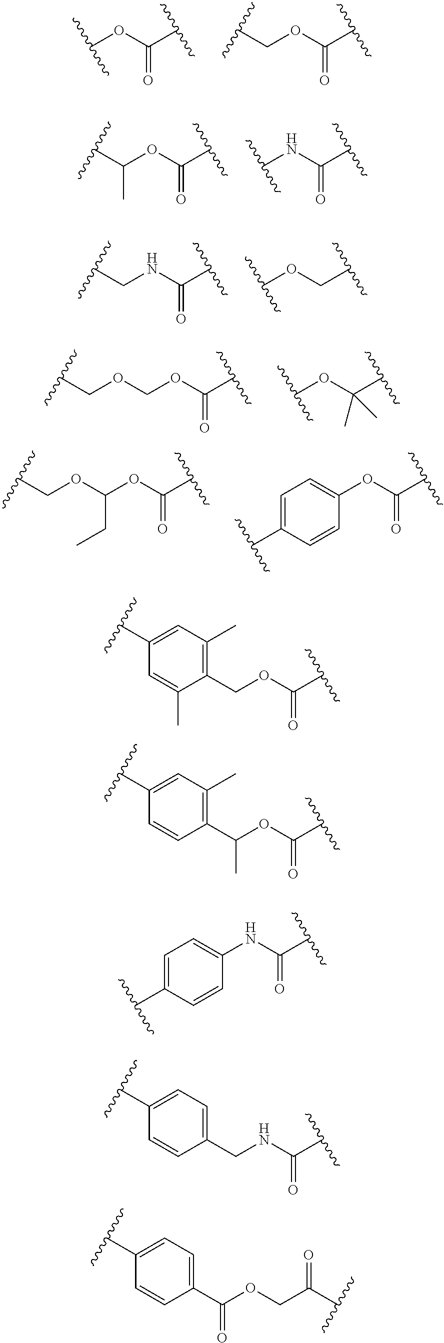

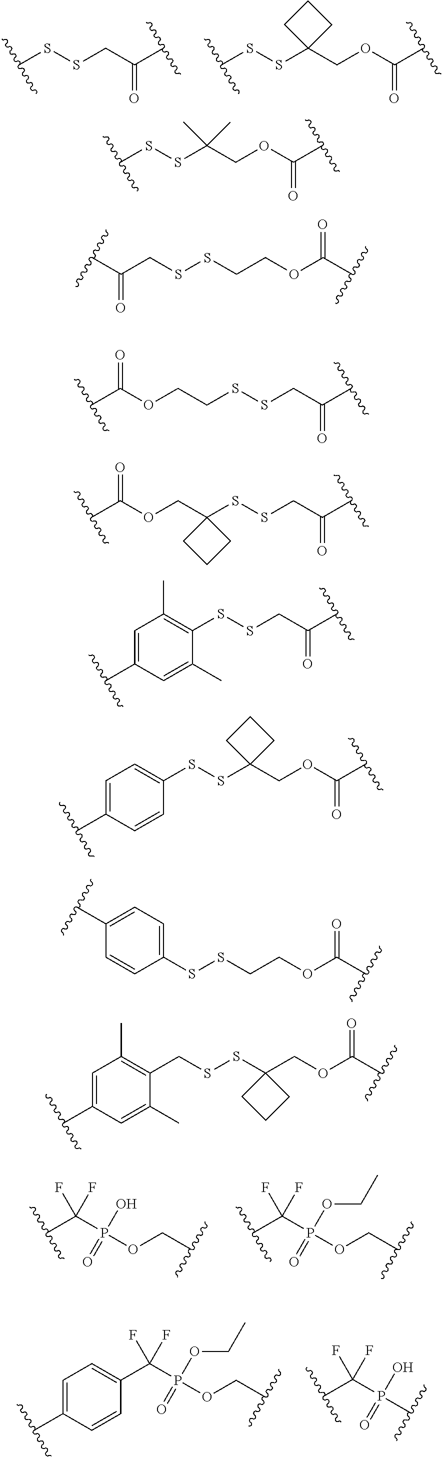

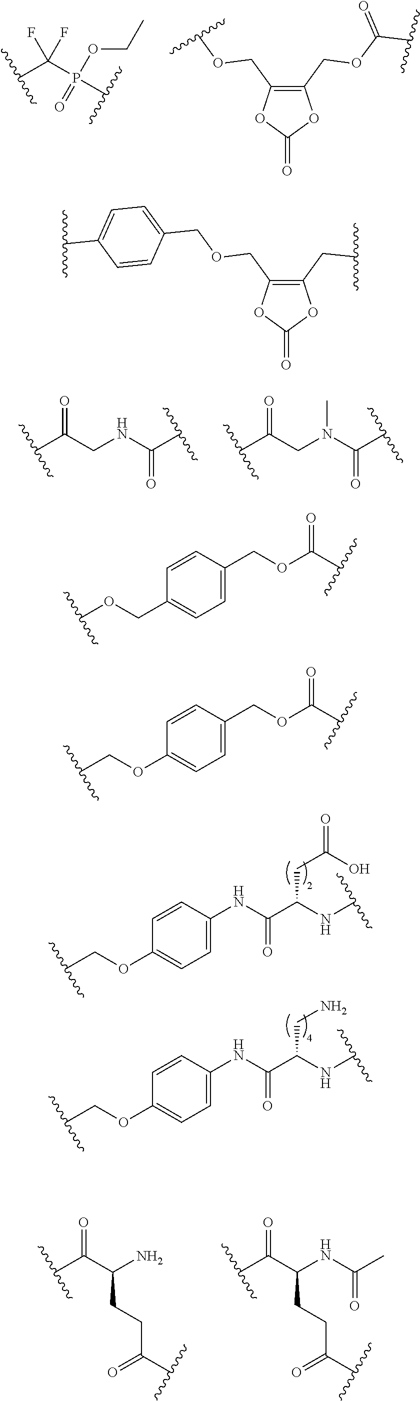

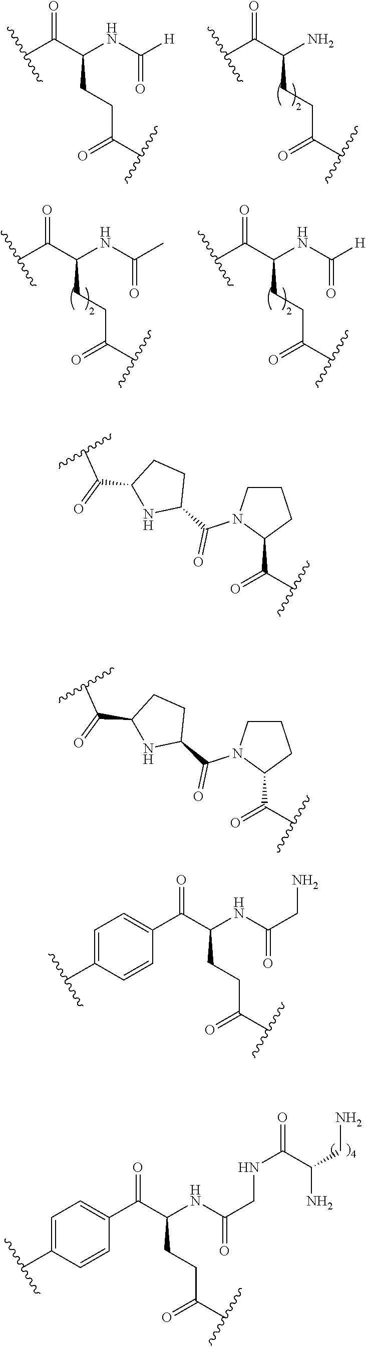

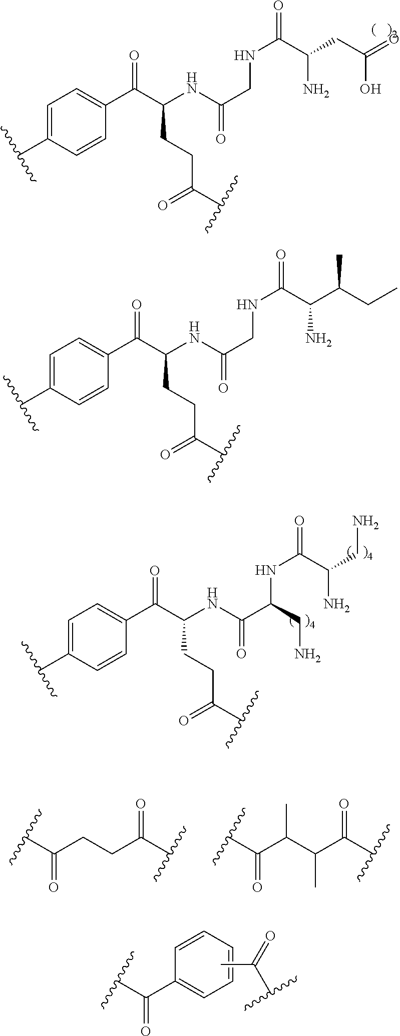

In another alternative preferred embodiment, a Z group illustrated above, at its left side therein, incorporate additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ᵣNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups.

In another embodiment, the compound of Formula I-P-1 is a compound of Formula IV-P:

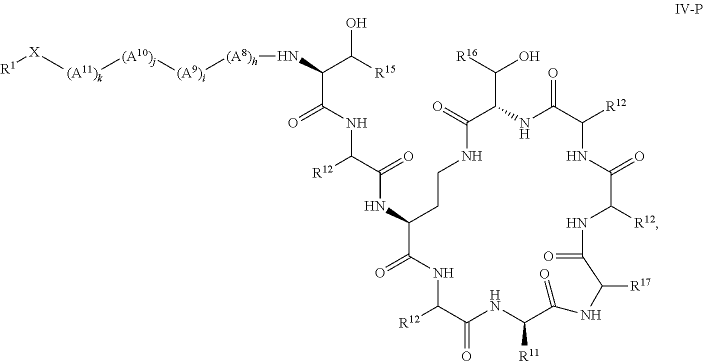

or a pharmaceutically acceptable salt, solvate, or hydrate thereof, wherein
X is selected from the following structures and is connected to R at the left side of X:
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)₁)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰))ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁹R¹⁰)(CR⁷R¹)ᵣNHC(═O) (CR⁹R¹⁰)ₛNCH(NH₂)COOH]CH₂CH₂C(═O),
C(═O)N[(CR⁵R⁶)ₚ(CR⁷R⁸)N(C₁₋₆alkyl)C(═O)(CR⁹R⁴)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O),
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O);
R¹¹ is C₁₋₁₂alkyl, CH(CH₃)₂, CH₂aryl, or CH₂Ph;
R¹² is CH₂NH₂, CH₂CH₂NH₂, or CH₂CH₂CH₂CH₂NH₂;
R¹⁵, R¹⁷ and R¹⁷ are independently H, Me, or C₁₋₁₂alkyl; and
wherein other groups and integers in the compound of Formula IV-P are selected as defined above for the compounds of Formula I-P-1. In one embodiment of Formula IV-P, R¹² is hydrogen.

Non-limiting examples of R¹ include:

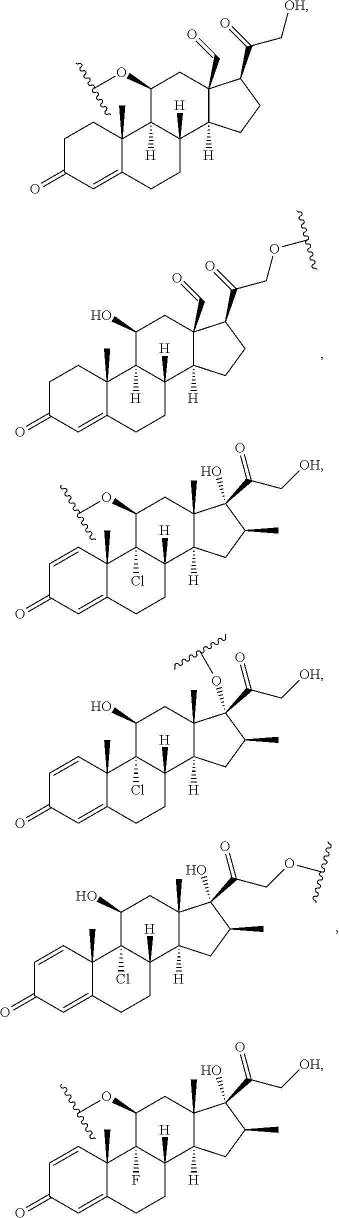

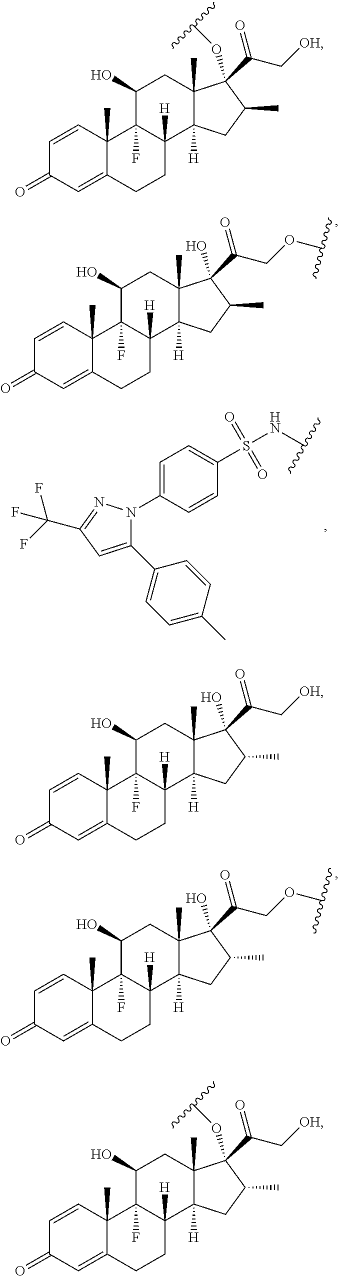

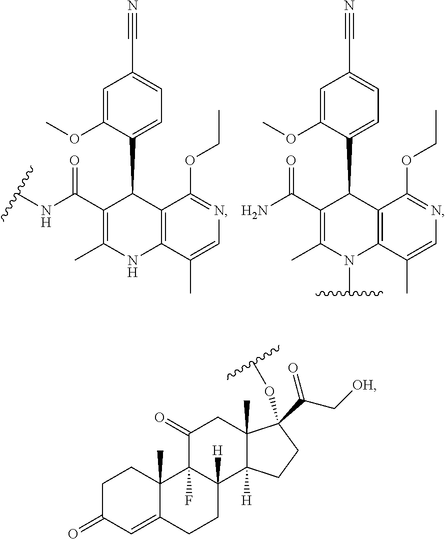

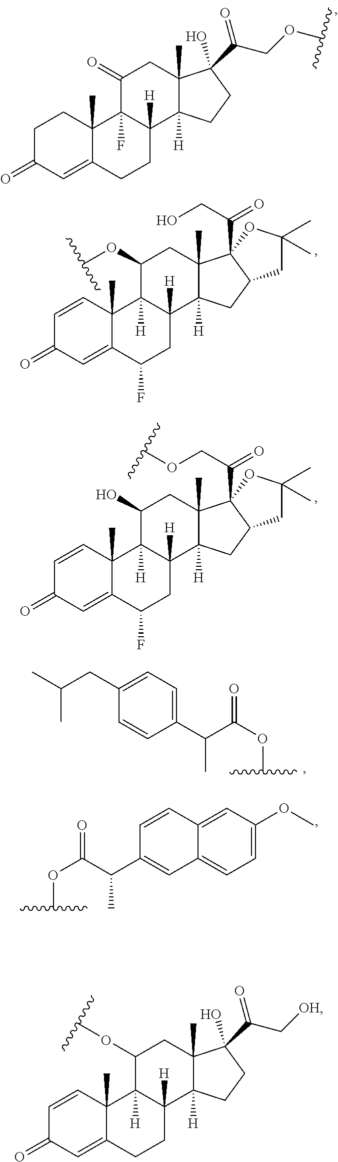

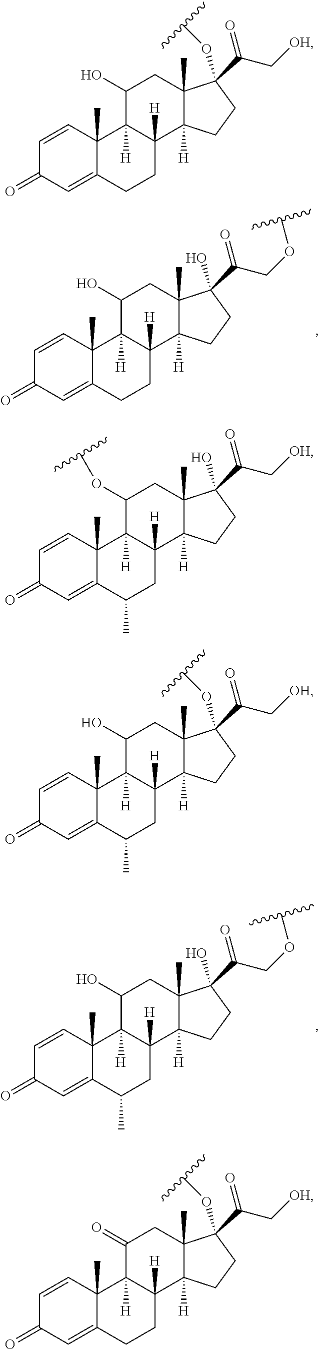

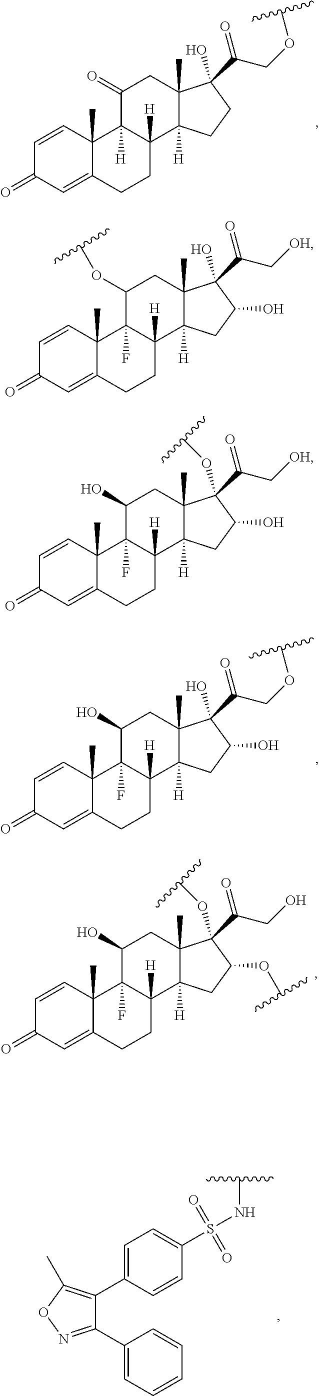

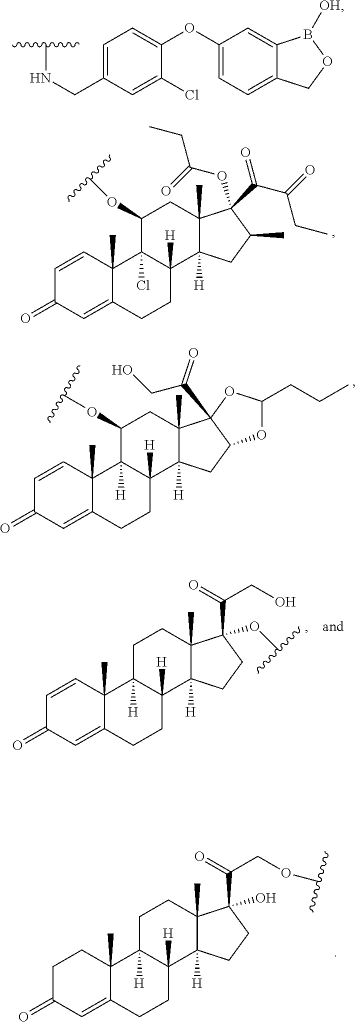

In one preferred embodiment, group R¹ in a compound of Formula IV-P is derived from subtraction of H from an OH group in structure R¹(H) wherein R¹(H) is selected from the structures below:

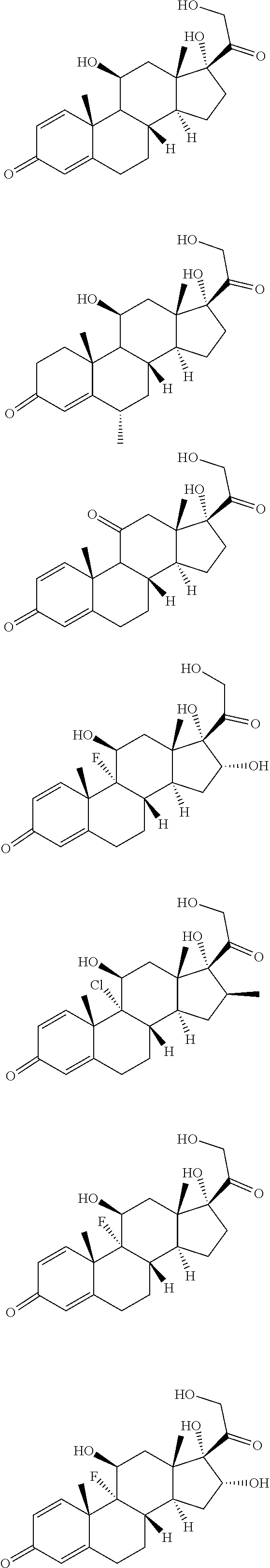

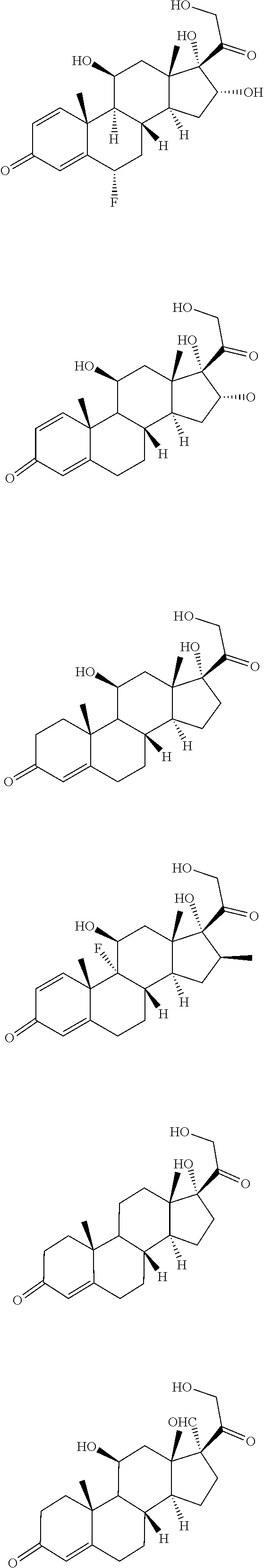

In an alternative preferred embodiment, group R¹ in a compound of Formula I-P1, I-P-2, I-1, 1-2, Ia, Ib, Ic, II-P-1, II-P-2, III-P, III, or IV-P is derived from subtraction of a H from an OH group in structure HR¹ wherein HR¹ is selected from the structures below:

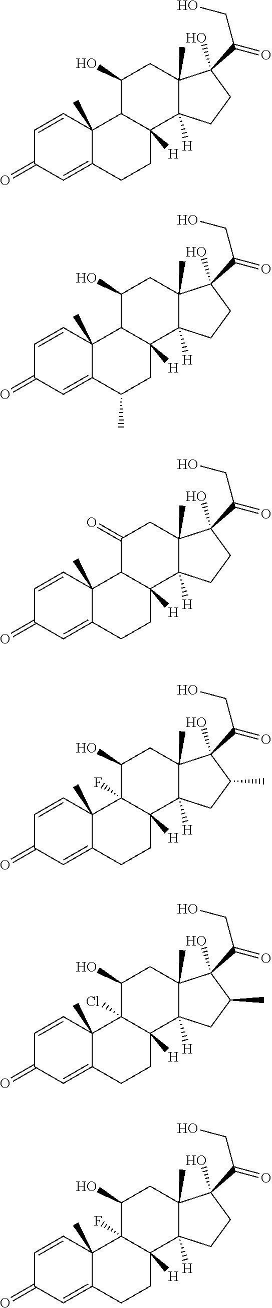 [SMILES: C[C@]12C=CC(=O)C=C1CC[C@@H]1C2[C@@H](O)C[C@@]2(C)[C@H]1CC[C@]2(O)C(=O)CO ; C[C@H]1C[C@@H]2C([C@@H](O)C[C@@]3(C)[C@H]2CC[C@]3(O)C(=O)CO)[C@@]2(C)C=CC(=O)C=C12 ; C[C@]12C=CC(=O)C=C1CC[C@@H]1C2C(=O)C[C@@]2(C)[C@H]1CC[C@]2(O)C(=O)CO ; C[C@@H]1C[C@H]2[C@@H]3CCC4=CC(=O)C=C[C@]4(C)[C@@]3(F)[C@@H](O)C[C@]2(C)[C@@]1(O)C(=O)CO ; C[C@H]1C[C@H]2[C@@H]3CCC4=CC(=O)C=C[C@]4(C)[C@@]3(Cl)[C@@H](O)C[C@]2(C)[C@@]1(O)C(=O)CO ; C[C@]12C=CC(=O)C=C1CC[C@H]1[C@@H]3CC[C@](O)(C(=O)CO)[C@@]3(C)C[C@H](O)[C@@]12F]

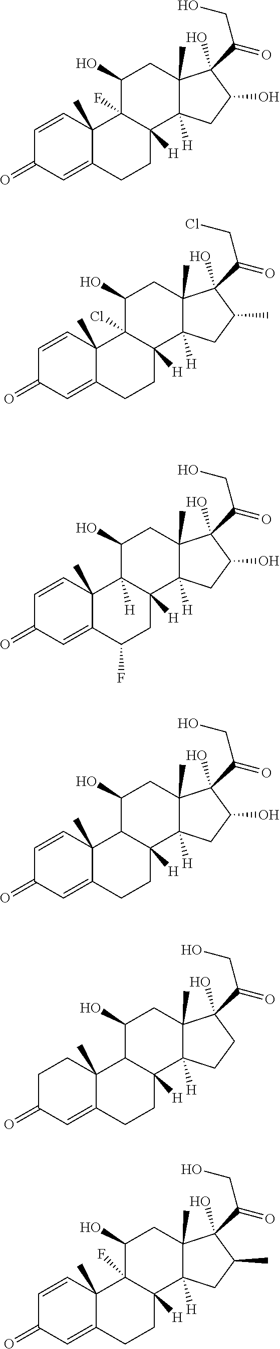

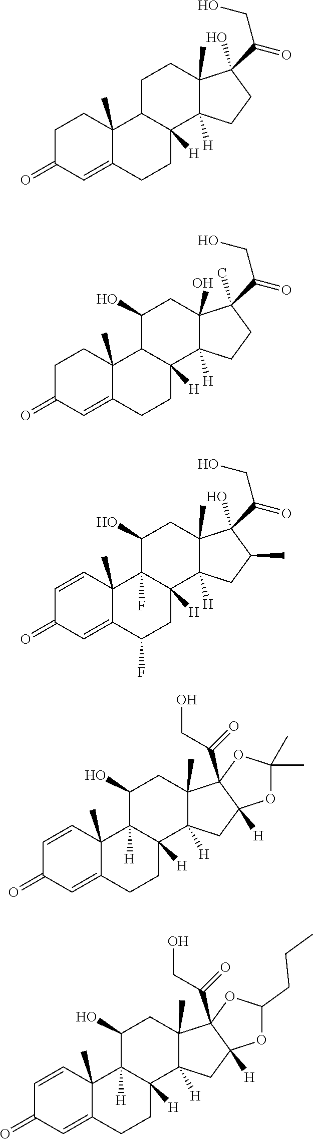

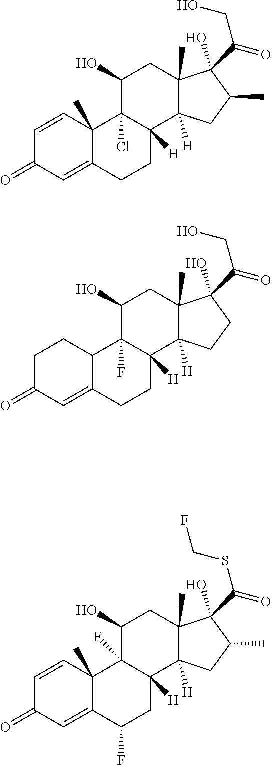

In another preferred embodiment, group R in a compound of any of the Formulas I-P-1, I-P-2, I-1, I-2, Ia, Ib, Ic, II-P-1, II-P-2, III-P, III, or IV-P is derived from subtraction of a H from an OH group in structures HR¹ wherein HR¹ is selected from the structures below:

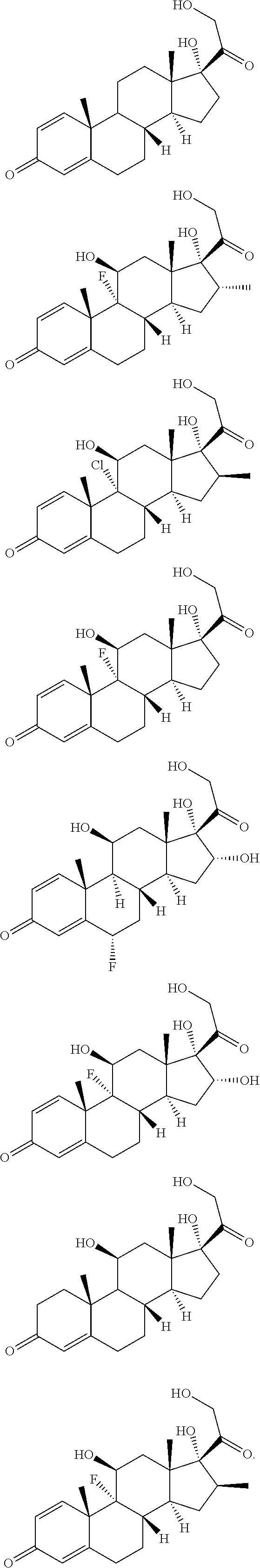

Some preferred compounds of Formula I-P-1 or T-P-2 are exemplified below, including any salt thereof, such as a hydrochloride, a sulfate, or another pharmaceutically acceptable salt.

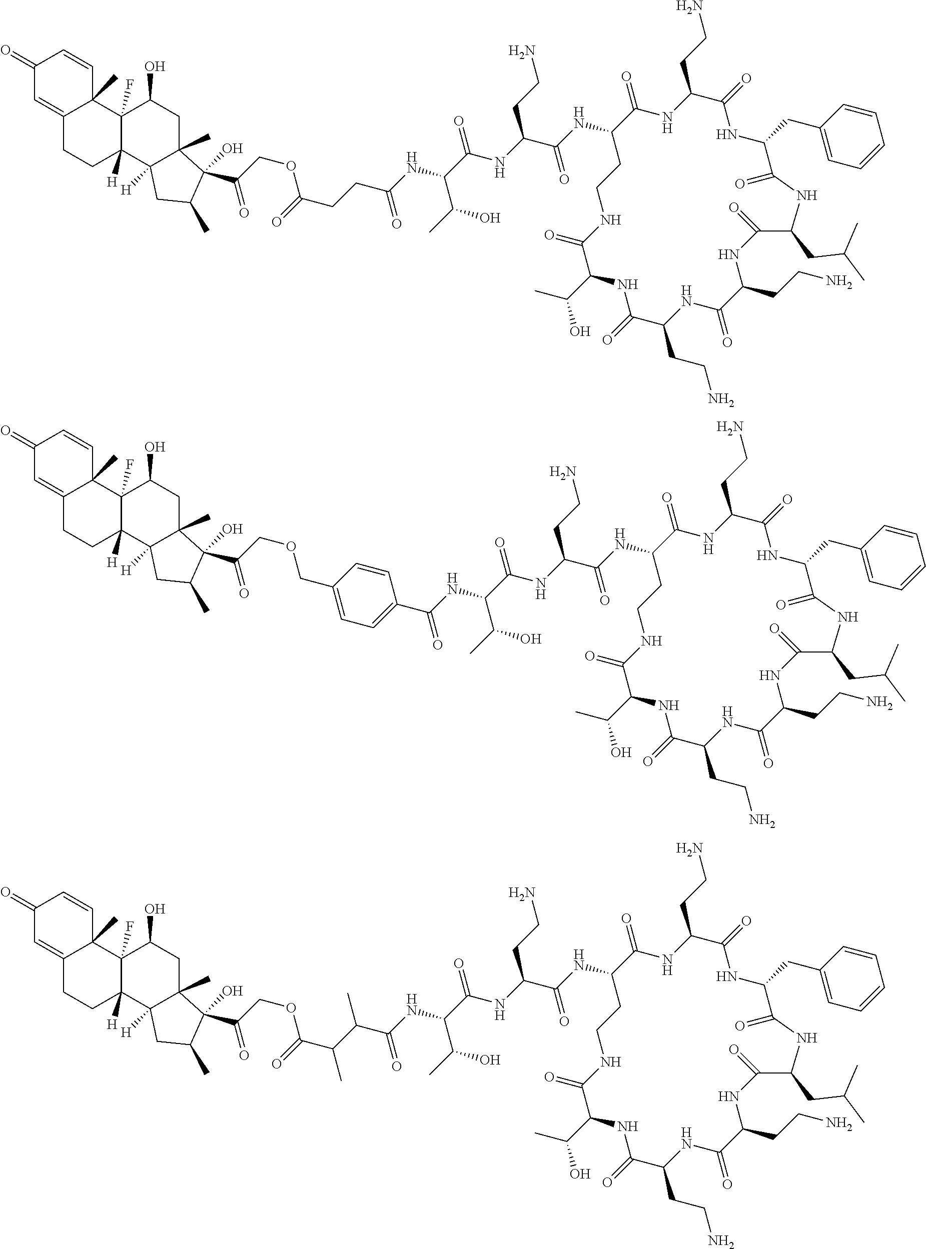

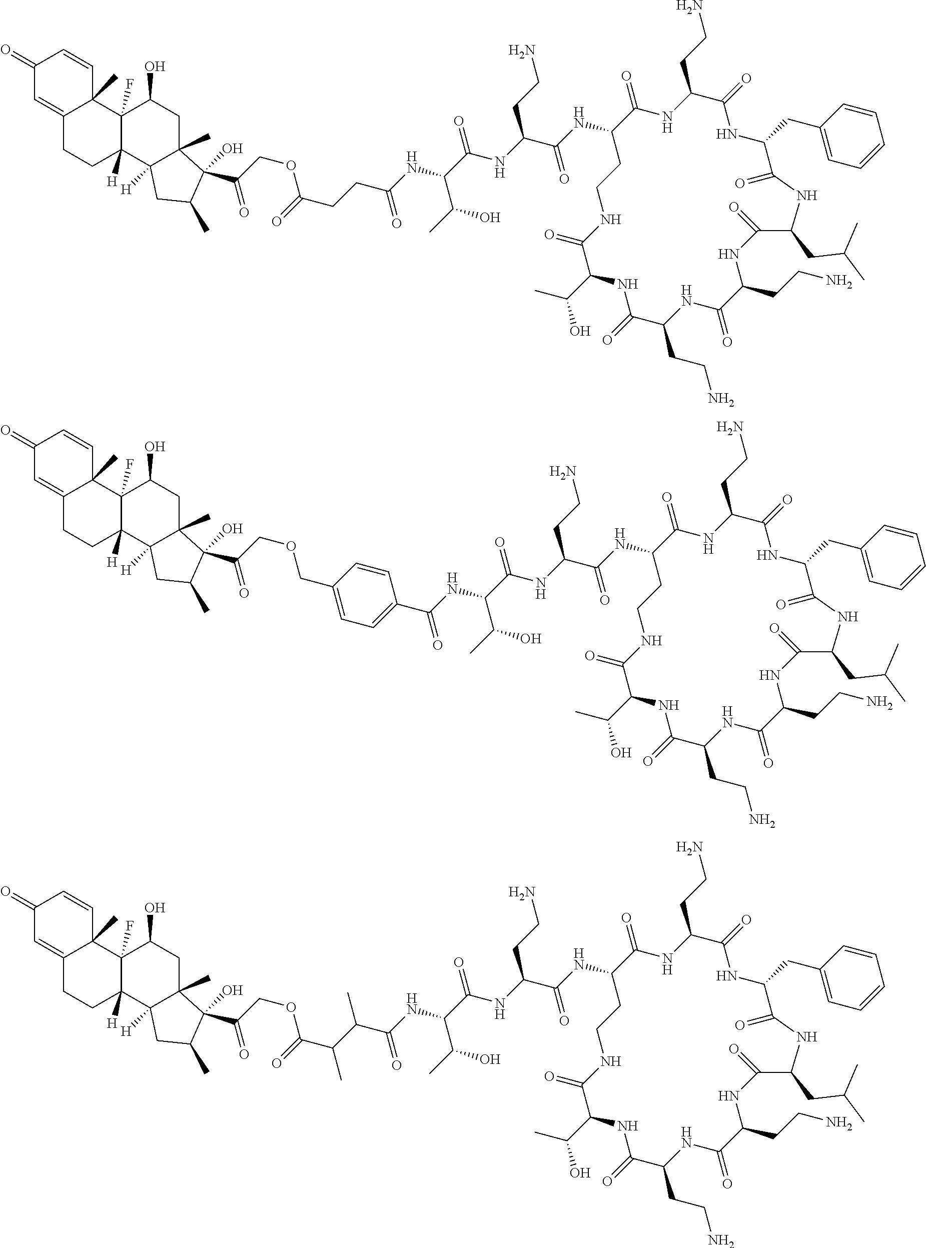

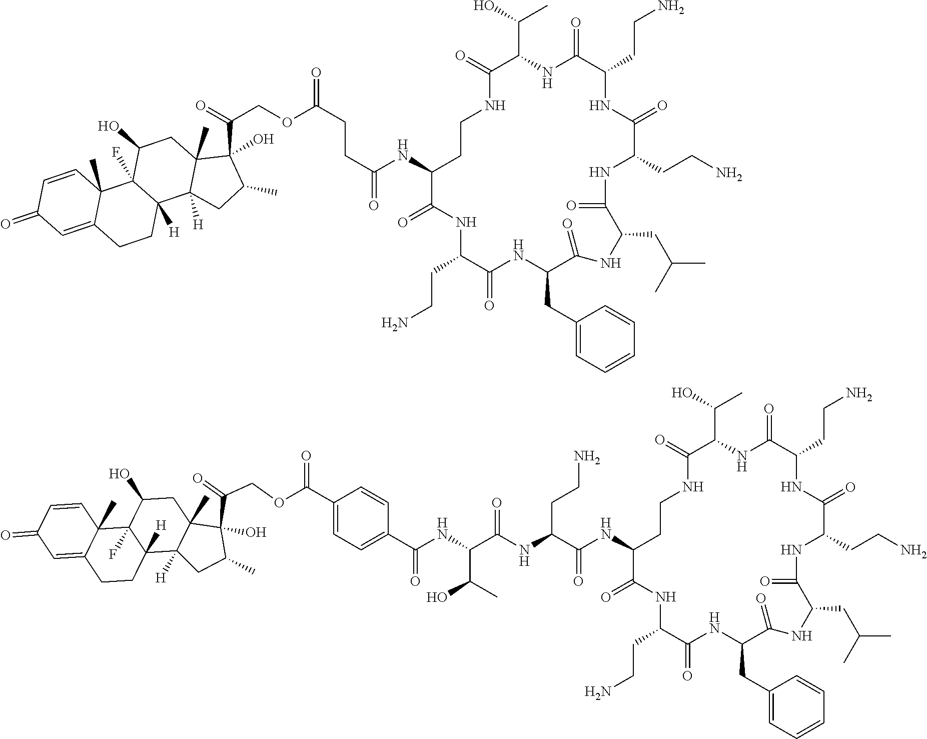

Some preferred compounds of the present invention are exemplified below, including any salt thereof, such as a hydrochloride, a sulfate, or another pharmaceutically acceptable salt.

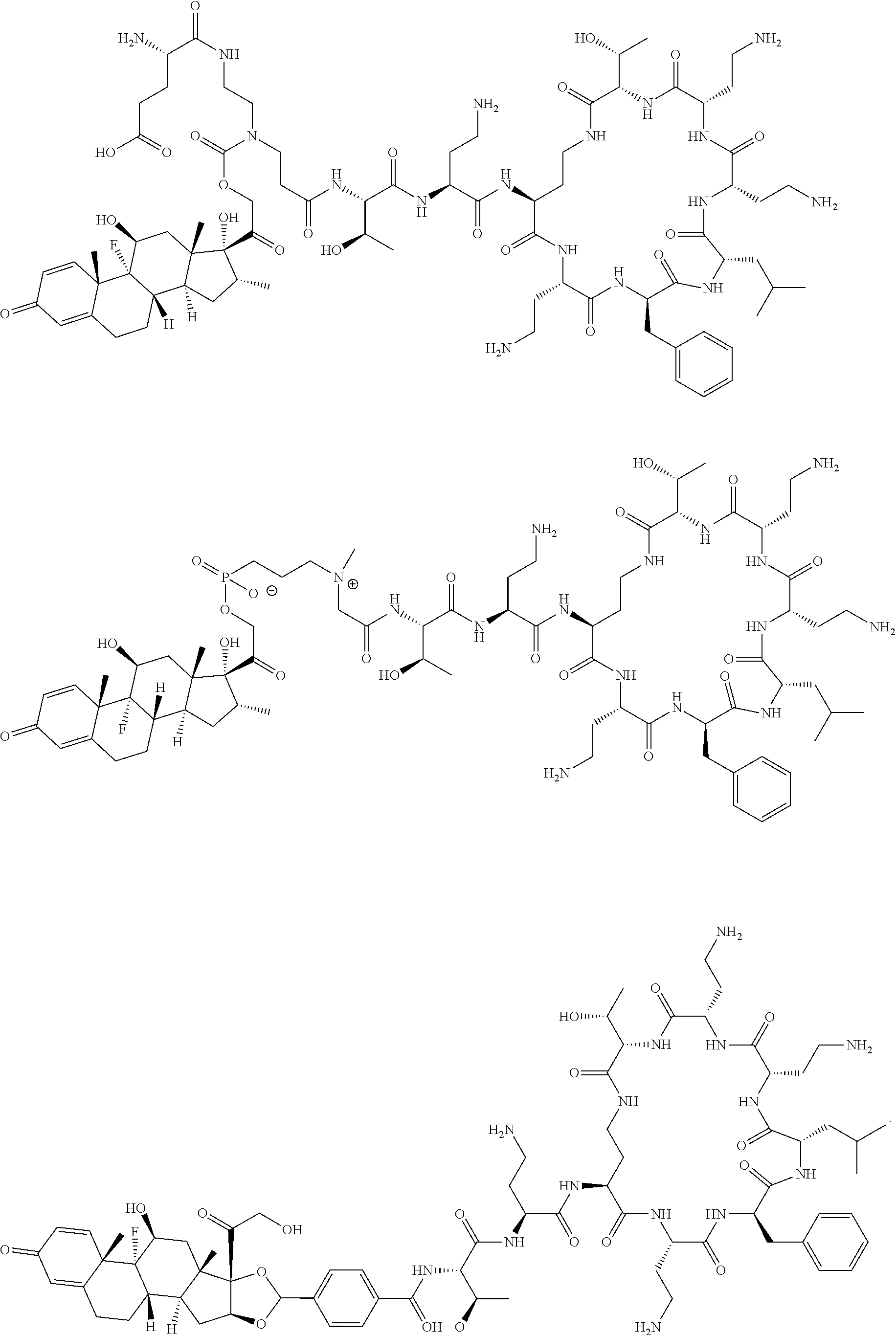

Additional preferred compounds of Formulas I, Ia, Ib, or Ic are exemplified below, including any salt thereof, such as a hydrochloride, a sulfate, or another pharmaceutically acceptable salt.

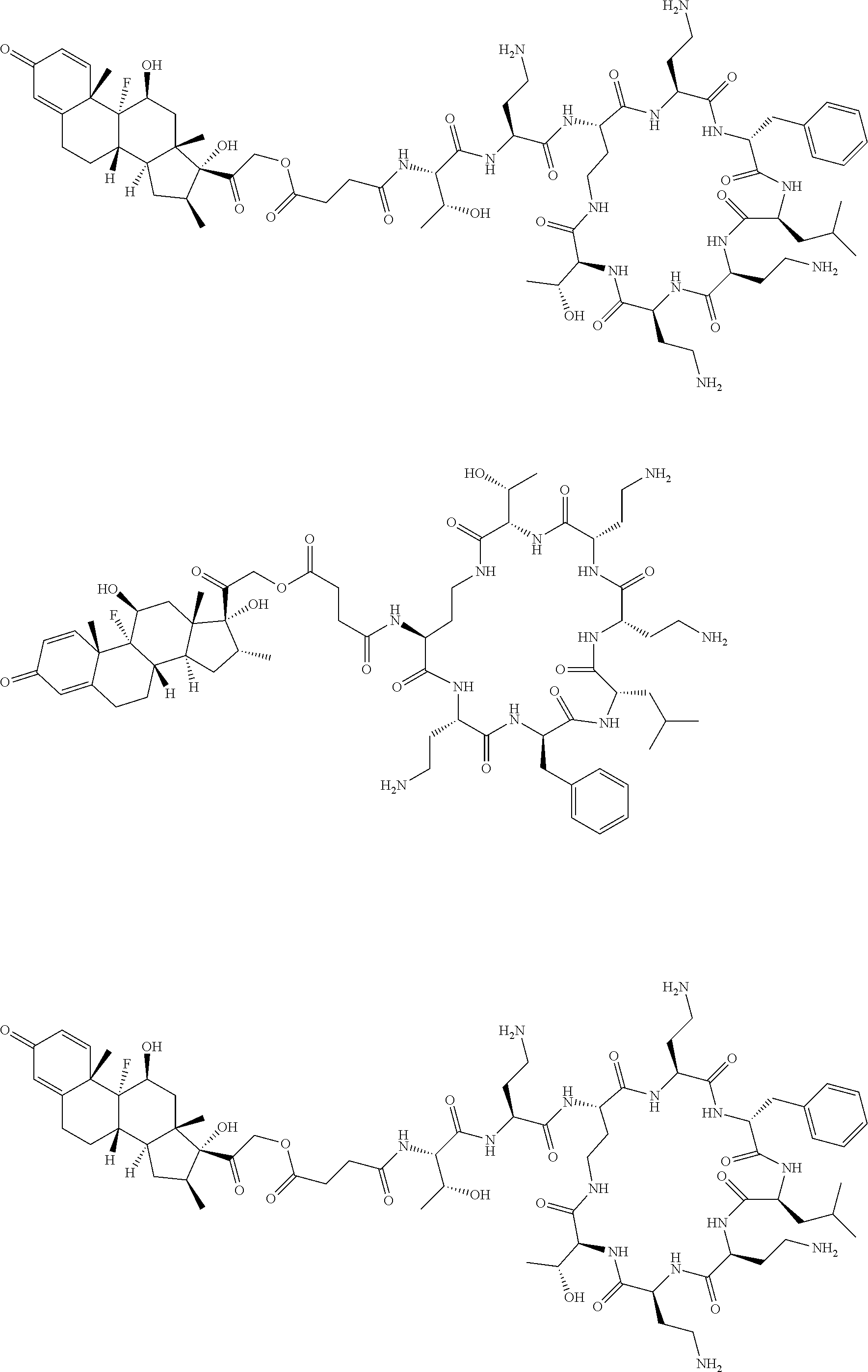

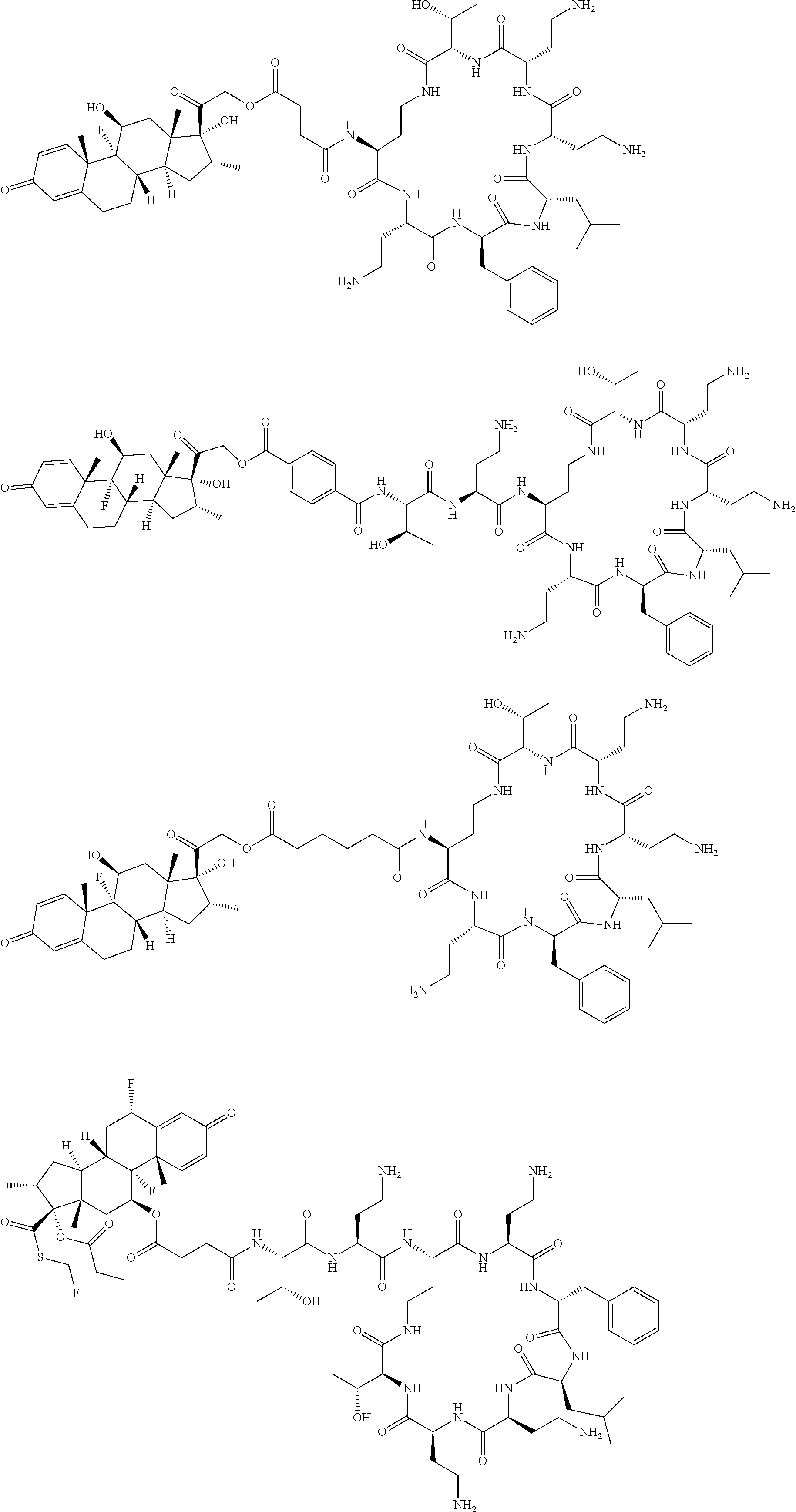

Additional preferred compounds of Formulas I, Ia, Ib, or Ic are exemplified below, including any salt thereof, such as a hydrochloride, a sulfate, or another pharmaceutically acceptable salt.

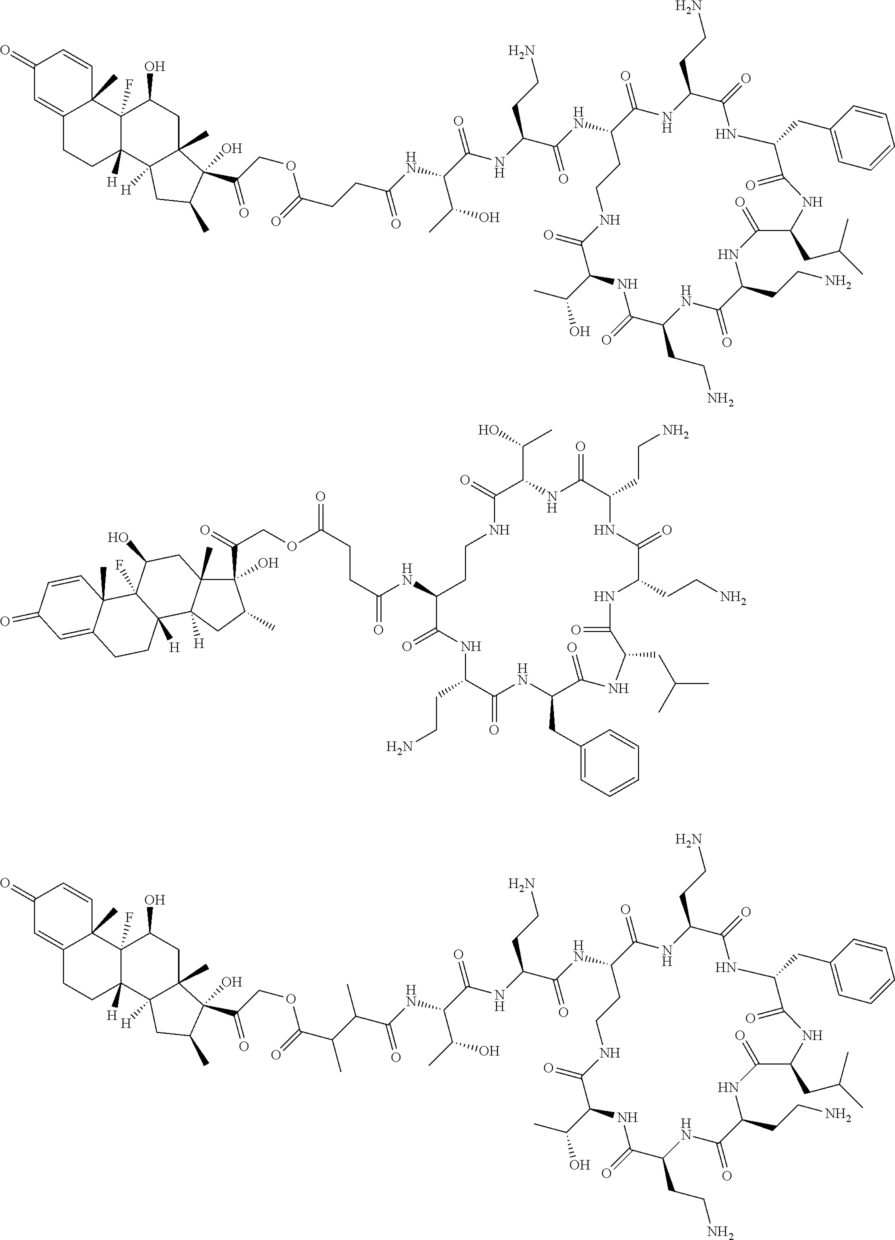

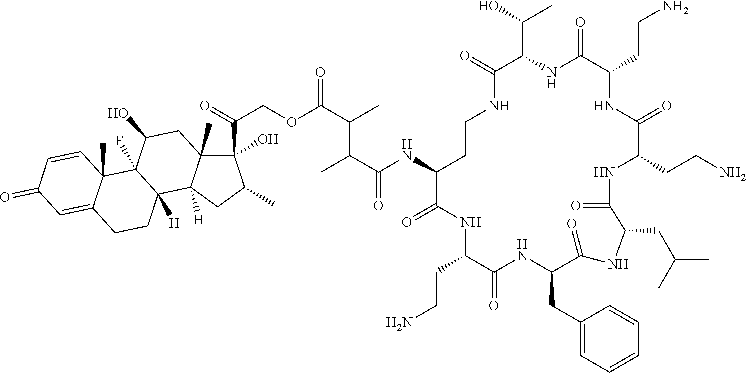 [SMILES: CC(C)C[C@@H]1NC(=O)[C@@H](Cc2ccccc2)NC(=O)[C@H](CCN)NC(=O)[C@@H](NC(=O)C(C)C(C)C(=O)OCC(=O)[C@@]2(O)[C@H](C)C[C@H]3[C@@H]4CCC5=CC(=O)C=C[C@]5(C)[C@@]4(F)[C@@H](O)C[C@@]32C)CCNC(=O)[C@H]([C@@H](C)O)NC(=O)[C@H](CCN)NC(=O)[C@H](CCN)NC1=O]

In one preferred embodiment, provided is a compound of any one of Formulas I-P-1, I-P-2, Ia, Ib, Ic, I-1, I-2, II-P-1, II-P-2, III-P, III, or IV-P, or any embodiments therein, or a pharmaceutically acceptable salt, solvate, or hydrate thereof that possesses anti-inflammatory activity; or therapeutic effects for kidney diseases, as determined by (i) alleviating or slowing kidney cytokine release such as TNF-α, IL-6, or IL-12; (ii) reducing one or more biomarkers such as protein levels, blood urea nitrogen, and serum creatinine, or by (iii) improving a condition of a patient in need of therapy, or of a mammal in animal tests.

In some or any embodiments, a compound of any one of Formulas I-P-1, I-P-2, I-1, I-2, Ia, Ib, Ic, II-P-1, II-P-2, III-P, III, or IV-P, or any embodiments therein, or a pharmaceutically acceptable salt, solvate, or hydrate thereof, is useful for therapy of an inflammation, wherein the inflammation is a renal inflammation, or inflammation diseases in a kidney injury or dysfunction, or an inflammation induced by a nephrotoxic substance, including a pharmaceutical nephrotoxic substance, such as anticancer, anti-diabetes, anti-infective, or another chemotherapeutic substance.

In some or any embodiments, a compound of any one of Formulas Ia, Ib, Ic, I-1, 1-2, II-P-2, III-P, III, or IV-P, or any embodiments therein, possesses an enhanced anti-inflammatory, immunomodulating, or nephro-protective effect when compared to a similar dose of a free agent or drug HR¹ incorporated into said compound as determined by in vitro or in vivo test(s) for anti-inflammatory, immunomodulating, or nephro-protective activity. In some or any embodiments, a compound of Formulas I-P-1, I-P-2, or II-P-1, or any embodiments therein, possesses an enhanced anti-inflammatory, immunomodulating, or nephro-protective effect when compared to a similar dose of a free agent or drug HR¹ and/or (H)ₒR² incorporated into said compound as determined by in vitro or in vivo test(s) for anti-inflammatory, immunomodulating, or nephro-protective activity.

In some or any embodiments, a compound of any one of Formulas I-P-1, I-P-2, la, Ib, Ic, I-1, 1-2, II-P-1, II-P-2, III-P, III, or IV-P, or any embodiments therein, wherein when administered to a mammal, said compound exhibits preferential accumulation in kidneys, with a ratio for its molar concentration in kidneys compared to that in blood of between about 5 and 500.

In some or any embodiments, a compound of any one of Formulas I-P-1, I-P-2, Ia, Ib, Ic, I-1, I-2, II-P-1, II-P-2, III-P, III, or IV-P, or any embodiments therein, wherein when administered to a mammal, said compound exhibits preferential accumulation in kidneys, with a ratio for its molar concentration in kidneys compared to that in blood of at least about 20.

In some or any embodiments, a compound of any one of Formulas Ia, Ib, Ic, I-1, 1-2, II-P-2, III-P, III, or IV-P, or any embodiments therein, wherein when administered to a mammal at a dosing (expressed in molar amount) equal to a standard therapeutic dosing (in molar amount) of an agent HR¹, said compound exhibits about 1.5- to 15-fold higher loading (tissue concentration) and/or drug exposure (area-under-the-curve, AUC) of agent HR¹ in kidneys, as compared to the standard therapeutic dosing of free drugs HR¹. In some or any embodiments, a compound of any one of Formulas I-P-1, I-P-2, or 1-1, 1-2, II-P-1, or any embodiments therein, wherein when administered to a mammal at a dosing (expressed in molar amount) equal to a standard therapeutic dosing (in molar amount) of an agent HR¹ and/or (H)ₒR², said compound exhibits about 1.5- to 15-fold higher loading (tissue concentration) and/or drug exposure (area-under-the-curve, AUC) of agent HR¹ and/or (H)ₒR² in kidneys, as compared to the standard therapeutic dosing of free drugs HR¹ and/or (H)ₒR².

In some or any embodiments, a compound of any one of Formulas Ia, Ib, Ic, I-1, I-2, II-P-2, III-P, III, or IV-P, or any embodiments therein, wherein when administered to a mammal at a dosing (expressed in molar amount) equal to a standard therapeutic dosing (in molar amount) of agent HR¹, exhibits about 1.5- to 15-fold higher efficacy, as compared to the standard therapeutic dosing of agent HR¹, with said therapeutic effect determined as a slowed, stopped, or reversed progression of inflammation (as determined per changes in cytokine release, and/or by using biochemical biomarkers for disease monitoring, or similar methods). In some or any embodiments, a compound of Formulas I-P-1, I-P-2, or II-P-1, or any embodiments therein, wherein when administered to a mammal at a dosing (expressed in molar amount) equal to a standard therapeutic dosing (in molar amount) of agent HR¹ and/or (H)ₒR², exhibits about 1.5- to 15-fold higher efficacy, as compared to the standard therapeutic dosing of agent HR¹, with said therapeutic effect determined as a slowed, stopped, or reversed progression of inflammation (as determined per changes in cytokine release, and/or by using biochemical biomarkers for disease monitoring, or similar methods).

In some or any embodiments, a compound of any one of Formulas Ia, Ib, Ic, I-1, I-2, II-P-2, III-P, III, or IV-P, or any embodiments therein, and wherein when it is administered to a mammal at a dosing (expressed in molar amount) equal to a standard therapeutic dosing (in molar amount) of agent HR¹, exhibits at least 2-fold higher efficacy, as compared to the standard therapeutic dosing of agent HR¹, with said therapeutic effect determined as a slowed, stopped, or reversed progression of inflammation or a kidney injury (as determined by levels of a cytokine release, and/or by using biochemical biomarkers for inflammation monitoring, or by radiography, or by magnetic resonance imaging, or alike). In some or any embodiments, a compound of Formulas I-P-1, I-P-2, or II-P-1, or any embodiments therein, and wherein when it is administered to a mammal at a dosing (expressed in molar amount) equal to a standard therapeutic dosing (in molar amount) of agent HR¹ and/or (H)ₒR², exhibits at least 2-fold higher efficacy, as compared to the standard therapeutic dosing of agent HR¹, with said therapeutic effect determined as a slowed, stopped, or reversed progression of inflammation or a kidney injury (as determined by levels of a cytokine release, and/or by using biochemical biomarkers for inflammation monitoring, or by radiography, or by magnetic resonance imaging, or alike).

In some or any embodiments, a compound of any one of Formulas Ia, Ib, Ic, I-1, I-2, II-P-2, III-P, III, or IV-P, or any embodiments therein, wherein when administered to a mammal at a dosing (expressed in molar amount) equal to a standard therapeutic dosing (in molar amount) of an agent HR¹, exhibits at least 2-fold reduced rate of adverse effects and/or off-target toxicity manifestation, as compared to the standard therapeutic dosing of HR, as determined by medical observations of a mammal under therapy, a blood cells count, a tissue biopsy, and/or by analysis of biochemical biomarkers, or similar method. In some or any embodiments, a compound of Formulas I-P-1, I-P-2, or II-P-1, or any embodiments therein, wherein when administered to a mammal at a dosing (expressed in molar amount) equal to a standard therapeutic dosing (in molar amount) of an agent HR and/or (H)ₒR², exhibits at least 2-fold reduced rate of adverse effects and/or off-target toxicity manifestation, as compared to the standard therapeutic dosing of HR¹ and/or (H)ₒR², as determined by medical observations of a mammal under therapy, a blood cells count, a tissue biopsy, and/or by analysis of biochemical biomarkers, or similar method.

In some embodiments and aspects, a compound provided herein may be used in a combination with an adjunct agent, to act synergistically and/or enhance therapeutic effects of said compound itself, or of an adjunct agent, or both. Such adjunct agents include other anticancer or immunomodulating agent(s), such as a monoclonal antibody agent, or another anti-inflammatory agent(s), or another anticancer or antibacterial agent, or humanized antibody.

In some embodiments and aspects is provided a method for the treatment of an inflammation-associated kidney disease in a mammal comprising administering to the mammal a therapeutically effective amount of a compound of any of the Formulas I-P-1, I-P-2, I, Ia, Ib, Ic, I-1, 1-2, II-P-1, II-P-2, III-P, III, or IV-P.

In one such aspect, a compound provided herein has modest or no anti-inflammatory activity in vitro, but exhibits high anti-inflammatory efficacy when administered to a mammal in need of such a therapy.

In some or any embodiments is provided a pharmaceutical composition comprising a therapeutically effective amount of a compound of a compound of Formulas I-P-1, I-P-2, I, Ia, Ib, Ic, I-1, I-2, II-P-1, II-P-2, III-P, III, or IV-P, or as defined in any of the embodiments described herein, and a pharmaceutically acceptable carrier.

### General Synthetic Methods

The compounds of described herein can be prepared in accordance with one or more of methods described, for example, in references below. General syntheses of certain related starting materials have been described in the literature. For example, the preparation of Boc-protected polymyxin nonapeptide was described by O'Dowd et al. in Tetrahedron Lett. 2007, vol. 48, p. 2003. Additional protected polymyxin B nonapeptide and colistin nonapeptide derivatives can be prepared as described by Okimura et al. in Chem. Pharm. Bull. 2007, vol. 55, pp. 1724-1730. Likewise, the general peptide acylation chemistry was described in the ref Tetrahedron Lett. 2007, vol. 48, pp. 2003-2005.

Additional general methods suitable for preparation of compounds of Formulas I-P-1, I-P-2, I, Ia, Ib, Ic, I-1, 1-2, II-P-1, II-P-2, III-P, III, or IV-P have been described in publications WO 2021/150792, WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687.

Methods suitable for incorporation of suitable enzymatically and/or chemically cleavable groups X, Y, and Z (and additional spacers/linkers in same) in compounds of Formulas I-P-1, I-P-2, I, Ia, Ib, Ic, I-1, 1-2, II-P-1, II-P-2, III-P, III, or IV-P have been described in generally related synthetic art for preparation of ADCs and other agents, such as reported, for example, in publications US 20170355769; J. Am. Chem. Soc. 2018, vol. 140, p. 1617; Bioconjugate Chem. 2016, vol. 27, p. 1606; Bioconjugate Chem. 2016, vol. 27, p. 1645; Bioconjugate Chem. 2015, vol. 26, p. 919; Mol. Pharmaceutics 2015, vol. 12, p. 1813; ACS Med Chem. Lett. 2017, vol. 8, p. 1037; ACS Med. Chem. Lett. 2016, vol. 7, p. 983; Org. Process Res. Dev. 2019, vol. 23, p. 2647; Bioconjugate Chem. 2016, vol. 27, p. 1880; Bioconjugate Chem. 2017, vol. 28, p. 620; Org. Process Res. Dev. 2018, vol. 22, p. 286; Bioconjugate Chem. 2015, vol. 26, p. 2216; J. Med. Chem. 2014, vol. 57, p. 6949; Bioconjugate Chem. 2018, vol. 29, p. 1155; J Am. Chem. Soc. 2015, vol. 137, p. 3229; Mol. Pharmaceutics 2018, vol. 15, p. 2384; ACS Med. Chem. Lett. 2016, vol. 7, p. 988; Chem. Biodiversity 2019, vol. 16, e1800520; Nature Commun.|2018, vol. 9, p. 2512; Mol. Pharmaceutics 2011, vol. 8, p. 901; ACS Med. Chem. Lett. 2019, vol. 10, p. 1393; J. Nat. Prod. 2017, vol. 80, p. 2447; ACS Med. Chem. Lett. 2019, vol. 10, p. 1674; Pharmaceutics 2013, vol. 5, p. 220; and other references cited in these publications.

Specific methods, amino acid reagents, as well as linker/spacer structures described in above literature are directly adaptable to prepare compounds of Formulas I-P-1, I-P-2, I, Ia, Ib, Ic, I-1, I-2, II-P-1, II-P-2, III-P, III, or IV-P, by straightforward variations in specific reagents and protection/deprotection schemes, obvious to one skilled in synthetic organic chemistry.

Additional syntheses of specific compounds described herein are illustrated by various synthetic Schemes for Examples below, likewise adaptable to preparation of additional compounds provided herein.

### EXAMPLES

Embodiments described herein are described in the following examples, which are meant to illustrate and not limit the scope of this disclosure. Common abbreviations well-known to those with ordinary skills in the synthetic art used throughout.

### Abbreviations

NMR: 400 MHz ¹H NMR spectra (δ, ppm) recorded in D₂O unless specified otherwise.
LCMS: liquid chromatography mass-spectroscopy analysis.
MS: mass-spectroscopy data (m/z) for a positive ionization method.
Chromatography: silica gel chromatography using organic solvents unless specified otherwise.
TLC: thin-layer chromatography.
HPLC: reverse-phase high-performance chromatography using commercial C18 columns.
TES: Et₃SiH.
TFA: CF₃COOH.
EA: EtOAc or ethyl acetate.
CDI: carbonyldiimidazole.
EDC: 1-ethyl-3-(3-dimethylaminopropyl)carbodiimide.
DMAP 4-dimethylaminopyridine.
DIPEA: N,N-diisopropylethylamine.
HATU: 1-[bis(dimethylamino)methylene]-1H-1,2,3-triazolo[4,5-b]pyridinium 3-oxide hexafluorophosphate, hexafluorophosphate azabenzotriazole tetramethyl uranium.
Cs₂CO₃: cesium carbonate.
MeOH: methanol.
ACN: MeCN.
DMF: N,N-dimethylformamide.
DCC: N,N′-dicyclohexylcarbodiimide.
DCE: 1,2-dichloroethane.
DCM: 1,2-dichloromethane.
NMP: N-methyl pyrrolidinone.
PE: hexanes or light petroleum ether.
T3P: tris-propylphosphonic anhydride.
C18 chromatography: reverse phase chromatography using a gradient of 0.1% TFA in water and acetonitrile (ACN).
PMBN(Boc)₄: H-Thr-Dab(Boc)-cyclo[Dab(Boc)-Dab(Boc)-D-Phe-Leu-Dab(Boc)-Dab(Boc)-Thr].
PMBH(Boc)₃: H-cyclo[Dab(Boc)-Dab(Boc)-D-Phe-Leu-Dab(Boc)-Dab(Boc)-Thr].
Dab(Boc)PMBN(Boc)₄: Dab(Boc)-Thr-Dab(Boc)-cyclo[Dab(Boc)-Dab(Boc)-D-Phe-Leu-Dab(Boc)-Dab(Boc)-Thr]{same as H-Dab(Boc)-Thr-Dab(Boc)-cyclo[Dab(Boc)-Dab(Boc)-D-Phe-Leu-Dab(Boc)-Dab(Boc)-Thr]}.
Dex-Pnp: 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (4-nitrophenyl) carbonate.
Ar: argon
r.t. or RT: room temperature.

Other reagent abbreviations are just as employed in common synthetic literature, including the American Chemical Society list of abbreviations, such as found, for example, in the Journal of Organic Chemistry; or in the Journal of Peptide Chemistry. Unless specified otherwise, all reagents were either from commercial sources, or made by conventional methods described in available literature.

### Example 1

Synthesis of the Compound of Example 1:

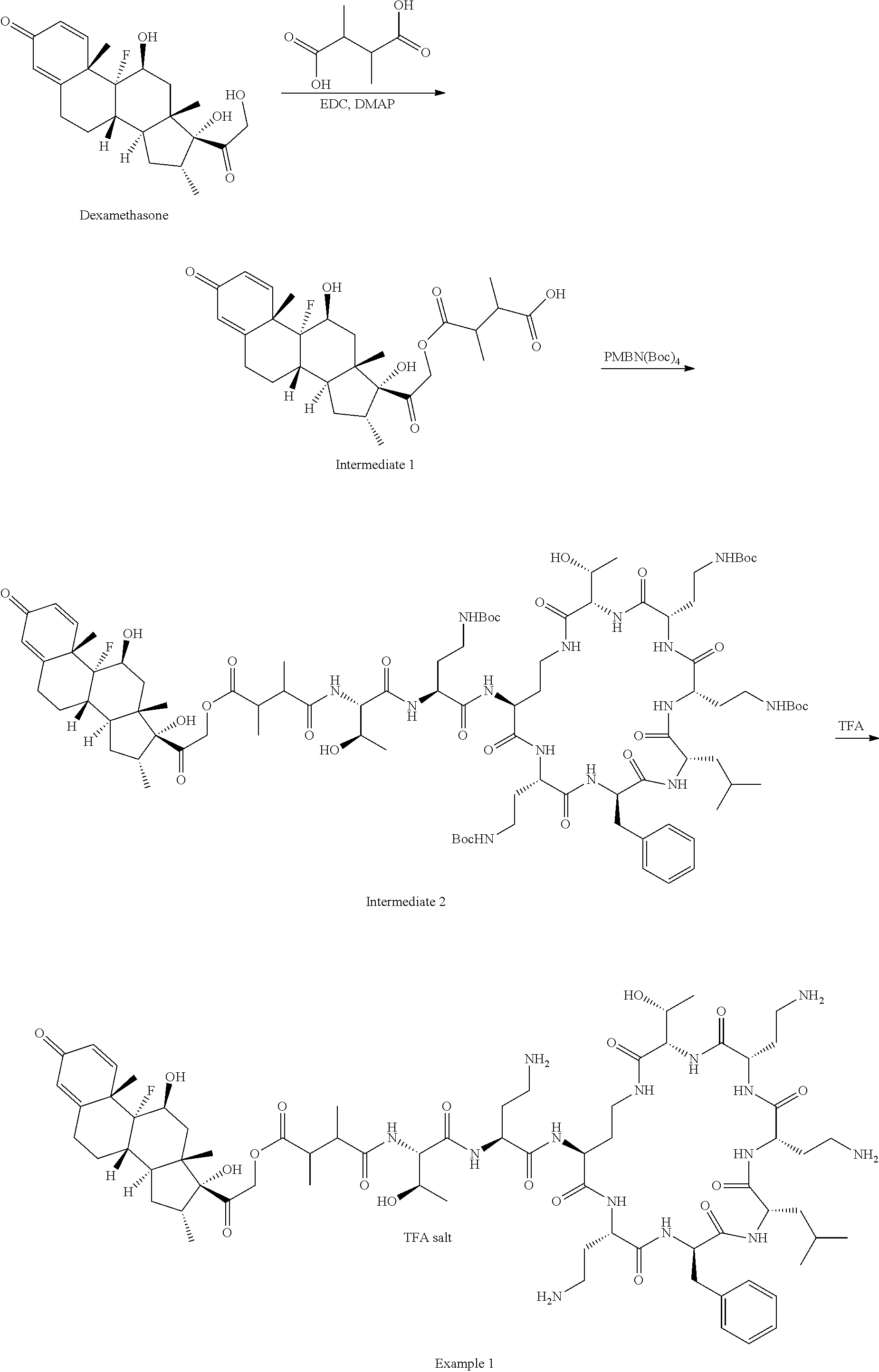

Intermediate 1. DCM (2.5 mL) was added with stirring to dexamethasone (101.1 mg, 0.26 mmol), 2,3-dimethylsuccinic acid (49.7 mg, 0.34 mmol), EDC (58.2 mg, 0.30 mmol) and DMAP (48.2 mg, 0.39 mmol) at rt. The mixture turned clear after 1 h, and it was then stirred at rt overnight. The mixture was evaporated under vacuum. Residue was dissolved in ACN (2 mL) and the product purified by HPLC (gradient 0-100% ACN-water with 0.05% TFA) to afford Intermediate 1 (102 mg) as white solid, yield 76%. LCMS: 521.26 [M+H]⁺.

Intermediate 2. DIPEA (0.1 mL, 0.57 mmol) was added to a mixture of Intermediate 1 (102 mg, 0.196 mmol), PMBN(Boc)₄ (253.5 mg, 0.186 mmol), HATU (89.4 mg, 0.235 mmol) in DMF (2.5 mL). The resulting mixture was stirred at room temperature overnight. LCMS detected the desired product. The mixture was then purified by HPLC (gradient 0-100% ACN-water with 0.05% TFA) to give Intermediate 2 (146 mg) as white powder, yield 42%. LCMS: 1866.34 [M+H]⁺.

Compound of Example 1. TFA (1 mL) was added to a solution of Intermediate 2 (146 mg, 0.078 mmol) in DCM (4 mL). The resulting mixture was stirred at r.t. for 3 h. Volatiles were evaporated under vacuum, the crude product was dissolved in H₂O/ACN (1 mL/1 mL), and purified by HPLC (gradient 0-60% water-ACN with 0.05% TFA) to afford to the Compound of Example 1 as its TFA salt (116.9 mg). NMR: 7.45 (d, J=10.4 Hz, 1H), 7.34-7.18 (m, 5H), 6.35 (dd, J=10.4 Hz, 1.6 Hz, 1H), 6.16 (s, 111), 5.08-4.87 (m, 2H), 4.52 (t, J=8.4 Hz, 1H), 4.45-4.39 (m, 2H), 4.34-4.31 (m, 111), 4.26-4.13 (m, 7H), 3.30-3.24 (m, 1H), 2.74-2.38 (m, 1H), 2.16-1.63 (m, 15H), 1.48 (s, 3H), 1.42-1.32 (m, 2H), 1.17-1.11 (m, 12H), 0.91 (s, 311), 0.80 (d, J=7.2 Hz, 3H), 0.70 (d, J=6.4 Hz, 311), 0.63 (d, J=6.0 Hz, 3H). MS: 1466.80 [M+H]⁺.

### Example 2

Synthesis of the Compound of Example 2:

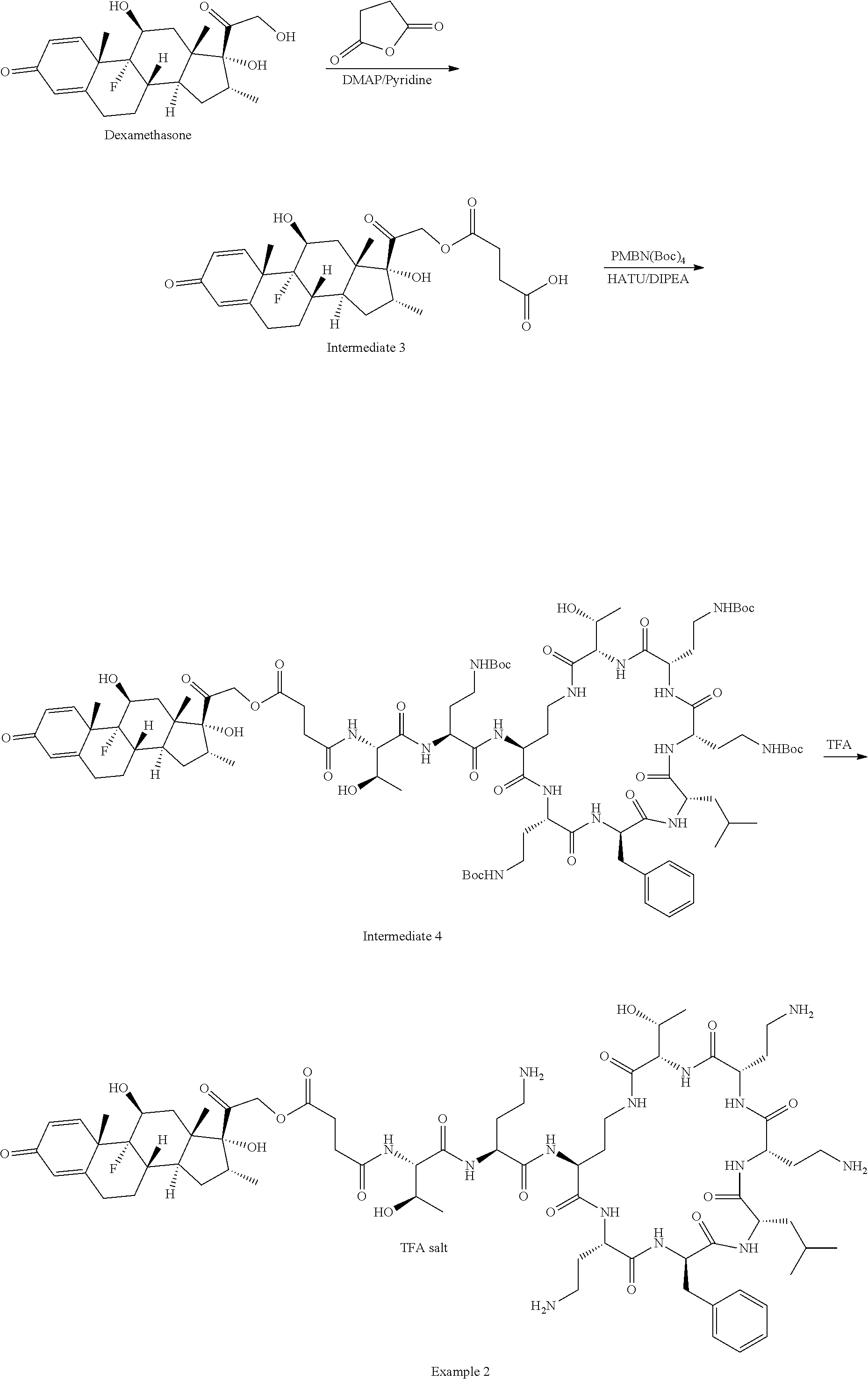

Intermediate 3. To a solution of dexamethasone (105.0 mg, 0.268 mmol) in anhydrous pyridine (2 mL) was added DMAP (71.8 mg, 0.588 mmol) followed by succinic anhydride (117.4 mg, 1.173 mmol) at rt. The mixture was stirred for 2 h under Ar, and then poured into 20 mL of 1M HCl, and filtered. The resulting white solid was washed with water (5 mL), and then dried under vacuum overnight to afford Intermediate 3 (120.5 mg, yield 91%). LCMS: 493.07 [M+H]⁺.

Intermediate 4. A mixture of Intermediate 3 (120.5 mg, 0.24 mmol), PMBN(Boc)₄ (339.2 mg, 0.25 mmol), HATU (111.0 mg, 0.29 mmol) in DMF (25 mL) and DIPEA (0.1 mL, 0.57 mmol) was stirred at rt overnight. The mixture was then purified by HPLC (gradient water-MeOH 0˜100%) to afford crude Intermediate 4 (181 mg) as a white powder. LCMS: 1837.91 [M+H]⁻.

Compound of Example 2. TFA (0.2 mL) was added to a solution of Intermediate 4 (140 mg, about 50% purity, 0.076 mmol) in DCM (2.5 mL). The resulting mixture was stirred at r.t. for 3 h. The mixture was stirred for 1 h, then volatiles were evaporated under reduced pressure, and the crude product was purified by HPLC (0.05% TFA in ACN-water 0-100%) to afford the Compound of Example 2. NMR: 7.39 (d, J=10.4 Hz, 1H), 7.30-7.21 (m, 5H), 6.28 (dd, J=10.4 Hz, 1.6 Hz, 1H), 6.07 (s, 1H), 5.11-5.06 (m, 1H), 4.50-4.07 (m, 12H), 3.60-3.54 (m, 2H), 3.08-1.18 (m, 57H), 1.00 (s, 3H), 0.91-0.68 (m, 10H). MS: 1437.75 [M+H]⁺.

### Example 3

Synthesis of the Compound of Example 3:

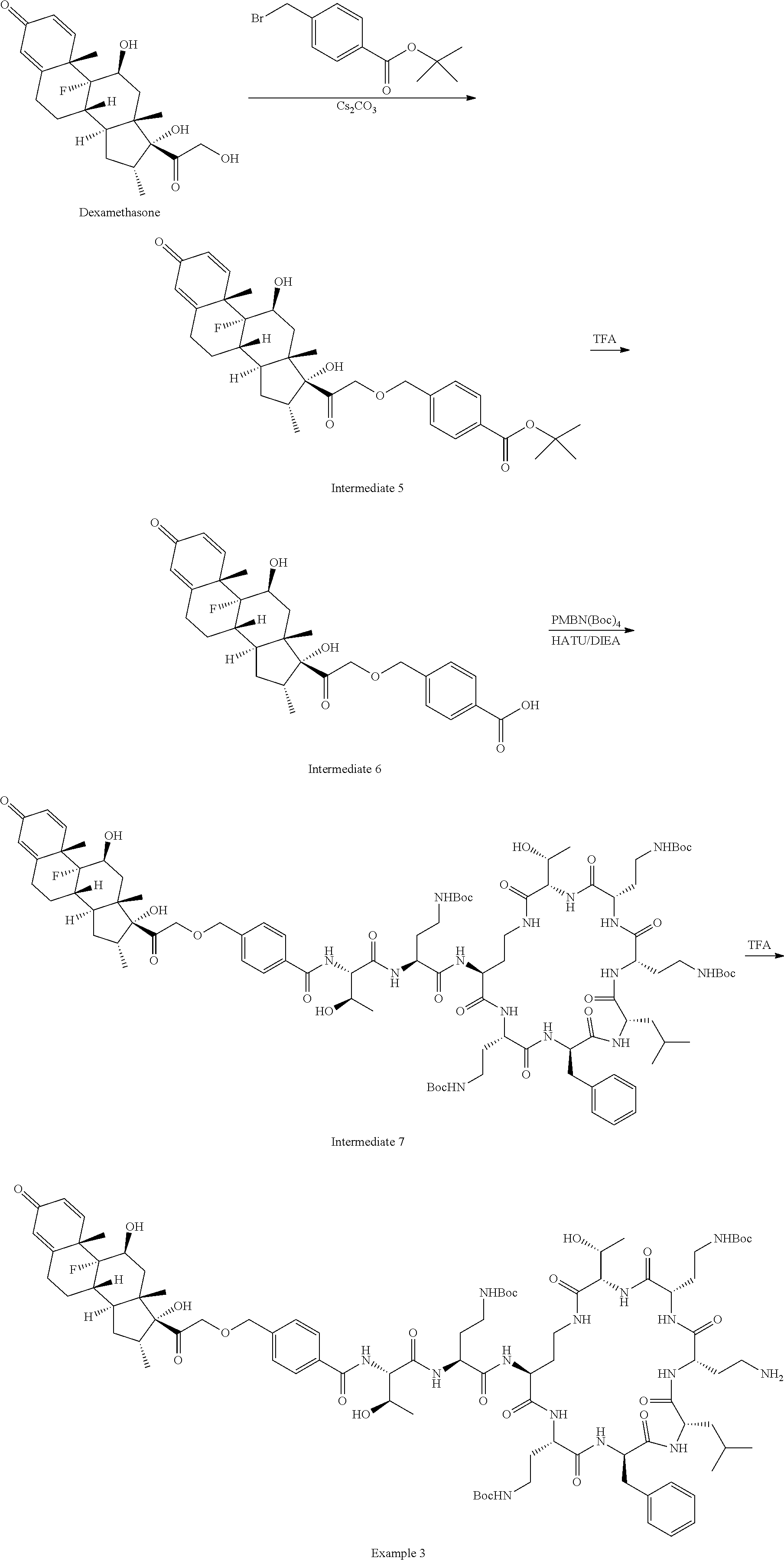

Intermediate 5. ACN (10 mL) was added to dexamethasone (239.8 mg, 0.61 mmol), Cs₂CO₃ (234.0 mg, 0.723 mmol), and tert-butyl 4-(bromomethyl)benzoate (171.8 mg, 0.63 mmol) at rt under Ar, and the mixture was stirred at 65° C. for 4 h. The mixture was filtered and evaporated under vacuum, and the product was purified by flash column (silica gel, gradient 0-45% PE-EtOAc) to afford Intermediate 5 (116 mg) as white solid, yield 76%. LC/MS: 583.01 [M+H]⁺.

Intermediate 6. TFA (1 mL) was added to Intermediate 5 (110 mg, 0.188 mmol) in DCM (4 mL). and the mixture was stirred for 2 h. The mixture was evaporated under vacuum, and the crude product was taken to the next step directly. LCMS: 527.16 [M+H]⁺.

Intermediate 7. DIPEA (0.08 mL, 0.46 mmol) was added to a mixture of Intermediate 6 (about 0.188 mmol), PMBN(Boc)₄ (260 mg, 0.19 mmol), HATU (89 mg, 0.23 mmol) in DMF (2.5 mL). The resulting mixture was stirred at rt overnight. The mixture was diluted with 10 mL of EtOAc, and organic layer was washed with water (5 mL), brine (10 mL), dried over Na₂SO₄. Volatiles were removed under vacuum, and the crude product was purified by flash chromatography (silica gel, gradient DCM-MeOH 0˜12%) to give Intermediate 7 (293.8 mg) as colorless solid, yield 83% in two steps. LCMS: 1871.52 [M+H]⁺.

Compound of Example 3. TFA (1 mL) was added to a solution of Intermediate 7 (293.8 mg, 0.157 mmol) in DCM (4 mL). The resulting mixture was stirred at r.t. for 2 h. Volatiles were evaporated under vacuum, and the crude product was purified by HPLC (0.05% TFA in ACN-water 0-45%) to afford the Compound of Example 3. NMR: 7.80 (d, J=8.0 Hz, 2H), 7.49 (d, J=8.0 Hz, 2H), 7.40 (d, J=10.0 Hz, 1H), 7.34-7.25 (m, 3H), 7.19-7.17 (m, 2H), 6.33-6.30 (m, 1H), 6.13 (s, 1H), 4.69-4.08 (m, 15H), 3.25-1.18 (m, 1H), 3.06-2.94 (m, 7H), 2.80-2.59 (m, 3H), 2.50-2.36 (m, 2H), 2.18-1.66 (m, 13H), 1.46 (s, 3H), 1.42-1.31 (m, 2H), 1.25 (d, J=6.4 Hz, 3H), 1.19-1.13 (m, 2H), 1.09 (d, J=6.4 Hz, 3H), 0.84-0.76 (m, 6H), 0.70 (d, J=6.4 Hz, 3H), 0.63 (d, J=7.0 Hz, 3H). MS: 1471.74 [M+H]⁺.

### Example 4

Synthesis of the Compound of Example 4:

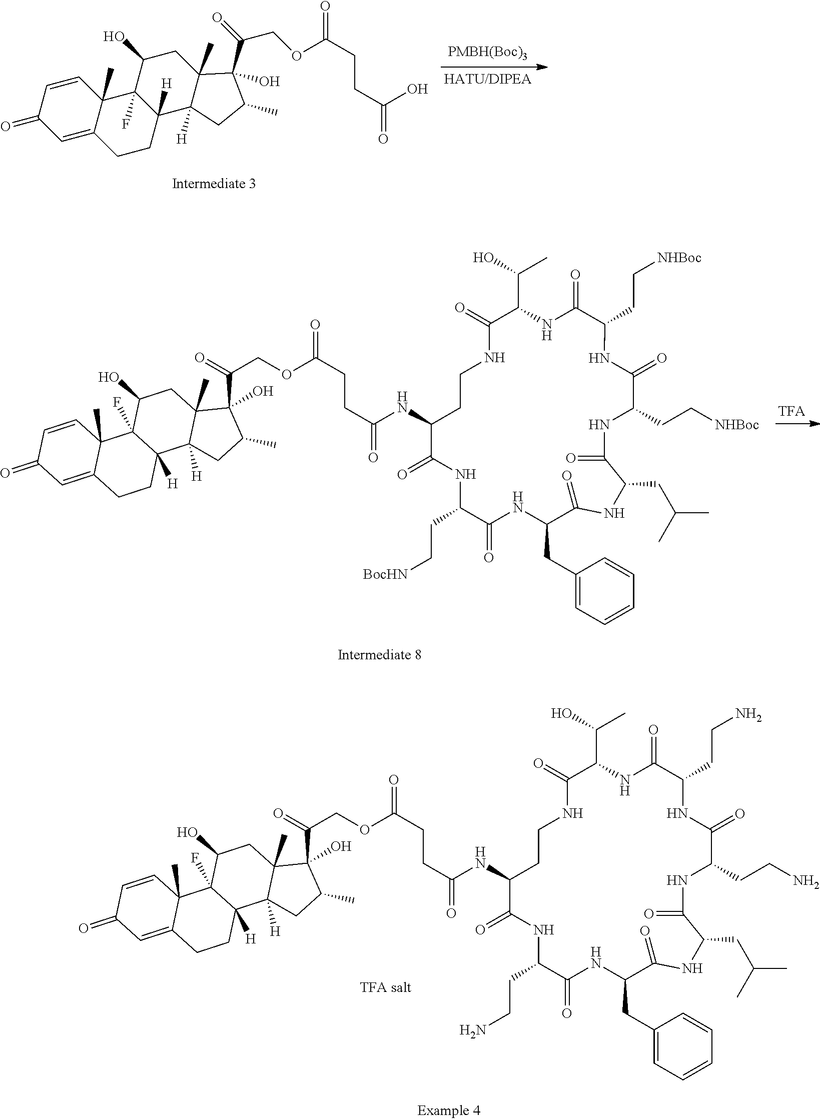

Compound of Example 4. Compound of Example 4 was made according to a similar procedure to the Compound of Example 2 except that Intermediate 3 was coupled with PMBH(Boc)₃ instead of PMBN(Boc)₄ used for Example 2. NMR: 7.44 (d, J=10.4 Hz, 1H), 7.25-7.34 (m, 3H), 7.19 (d, J=6.8 Hz, 2H), 6.334 (dd, J=10 Hz, 1.6 Hz, 1H), 6.15 (s, 1H), 4.98 (d and d, J=18 Hz, 2H), 4.42-4.51 (m, 2H), 4.10-4.32 (m, 7H), 3.29-3.36 (m, 1H), 2.37-3.11 (m, 18H), 1.76-2.22 (m, 12H), 1.60 (d, J=14 Hz, 1H), 1.47 (s, 3H), 1.28-1.42 (m, 3H), 1.17-1.23 (m, 1H), 1.12 (d, J=6 Hz, 3H), 0.90 (s, 3H), 0.80 (d, J=7.2 Hz, 3H), 0.68-0.69 (m, 3H), 0.61 (d, J=5.6 Hz, 3H). MS: 1236.76 [M+H]⁺.

### Example 5

Synthesis of the Compound of Example 5:

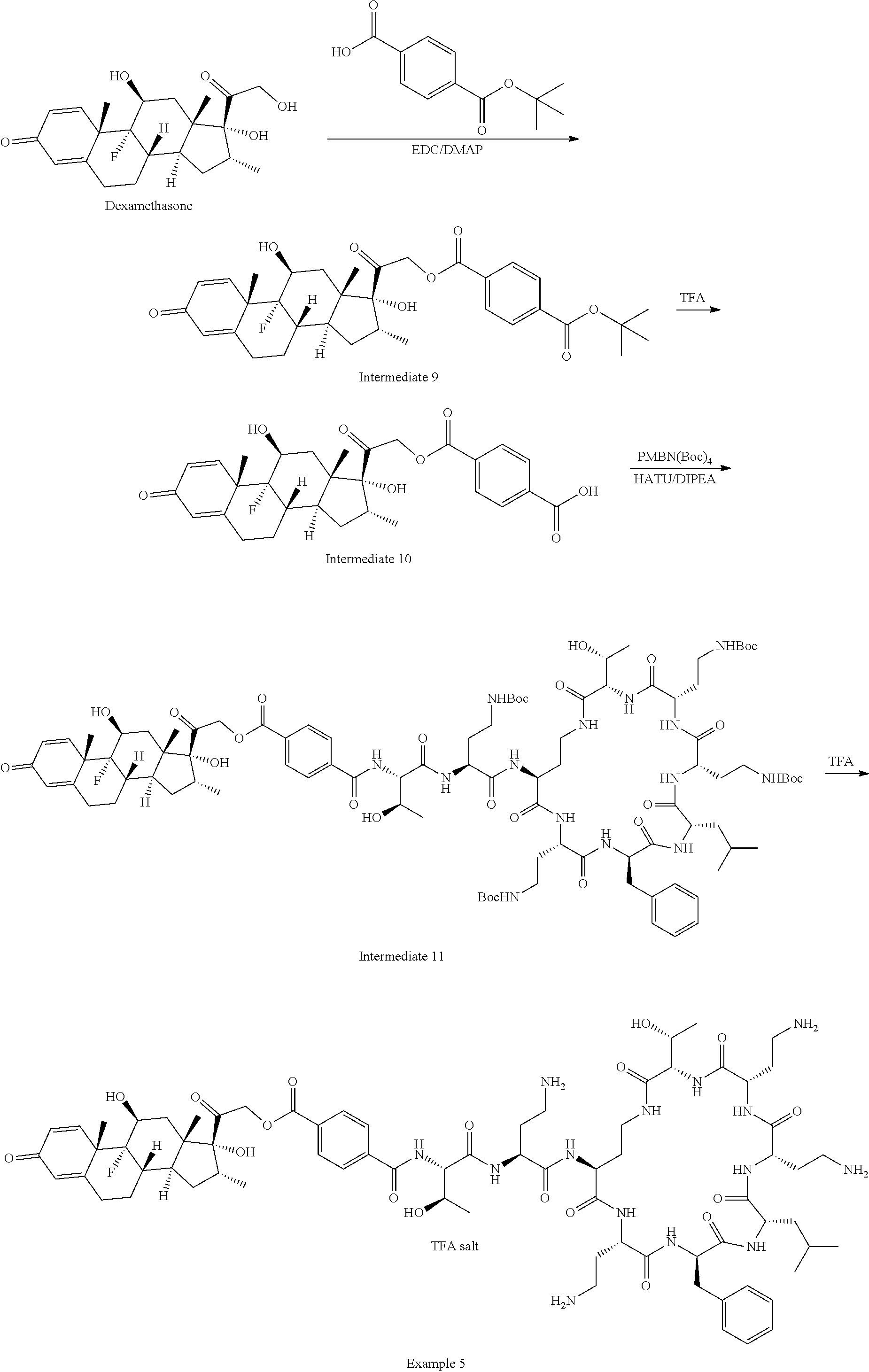

Compound of Example 5. Compound of Example 5 was analogously to procedure for the preparation of the Compound of Example 2 except that 4-(tert-butoxycarbonyl)benzoic acid was used instead of succinic anhydride. NMR: 8.12 (d, J=8.4 Hz, 2H), 7.90 (d, J=8.4 Hz, 2H), 7.46 (d, J=10 Hz, 1H), 7.25-7.34 (m, 3H), 7.19 (d, J=6.8 Hz, 2H), 6.34 (dd, J=1.6 Hz, 10 Hz, 1H), 6.15 (s, 1H), 5.17-5.31 (m, 2H), 4.09-5.52 (m, 12H), 3.17-3.30 (m, 1H), 2.99-3.04 (m, 10H), 2.37-2.82 (m, 5H), 1.71-2.22 (m, 15H), 1.48 (s, 3H), 1.31-1.44 (m, 3H), 1.25 (d, J=6.4 Hz, 3H), 1.17-1.22 (m, 1H), 1.07 (d, J=6.0 Hz, 3H), 0.96 (s, 3H), 0.83 (d, J=7.2 Hz, 3H), 0.70 (d, J=6.0 Hz, 3H), 0.63 (d, J=6.0 Hz, 3H). MS: 1486.02 [M+H]⁺.

### Example 6A

Synthesis of the Compound of Example 6:

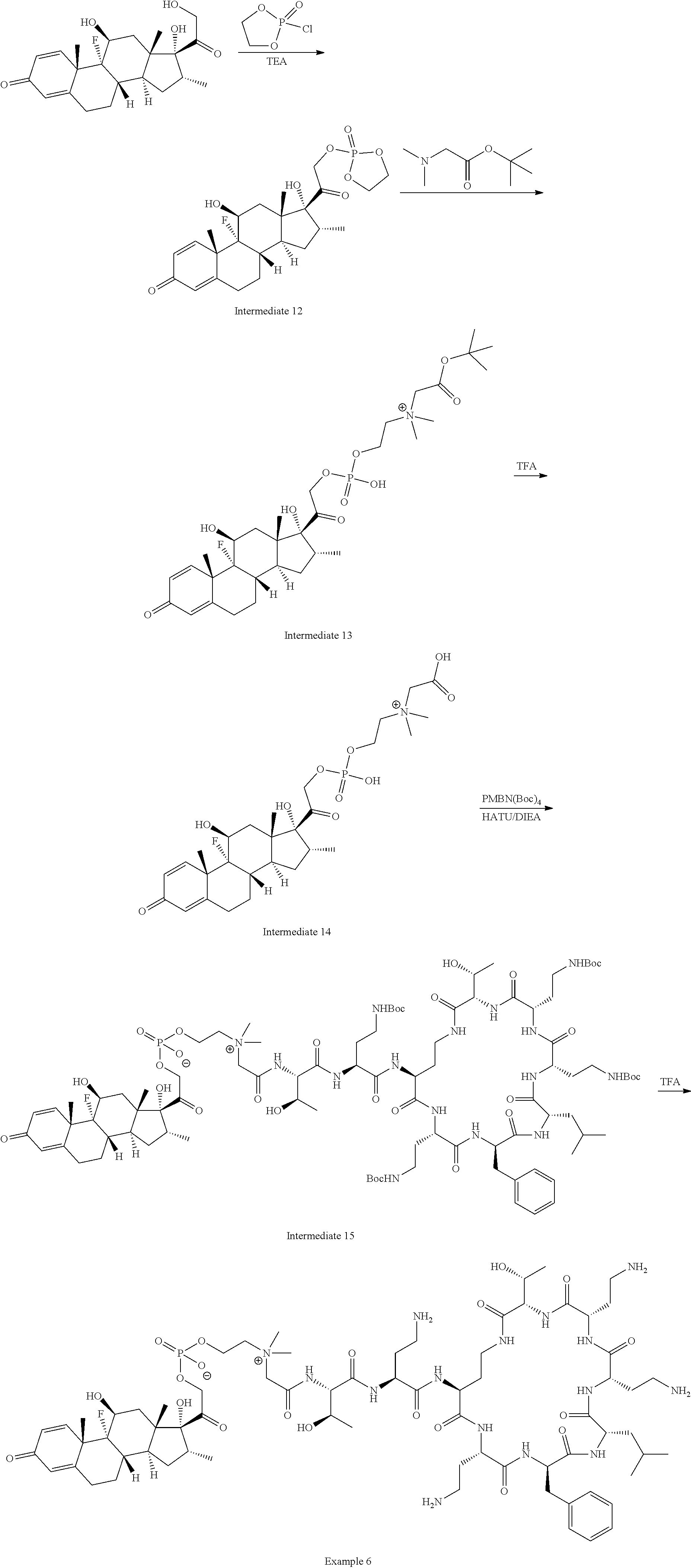

Compound of Example 6. Compound of Example 6 is made according to a synthetic procedure above starting from dexamethasone.

### Example 6B

Alternative Synthesis of the Compound of Example 6:

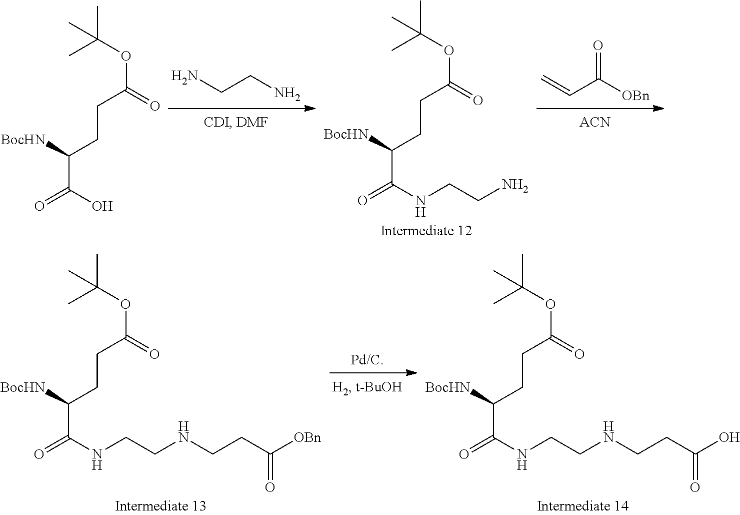

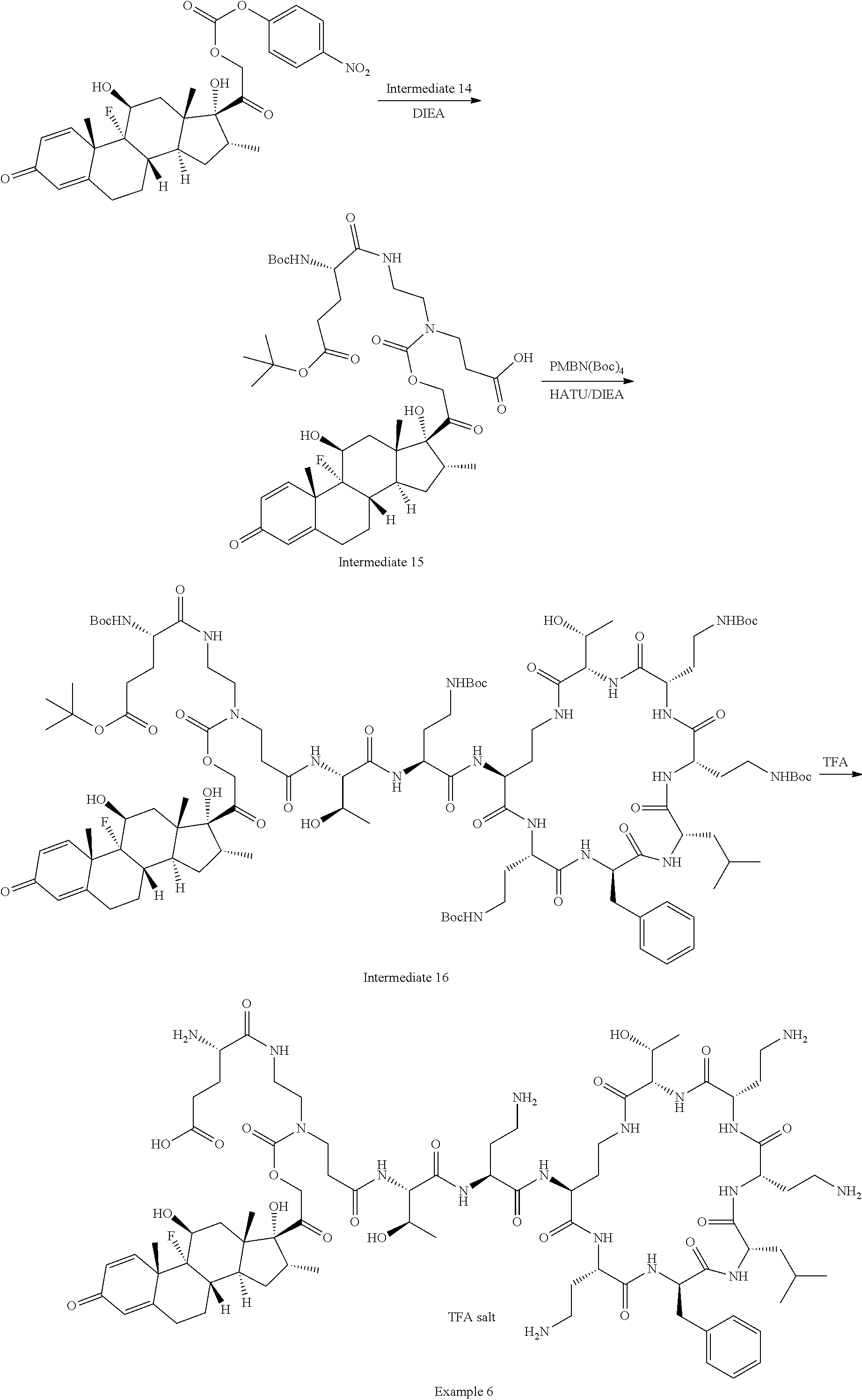

Intermediate 12. The mixture of (S)-5-(tert-butoxy)-2-((tert-butoxycarbonyl)amino)-5-oxopentanoic acid (505 mg, 1.67 mmol) and CDI (324 mg, 2 mmol) in DMF (4 mL) was stirred at r.t. for 1.5 h, then ethylene-1,2-diamine (1.1 mL, 16.7 mmol) was added. The mixture was stirred at r.t. for 1.5 h and distributed between DCM (10 mL) and brine (10 mL). It was then extracted with DCM (60 mL), organic layer was washed with water (2×10 mL), brine (10 mL), and dried (Na₂SO₄). Solvent was evaporated under vacuum to afford crude Intermediate 12 (0.62 g). MS: 346.16 [M+H]⁺.

Intermediate 13. The mixture of Intermediate 12 (0.62 g, 1.67 mmol) and benzyl acrylate (0.24 g, 1.5 mmol) in ACN (4 mL) was stirred at 45° C. under Ar for 36 h. The volatiles were removed under vacuum, and the residue was purified by HPLC (gradient ACN-water 0˜80%) to give Intermediate 13 (0.5 g). MS: 508.31 [M+H]⁺.

Intermediate 14. The suspension of Intermediate 13 (0.5 g, 0.99 mmol) and Pd/C (0.15 g) in t-BuOH (5 mL) was stirred at rt under H₂ for 4.5 h. The mixture was filtered and dried under vacuum to give the Intermediate 14 (0.42 g). MS: 418.25 [M+H]⁺.

Intermediate 15. Intermediate 14 (190 mg, crude) and Dex-Pnp (254 mg, 0.46 mmol) were dissolved in THF (5 mL), and DIEA (117.5 mg, 0.91 mmol) was added. The mixture was stirred at rt for 2 days. The mixture was concentrated and purified by HPLC (0.05% TFA in ACN-water 5-60%) to afford Intermediate 15 (180 mg) as white solid, MS: 836.14 [M+H]⁺.

Intermediate 16. To a solution of Intermediate 15 (100 mg, 0.12 mmol) in DMF (3 mL) was added DIEA (77 mg, 0.60 mmol), followed by T3P (150 mg, 0.24 mmol, w/w 50% in EA). The resulting mixture was stirred at rt for 30 min. PMBN-Boc4 (160 mg, 0.12 mmol) was added. The mixture was stirred at rt overnight. The mixture was diluted with EtOAc (10 mL) and washed with water (10 mL), the organic phase was dried (Na₂SO₄) and evaporated under vacuum to afford Intermediate 16 (200 mg). MS: 1091.17 [M+2H]²⁺.

Compound of Example 6. Compound of Example 6 was made according to a similar procedure to the preparation of the Compound of Example 1. ¹H NMR: 7.45 (d, J=10.1 Hz, 1H), 7.30 (dq, J=14.6, 7.3 Hz, 3H), 7.19 (d, J=7.1 Hz, 2H), 6.34 (dd, J=10.1, 1.6 Hz, 1H), 6.15 (s, 1H), 4.91 (d, J=12.5 Hz, 1H), 4.51 (t, J=8.2 Hz, 1H), 4.45-4.39 (m, 2H), 4.32 (d, J=9.0 Hz, 1H), 4.19 (ddd, J=21.2, 14.3, 4.2 Hz, 8H), 3.96 (dt, J=23.4, 6.5 Hz, 1H), 3.67-3.20 (m, 8H), 3.11-2.91 (m, 10H), 2.87-2.35 (m, 10H), 2.22-2.00 (m, 10H), 1.95-1.72 (m, 6H), 1.62 (d, J=13.2 Hz, 1H), 1.48 (s, 3H), 1.43-1.32 (m, 3H), 1.25-1.07 (m, 7H), 0.91 (d, J=4.0 Hz, 3H), 0.80 (d, J=6.8 Hz, 3H), 0.69 (d, J=5.7 Hz, 3H), 0.62 (d, J=5.9 Hz, 3H). MS: 1624.87 [M+H]⁺.

### Example 7A

Synthesis of the Compound of Example 7:

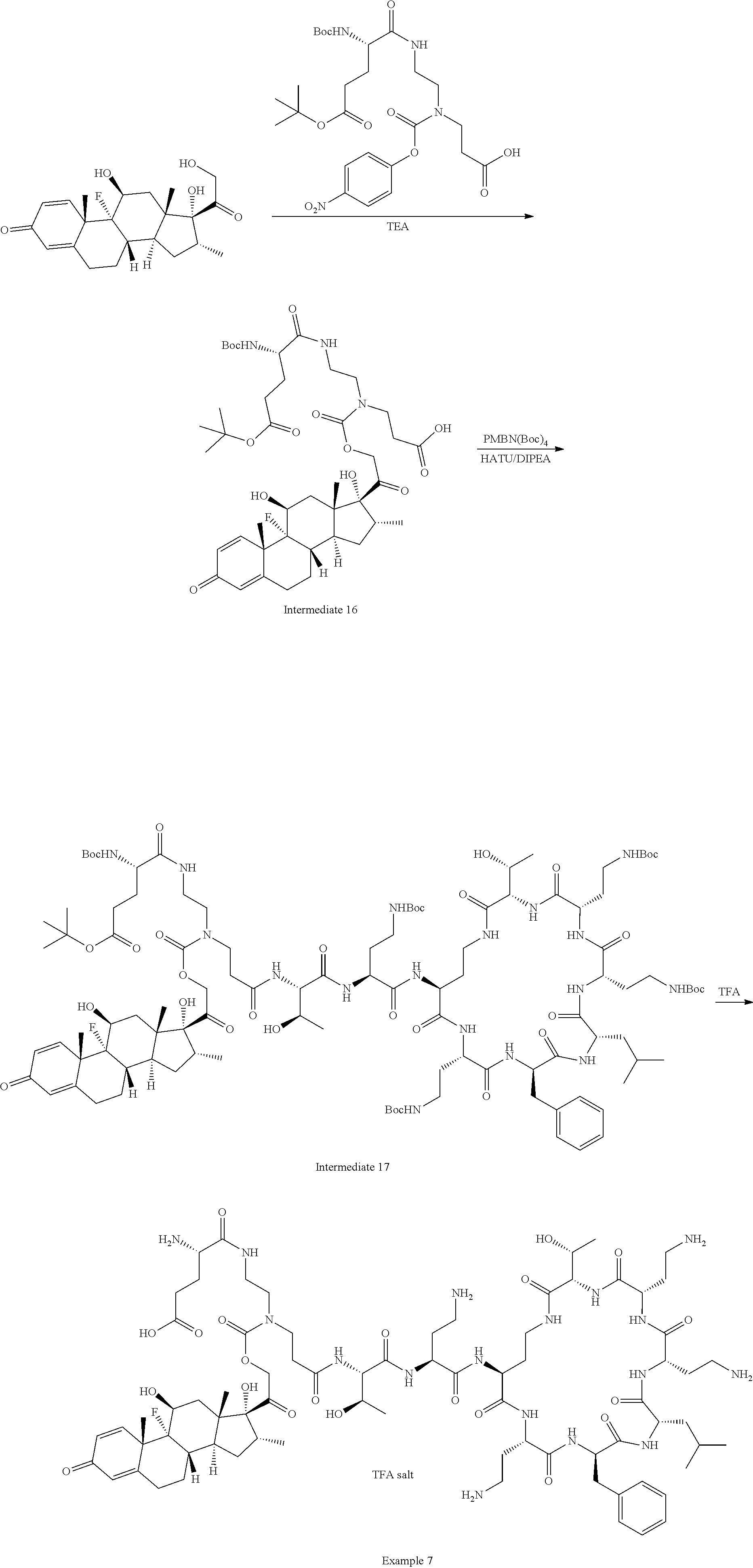

Compound of Example 7. Compound of Example 7 is made according to a synthetic procedure above starting from dexamethasone.

### Example 7B

Synthesis of the Compound of Example 7:

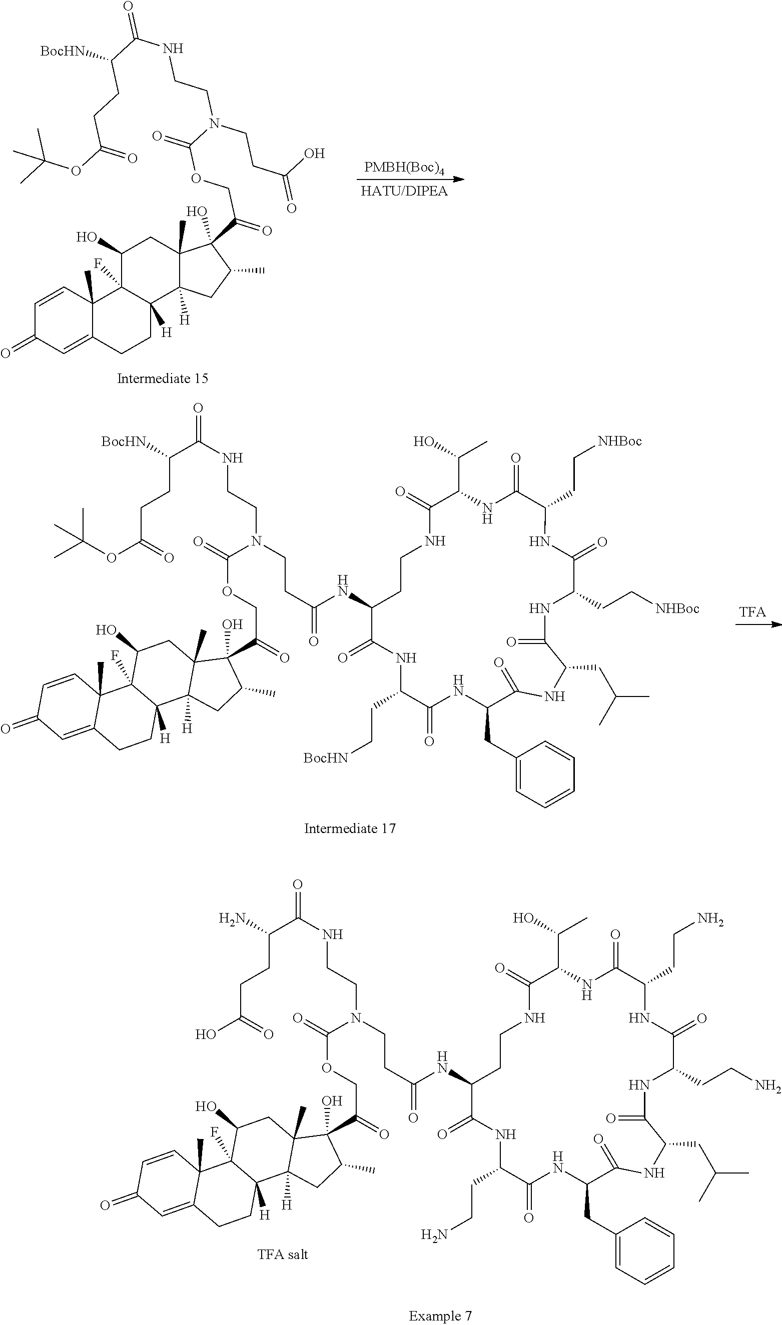

Compound of Example 7. Compound of Example 7 was made analogously to the procedure for the Compound of Example 6 except that Intermediate 15 was coupled with PMBH(Boc)₃ instead of PMBN(Boc)₄. ¹H NMR: 7.43 (dd, J=10.1, 4.6 Hz, 1H), 7.33-7.22 (m, 3H), 7.17 (d, J=7.5 Hz, 2H), 6.32 (d, J=10.1 Hz, 1H), 6.13 (s, 1H), 5.03 (d, J=18.2 Hz, 1H), 4.90 (s, 1H), 4.51-4.40 (m, 1H), 4.29 (d, J=10.1 Hz, 1H), 4.24-4.03 (m, 6H), 3.92 (dd, J=24.1, 17.9 Hz, 1H), 3.67-3.51 (m, 2H), 3.51-3.14 (m, 6H), 3.12-2.86 (m, 9H), 2.80 (ddd, J=15.5, 9.7, 6.2 Hz, 1H), 2.76-2.32 (m, 8H), 2.25-1.93 (m, 9H), 1.93-1.53 (m, 7H), 1.46 (s, 3H), 1.42-1.24 (m, 3H), 1.17 (s, 1H), 1.09 (s, 3H), 0.88 (d, J=12.0 Hz, 3H), 0.77 (d, J=7.1 Hz, 3H), 0.71-0.65 (m, 3H), 0.60 (t, J=6.0 Hz, 3H), MS: 1423.77 [M+H]⁺.

### Example 8

Synthesis of the Compound of Example 8:

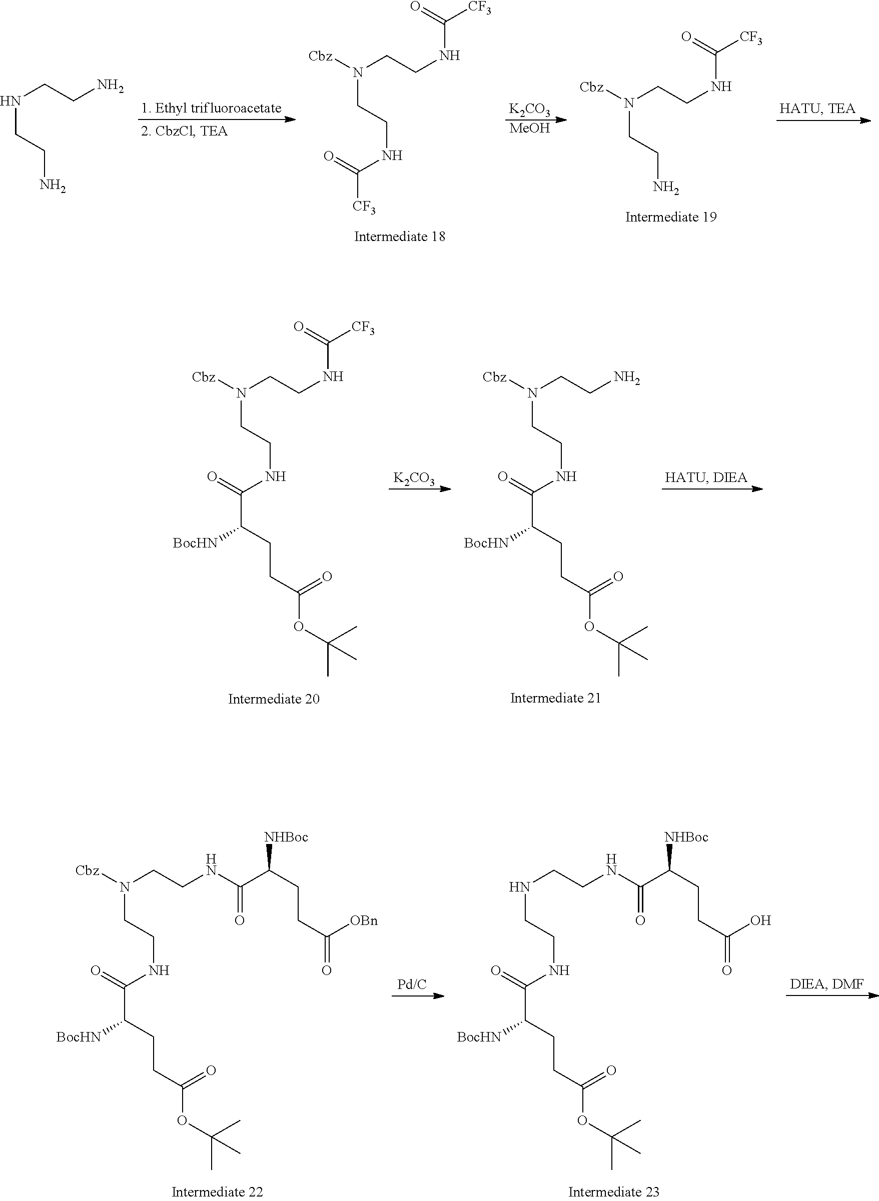

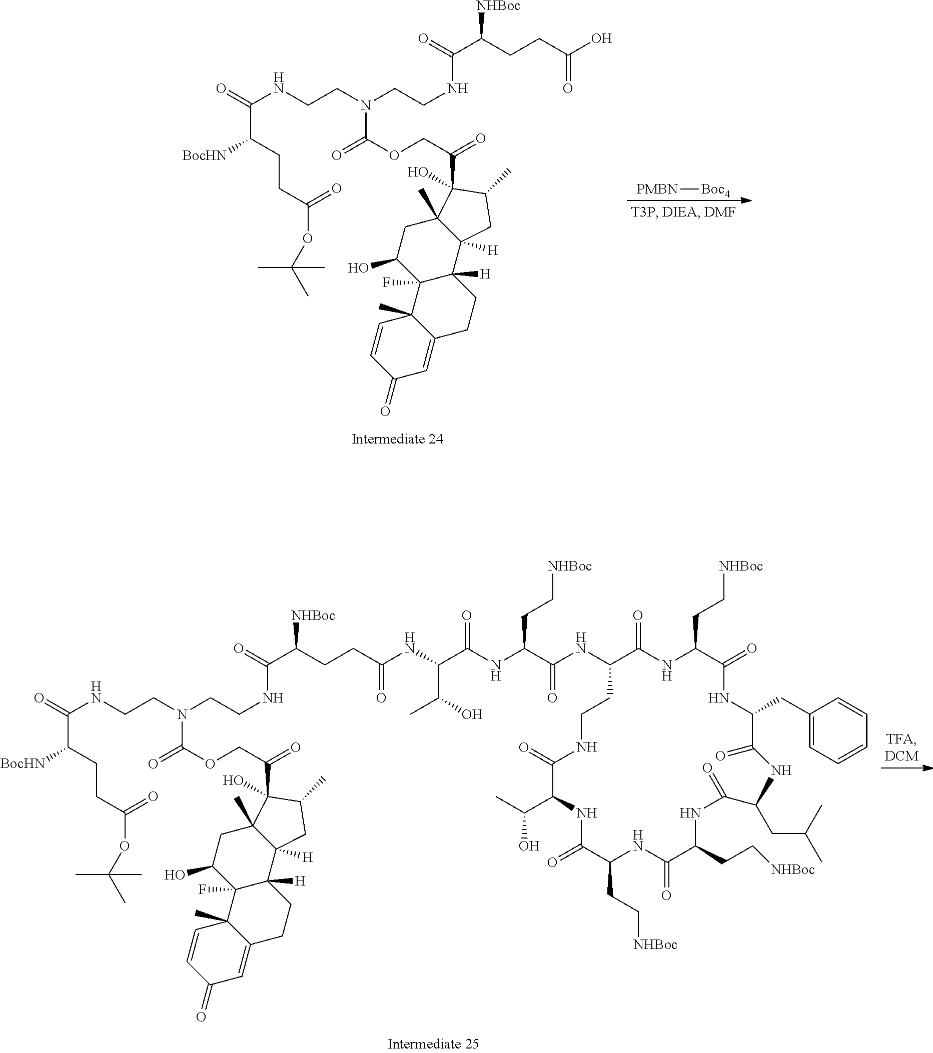

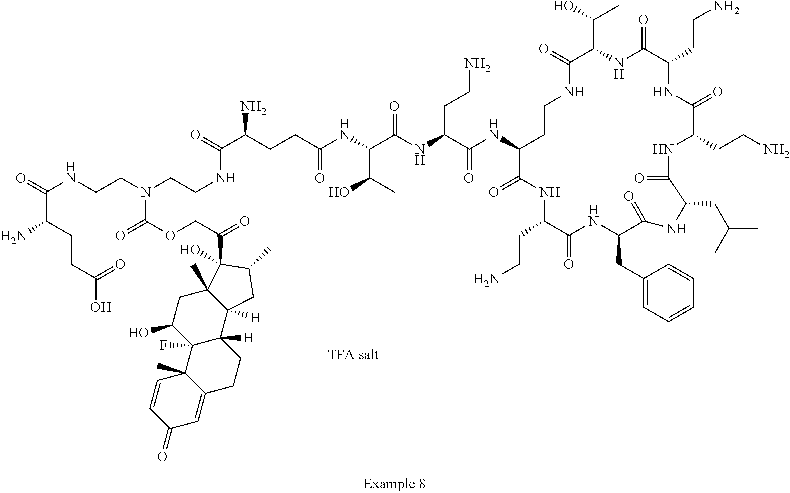 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC[C@H](N)C(=O)NCCN(CCNC(=O)[C@@H](N)CCC(=O)O)C(=O)OCC(=O)[C@@]2(O)[C@H](C)C[C@H]3[C@@H]4CCC5=CC(=O)C=C[C@]5(C)[C@@]4(F)[C@@H](O)C[C@@]32C)[C@@H](C)O)CCNC(=O)[C@H]([C@@H](C)O)NC(=O)[C@H](CCN)NC(=O)[C@H](CCN)NC1=O]

Intermediate 18. N¹-(2-aminoethyl)ethylene-1,2-diamine (3.35 g, 32 mmol) was dissolved in DCM (30 mL) and cooled to 0° C. Ethyl trifluoroacetate (9.69 g, 68 mmol) in DCM (10 mL) was slowly added. After 1 h, TEA (3.87 g, 384 mmol) was added, followed by CbzCl (6.0 g, 35.2 mmol) in DCM (10 mL) at 0° C. The mixture was stirred at rt for 16 h, then diluted with water and DCM. Organic layer was separated and evaporated under vacuum, and the crude product was purified by silica gel chromatography to afford Intermediate 18 (2.9 g) as white solid, yield 21%. MS: 430.0 [M+H]⁺.

Intermediate 19. To a solution of Intermediate 18 (2.0 g, 4.7 mmol) in MeOH (152 mL) was added K₂CO₃ (256 mg, 1.9 mmol). The mixture was stirred at 65° C. overnight. The solvent was removed under vacuum, and the product was purified by HPLC to afford Intermediate 19 (490 mg) as colorless oil. MS: 334.2 [M+H]⁺.

Intermediate 20. DIPEA (561 mg, 4.35 mmol) and HATU (771 mg, 2.03 mmol) was added to a mixture of (S)-5-(tert-butoxy)-2-((tert-butoxycarbonyl)amino)-5-oxopentanoic acid (527 mg, 1.74 mmol) in DMF (5 mL) at 0° C., and then Intermediate 19 (483 mg, 1.45 mmol) was added. The resulting mixture was stirred at rt for 2 h. The mixture was diluted with EtOAc (20 mL), washed with water (20 mL) and evaporated under vacuum. The residue was purified by HPLC (0.1% TFA in gradient ACN-water 0-80%) to afford Intermediate 20 (355 mg) as colorless oil. MS: 619.1 [M+H]⁺.

Intermediate 21. To a solution of Intermediate 20 (355 g, 0.57 mmol) in MeOH (5 mL)/H₂O (0.5 mL) was added K₂CO₃ (79 mg, 0.57 mmol). The mixture was stirred at rt overnight. The mixture was diluted with EtOAc (10 mL), washed with water (10 mL), and evaporated under vacuum. The residue was purified by HPLC (0.1% TFA in gradient ACN-water 0-60%) to afford Intermediate 21 (188 mg) as colorless oil. MS: 523.31 [M+H]⁺.

Intermediate 22. To a solution of (S)-5-(benzyloxy)-2-((tert-butoxycarbonyl)amino)-5-oxopentanoic acid (146 mg, 0.43 mmol) in DMF (4 mL) at 0° C. were added HATU (178 mg, 0.4 mmol) and DIEA (139 mg, 1.08 mmol), the mixture was stirred at 0° C. for 20 min. Then Intermediate 21 (188 mg, 0.36 mmol) was then added, and the mixture was stirred at rt for overnight. The mixture was diluted with 20 mL of EtOAc, washed with 20 mL of water and evaporated under vacuum. The residue was purified by HPLC (0.1% TFA ACN-water 0-90%) to afford Intermediate 22 (210 mg) as colorless oil. MS: 842.3 [M+H]⁺.

Intermediate 23. To a solution of Intermediate 22 (85 mg, 0.10 mmol) in EA (3 mL) was added Pd/C (10 mg, 10%), and the mixture was then stirred under H₂ atmosphere overnight. It was then filtered through Celite and evaporated under vacuum to afford Intermediate 23 (59.5 mg) as colorless oil. MS: 618.4 [M+H]⁺.

Intermediate 24. DIEA (31 mg, 0.24 mmol) was added to a solution of Intermediate 23 (49.5 mg, 0.08 mmol) and 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (4-nitrophenyl) carbonate (53.6 mg, 0.096 mmol) dissolved in DMF (2 mL). The mixture was stirred at rt for 3 days, then diluted with EtOAc (10 mL), washed with water (10 mL), and evaporated under vacuum. The residue was purified by HPLC (0.1% TFA in gradient ACN-water 0-80%) to afford Intermediate 24 (30 mg) as colorless oil. MS: 1036.2 [M+H]⁺.

Intermediate 25. To a solution of Intermediate 24 (20 mg, 0.019 mmol) in DMF (2 mL) was added T3P (50% in EtOAc, 24 mg, 0.038 mmol), followed by DIEA (12 mg, 0.096 mmol) and PMBN-Boc₄ (26 mg, 0.019 mmol). The mixture was stirred at rt for overnight. The mixture was diluted with EtOAc (10 mL), washed with water (10 mL), and evaporated under vacuum. The residue was purified by HPLC (0.1% TFA in gradient ACN-water 0-80%) to afford Intermediate 24 (30 mg) as colorless oil. MS: 1191.1 [M+2H]²⁺.

Compound of Example 8. To a solution of Intermediate 24 (30 mg, 0.0126 mmol) in DCM (1 mL) was added TFA (0.5 mL). The resulting mixture was stirred at room temperature for 16 hrs. The mixture was concentrated and purified by HPLC (0.05% TFA in gradient ACN-water 5-30%) to afford Compound of Example 8 (7.07 mg) as white solid. ¹H NMR: 7.45 (d, J=10.3 Hz, 1H), 7.31 (d, J=6.8 Hz, 2H), 7.28 (d, J=7.3 Hz, 1H), 7.19 (d, J=7.4 Hz, 2H), 6.35 (d, J=10.1 Hz, 1H), 6.15 (s, 1H), 5.01 (d, J=18.4 Hz, 1H), 4.90 (d, J=18.1 Hz, 1H), 4.50 (dd, J=13.9, 8.3 Hz, 1H), 4.46-4.40 (m, 2H), 4.32 (d, J=9.4 Hz, 1H), 4.26 (d, J=4.5 Hz, 1H), 4.23-4.11 (m, 8H), 4.01-3.88 (m, 2H), 3.52-3.33 (m, 8H), 3.25 (dt, J=15.3, 7.6 Hz, 1H), 3.10-2.98 (m, 10H), 2.95-2.89 (m, 1H), 2.85-2.77 (m, 1H), 2.72 (s, 1H), 2.68-2.60 (m, 1H), 2.50-2.33 (m, 6H), 2.09 (ddd, J=23.8, 20.8, 10.5 Hz, 13H), 1.87 (s, 3H), 1.81-1.71 (m, 2H), 1.61 (d, J=13.6 Hz, 1H), 1.48 (s, 3H), 1.45-1.32 (m, 3H), 1.13 (dd, J=16.8, 6.3 Hz, 6H), 0.91 (s, 3H), 0.80 (d, J=6.8 Hz, 3H), 0.69 (d, J=5.7 Hz, 3H), 0.62 (d, J=5.0 Hz, 3H). MS: 1724.9 [M+H]⁻.

### Example 9

Synthesis of the Compound of Example 9:

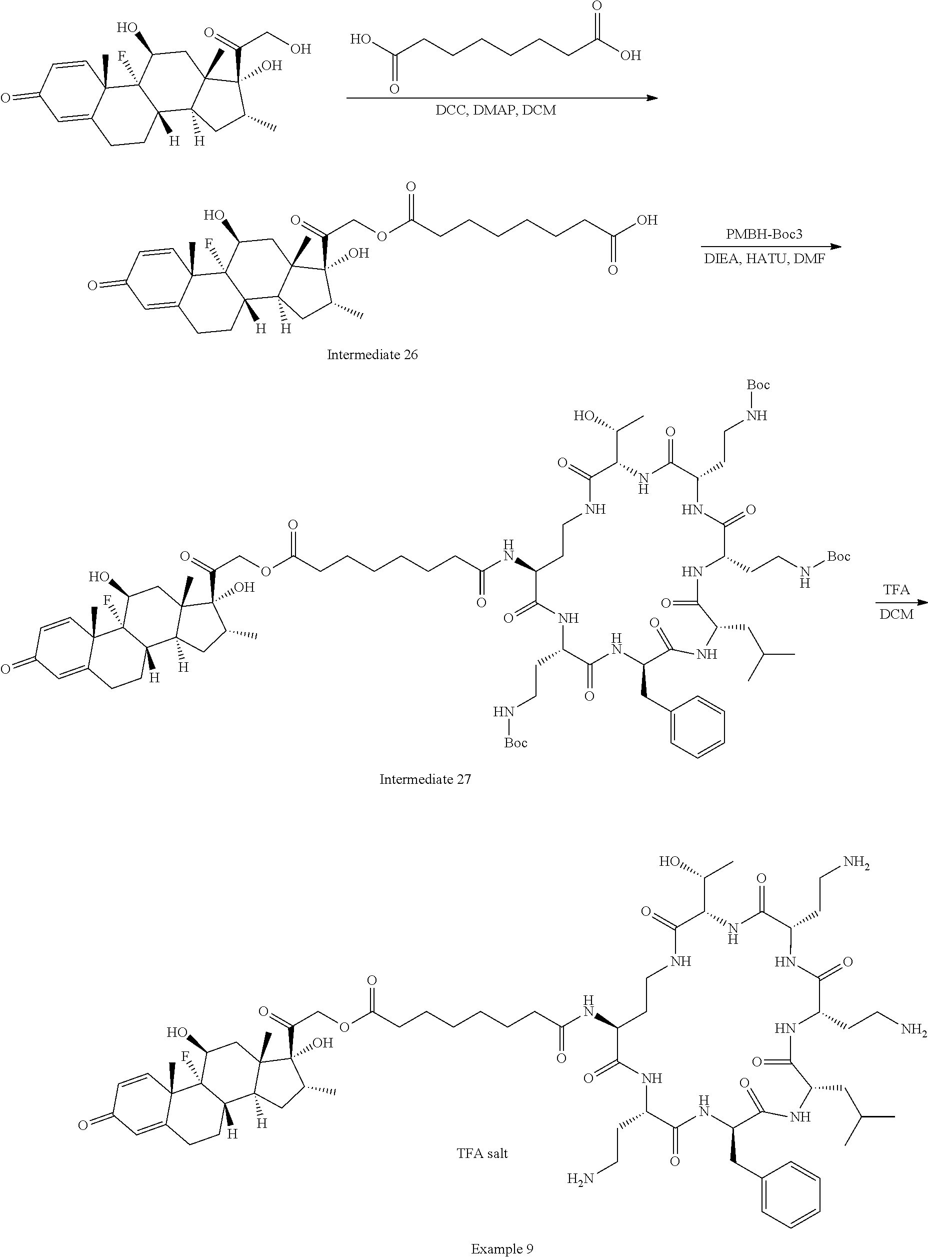

Compound of Example 9. Compound of Example 9 was made analogously to the procedure to the Compound of Example 1 started from octanedioic acid except that Intermediate 26 was coupled with PMBH(Boc)₃ instead of PMBN(Boc)₄. ¹H NMR (CD₃OD): 7.30 (d, J=10.1 Hz, 1H), 7.24-7.10 (m, 5H), 6.19 (dd, J=10.1, 1.8 Hz, 1H), 5.98 (s, 1H), 4.92-4.80 (m, 2H), 4.45 (dd, J=9.4, 4.4 Hz, 1H), 4.31 (dd, J=10, 1, 6.3 Hz, 1H), 4.26-4.14 (m, 3H), 4.10-3.92 (m, 4H), 3.54 (br d, J=7.6 Hz, 1H), 3.02-2.93 (m, 5H), 2.92-2.84 (m, 3H), 2.62 (d, J=6.4 Hz, 1H), 2.44-2.26 (m, 5H), 2.26-2.10 (m, 4H), 2.09 (br s, 4H), 1.89-1.73 (m, 4H), 1.64 (d, J=12.0 Hz, 1H), 1.61-1.52 (m, 5H), 1.49 (s, 3H), 1.47-1.37 (m, 2H), 1.37-1.24 (m, 5H), 1.24-1.17 (m, 2H), 1.15-1.08 (m, 4H), 0.91 (s, 3H), 0.76 (d, J=73 Hz, 4H), 0.68-0.54 (m, 6H). MS: 647.4 [M+2H]²⁺.

### Example 10

Synthesis of the Compound of Example 10:

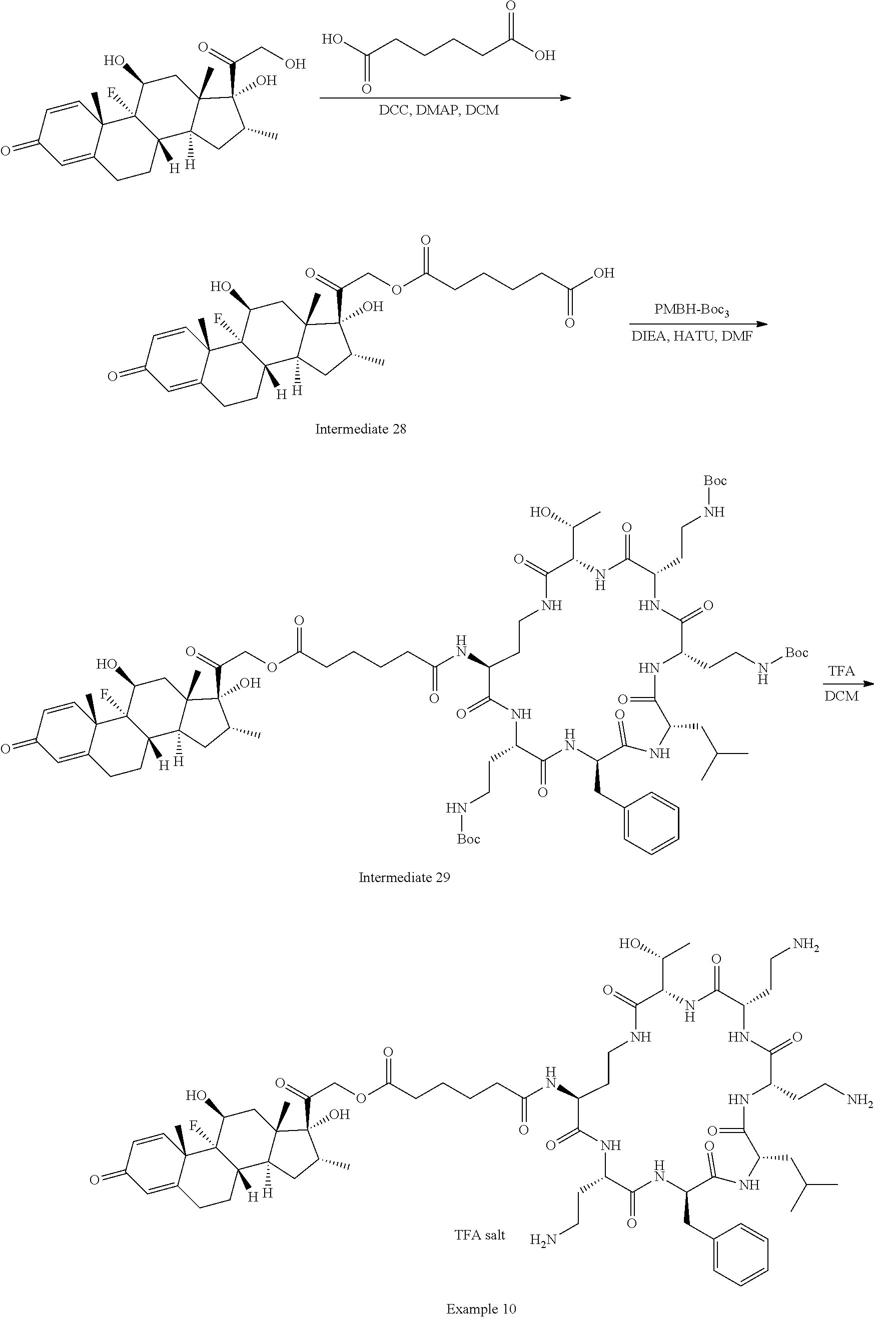

Compound of Example 10. Compound of Example 10 was made analogously to the procedure to the Compound of Example 1, except started from adipic acid, and that Intermediate 29 was coupled with PMBH(Boc)₃ instead of PMBN(Boc)₄. ¹H NMR (CD₃OD): 7.32 (d, J=10.1 Hz, 1H), 7.11-7.26 (m, 5H), 6.20 (dd, J=10.1, 1.8 Hz, 1H), 6.00 (s, 1H), 4.83-4.99 (m, 2H), 4.42-4.49 (m, 1H), 4.28-4.36 (m, 1H), 4.15-4.27 (m, 3H), 3.93-4.14 (m, 4H), 3.51-3.62 (m, 1H), 2.86-3.06 (m, 9H), 2.76-2.86 (m, 1H), 2.55-2.69 (m, 1H), 2.36-2.45 (m, 3H), 2.31 (dd, J=11.1, 5.5 Hz, 2H), 2.19-2.27 (m, 3H), 2.09-2.19 (m, 2H), 1.98-2.08 (m, 3H), 1.75-1.90 (m, 4H), 1.52-1.71 (m, 6H), 1.48-1.52 (m, 3H), 1.35-1.47 (m, 2H), 1.17-1.31 (m, 2H), 1.08-1.17 (m, 4H), 0.92 (s, 3H), 0.72-0.81 (m, 3H), 0.55-0.69 (m, 5H). MS: 633.4 [M+2H]².

### Utility and Testing

The compounds provided herein exhibit potent in vivo efficacy against a variety of inflammation-associated kidney disorders, including CKD and AKI, post-surgery inflammation (such as in kidney transplantation surgery), or an inflammation induced by anticancer or antibacterial therapy, or in treatment of diabetes, or same due to an exposure to nephrotoxic substances in the environment. Therefore, these agents are useful for therapy of such renal diseases.

In vitro activity of compounds provided herein may be assessed by testing procedures using standard bioassays used for evaluation of active entities (payloads, such as HR¹) incorporated (conjugated) within the composition provided herein. Selection of specific assay(s) is determined from a known or anticipated mode-of-action of the payload structure. For example, conjugates incorporating anti-inflammatory vanin-1 inhibitors may be tested in human vanin-1 enzymatic assay as described in publication PCT WO 2020/114943.

Preferential renal targeting validating the delivery of compounds provided herein into kidneys (or at the site of a kidney injury) is evaluated, for example, by pharmacokinetic (PK) tests, such as in a standard rodent PK test. PK data are generally used to establish the key parameters predictive of the therapy outcome. Thus, a drug concentration (C) at given time points, drug concentration at the target tissue(s) (C_{Target}), area under the curve (AUC) for a plot monitoring the change in the systemic drug concentration over time and other parameters are generally predictive of therapeutic efficacy. For example, drug concentration at an organ affected by cancer is important for effective action of anticancer agents (as described, for example, by Zhang et al. in Drug Metabolism and Disposition. 2019, vol. 47, p. 1122).

Representative compounds provided herein were tested in a rodent PK model of intravenous administration performed analogously to methods described in the monograph Current Protocols in Pharmacology, 2005, 7.1.1-7.1.26, John Wiley & Sons, Inc. Illustrative mouse PK data for the compounds of Examples 1 and 2 are summarized in the Table 1 below. As clear from the data, these compounds exhibit a surprising ability to target kidneys, confirmed by high levels of preferential concentrations in kidney tissues, the organ for a targeted therapy.

TABLE 1
Enhanced delivery of anti-inflammatory and immunomodulating
agent dexamethasone with exemplary compounds
(dexamethasone levels in kidneys at T = 3 h).

| | | | Enhanced |
|---|---|---|---|
| | | | dexamethasone |
| | Dexamethasone | Kidncy | levels in kidney |
| | dosing | tissue | expressed per |
| | equivalent ^{a}, | concentration, | ratio vs. dosing of |
| EXAMPLES | μmol/kg | ng/g | free dexamethasone |
| Dexamethasone | 2.6^{b} | 13.0 | 1 |
| Example 1 | 2.1^{c} | 122.2 | 9.4 |
| Example 2 | 2.2^{d} | 93.4 | 7.2 |

^{a} Dosed by an injection into a mouse tail vein.
^{b}Dose of 1 mg/kg expressed in μmol/kg per molecular weight (MW) of 392.47 Daltons.
^{c}Dose of 4 mg/kg expressed in μmol/kg per MW of 1,921.79 Daltons for the compound of Example 1 (uncorrected for solvated water content, as tested).
^{d}Dose of 4 mg/kg expressed in μmol/kg per MW of 1,893.73 Daltons for the compound of Example 2 (uncorrected for solvated water content, as tested).

Remarkably, the administration of the compounds of Examples 1 and 2 (comprising conjugated forms of dexamethasone) resulted in kidney dexamethasone levels (measured as drug concentration in the kidney tissue) that were about 9- and 7-fold, respectively, greater than dexamethasone kidney levels achieved through injection of dexamethasone in its (unconjugated) drug form.

Even more surprisingly, this beneficial enhancement is observed with the total amount of dexamethasone administered through exemplary compounds herein in amounts (dosing) much lower than that administered as the free (unconjugated) drug. Since the drug levels at the site affected by a disease directly determine a therapeutic efficacy of a pharmaceutical agent, the data illustrated in Table 1 indicate the greatly enhanced therapeutic potential (in vivo activity) of the novel compositions provided herein.

Furthermore, based on the PK data, the release of dexamethasone administered in the form of compounds of Examples 1 and 2 persisted for at least 24 h (T=24 h, the last time point evaluated), with the kidney drug concentrations determined at 21.5 and 18.5 ng/g for Examples 1 and 2, respectively. In a striking contrast, the drug levels achieved from a standard injection of unconjugated drug dexamethasone were virtually undetectable after only 6 h. This surprising and beneficial effect for the compounds described herein illustrates additional potential for an improved therapy of inflammation-associated diseases with the compounds provided herein.

The data illustrated in the Table 1 indicate that a less frequent dosing of such agents is feasible, as compared to a standard administration of dexamethasone. Based on this data, the compounds of Examples 1 and 2 may be dosed much less frequently, for example, once-daily, once-more than a day, or once-weekly. This advantage greatly benefits patients in need of such therapy. In addition, this useful feature provides significant pharmacoeconomic benefits due to minimized need in medical procedures at a hospital providing the treatment, such as an intravenous administration.

In vivo activity of compounds provided herein can be assessed by testing procedures such described, for example, for evaluation of anti-inflammatory and immunomodulating effect of corticosteroid agents by Chen et al. in Inflammopharmacology, 2018 26, pp. 1331-1338; or for testing in rodent models of nondiabetic and diabetic chronic nephropathies described by Perico et al. in Kidney International, 2005, vol. 68, Supplement 98, pp. S21-S24, by Yang, et al. in Process Biochemistry, 2019, Vol. 83, pp. 198-205, and by Remuzzi et al. in Kidney International, 2002, vol. 62, pp. 885-894, as well as in additional testing methods in references cited therein.

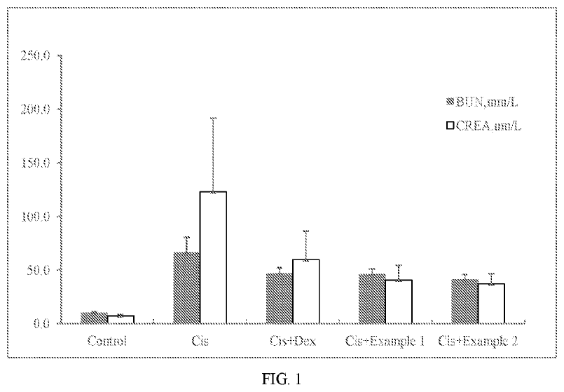

Specifically, in one of the tests, cisplatin (20 mg/kg) was administrated to C57BL/6 mouse (male, weight range 20-25 g) by intraperitoneal injection to induce acute kidney injury. After 1 h, testing articles (saline, or dexamethasone, or a compound of Example 1 or 2) were given by intravenously. On day 3, blood CREA (creatinine) and BUN (blood urea nitrogen) were determined. Illustrative in vivo efficacy data for the compounds of Examples 1 and 2 are summarized in the FIG. 1 below.

As shown in FIG. 1, in this animal model, acute kidney injury (AKI) was induced by a single dose of cisplatin at 20 mg/kg, confirmed by increases in both blood urea nitrogen (BUN) and creatinine (CREA) levels, used as the standard biomarkers for AKI.

As apparent from the BUN and CREA values in FIG. 1., the compounds of Examples 1 and 2 possess greatly increased anti-inflammatory efficacy in the AKI model, as compared to dexamethasone. Surprisingly, the enhanced efficacy was achieved for the compounds of Examples 1 and 2 only at about 20% dexamethasone equivalent dosing, as compared to dosing for the free (unconjugated) drug. In other words, the compounds described herein increased in vivo efficacy about 4-5-fold, as compared to dexamethasone. This surprising effect is in line with greatly enhanced exposure of dexamethasone levels in kidney from the compounds described herein, as described above in the pharmacokinetic study (Table 1).

While possessing a high efficacy in mammals, certain compounds provided herein exhibit little or no toxic against normal kidney cells, both in vitro (such as in human kidney cell HK-2 assay) and in a live mammal (rodent) model.

A safe profile of the compounds described herein may be further established in biomarker assays predictive of nephrotoxicity. Several such assays (including NGAL assay) have been described, for example, by Keirstead et al. in Toxicol. Sci. 2014, vol. 137, pp. 278-291.

Thus, certain compounds described herein exhibit high efficacy in the therapy of inflammation-associated renal disorders, but do not suffer from excessive off-target toxicity limiting a standard therapy of such diseases.

Therefore, novel compounds and compositions provided herein potentially provide a long thought-after safer and effective targeted therapy for kidney diseases and renal injury, including CKD, AKI, and renal injuries encountered in kidney transplantations,

### Administration and Pharmaceutical Formulations

In general, the compounds provided herein can be administered in a therapeutically effective amount by any of the accepted modes of administration for agents that serve similar utilities. By way of example, compounds provided herein may be administered orally, parenterally, transdermally, topically, rectally, or intranasally, or by way of intra-tumoral administration. The actual amount of a compound provided herein, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

The data obtained from the cell culture assays and animal studies can be used in formulating a range of dosage for use in humans. The dosage of such compounds lies preferably within a range of circulating concentrations that include therapeutic efficacy with little or no toxicity. The dosage may vary within this range depending upon the dosage form employed and the route of administration utilized. For any compound used in the method provided herein, the therapeutically effective dose can be estimated initially from animal models. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans.

When employed as pharmaceuticals, the compounds provided herein are usually administered in the form of pharmaceutical compositions. These compounds can be administered by a variety of routes including oral, parenteral, transdermal, topical, rectal, and intranasal.

Compounds provided herein are effective as injectable, oral, inhalable, topical, or intra-tumor administration compositions. Such compositions are prepared in a manner well known in the pharmaceutical art and comprise at least one active compound.

This disclosure also includes pharmaceutical compositions which contain, as the active ingredient, one or more of the compounds provided herein above associated with pharmaceutically acceptable carriers. In making the compositions of this disclosure, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions are preferably formulated in a unit dosage form, each dosage containing from about 0.1 to about 2000 mg, more usually about 1 to about 900 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound provided herein above is employed at no more than about 20 weight percent of the pharmaceutical composition, more preferably no more than about 15 weight percent, with the balance being pharmaceutically inert carrier(s).

An active compound is effective over a wide dosage range and is generally administered in a pharmaceutically or therapeutically effective amount. It, will be understood, however, that the amount of the compound actually administered can be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 mg/kg to about 250 mg/kg, more preferably about 1.0 mg/kg to about 50 mg/kg of body weight/day.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient described herein.

The tablets or pills described herein may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions described herein may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Additionally, liposomal formulations of compounds described herein may be used, for example, to enhance therapeutic effect against certain infections, such as pneumonia or ling infections.

Intra-tumoral administration of compounds provided herein employs solutions or gels thereof prepared in suitable aqueous solutions containing appropriate excipient additives, such as dextrose, polyethylene glycol, cremophore, cyclodextrin, and alike.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

Other suitable formulations for use can be found in Remington's Pharmaceutical Sciences, Mace Publishing Company, Philadelphia, PA, 17th ed. (1985).

Optionally, the compounds described herein may be co-administered with additional agents, including antioxidants, such as ascorbic acid, or megalin-receptor inhibitors generally known to attenuate adverse effects of polymyxin drugs.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference. Optionally, the compounds described herein could be administered as nanomicells, or nanomaterials-encapsulated compositions, prepared as described, for example, by Taki et al. in Pharmaceut., 2012, vol. 3, p. 1092.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the present disclosure has been described herein in conjunction with a preferred aspect, a person with ordinary skills in the art, after reading the foregoing specification, can affect changes, substitutions of equivalents and other types of alterations to the disclosure as set forth herein. Each aspect described above can also have included or incorporated therewith such variations or aspects as disclosed in regard to any or all of the other aspects. The present disclosure is also not to be limited in terms of the particular aspects described herein, which are intended as single illustrations of individual aspects provided herein. Many modifications and variations of this disclosure can be made without departing from its spirit and scope, as will be apparent to those skilled in the art. Functionally equivalent methods within the scope of this disclosure, in addition to those enumerated herein, will be apparent to those skilled in the art from the foregoing descriptions. It is to be understood that this disclosure is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A compound of Formula II-P-2: 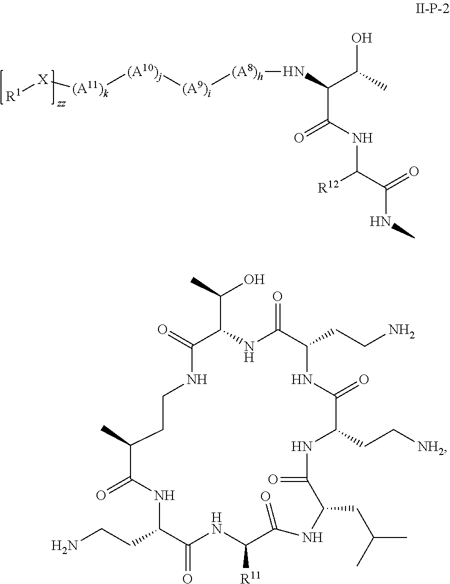
or a pharmaceutically acceptable salt, solvate, or hydrate thereof wherein:
R¹ is selected from alkyl, aryl, biaryl, heteroaryl, heteroarylaryl, and arylheteroaryl; or
R¹ is a residue attached to X formed by subtracting a single atom from respective parent (precursor) structure (H)ₙR¹ at any one of the following H-containing group(s) independently selected from NH, OH, SH, C(═O)OH, CONH, SO₂NH, and S(═O)NH when present in (H)ₙR¹; and wherein (H)ₙR¹ is selected from:
a) an anti-inflammatory, an immunomodulating, a nephro-protective, and a compound possessing an activity or capable of inducing an activity that modulates gluococorticoid receptor (GR) activity, or reduces the levels of proteinuria/creatinine, or modulates a cytokine release, or otherwise exhibits a biological activity against inflammation-associated kidney diseases, or a kidney injury; or
b) a structure selected from aldosterone, AN3485, beclomethasone, beclomethasone propionate, betamethasone, budesonide, budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, or valdecoxib; or a variant thereof,
c) a glucocorticoid structure connected to X through a residue of an OH group present within the structure (H)ₙR¹; and
A⁸ through A¹¹ are optional amino acid residues unsubstituted or substituted at any N atom, and when present, are independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers h-k are independently selected from 0, 1, and 2, and wherein
when any of integers h through k is 0, then any of the two groups adjacent to a respective absent group (according to the integer 0 at said absent group) are connected to each other directly; and wherein
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ₛC(═O),
P(═O)(OH)CF₂, P(═O)(OH)CF₂(CR⁷R⁸)ₛC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣ OC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚ OC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O);
or any variant of above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein
R⁶, R⁷, R¹, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁶ and R⁷, ii) R⁵ and R⁶, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle;
integers p, r, and s are independently selected from 0, 1, and 2; and wherein
when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and wherein
when fragments (CR⁵R⁶)ₚ(CR⁷R⁸)ᵣ or (OCR⁵R⁶)ₚ(CR⁷R⁸)ᵣ are present, then [p+r]≥1; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³, with the following structures:
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O);
or any variant of the above X groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
each optional divalent group X independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O)ₛN(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups;
R¹¹ is CH₂CH(CH₃)₂ or CH₂Ph;
R¹² is CH₂NH₂ or CH₂CH₂NH₂, or hydrogen; and
integer zz is 1 or 2.

2. A compound of Formula II-P-1: 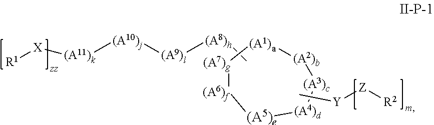
or a pharmaceutically acceptable salt, solvate, or hydrate thereof wherein:
R¹ and R² are optional groups, with at least one of the groups R¹ and R² being present in the Formula II-P-1; and
R¹ and R² are independently selected from alkyl, aryl, biaryl, heteroaryl, heteroarylaryl, and arylheteroaryl; or
R¹ and R² are residues independently attached to X and Z, respectively, formed by subtracting a single H atom from respective parent (precursor) structure(s) (H)ₙR¹ and (H)ₒR² at any one of the following H-containing group(s) independently selected from NH, OH, SH, C(═O)OH, CONH, SO₂NH, and S(═O)NH when present in (H)ₙR¹ and (H)ₒR²; and wherein
a) (H)ₙR¹ and (H)ₒR² are independently an anti-inflammatory compound, an immunomodulating compound, or a nephro-protective compound, or a compound possessing an activity or capable of inducing an activity that modulates glucocorticoid receptor (GR) activity, or reduces the levels of proteinuria/creatinine, or modulates a cytokine release, or otherwise exhibits a biological activity against inflammation-associated kidney diseases, or a kidney injury; or
b) (H)ₙR¹ and (H)ₒR² are independently selected from aldosterone, AN3485, beclomethasone, beclomethasone propionate, betamethasone, budesonide, budesonide metabolite, celecoxib, 11-deoxycortisole, dexamethasone, hydrocortisone, finerenone, fludrocortisone, flunisolide, flunisolide metabolite, fluticasone momethasone, ibuprofene, naproxene, prednisolone, methyl prednisolone, prednisone, triamcinolone, or valdecoxib; or a variant thereof;
c) (H)ₙR¹ and (H)ₒR² are independently compound(s) effective for a kidney disease therapy; or
d) (H)ₙR¹ is a glucocorticoid structure connected to X at an O or N atom(s), where at least one of these atoms is present within the structure (H)ₙR¹; and
when R¹ is absent, then the fragment R¹X is R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when R² is absent, then R²Z is R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂;
integers n and o are independently selected from 0, 1, 2, 3, 4, 5, 6, and 7, such that [n+o]≥1; and
A¹ through A¹¹ are optional amino acid residues, unsubstituted or substituted at any N atom, and are independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers a through k, m and zz are independently selected from 0, 1, and 2, and wherein
[m+zz]≥1; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, and heterocyclyl; or the group A⁸ is directly connected to the group Y; and
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O)N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ₛC(═O),
P(═O)(OH)CF₂, P(═O)(OH)CF₂(CR⁷R⁸)ₛC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O-C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O);
or any variant of the above groups formed by repositioning(s), addition(s), or deletion(s) of the fragments C(═O), OC(═O), N(R⁴)C(═O), P(═O)(OCR⁵R⁶)CF₂, P(═O)(OH)CF₂, and/or C(═O)N(R⁴)SO₂ therein; and wherein
R⁵, R⁶, R⁷, R⁸, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl
R⁴ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁵ and R⁶, ii) R⁶ and R⁷, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and wherein
when fragments (CR⁵R⁶)ₚ(CR⁷R⁸)ᵣ or (OCR⁵R⁶)ₚ(CR⁷R⁸)ᵣ are present, then [p+r]≥1; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³, with the following structures:
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O);
or any variant of the above X groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups;
optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂C₀₀C₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)C₀₀C₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O);
or any variant of above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein;
alternatively, the optional group Z is comprised of the following structures, optionally connected to one to two amino acid residue(s) A¹² or A¹³, with the following structures:
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁴), N(R⁴)C(═O)(C₁₋₁₂alkylene),
(C₁₋₁₂alkylene)N(R⁴)C(═O), C(═O) N(R⁴)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)CR⁵═CR⁷—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N(R⁴)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁴)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁴)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁴)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁴)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH(NH₂)CH₂CH₂COOH]CH₂CH₂C(═O),
C(═O)N{CH₂CH₂NHC(═O)—[(S)—CH(NH₂)]CH₂CH₂COOH}CH₂CH₂C(═O),
C(═O)N{CH₂CH₂NHC(═O)—[(R)—CH(NH₂)]CH₂CH₂COOH}CH₂CH₂C(═O),
C(═O)N[CH₂CH₂OC(═O)CH(NH₂)CH₂CH₂COOH]CH₂CH₂C(═O),
C(═O)N[CH₂CH₂NHC(═O)CH(OH)CH₂CH₂COOH]CH₂CH₂C(═O),
C(═O)N[CH₂CH₂NHC(═O)CH(NH₂)CH₂COOH]CH₂CH₂C(═O),
C(═O)N{CH₂CH₂NHC(═O)—[(S)—CH(NH₂)]CH₂COOH}CH₂CH₂C(═O),
C(═O)N{CH₂CH₂NHC(═O)—[(R)—CH(NH₂)]CH₂COOH}CH₂CH₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N[CH₂CH₂NHC(═O)—{(S)—CH[NHC(═O)H]}CH₂CH₂COOH]CH₂CH₂C(═O),
C(═O)N{CH₂CH₂NHC(═O)—{(S)—CH[NHC(═O)Me]}CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH(NH₂)CH₂CH₂COOH]CH₂CH₂C(═O)OCH₂CH₂C(═O),
C(═O)N[CH₂CH₂NHC(═O)CH(NH₂)CH₂CH₂COOH]CH₂CH₂C(═O)OCH(Me)CH(Me)C(═O),
C(═O)N[CH₂CH₂NHC(═O)CH(NH₂)CH₂CH₂COOH]CH₂CH₂C(═O)OC(Me)₂C(Me)₂C(═O),
C(═O)N[CH₂CH₂NHC(═O)CH(NH₂)CH₂CH₂COOH]CH₂CH₂C(═O)O—C₃₋₆cycloalkylene-C(═O),
C(═O)N{CH₂CH₂NHC(═O)CH[NH(A¹)]CH₂CH₂COOH}CH₂CH₂C(═O)OCH₂CH₂C(═O),
C(═O)N[CH₂CH₂NHC(═O)CH(NH₂)CH₂CH₂COOH]CH₂CH₂OC(═O)CH₂CH₂C(═O),
C(═O)N[CH₂CH₂NHC(═O)CH(NH₂)CH₂CH₂COOH]CH₂CH(Me)OC(═O)CH₂CH₂C(═O),
C(═O)N[CH₂CH₂NHC(═O)CH(NH₂)CH₂CH₂COOH]CH₂C(Me)₂OC(═O)CH₂CH₂C(═O),
C(═O)N[CH₂CH₂NHC(═O)CH(NH₂)CH₂CH₂COOH]CH₂CH₂OC(═O)—C₃₋₆cycloalkylene-C(═O),
C(═O)N{CH₂CH₂NHC(═O)CH[NH(A¹)]CH₂CH₂COOH}CH₂CH₂OC(═O)CH₂CH₂C(═O),
C(═O)N[CH₂CH₂NHC(═O)CH(NH₂)CH₂CH₂COOH]CH₂CH₂OC(═O)C(Me)CH(Me)C(═O),
C(═O)N[CH₂CH₂NHC(═O)CH(NH₂)CH₂CH₂COOH]CH₂CH₂OC(═O)C(Me)₂C(Me)₂C(═O),
C(═O)N{CH₂CH₂NHC(═O)CH[NHC(═O)H]CH₂CH₂COOH}CH₂CH₂C(═O),
C(═O)N{CH₂CH₂NHC(═O)CH[NHC(═O)Me]CH₂CH₂COOH}CH₂CH₂C(═O),
C(═O)N{CH₂CH₂NHC(═O)CH[NHC(═O)C₁₋₆alkyl]CH₂CH₂COOH}CH₂CH₂C(═O),
C(═O)N{CH₂CH₂NHC(═O)CH[NH(A¹)]CH₂CH₂COOH}CH₂CH₂C(═O),
C(═O)N[CH₂CH₂NHC(═O)CH(NH₂)CH₂CH₂COOC₁₋₆alkyl]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O),
C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂) COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O); or any variant of the above Z groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A¹⁴, A⁵, A⁶, A⁷ or A⁸.

3. The compound of claim 2, of Formula I-1: 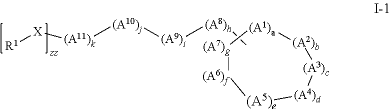
or a pharmaceutically acceptable salt, solvate, or hydrate thereof; wherein the integer zz is 1 or 2.

4. The compound of claim 2, or a pharmaceutically acceptable salt, solvate, or hydrate thereof, wherein
the integers a through g are each 1; and
A¹ is Thr or Ser;
A², A³ A⁶, and A⁷ are independently selected from Dab, Dap, Ser, and Thr;
A¹⁴ is Leu or Ile; and
A⁵ is Phe, D-Phe, Bip, D-Bip, Val, or D-Val.

5. The compound of claim 3, of Formula III: 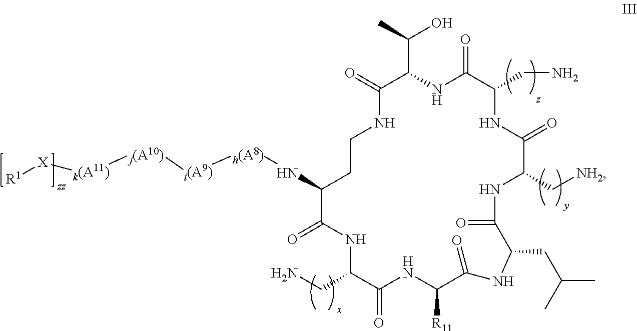
or a pharmaceutically acceptable salt, solvate, or hydrate thereof wherein:
integer x, y, z are independently selected from 1, 2 and 3;
integer zz is 1 or 2; and
R¹¹ is CH₂CH(CH₃)₂ or CH₂Ph.

6. The compound of claim 1, of Formula II-P-2: 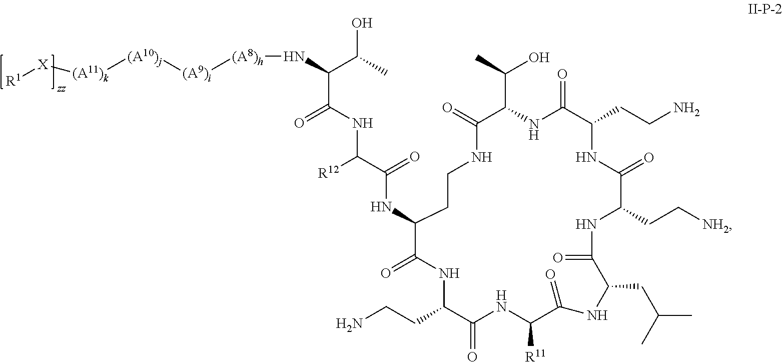
or a pharmaceutically acceptable salt, solvate, or hydrate thereof, wherein:
R¹¹ is CH₂Ph; and
R¹² is CH₂NH₂ or CH₂CH₂NH₂, or hydrogen; and
integer zz is 1.

7. The compound of claim 5, of Formula Ic: 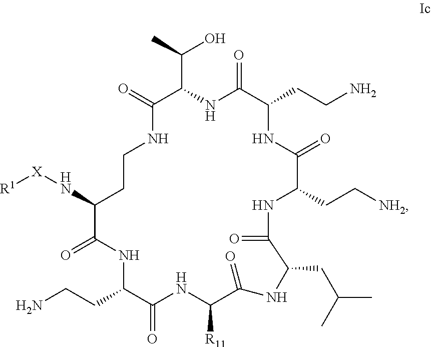
or a pharmaceutically acceptable salt, solvate, or hydrate thereof, wherein:
R¹¹ is CH₂CH(CH₃)₂ or CH₂Ph.

8. The compound of claim 1, or a pharmaceutically acceptable salt, solvate, or hydrate thereof, wherein each X is independently selected from the structures below, wherein either the left side or the right side of X depicted below is connected to R¹:

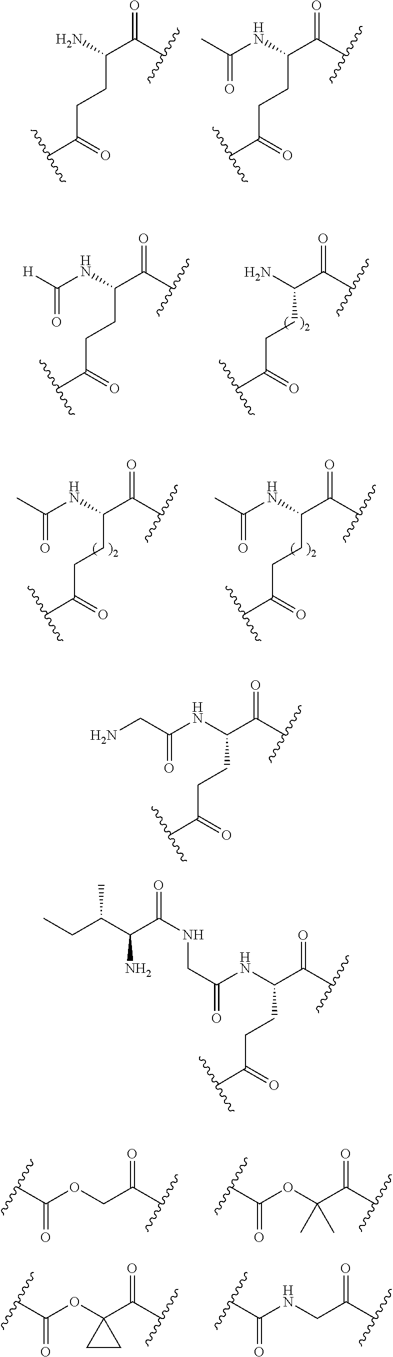

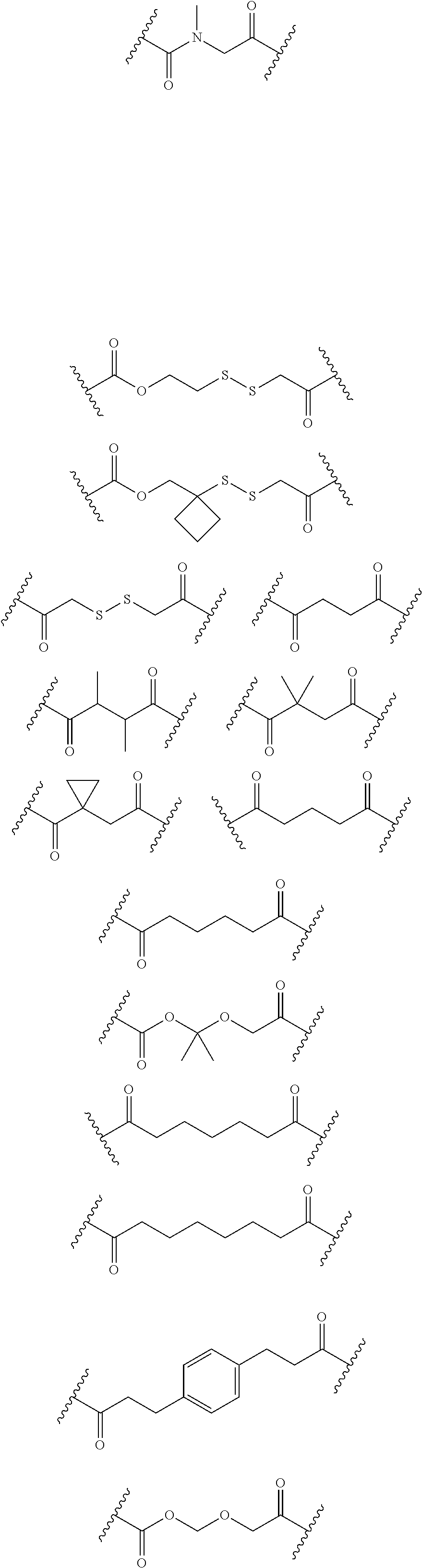

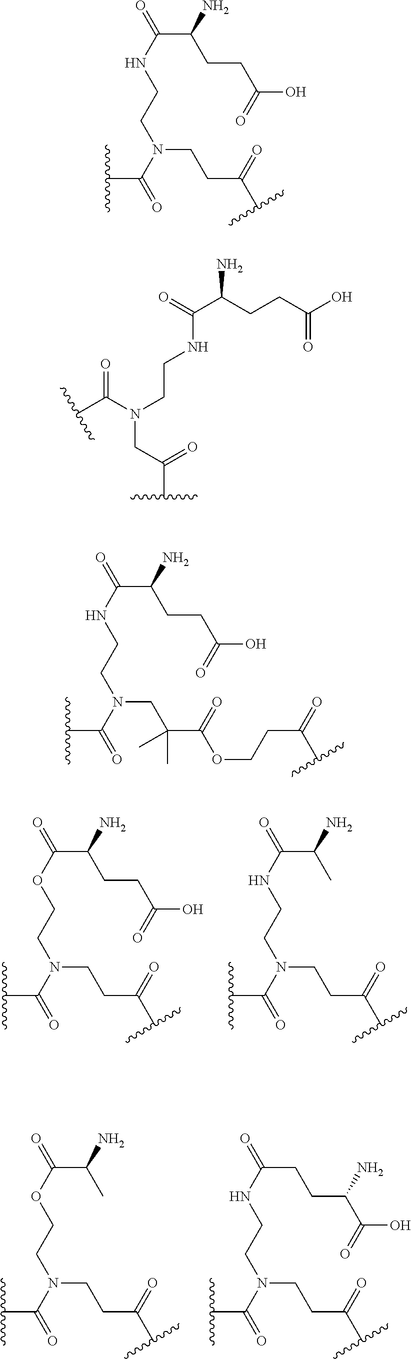

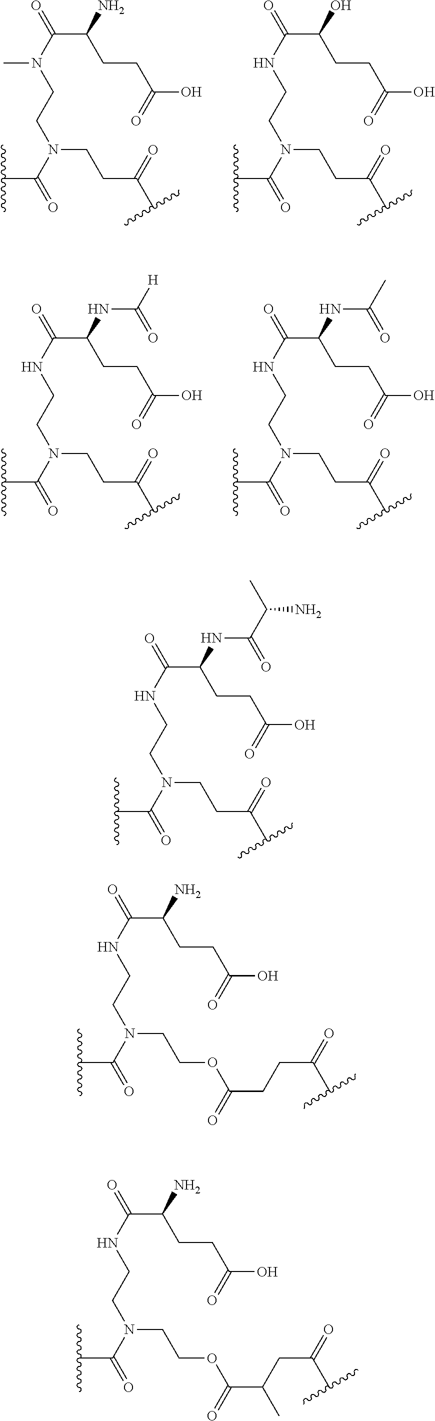

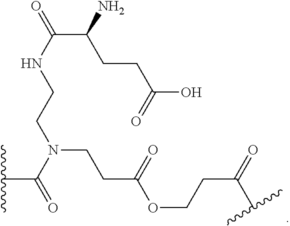 [SMILES: *C(=O)CCOC(=O)CCN(CCNC(=O)[C@@H](N)CCC(=O)O)C(*)=O]

9. (canceled)

10. (canceled)

10. The compound of claim 2, according to formula III-P: 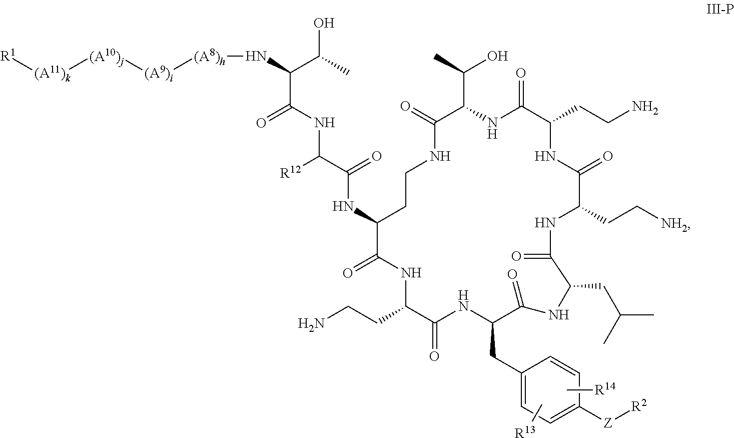
or a pharmaceutically acceptable salt, solvate, or hydrate thereof, wherein R¹³ and R¹⁴ are independently selected from H, halo, NH₂, CN, OH, OC₁₋₁₄alkyl, O-aryl, NH(C₁₋₆alkyl), NH(OC₁₋₆alkyl), C₁₋₁₄alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄alkyl.

11. The compound of claim 10, or a pharmaceutically acceptable salt, solvate, or hydrate thereof, wherein Z is selected from the structures below, wherein the right side of Z is connected to R²:

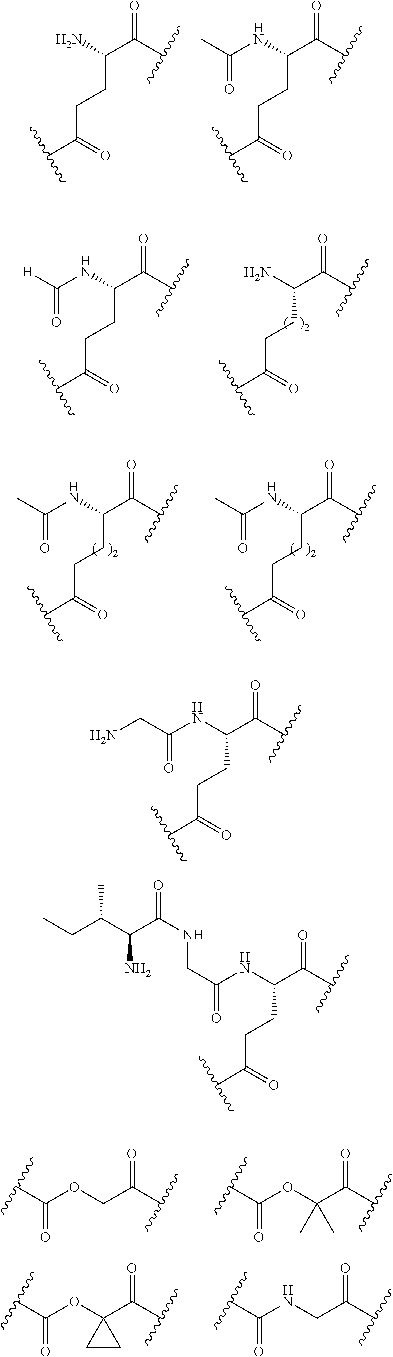

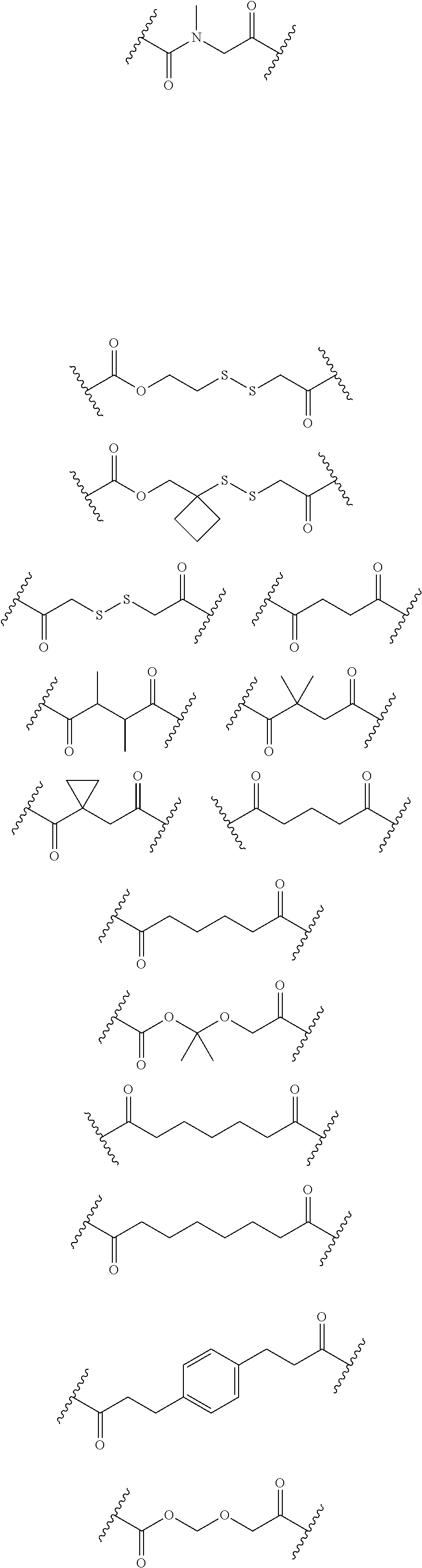

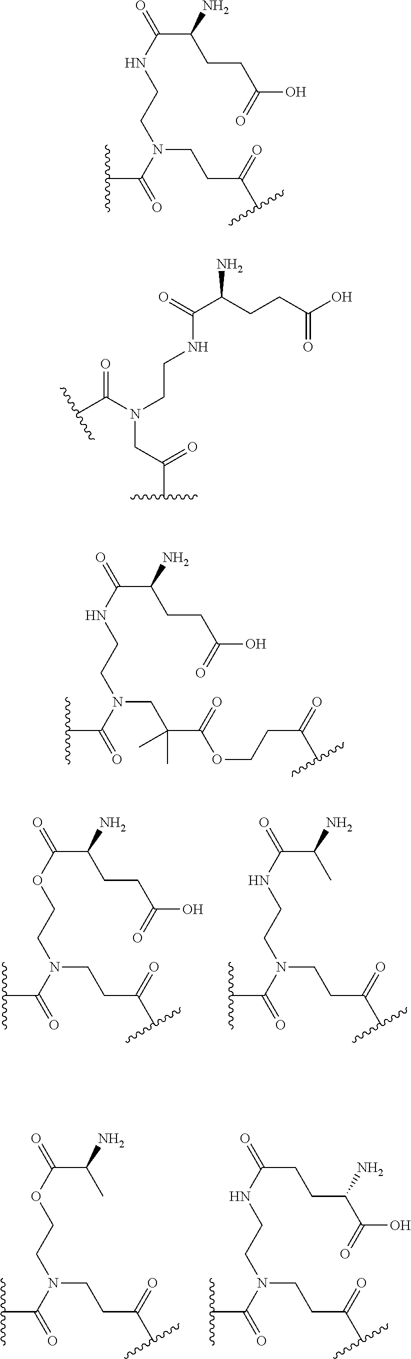

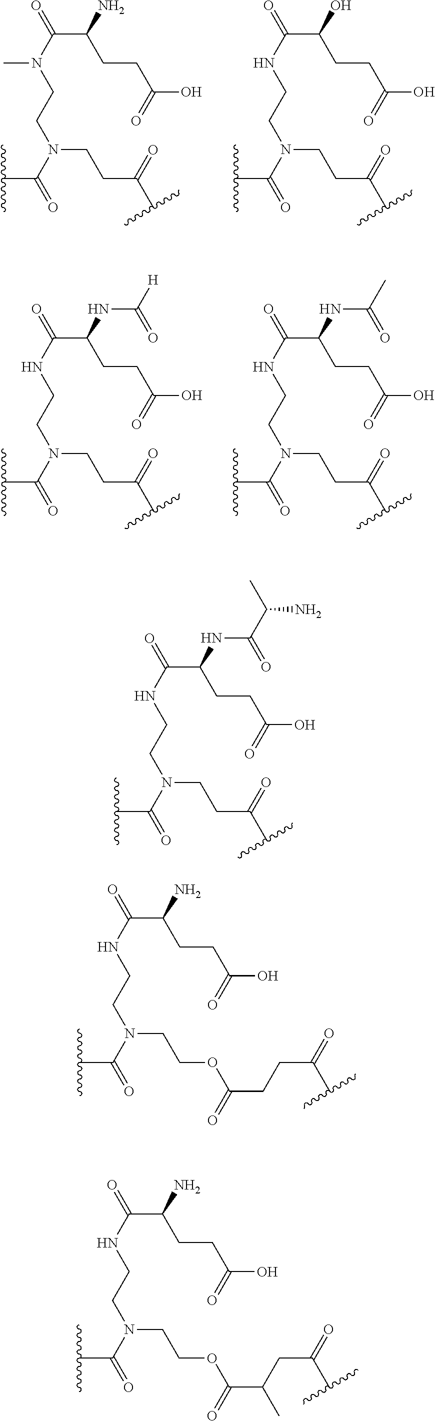

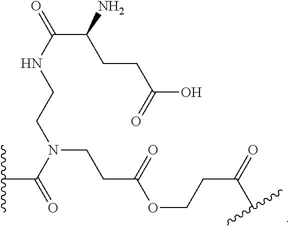 [SMILES: *C(=O)CCOC(=O)CCN(CCNC(=O)[C@@H](N)CCC(=O)O)C(*)=O]

12. The compound claim 1, or a pharmaceutically acceptable salt, solvate, or hydrate thereof, wherein R¹ is derived from subtraction of H from an OH group in R¹(H), and wherein R¹(H) is selected from the structures below:

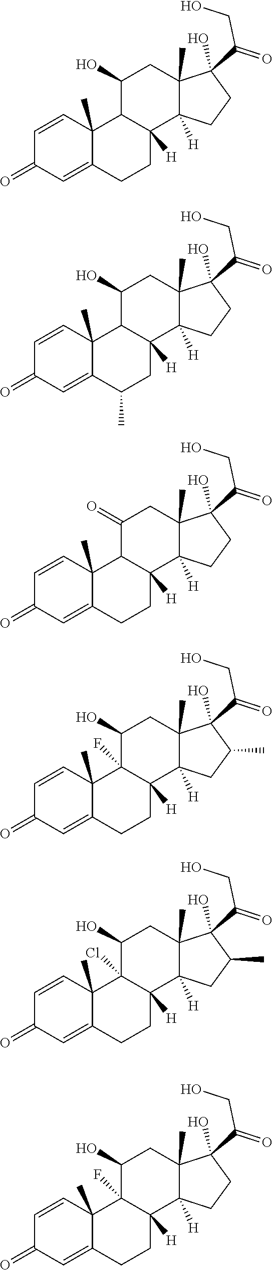 [SMILES: C[C@]12C=CC(=O)C=C1CC[C@@H]1C2[C@@H](O)C[C@@]2(C)[C@H]1CC[C@]2(O)C(=O)CO ; C[C@H]1C[C@@H]2C([C@@H](O)C[C@@]3(C)[C@H]2CC[C@]3(O)C(=O)CO)[C@@]2(C)C=CC(=O)C=C12 ; C[C@]12C=CC(=O)C=C1CC[C@@H]1C2C(=O)C[C@@]2(C)[C@H]1CC[C@]2(O)C(=O)CO ; C[C@@H]1C[C@H]2[C@@H]3CCC4=CC(=O)C=C[C@]4(C)[C@@]3(F)[C@@H](O)C[C@]2(C)[C@@]1(O)C(=O)CO ; C[C@H]1C[C@H]2[C@@H]3CCC4=CC(=O)C=C[C@]4(C)[C@@]3(Cl)[C@@H](O)C[C@]2(C)[C@@]1(O)C(=O)CO ; C[C@]12C=CC(=O)C=C1CC[C@H]1[C@@H]3CC[C@](O)(C(=O)CO)[C@@]3(C)C[C@H](O)[C@@]12F]

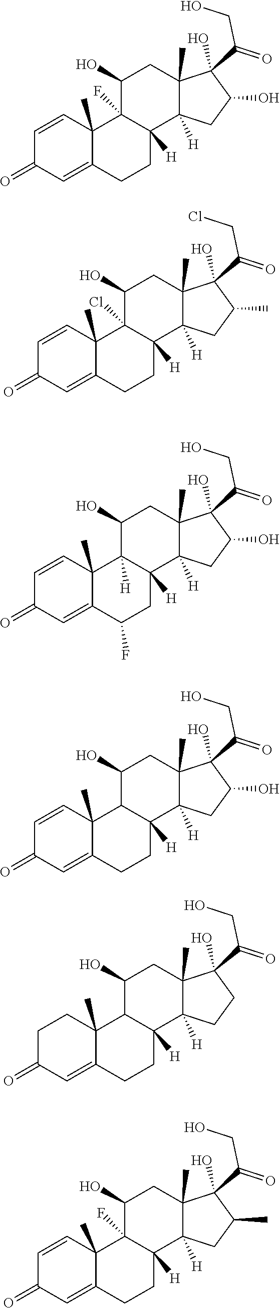

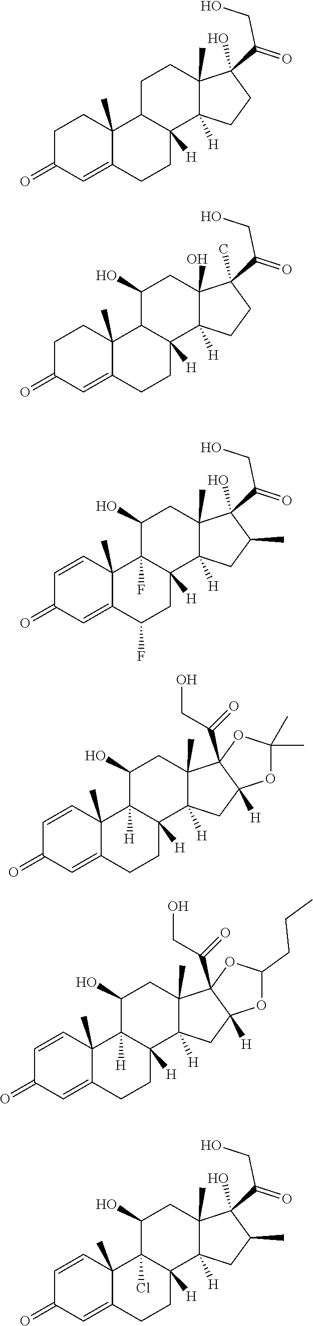

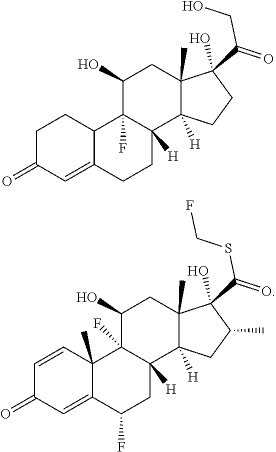

13. The compound of claim 12, wherein R¹ is derived from subtraction of a H from the primary alcohol CH₂OH group in the structure R¹(H).

14. The compound of claim 2, or a pharmaceutically acceptable salt, solvate, or hydrate thereof, selected from a structure below:

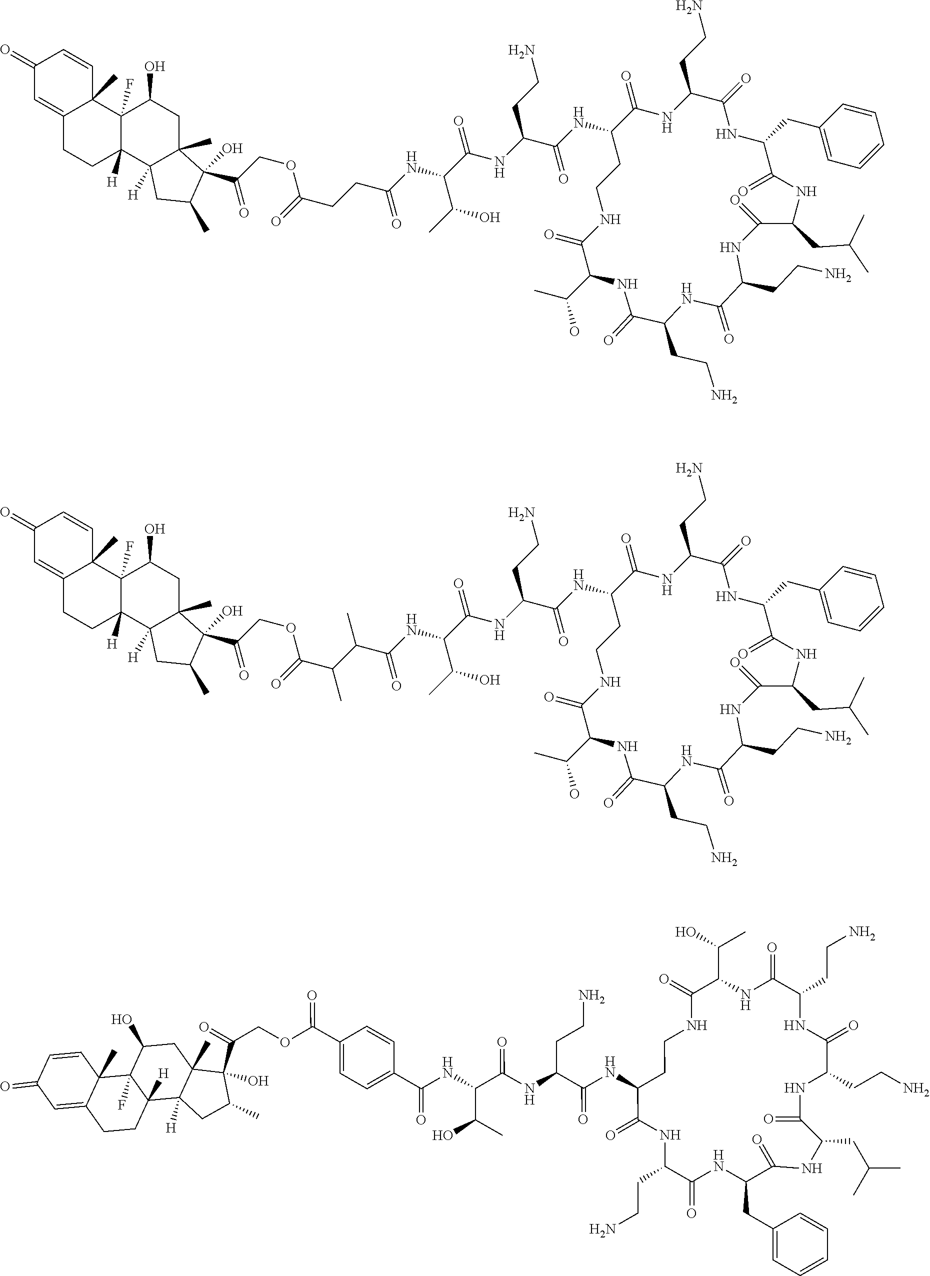

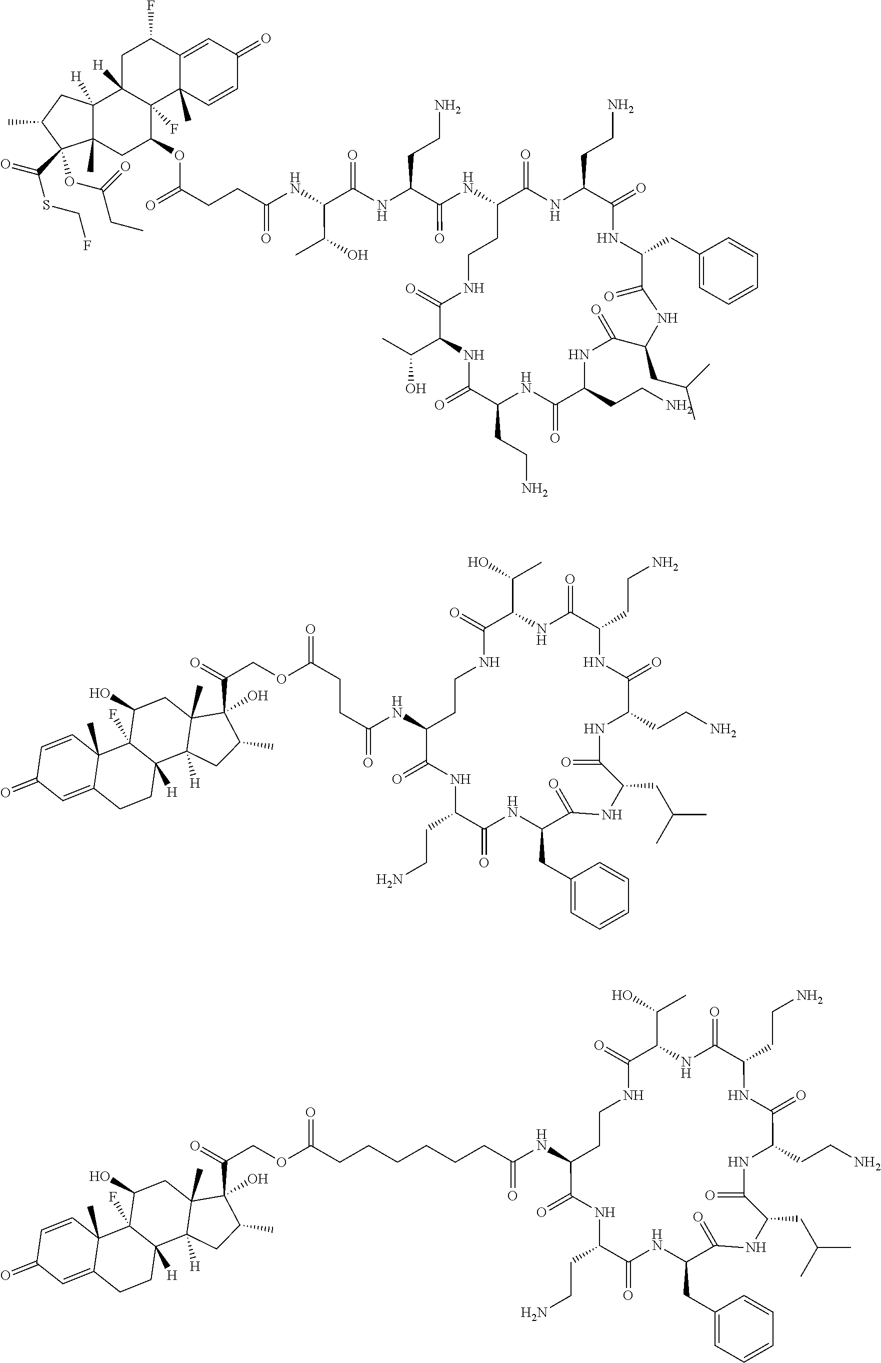

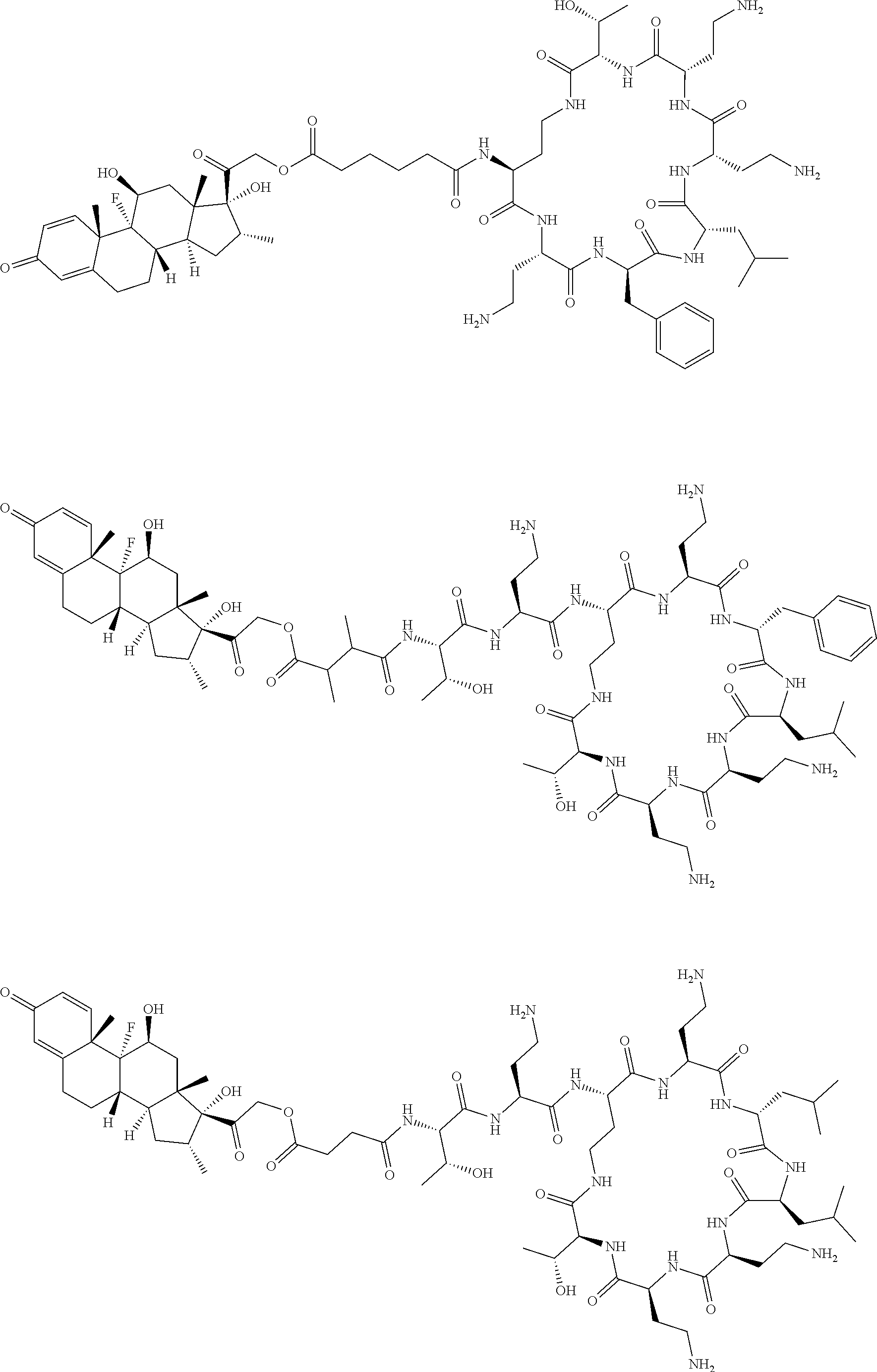

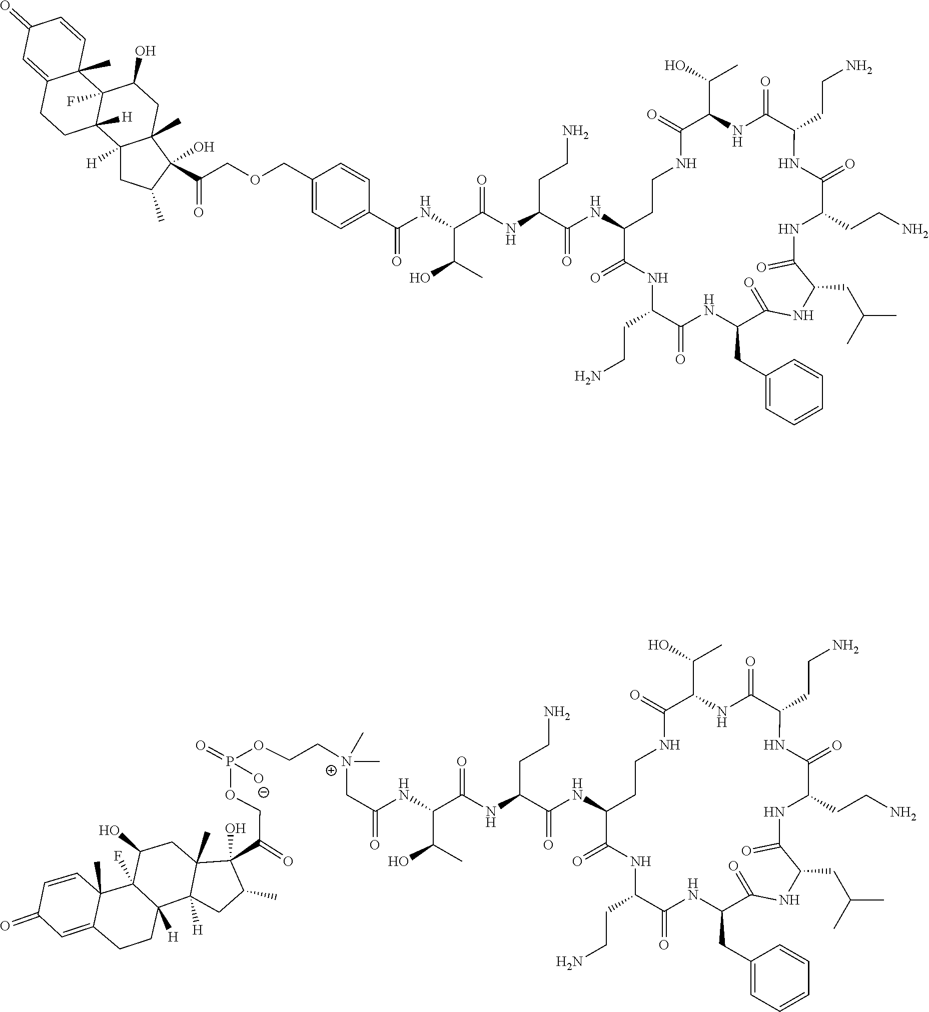

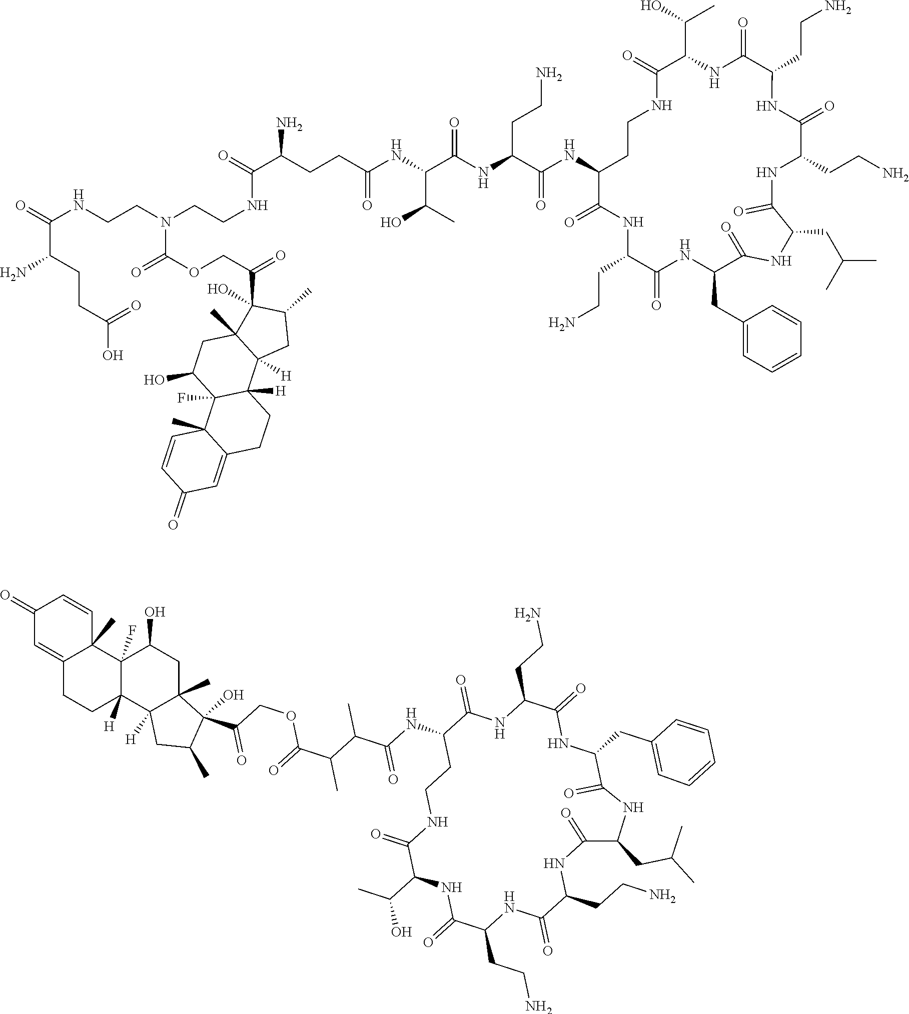

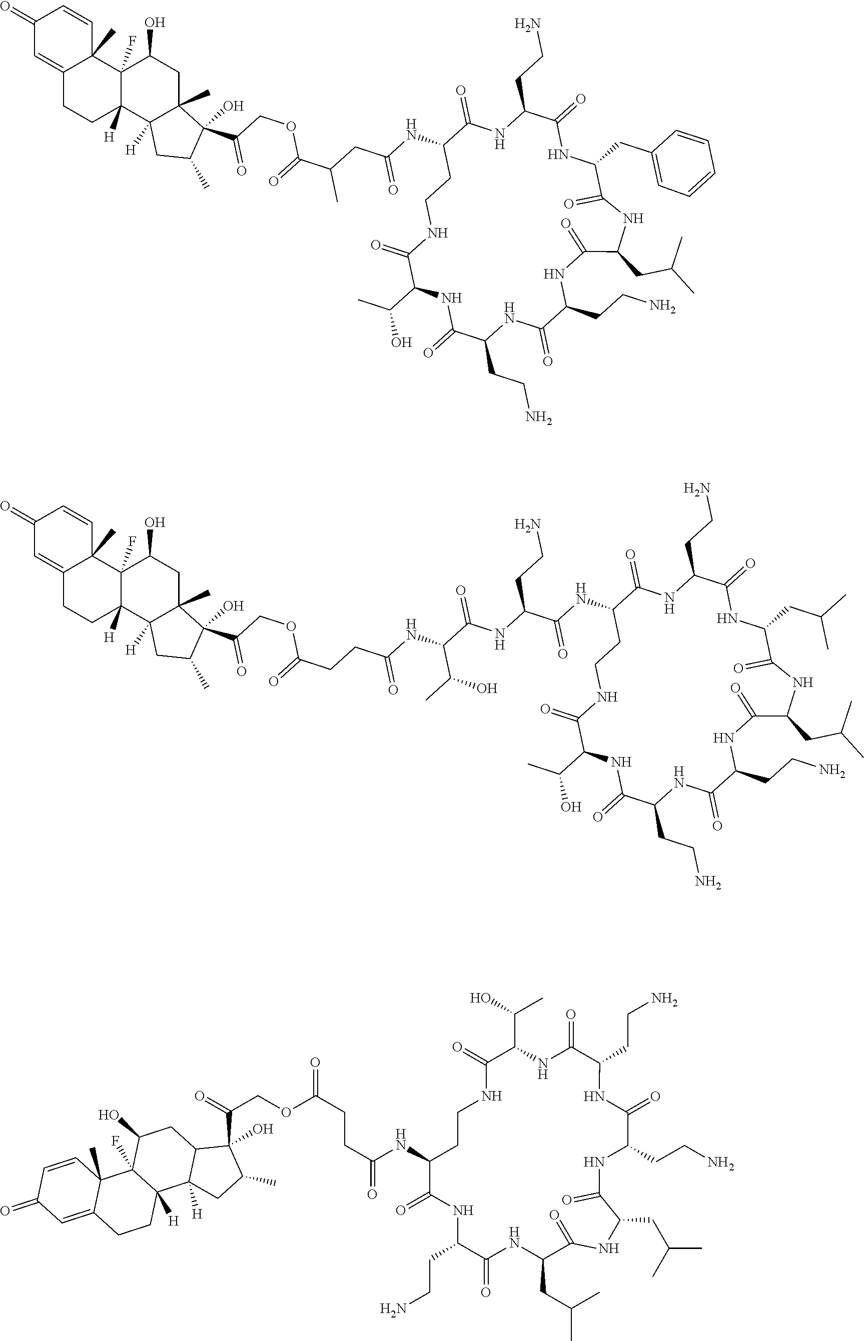

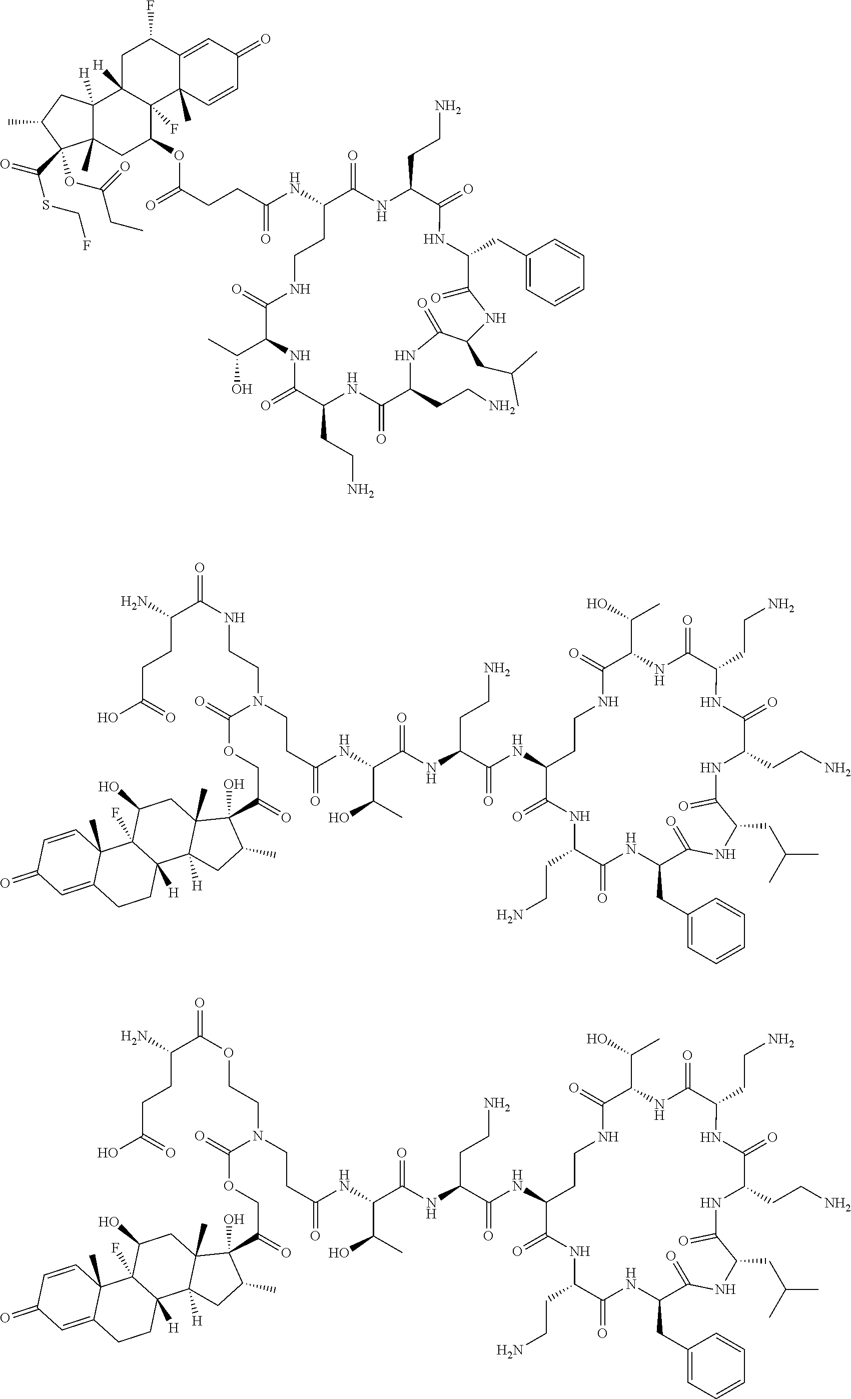

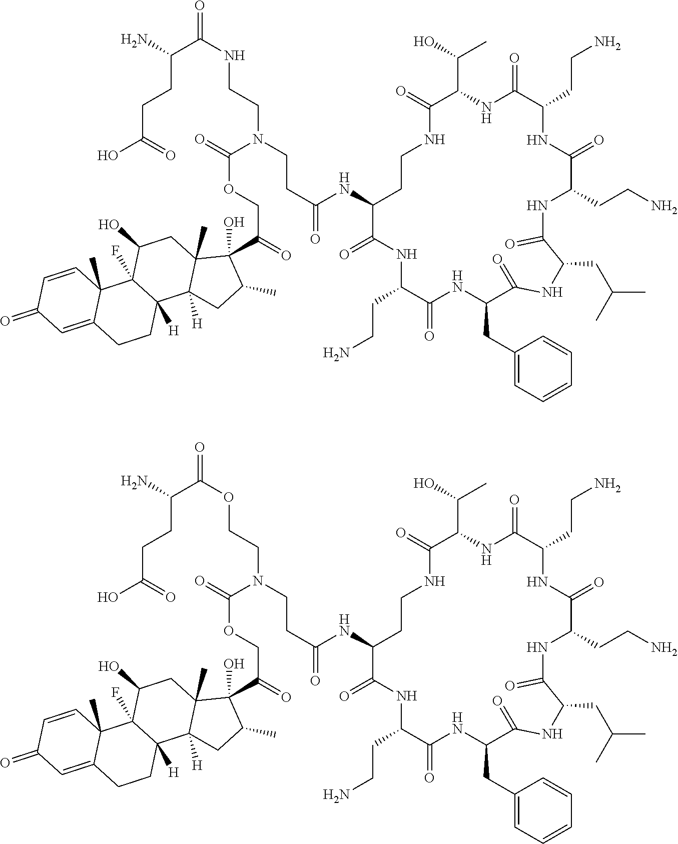

15. (canceled)

16. (canceled)

17. (canceled)

18. (canceled)

19. The compound of claim 2, or a pharmaceutically acceptable salt, solvate, or hydrate thereof, that possesses anti-inflammatory activity, or a therapeutic effect for a kidney disease, as determined by (i) alleviating or slowing kidney cytokines release such as TNF-α, IL-6, IL-12; (ii) reducing one or more biomarker, optionally wherein the one or more biomarkers are selected from protein level(s), blood urea nitrogen, and serum creatinine; or, (iii) improving a condition of a patient in need of therapy, or of a mammal in animal tests optionally, wherein the anti-inflammatory activity is a therapy for renal inflammation, inflammation diseases in kidney injury or dysfunction, or inflammation induced by a nephrotoxic substance, including a pharmaceutical nephrotoxic substance, such as anticancer, anti-diabetes, anti-infective, or another chemotherapeutic substance.

20. (canceled)

21. The compound of claim 2, or a pharmaceutically acceptable salt, solvate, or hydrate thereof, that possesses an enhanced anti-inflammatory, immunomodulating, or nephro-protective effect when compared to a similar dose of a free agent or drug (H)ₙR¹ and/or (H)ₒR² incorporated into said compound, as determined by in vitro or in vivo test(s) for anti-inflammatory, immunomodulating, or nephro-protective activity.

22. The compound claim 2, or a pharmaceutically acceptable salt, solvate, or hydrate thereof, wherein when administered to a mammal, said compound exhibits preferential accumulation in kidneys, with a ratio for its molar concentration in kidneys compared to that in blood of between about 5 and 500.

23. (canceled)

24. The compound of claim 2, or a pharmaceutically acceptable salt, solvate, or hydrate thereof, wherein when administered to a mammal at a dosing (expressed in molar amount) equal to a standard therapeutic dosing (in molar amount) of an agent (H)ₙR¹ and/or (H)ₒR², said compound exhibits about 1.5- to 15-fold higher loading (tissue concentration) and/or drug exposure (area-under-the-curve, AUC) of agent (H)ₙR¹ and/or (H)ₒR² in kidneys, as compared to the standard therapeutic dosing of free drugs (H)ₙR¹ and/or (H)ₒR².

25. (canceled)

26. (canceled)

27. The compound of claim 2, or a pharmaceutically acceptable salt, solvate, or hydrate thereof, wherein when administered to a mammal at a dosing (expressed in molar amount) equal to a standard therapeutic dosing (in molar amount) of an agent (H)ₙR¹ and/or (H)ₒR², exhibits at least 2-fold reduced rate of adverse effects and/or off-target toxicity manifestation, as compared to the standard therapeutic dosing of (H)ₙR¹ and/or (H)ₒR², as determined by medical observations of a mammal under therapy, a blood cells count, a tissue biopsy, and/or by analysis of biochemical biomarkers, or similar method.

28. A pharmaceutical composition comprising a therapeutically effective amount of a compound of claim 2, or a pharmaceutically acceptable salt, solvate, or hydrate thereof, and a pharmaceutically acceptable carrier.

29. A method for treating inflammation in a mammal comprising administering to the mammal a therapeutically effective amount of a compound of claim 2, or a pharmaceutically acceptable salt, solvate, or hydrate thereof.

30. The method according to claim 29, wherein the compound, or a pharmaceutically acceptable salt, solvate, or hydrate thereof, or wherein the pharmaceutical composition is administered to the mammal parenterally, transdermally, orally, intranasally, topically, rectally, or via an intra-tumoral administration in a pharmaceutical composition.

31. The method of claim 29, wherein the renal inflammation is the chronic kidney disease (CKD), systemic lupus erythematosus (SLE), a nephritis, acute kidney injury (AKI), diabetic nephropathy, chronic glomerulonephritis, or an inflammation in a kidney transplant surgery.

32. The compound of claim 2, or a pharmaceutically acceptable salt, solvate, or hydrate thereof, wherein each X is independently selected from the structures below, wherein either the left side or the right side of X depicted below is connected to R¹:

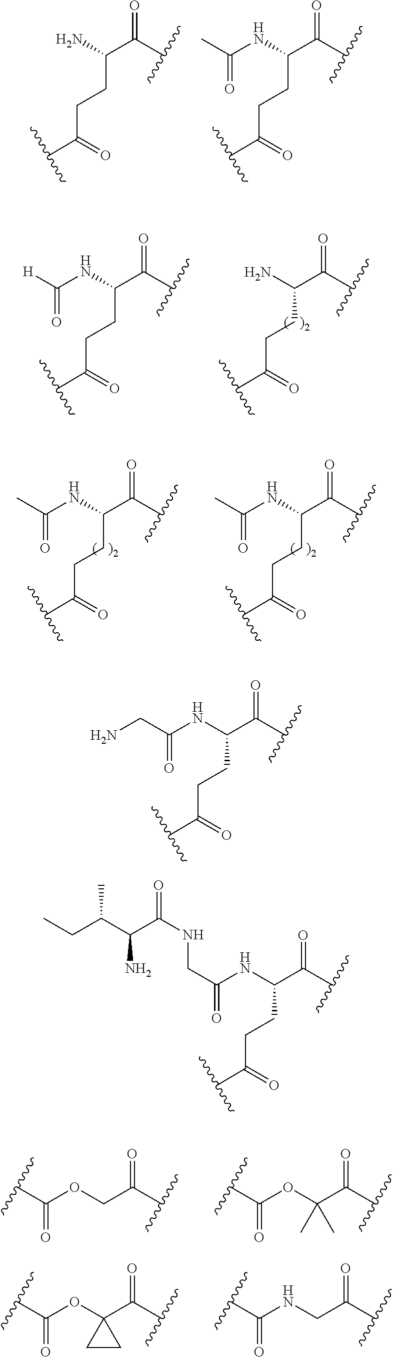

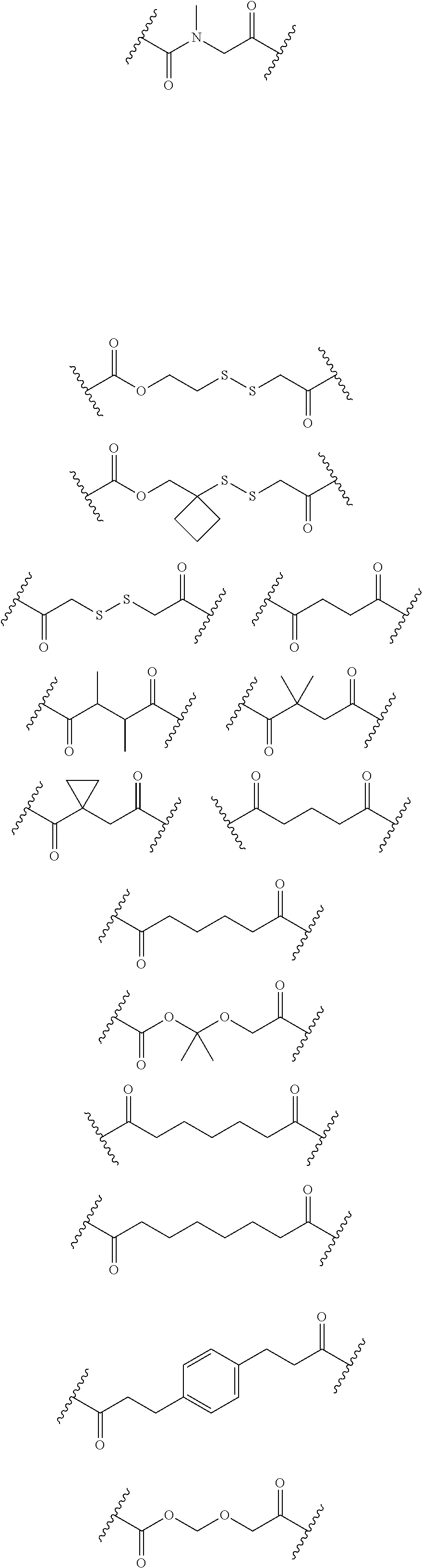

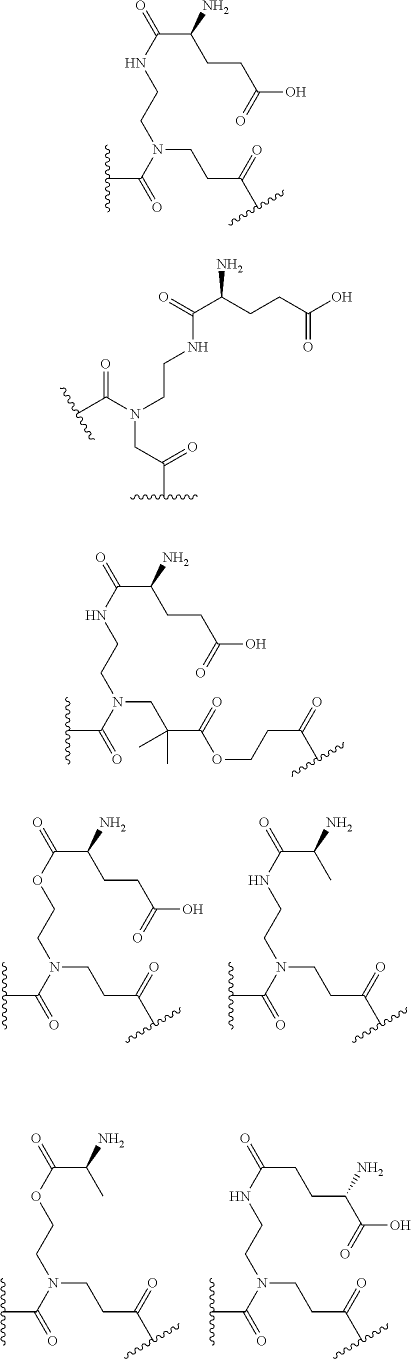

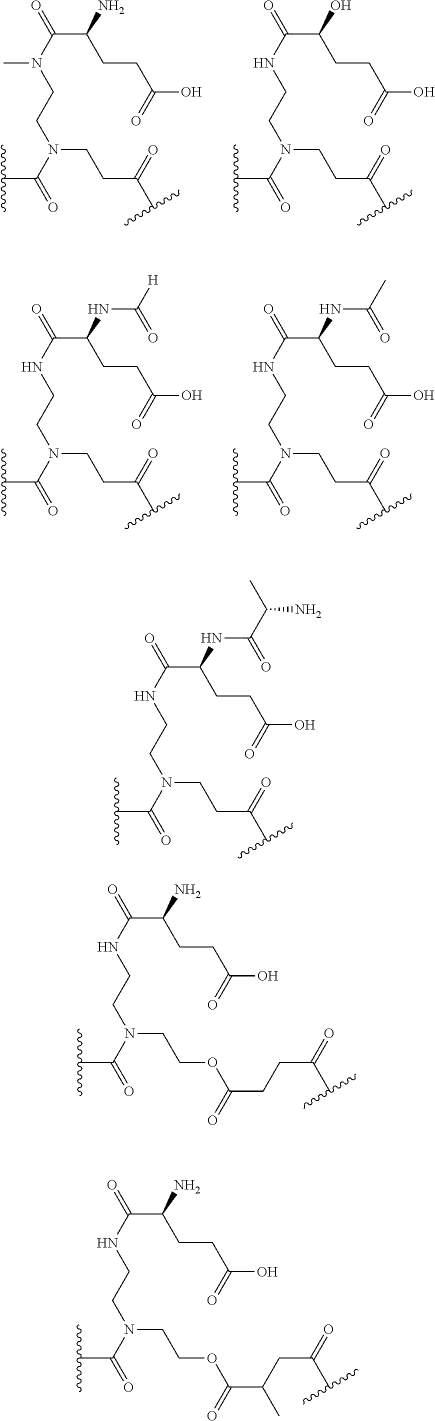

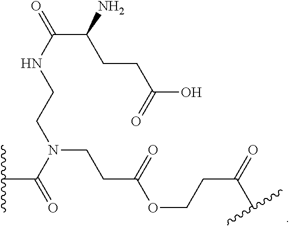 [SMILES: *C(=O)CCOC(=O)CCN(CCNC(=O)[C@@H](N)CCC(=O)O)C(*)=O]

33. The compound of claim 1, or a pharmaceutically acceptable salt, solvate, or hydrate thereof, wherein each X, either at its left or right side therein, independently incorporates additional divalent groups selected from C₁₋₁₂alkylene, C₂₋₁₂alkenylene, C₂₋₁₂alkynylene, (CH₂)ₚO(CH₂)ᵣO(CH₂)C(═O), (CH₂)ₚO(CH₂)ᵣO(CH₂)ₛOC(═O), (CH₂)ₚO(CH₂)ᵣO(CH₂)NHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups.

34. The compound of claim 2, or a pharmaceutically acceptable salt, solvate, or hydrate thereof, wherein each X, either at its left or right side therein, independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups.

35. The compound claim 2, or a pharmaceutically acceptable salt, solvate, or hydrate thereof, wherein R¹ is derived from subtraction of H from an OH group in R¹(H), and wherein R¹(H) is selected from the structures below:

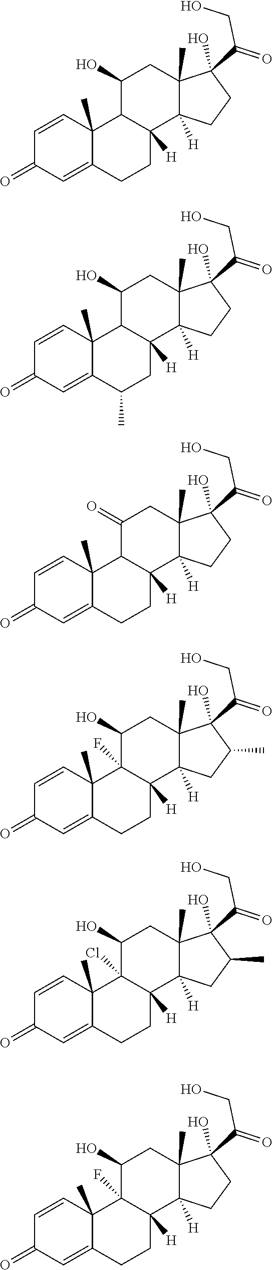 [SMILES: C[C@]12C=CC(=O)C=C1CC[C@@H]1C2[C@@H](O)C[C@@]2(C)[C@H]1CC[C@]2(O)C(=O)CO ; C[C@H]1C[C@@H]2C([C@@H](O)C[C@@]3(C)[C@H]2CC[C@]3(O)C(=O)CO)[C@@]2(C)C=CC(=O)C=C12 ; C[C@]12C=CC(=O)C=C1CC[C@@H]1C2C(=O)C[C@@]2(C)[C@H]1CC[C@]2(O)C(=O)CO ; C[C@@H]1C[C@H]2[C@@H]3CCC4=CC(=O)C=C[C@]4(C)[C@@]3(F)[C@@H](O)C[C@]2(C)[C@@]1(O)C(=O)CO ; C[C@H]1C[C@H]2[C@@H]3CCC4=CC(=O)C=C[C@]4(C)[C@@]3(Cl)[C@@H](O)C[C@]2(C)[C@@]1(O)C(=O)CO ; C[C@]12C=CC(=O)C=C1CC[C@H]1[C@@H]3CC[C@](O)(C(=O)CO)[C@@]3(C)C[C@H](O)[C@@]12F]

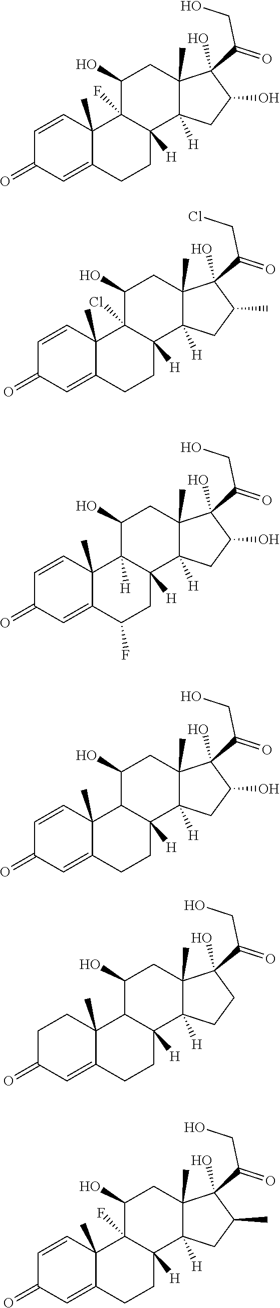

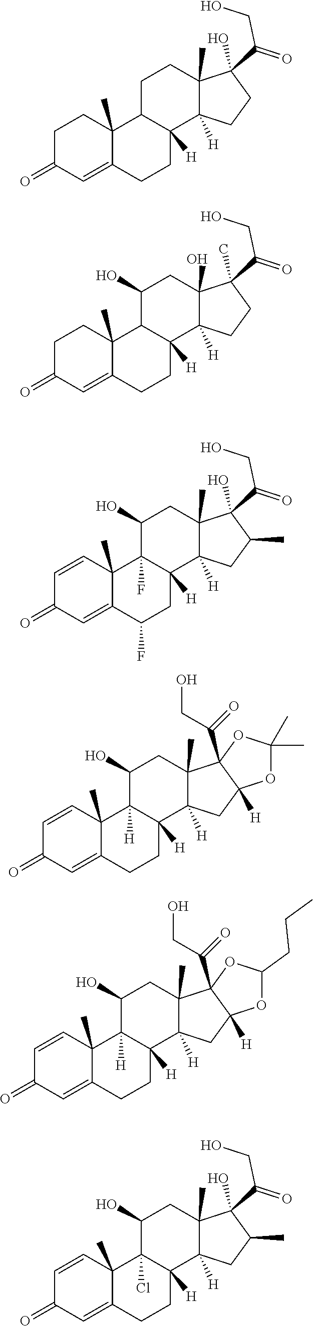

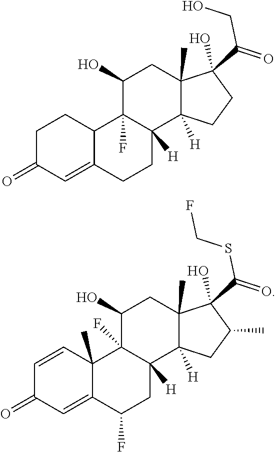

36. A method for treating inflammation in a mammal comprising administering to the mammal a therapeutically effective amount of a pharmaceutical composition of claim 28.
